SEQUENCE LISTING (34 sequences)

SEQ ID NO: 1 - protein, 5 aa, Artificial
  Synthetic Polypeptide
PXXXX

SEQ ID NO: 2 - protein, 8 aa, Artificial
  Synthetic Polypeptide
IPENFFGV

SEQ ID NO: 3 - protein, 8 aa, Artificial
  Synthetic Polypeptide
BPYGLGSP

SEQ ID NO: 4 - protein, 8 aa, Artificial
  Synthetic Polypeptide
HPSAFSEA

SEQ ID NO: 5 - protein, 8 aa, Artificial
  Synthetic Polypeptide
GPQGLLGA

SEQ ID NO: 6 - protein, 8 aa, Artificial
  Synthetic Polypeptide
GPAGLSVL

SEQ ID NO: 7 - protein, 8 aa, Artificial
  Synthetic Polypeptide
GPAGIVTK

SEQ ID NO: 8 - protein, 8 aa, Artificial
  Synthetic Polypeptide
DAASLLGL

SEQ ID NO: 9 - protein, 8 aa, Artificial
  Synthetic Polypeptide
RPAVMTSP

SEQ ID NO: 10 - protein, 8 aa, Artificial
  Synthetic Polypeptide
PPGAYHGA

SEQ ID NO: 11 - protein, 8 aa, Artificial
  Synthetic Polypeptide
LRAYLLPA

SEQ ID NO: 12 - protein, 8 aa, Artificial
  Synthetic Polypeptide
SPYELKAL

SEQ ID NO: 13 - protein, 8 aa, Artificial
  Synthetic Polypeptide
TAAALTSC

SEQ ID NO: 14 - protein, 8 aa, Artificial
  Synthetic Polypeptide
GPEGLRVG

SEQ ID NO: 15 - protein, 8 aa, Artificial
  Synthetic Polypeptide
GHARLVHV

SEQ ID NO: 16 - protein, 8 aa, Artificial
  Synthetic Polypeptide
QPVGINTS

SEQ ID NO: 17 - protein, 8 aa, Artificial
  Synthetic Polypeptide
ELGTYNVI

SEQ ID NO: 18 - protein, 8 aa, Artificial
  Synthetic Polypeptide
DVAQFVLY

SEQ ID NO: 19 - protein, 8 aa, Artificial
  Synthetic Polypeptide
DVANYNFF

SEQ ID NO: 20 - protein, 8 aa, Artificial
  Synthetic Polypeptide
HPVGLLAR

SEQ ID NO: 21 - protein, 8 aa, Artificial
  Synthetic Polypeptide
KPQQFFGL

SEQ ID NO: 22 - protein, 8 aa, Artificial
  Synthetic Polypeptide
IPVSLRSG

SEQ ID NO: 23 - protein, 8 aa, Artificial
  Synthetic Polypeptide
HVLNLRST

SEQ ID NO: 24 - protein, 8 aa, Artificial
  Synthetic Polypeptide
DPESIRSE

SEQ ID NO: 25 - protein, 8 aa, Artificial
  Synthetic Polypeptide
DPLEFKSH

SEQ ID NO: 26 - protein, 8 aa, Artificial
  Synthetic Polypeptide
RPIPITAS

SEQ ID NO: 27 - protein, 8 aa, Artificial
  Synthetic Polypeptide
RVLGLKAH

SEQ ID NO: 28 - protein, 8 aa, Artificial
  Synthetic Polypeptide
KVLNLTDN

SEQ ID NO: 29 - protein, 8 aa, Artificial
  Synthetic Polypeptide
PPEALRGI

SEQ ID NO: 30 - protein, 8 aa, Artificial
  Synthetic Polypeptide
IVAMLRAP

SEQ ID NO: 31 - protein, 8 aa, Artificial
  Synthetic Polypeptide
TAAAITGA

SEQ ID NO: 32 - protein, 5 aa, Artificial
  Synthetic Polypeptide
PLGXL

SEQ ID NO: 33 - protein, 6 aa, Artificial
  Synthetic Polypeptide
PLGXYL

SEQ ID NO: 34 - protein, 4 aa, Artificial
  Synthetic Polypeptide
PGXL

(19) United States
(12) Patent Application Publication
(10) Pub. No.: US 2005/0106100 A1
(43) Pub. Date: May 19, 2005

(54) Compounds containing matrix metalloproteinase substrates and methods of their use

(76) Inventors: Thomas D. Harris, Salem, NH (US); Padmaja Yalamanchili, Weston, MA (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/931,627

(22) Filed: Sep. 1, 2004

Related U.S. Application Data
(60) Provisional application No. 60/499,960, filed on Sep. 3, 2003.
(60) Provisional application No. 60/499,966, filed on Sep. 3, 2003.

(51) Int. Cl. A61K 51/00; A61K 49/00; C07K 16/46

(52) U.S. Cl. 424/1.49; 424/9.34; 530/391.1

(57) ABSTRACT

Compounds for use in a diagnostic agent for detecting, imaging, and/or monitoring a pathological disorder associated with matrix metalloproteinase activity at a site of interest in a patient are disclosed. Compositions and kits containing the compounds are also disclosed. In addition, methods of detecting, imaging, and/or monitoring the presence of matrix metalloproteinase or a pathological disorder associated with matrix metalloprotainase activity in a patient are disclosed.

### CROSS REFERENCE TO RELATED APPLICATION

The non-provisional application claims priority from two provisional applications U.S. Ser. No. 60/499,960 filed Sep. 3, 2003 and 60/499,966 filed Sep. 3, 2003.

The present disclosure is directed to diagnostic agents. More specifically, the disclosure is directed to compounds, diagnostic agents, compositions, and kits for detecting and/or imaging and/or monitoring a pathological disorder associated with matrix metalloproteinase activity. In addition, the disclosure is directed to methods of detecting and/or imaging and/or monitoring the presence of matrix metalloproteinase or a pathological disorder associated with matrix metalloproteinase activity in a patient.

Matrix metalloproteinases (MMPs) are a family of structurally related zinc-containing enzymes that mediate the integrity of extracellular matrix (Chem. Rev., 1999, 99, 2735-2776). They are excreted by a variety of connective tissue and pro-inflammatory cells such as fibroblasts, osteoblasts, macrophages, neutrophils, lymphocytes, and endothelial cells. There is now a body of evidence that matrix metalloproteinases (MMPs) are important in the uncontrolled breakdown of connective tissue, including proteoglycan and collagen, leading to resorption of the extracellular matrix. This is a feature of a number of cardiovascular pathological conditions, such as atherosclerosis, heart failure, restenosis, and reperfusion injury. Normally, these catabolic enzymes are tightly regulated at the level of their synthesis as well as at their level of extracellular activity through the action of specific inhibitors, such as α-2-macroglobulins and TIMP (tissue inhibitor of metalloproteinase), which form inactive complexes with the MMPs. Therefore, extracellular matrix degradation and remodeling are regulated by the relative expression of TIMPs and MMPs. The MMPs are classified into several families based on their domain structure: matrilysin (minimal domain, MMP-7), collagenase (hemopexin domain, MMP-1, MMP-8, MMP-13), gelatinase (fibronectin domain, MMP-2, MMP-9), stromelysin (hemopexin domain, MMP-3, MMP-10, MMP-11), and metalloelastase (MMP-12). In addition, the transmembrane domain family (MT-MMPs) has been recently discovered and includes MMP-14 through MMP-17.

The ability to detect increased levels of MMPs in the heart would be extremely useful for the detection of tissue degradation that occurs in many heart conditions. The composition and vulnerability of atheromatous plaque in the coronary arteries has recently been recognized as a key determinant in thrombus-mediated acute coronary events, such as unstable angina, myocardial infarction, and death (Circulation, 1995, 92: 657-671). Among the many components involved in the inflammatory atheromatous plaque are macrophages that secrete the matrix metalloproteinases (Circulation, 1996, 94: 2013-2020). The MMPs are a family of enzymes that cleave the usually protease-resistant fibrillar extracellular matrix components of the heart, such as collagen. These extracellular matrix proteins confer strength to the fibrous cap of atheroma (Circulation, 1995, 91: 2844-2850).

Macrophages that accumulate in areas of inflammation such as atherosclerotic plaques release these MMPs that degrade connective tissue matrix proteins (Falk, 1995). In fact, studies have demonstrated that both the metalloproteinases and their mRNA are present in atherosclerotic plaques (Am. J. Physiol., 1998, 274:H1516-1523; Circ. Res. 1995, 77: 863-868; Proc. Natl. Acad. Sci., 1991, 88: 8154-8158), particularly in the vulnerable regions of human atherosclerotic plaques (J. Clin. Invest., 1994, 94: 2493-2503). Amongst the metalloproteinases that may be released by macrophages present at the site of human atheroma are interstitial collagenase (MMP-1), gelatinases A and B (MMP-2 and MMP-9, respectively) and stromelysin (MMP-3) (Circulation, 1994, 90: 775-778). Although all MMPs may be elevated at the site of human atheroma, it has been suggested that gelatinase B may be one of the most prevalent MMPs in the plaque because it can be expressed by virtually all activated macrophages (Circulation, 1995, 91: 2125-2131). The MMP-9 has also been shown to be more prevalent in atherectomy material from unstable angina relative to stable angina patients (Circulation, 1995, 91: 2125-2131).

The left ventricular extracellular matrix, containing a variety of collagens and elastin, is also proposed to participate in the maintenance of left ventricle (LV) geometry. Therefore, alterations in these extracellular components of the myocardium may influence LV function and be a marker of progressive changes associated with LV degeneration and ultimately heart failure (CoAm. J. Physiol., 1998, 274:H1516-1523).

In congestive heart failure (CHF), the relationship of CHF state to MMP activity in the LV remains somewhat unclear, at least in the clinical setting. In pre-clinical models of CHF, however, the functional changes in the LV have been correlated with increased MMP activity. For example, in a pig model of CHF, the decrease in LV function was observed to coincide with a marked increase in MMP-1 (˜300%), MMP-2 (˜200%), and MMP-3 (500%) (Am. J. Physiol., 1998, 274:H1516-1523). Moderate ischemia and reperfusion in a pig model has been demonstrated to selectively activate MMP-9 (Circulation, 1999, 100 Suppl. 1, I-12). Similarly, in a dog model of CHF the levels of gelatinases (e.g. MMP-2 and MMP-9) were found to be elevated in severe heart failure (Can. J Cardiol., 1994, 10: 214-220). The levels of MMP-2 and MT1-MMP (membrane type MMP, MMP-14) were found to be increased in biopsy samples of human myocytes from patients suffering from dilated cardiomyopathy (Circulation, 1999, 100 Suppl. 1, I-12).

Pathologically, MMPs have been identified as associated with several disease states. For example, anomalous MMP-2 levels have been detected in lung cancer patients, where it was observed that serum MMP-2 levels were significantly elevated in stage 1V disease and in those patients with distant metastases as compared to normal sera values (Cancer Res., 1992, 53: 4548). Also, it was observed that plasma levels of MMP-9 were elevated in patients with colon and breast cancer (Cancer Res., 1993, 53: 140).

Elevated levels of stromelysin (MMP-3) and interstitial collagenase (MMP-1) have been noted in synovial fluid derived from rheumatoid arthritis patients as compared to post-traumatic knee injury (Arth. Rheum., 1992, 35: 35). Increased levels of mRNA expression for collagenase type I (MMP-1) and collagenase type IV (MMP-2) have been shown to be increased in ulcerative colitis as compared to Crohn's disease and controls (Gastroenterology, 1992, Abstract 661). Furthermore, increased immuno-histochemical expression of the gelatinase antigen in a rabbit model of chronic inflammatory colitis has been demonstrated (Gastroenterology, 1992, Abstract 591).

It has been shown that the gelatinase MMPs are most intimately involved with the growth and spread of tumors. It is known that the level of expression of gelatinase is elevated in malignancies, and that gelatinase can degrade the basement membrane that leads to tumor metastasis. Angiogenesis, required for the growth of solid tumors, has also recently been shown to have a gelatinase component to its pathology. Furthermore, there is evidence to suggest that gelatinase is involved in plaque rupture associated with atherosclerosis. Other conditions mediated by MMPs are restenosis, MMP-mediated osteopenias, inflammatory diseases of the central nervous system, skin aging, tumor growth, osteoarthritis, rheumatoid arthritis, septic arthritis, corneal ulceration, abnormal wound healing, bone disease, proteinuria, aneurysmal aortic disease, degenerative cartilage loss following traumatic joint injury, demyelinating diseases of the nervous system, cirrhosis of the liver, glomerular disease of the kidney, premature rupture of fetal membranes, inflammatory bowel disease, periodontal disease, age-related macular degeneration, diabetic retinopathy, proliferative vitreoretinopathy, retinopathy of prematurity, ocular inflammation, keratoconus, Sjogren's syndrome, myopia, ocular tumors, ocular angiogenesis/neo-vascularization, and corneal graft rejection. For recent reviews, see: Research Focus, 1996, Vol. 1, 16-26; Curr. Opin. Ther. Patents 1994, 4(1): 7-16; Curr. Medicinal Chem., 1995, 2: 743-762; Exp. Opin. Ther. Patents, 1995, 5(2): 1087-110; and Exp. Opin. Ther. Patents, 1995, 5(12): 1287-1196.

Diagnostic agents targeted to one or more MMPs would be useful for detecting and monitoring the degree of extracellular matrix degradation in degradative disease processes. Diagnostic agents containing a ligand directed at one or more MMPs (e.g. MMP-1, MMP-2, MMP-3, MMP-9) will localize a diagnostic imaging probe to the site of pathology for the purpose of non-invasive imaging of these diseases.

For example, it is known to conjugate an MMP inhibitor to an imaging agent for detecting and monitoring MMP levels. See, for example, International Publication No. WO 01/60416. However, such targeting usually involves a one-to-one interaction between the conjugated imaging agent and the MMP, which is often present in relatively low concentrations. Consequently, the number of targeted imaging probe molecules that accumulate in a particular tissue using this approach is limited and thereby limits the sensitivity of the method.

To avoid this sensitivity limitation, an MMP substrate can be conjugated to an imaging agent for detecting and monitoring MMP levels. Because multiple conjugated imaging agents may interact with each molecule of MMP, there is an amplification of the concentration of imaging agent in the area of interest in the patient. It would be beneficial to develop diagnostic agents that would be useful in the methods of detecting and/or imaging and/or monitoring the presence of matrix metalloproteinase or a pathological disorder associated with matrix metalloproteinase activity in a patient, especially those with greater specificity and sensitivity and those which use different trapping mechanisms. Compounds that localize in areas of MMP activity will allow detection and localization of these diseases that are associated with altered MMP levels relative to normal tissue.

In one embodiment, the disclosure is directed to compounds comprising:
a. at least one targeting moiety;
b. an optional chelator; and
c. a masked trapping moiety; and
d. an optional linking group;
   or a pharmaceutically-acceptable derivative thereof;
wherein said targeting moiety is a matrix metalloproteinase substrate;
wherein said chelator is capable of conjugating to a diagnostic component;
wherein said masked trapping moiety is capable of being unmasked to form an unmasked trapping moiety;
wherein said unmasked trapping moiety is capable of being immobilized at a site of interest in a patient;
wherein, in use, said immobilization of said compound is accomplished through an interaction between said unmasked trapping moiety and a substance associated with a pathological disorder associated with matrix metalloproteinase activity at said site of interest in said patient;
provided that said interaction is non-receptor mediated; and
provided that, in use, when said substance is a protein, said interaction is a covalent bond.

In another embodiment, the disclosure is directed to compounds comprising:
a. at least one targeting moiety;
b. an optional chelator; and
c. a masked trapping moiety; and
d. an optional linking group;
   or a pharmaceutically-acceptable derivative thereof;
wherein said targeting moiety is a matrix metalloproteinase substrate;
wherein said chelator is capable of conjugating to a diagnostic component;
wherein said masked trapping moiety is capable of being unmasked to form an unmasked trapping moiety;
wherein said unmasked trapping moiety is capable of being immobilized at a site of interest in a patient;
wherein, in use, said immobilization of said compound is accomplished through an interaction between said unmasked trapping moiety and a substance associated with a pathological disorder associated with matrix metalloproteinase activity at said site of interest in said patient;
provided that said interaction is non-receptor mediated; and
provided that, in use the signal from said diagnostic component is substantially unchanged before and after said unmasked trapping moiety is immobilized.

In another embodiment the present disclosure provides a method of preparing a 1,2-dicarbonyl compound, the method comprising:
a. reacting a compound as described above with MMP;
b. reacting the product of step a with APN to form an α-aminoketone; and
c. oxidizing said α-aminoketone with serum amine oxidase.

In another embodiment, the disclosure is directed to diagnostic agents, comprising:
a. a compound as described above or a pharmaceutically acceptable derivative thereof, and
b. a diagnostic component.

In another embodiment, the disclosure is directed to compositions, comprising:
a. the compound or diagnostic agent as described above; and
b. a pharmaceutically-acceptable carrier.

In other embodiments, the disclosure is directed to kits for detecting and/or imaging and/or monitoring the presence of matrix metalloproteinase in a patient comprising:
a. the diagnostic agent as described above;
b. a pharmaceutically acceptable carrier; and
c. instructions for preparing detecting and/or imaging and/or monitoring the presence of matrix metalloproteinase in a patient.

In other embodiments, the disclosure is directed to methods of detecting, imaging, and/or monitoring the presence of matrix metalloproteinase in a patient, comprising the steps of:
a. administering to said patient the diagnostic agent described above; and
b. acquiring an image of a site of concentration of said diagnostic agent in the patient by a diagnostic imaging technique.

In another embodiment, the disclosure is directed to methods of detecting, imaging, and/or monitoring a pathological disorder associated with matrix metalloproteinase activity in a patient, comprising the steps of:
a. administering to said patient the diagnostic agent described above; and
b. acquiring an image of a site of concentration of said diagnostic agent in the patient by a diagnostic imaging technique.

In other embodiments, the disclosure is directed to methods of detecting, imaging, and/or monitoring atherosclerosis, including coronary atherosclerosis or cerebrovascular atherosclerosis, in a patient, comprising the steps of:
a. administering to said patient the diagnostic agent described above; and
b. acquiring an image of a site of concentration of said diagnostic agent in the patient by a diagnostic imaging technique.

In other embodiments, the disclosure is directed to methods of identifying a patient at high risk for transient ischemic attacks, stroke, acute cardiac ischemia, congestive heart failure, myocardial infarction or cardiac death by determining the degree of active atherosclerosis in a patient, comprising carrying out one of the methods described above.

In other embodiments, the disclosure is directed to methods of simultaneous imaging of cardiac perfusion and extracellular matrix degradation in a patient, comprising the steps of:
a. administering the diagnostic agent described above, wherein said diagnostic component is a gamma-emitting radioisotope or positron-emitting radioisotope; and
b. administering a cardiac perfusion compound, wherein said compound is radiolabeled with a gamma-emitting radioisotope or positron-emitting radioisotope that exhibits a gamma emission energy or positron emission energy that is spectrally separable from the gamma emission energy or positron emission energy of the diagnostic component conjugated to the targeting moiety in step a; and
c. acquiring, by a diagnostic imaging technique, simultaneous images of the sites of concentration of the spectrally separable gamma-emission energies or positron-emission energies of the compounds administered in steps a and b.

In another embodiment, the disclosure is directed to methods of detecting and/or imaging and/or monitoring a cancerous tumor in a patient, comprising the steps of:
a. administering to said patient the diagnostic agent described above; and
b. acquiring an image of a site of concentration of said diagnostic agent in the patient by a diagnostic imaging technique.

In other embodiments, the disclosure is directed to compositions comprising at least one compound containing an MMP substrate and/or diagnostic agent, and/or a pharmaceutically-acceptable carrier.

The number of carbon atoms in any particular group is denoted before the recitation of the group. For example, the term “C₆₋₁₀aryl” denotes an aryl group containing from six to ten carbon atoms, and the term “C₆₋₁₀aryl-C₁₋₁₀alkyl,” refers to an aryl group of six to ten carbon atoms attached to the parent molecular moiety through an alkyl group of one to ten carbon atoms.

The term “alkenyl,” as used herein, refers to a straight or branched chain hydrocarbon containing at least one carbon-carbon double bond.

The term “alkoxy,” as used herein, refers to an alkyl group attached to the parent molecular moiety through an oxygen atom.

The term “alkoxyalkyl,” as used herein, refers to an alkoxy group attached to the parent molecular moiety through an alkyl group.

The term “alkyl,” as used herein, refers to a group derived from a straight or branched chain saturated hydrocarbon.

The term “alkylaryl,” as used herein, refers to an alkyl group attached to the parent molecular moiety through an aryl group.

The term “alkylarylene,” as used herein, refers to a divalent arylalkyl group, where one point of attachment to the parent molecular moiety is on the alkyl portion and the other is on the aryl portion.

The term “alkylene,” as used herein, refers to a divalent group derived from a straight or branched chain saturated hydrocarbon.

As used herein, the phrase “amino acid residue” means a moiety derived from a naturally-occurring or synthetic organic compound containing an amino group (—NH₂), a carboxylic acid group (—COOH), and any of various side groups, especially any of the 20 compounds that have the basic formula NH₂CHRCOOH, and that link together by peptide bonds to form proteins or that function as chemical messengers and as intermediates in metabolism.

The term “aminocarboxylate,” as used herein, refers to —OC(O)NH₂.

As used herein, the terms “ancillary” or “co-ligands” refers to ligands that serve to complete the coordination sphere of the radionuclide together with the chelator or radionuclide bonding unit of the reagent. For radiopharmaceuticals comprising a binary ligand system, the radionuclide coordination sphere comprises one or more chelators or bonding units from one or more reagents and one or more ancillary or co-ligands, provided that there are a total of two types of ligands, chelators or bonding units. For example, a radiopharmaceutical comprised of one chelator or bonding unit from one reagent and two of the same ancillary or co-ligands and a radiopharmaceutical comprising two chelators or bonding units from one or two reagents and one ancillary or co-ligand are both considered to comprise binary ligand systems. For radiopharmaceuticals comprising a ternary ligand system, the radionuclide coordination sphere comprises one or more chelators or bonding units from one or more reagents and one or more of two different types of ancillary or co-ligands, provided that there are a total of three types of ligands, chelators or bonding units. For example, a radiopharmaceutical comprised of one chelator or bonding unit from one reagent and two different ancillary or co-ligands is considered to comprise a ternary ligand system.

Ancillary or co-ligands useful in the preparation of radiopharmaceuticals and in diagnostic kits useful for the preparation of said radiopharmaceuticals comprise one or more oxygen, nitrogen, carbon, sulfur, phosphorus, arsenic, selenium, and tellurium donor atoms. A ligand can be a transfer ligand in the synthesis of a radiopharmaceutical and also serve as an ancillary or co-ligand in another radiopharmaceutical. Whether a ligand is termed a transfer or ancillary or co-ligand depends on whether the ligand remains in the radionuclide coordination sphere in the radiopharmaceutical, which is determined by the coordination chemistry of the radionuclide and the chelator or bonding unit of the reagent or reagents.

The term “aryl,” as used herein, refers to a phenyl group, or a bicyclic fused ring system wherein one or more of the rings is a phenyl group. Bicyclic fused ring systems consist of a phenyl group fused to a monocyclic cycloalkenyl group, a monocyclic cycloalkyl group, or another phenyl group. The aryl groups of the present invention can be attached to the parent molecular moiety through any substitutable carbon atom in the group. Representative examples of aryl groups include, but are not limited to, anthracenyl, azulenyl, fluorenyl, indanyl, indenyl, naphthyl, phenyl, and tetrahydronaphthyl.

The term “arylalkyl,” as used herein, refers to an aryl group attached to the parent molecular moiety through an alkyl group.

The term “arylalkylaryl,” as used herein, refers to an arylalkyl group attached to the parent molecular moiety through an aryl group.

The term “arylalkylene,” as used herein, refers to a divalent arylalkyl group, where one point of attachment to the parent molecular moiety is on the aryl portion and the other is on the alkyl portion.

The term “arylene,” as used herein, refers to a divalent aryl group.

As used herein, the term “bacteriostat” means a component that inhibits the growth of bacteria in a formulation either during its storage before use of after a diagnostic kit is used to synthesize a diagnostic agent.

The term “buffer,” as used herein, refers to a substance used to maintain the pH of the reaction mixture from about 3 to about 10.

As used herein, the term “carbohydrate” means a polyhydroxy aldehyde, ketone, alcohol or acid, or derivatives thereof, including polymers thereof having polymeric linkages of the acetal type.

The term “carrier”, as used herein, refers to an adjuvant or vehicle that may be administered to a patient, together with the compounds and/or diagnostic agents of this disclosure which does not destroy the activity thereof and is non-toxic when administered in doses sufficient to deliver an effective amount of the diagnostic agent and/or compound.

The terms “chelator” and “bonding unit,” as used herein, refer to the moiety or group on a reagent that binds to a metal ion through one or more donor atoms.

The term “conjugated,” as used herein, refers to the formation of a chemical bond between two moieties.

The term “cyano,” as used herein, refers to —CN.

The term “cycloalkenyl,” as used herein, refers to a non-aromatic, partially unsaturated monocyclic, bicyclic, or tricyclic ring system having three to fourteen carbon atoms and zero heteroatoms. Representative examples of cycloalkenyl groups include, but are not limited to, cyclohexenyl, octahydronaphthalenyl, and norbornylenyl.

The term “cycloalkyl,” as used herein, refers to a saturated monocyclic, bicyclic, or tricyclic hydrocarbon ring system having three to fourteen carbon atoms and zero heteroatoms. Representative examples of cycloalkyl groups include, but are not limited to, cyclopropyl, cyclopentyl, bicyclo[3.1.1]heptyl, and adamantyl.

The term “cycloalkylene,” as used herein, refers to a divalent cycloalkyl group.

As used herein, the term “cyclodextrin” means a cyclic oligosaccharide. Examples of cyclodextrins include, but are not limited to, α-cyclodextrin, hydroxyethyl-α-cyclodextrin, hydroxypropyl-α-cyclodextrin, β-cyclodextrin, hydroxypropyl-β-cyclodextrin, carboxymethyl-β-cyclodextrin, dihydroxypropyl-β-cyclodextrin, hydroxyethyl-β-cyclodextrin, 2,6 di-O-methyl-β-cyclodextrin, sulfated-β-cyclodextrin, γ-cyclodextrin, hydroxypropyl-γ-cyclodextrin, dihydroxypropyl-γ-cyclodextrin, hydroxyethyl-γ-cyclodextrin, and sulfated γ-cyclodextrin.

As used herein, the term “diagnostic agent” refers to a compound that may be used to detect, image and/or monitor the presence and/or progression of a condition(s), pathological disorder(s) and/or disease(s).

The term “diagnostic component,” as used herein, refer to a portion or portions of a molecule that allow for the detection, imaging, and/or monitoring of the presence and/or progression of a condition(s), pathological disorder(s), and/or disease(s).

The term “diagnostic imaging technique,” as used herein, refers to a procedure used to detect a diagnostic agent.

The terms “diagnostic kit” and “kit”, as used herein, refer to a collection of components, termed the formulation, in one or more vials that are used by the practicing end user in a clinical or pharmacy setting to synthesize diagnostic agents. The kit provides all the requisite components to synthesize and use the diagnostic agents (except those that are commonly available to the practicing end user such as water or saline for injection), such as a solution of the diagnostic component, (for example, the radionuclide), equipment for heating during the synthesis of the diagnostic agent, equipment necessary for administering the diagnostic agent to the patient such as syringes and shielding (if required), and imaging equipment.

As used herein, the phrase “donor atom” refers to the atom directly attached to a metal by a chemical bond.

The term “endogenous,” as used herein, refers to a substance produced inside an organism or cell.

The term “heterocyclyl,” as used herein, refers to a five-, six-, or seven-membered ring containing one, two, or three heteroatoms independently selected from the group consisting of nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a phenyl group, a monocyclic cycloalkenyl group, a monocyclic cycloalkyl group, or another monocyclic heterocyclyl group. The heterocyclyl groups of the present invention can be attached to the parent molecular moiety through a carbon atom or a nitrogen atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “heterocyclylalkyl,” as used herein, refers to a heterocyclyl group attached to the parent molecular moiety through an alkyl group.

The term “heterocyclylalkylene,” as used herein, refers to a divalent heterocyclylalkyl group, where one point of attachment to the parent molecular moiety is on the heterocyclyl portion and the other is on the alkyl portion.

The term “heterocyclylene,” as used herein, refers to a divalent heterocyclyl group.

As used herein, the phrase “hydrophobic amino acid residue” means an amino acid residue, as defined above, that does not contain an ionized group(s) at physiological pH, and that leads to an increase in lipophilicity and inhibits diffusion of the compound containing the residue from the target, such as a lipid-laden coronary plaque. Examples of hydrophobic amino acid residues include, but are not limited to, glycine, alanine, valine, lucine, isoleucine, methionine, phenylalanine, tryptophan, tyrosine, and derivatives thereof.

The term “ligand,” as used herein, refers to an atom or molecule or radical or ion that forms a complex around a central atom.

The term “linking group,” as used herein, refers to a portion of a molecule that serves as a spacer between two other portions of the molecule. Linking groups may also serve other functions as described herein.

As used herein, the term “lyophilization aid” means a component that has favorable physical properties for lyophilization, such as the glass transition temperature, and is added to the formulation to improve the physical properties of the combination of all the components of the formulation for lyophilization.

The term “masked trapping moiety,” as used herein, refers to a molecule or portion thereof, which shows decreased binding affinity for a particular chemical functional group due to the presence of a masking group. Once the masking group is removed, an unmasked trapping is formed. The term “unmasked trapping moiety,” as used herein, refers to a molecule or portion thereof that displays increased binding affinity for a particular chemical functional group relative to the masked trapping moiety.

As used herein, the term “metallopharmaceutical” means a pharmaceutical comprising a metal. The metal is the origin of the imageable signal in diagnostic applications and the source of the cytotoxic radiation in radiotherapeutic applications.

As used herein, the phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The term “radiopharmaceutical,” as used herein, refers to a metallopharmaceutical in which the metal is a radioisotope.

As used herein, the term “reagent” means a compound of this disclosure capable of direct transformation into a diagnostic agent of this disclosure. Reagents may be utilized directly for the preparation of the diagnostic agents of this disclosure or may be a component in a kit of this disclosure.

The term “reducing agent,” as used herein, refers to a compound that reacts with a radionuclide (which is typically obtained as a relatively unreactive, high oxidation state compound) to lower its oxidation state by transferring electron(s) to the radionuclide, thereby making it more reactive.

As used herein, the phrase “solubilization aid” is a component that improves the solubility of one or more other components in the medium required for the formulation.

As used herein, the phrase “stabilization aid” means a component that is added to the metallopharmaceutical or to the diagnostic kit either to stabilize the metallopharmaceutical or to prolong the shelf-life of the kit before it must be used. Stabilization aids can be antioxidants, reducing agents or radical scavengers and can provide improved stability by reacting preferentially with species that degrade other components or the metallopharmaceutical.

The term “stable”, as used herein, refers to compounds which possess the ability to allow manufacture and which maintain their integrity for a sufficient period of time to be useful for the purposes detailed herein. Typically, the compounds of the present disclosure are stable at a temperature of 40° C. or less in the absence of moisture or other chemically reactive conditions for at least a week.

The term “sterile,” as used herein, means free of or using methods to keep free of pathological microorganisms.

The term “substrate,” as used herein, refers to a substance acted upon by an enzyme. In the present disclosure, a substrate is a substance upon which the enzyme matrix metallopreteinase acts upon.

The term “surfactant,” as used herein, refers to any amphiphilic material that produces a reduction in interfacial tension in a solution.

The term “pharmaceutically acceptable derivative,” as used herein, refers to any pharmaceutically acceptable salt, ester, salt of an ester, or other derivative of a compound of the disclosure that, upon administration to a recipient, is capable of providing (directly or indirectly) a compound of this disclosure or a metabolite or residue thereof. Typically, derivatives are those that increase the bioavailability of the compounds of the disclosure when such compounds are administered to a mammal (e.g., by allowing an orally administered compound to be more readily absorbed into the blood) or which enhance delivery of the parent compound to a biological compartment (e.g., the brain or lymphatic system) relative to the parent species.

As used herein, the phrase “polyalkylene glycol” means a polyethylene glycol, polypropylene glycol or polybutylene glycol having a molecular weight of less than about 5000, terminating in either a hydroxy or alkyl ether moiety.

As used herein, the phrase “transfer ligand” means a ligand that forms an intermediate complex with a metal ion that is stable enough to prevent unwanted side-reactions but labile enough to be converted to a metallopharmaceutical. The formation of the intermediate complex is kinetically favored while the formation of the metallopharmaceutical is thermodynamically favored. Transfer ligands useful in the preparation of metallopharmaceuticals and in diagnostic kits useful for the preparation of diagnostic radiopharmaceuticals include but are not limited to gluconate, glucoheptonate, mannitol, glucarate, N,N,N′,N′-ethylenediaminetetraacetic acid, pyrophosphate and methylenediphosphonate. In general, transfer ligands are comprised of oxygen or nitrogen donor atoms.

Asymmetric centers exist in the compounds of the present invention. These centers are designated by the symbols “R” or “S”, depending on the configuration of substituents around the chiral carbon atom. It should be understood that the invention encompasses all stereochemical isomeric forms of the present compounds, or mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Because double bonds exist in the present compounds, the disclosure contemplates various geometric isomers and mixtures thereof resulting from the arrangement of substituents around these double bonds. It should be understood that the disclosure encompasses both isomeric forms, and mixtures thereof. For carbon-carbon double bonds, the term “E” represents higher order substituents on opposite sides of the carbon-carbon double bond, and the term “Z” represents higher order substituents on the same side of the carbon-carbon double bond.

When any variable occurs more than one time in any substituent or in any formula, its definition on each occurrence is independent of its definition at every other occurrence. Thus, for example, if a group is shown to be substituted with 0-2 R²³, then said group may optionally be substituted with up to two R²³, and R²³ at each occurrence is selected independently from the defined list of possible R²³. Also, by way of example, for the group —N(R²⁴)₂, each of the two R²⁴ substituents on the nitrogen is independently selected from the defined list of possible R²⁴. Combinations of substituents and/or variables are permissible only if such combinations result in stable compounds. When a bond to a substituent is shown to cross the bond connecting two atoms in a ring, then such substituent may be bonded to any atom on the ring.

The compounds of the disclosure require at least two domains or components parts: at least one targeting moiety (“S”), wherein the targeting moiety is an MMP substrate; and at least one masked trapping moiety (“M-T”). The compounds of the disclosure may optionally comprise a chelator (“C”) capable of conjugating to a diagnostic component (“D”, alternatively referred to herein as the “reporter” or “imaging moiety”) and/or a linking group (“L”).

Because one molecule of MMP can hydrolyze multiple MMP substrate molecules, diagnostic agents of the disclosure have the advantage of inherent built-in amplification. The diagnostic agents of the disclosure typically meet the criteria of any diagnostic agent, including chemical stability, labeling with high purity, rapid blood clearance and favorable biodistribution. In addition, the diagnostic agents of the disclosure also typically meet the following special criteria:
(1) The diagnostic agent typically freely diffuses into and out of the target substance, such as coronary plaque.
(2) The diagnostic agent is typically stable to proteinases found in the blood and other non-target tissues.
(3) The diagnostic agent typically contains a masked trapping moiety that is unmasked by MMP digestion.
(4) The diagnostic agent is typically immobilized within the target substance, such as coronary plaque, and accumulates in the target substance to allow signal to increase over time.

The selectivity of the diagnostic agents of the disclosure is believed to derive from the higher concentration of MMPs in certain tissues, organs, or compartments within the body relative to normal tissues, organs, or compartments within the body, such as in vulnerable coronary plaques as compared to stable coronary plaques. The trapping mechanism is not required to be tissue specific. However, it is advantageous if the trapping mechanism is tissue specific, because it provides a double level of specifity, thereby providing a greater target-to-background signal.

In one embodiment of the present disclosure the signal of the diagnostic component does not substantially change when it is immobilized at the target in the patient. This means that the signal is not substantially enhanced upon binding of the molecule. As used in this context, “substantially” means that the signal is not changed by more than 20%. In another embodiment the signal is not changed by more than 10%. In another embodiment the signal is not changed by more than 5%. In another embodiment the signal is not changed by more than 1% and in another embodiment the signal is not changed more than 0%.

The diagnostic component may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

The diagnostic agent may be a MMP substrate linked to radioisotopes known to be useful for imaging by gamma scintigraphy or positron emission tomography (PET). Alternatively, the MMP targeting ligand may be bound to a single or multiple chelator moieties for attachment of one or more paramagnetic metal atoms. This would cause a local change in magnetic properties, such as relaxivity or susceptibility, at the site of tissue damage that could be imaged with magnetic resonance imaging systems. Alternatively, the MMP substrate may be bound to a phospholipid or polymer material used to encapsulate/stabilize microspheres of gas detectable by ultrasound imaging following localization at the site of tissue injury.

Suitable echogenic gases include a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include a carbon-11, nitrogen-13, fluorine-18, iodine-123, and iodine-125.

Suitable optical reporters include a fluorescent reporter and chemiluminescent groups.

Suitable radioisotopes include: ^{99m}Tc, ⁹⁵Tc, ¹¹¹In, ⁶²Cu, ⁶⁴Cu, ⁶⁷Ga, and ⁶⁸Ga. In a specific embodiment of the present disclosure suitable radioisotopes include 99mTc and ¹¹¹In.

Suitable paramagnetic metal ions include: Gd(III), Dy(III), Fe(III), and Mn(II).

Suitable x-ray absorbers include: Re, Sm, Ho, Lu, Pm, Y, Bi, Pd, Gd, La, Au, Au, Yb, Dy, Cu, Rh, Ag, and Ir.

When the diagnostic component is a radioisotope, the diagnostic agent may further comprise a first ancillary ligand and a second ancillary ligand capable of stabilizing the radioisotope. A large number of ligands can serve as ancillary or co-ligands, the choice of which is determined by a variety of considerations such as the ease of synthesis of the radiopharmaceutical, the chemical and physical properties of the ancillary ligand, the rate of formation, the yield, and the number of isomeric forms of the resulting radiopharmaceuticals, the ability to administer said ancillary or co-ligand to a patient without adverse physiological consequences to said patient, and the compatibility of the ligand in a lyophilized kit formulation. The charge and lipophilicity of the ancillary ligand will effect the charge and lipophilicity of the radiopharmaceuticals. For example, the use of 4,5-dihydroxy-1,3-benzenedisulfonate results in radiopharmaceuticals with an additional two anionic groups because the sulfonate groups will be anionic under physiological conditions. The use of N-alkyl substituted 3,4-hydroxypyridinones results in radiopharmaceuticals with varying degrees of lipophilicity depending on the size of the alkyl substituents.

The masked trapping moiety, M-T, is capable of being unmasked to form an unmasked trapping moiety, T, and is capable of being immobilized at said site of interest in the patient. The immobilization of said compound is accomplished through a non-receptor mediated interaction between the unmasked trapping moiety and a substance associated with a pathological disorder or interest. When the substance associated with a pathological disorder is other than a protein, cholesterol, or lipid, the interaction may be covalent or non-covalent, provided that it is not receptor-mediated.

The masked trapping moiety (M-T) “masks” (or decreases) the binding of the diagnostic agent to the substance associated with a pathological disorder within the tissue desired to be detected and/or imaged and/or monitored. Once the mask (M) of the masked trapping moiety (M-T) is removed to form the unmasked trapping moiety (T) by enzymatic cleavage, then the increased binding affinity of the agent is expressed. This results in the physical separation of at least two molecular fragments, one containing the unmasked trapping moiety and the targeting moiety(ies), and the other the mask portion of the masked trapping moiety.

The required and optional domains or parts of the compounds of the disclosure may be arranged in a variety of positions with respect to each other. While these domains can exist without any specific boundaries between them (e.g., the masked trapping moiety can be part of the targeting moiety(ies)), it is convenient to conceptualize them as separate units of the molecule. For example, the following structures are contemplated: 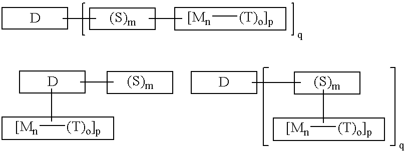
wherein
   S is the targeting moiety comprising the MMP substrate;
   D is the diagnostic component;
   M is the trapping moiety;
   T is the mask for the trapping moiety;
   each of m, n, o, p and q are the same or different and are greater than or equal to one. Generally m, n, o, p and q are less than five, and typically are equal to one.

It is contemplated that the compound may comprise a physiologically-compatible linking group that links the functional domains of the compounds. In one embodiment, the masked trapping moiety optionally comprises a physiologically-compatible linking group that links the masked trapping moiety to the other functional domains of the compounds of the disclosure. In general, the linking group does not contribute significantly to the binding or image enhancing functionality of the diagnostic agent. In some cases, the presence of the linking group may be preferred based on synthetic considerations. In other cases, the linking group may facilitate operation of the bioactivity at the masked trapping moiety. Examples of the linking groups include linear, branched, or cyclic alkyl, aryl, ether, polyhydroxy, polyether, polyamine, heterocyclic, aromatic, hydrazide, peptide, peptoid, or other physiologically compatible covalent linkages or combinations thereof.

In certain embodiments the compounds of the disclosure have about one to about ten targeting moieties. In another embodiment the compounds have about one to about five targeting moieties and in another embodiment the compounds have about one targeting moiety.

In the compounds of disclosure, the targeting moiety is a substrate of one or more MMPs, for example wherein the MMPs are selected from the group consisting of MMP-1, MMP-2, MMP-3, MMP-9, MMP-14 and combinations thereof. In another embodiment the MMPs are selected from the group consisting of MMP-2, MMP-9, MMP-14 and combinations thereof.

The MMP substrate comprises a peptide sequence. The peptide sequence may be derived from collagen, proteoglycan, laminin, fibronectin, gelatin, galectin-3, cartilage link protein, myelin basic protein, kallikrein 14, ladinin 1, endoglin, endothilin receptor, laminin α2 chain, phosphate regulating neutral endopeptidase, ADAM 2, demoglein 3, integrin β5, integrin βv, integrin β6, integrin βx, integrin β9, elastin, perlacan, entactin, vitronectin, tenascin, nidogen, dermatan sulfate, proTNF-α, aggrecan, transin, decorin, tissue factor pathway inhibitor, glycoprotein, NG2 proteoglycan, neurocan, PAI-3, big endothelin-1, brevican/BEHAB, decorin, FGFR-1, IGFBP-3, IL-1, α₂-macroglobulin, MCP-3, pregnancy zone protein, proMMP-1, proMMP-2, SPARC, Substance P, betaglycan or dentin.

In certain embodiments, the peptide sequence is Pro-X-X-Hy-(Ser/Thr) (SEQ ID NO: 1) at P₃ through P_{2′}, Gly-Leu-(Lys/Arg) at P₁ through P_{2′}, Arg residues at P₁ and P₂, IPEN-FFGV (SEQ ID NO: 2), BPYG-LGSP (SEQ ID NO: 3), HPSA-FSEA (SEQ ID NO: 4), GPQG-LLGA (SEQ ID NO: 5), GPAG-LSVL (SEQ ID NO: 6), GPAG-IVTK (SEQ ID NO: 7), DAAS-LLGL (SEQ ID NO: 8), RPAV-MTSP (SEQ ID NO: 9), PPGA-YHGA (SEQ ID NO: 10), LRAY-LLPA (SEQ ID NO: 11), SPYE-LKAL (SEQ ID NO: 12), TAAA-LTSC (SEQ ID NO: 13), GPEG-LRVG (SEQ ID NO: 14), GHAR-LVHV (SEQ ID NO: 15), QPVG-INTS (SEQ ID NO: 16), ELGT-YNVI (SEQ ID NO: 17), DVAQ-FVLY (SEQ ID NO: 18), DVAN-YNFF (SEQ ID NO: 19), HPVG-LLAR (SEQ ID NO: 20), KPQQ-FFGL (SEQ ID NO: 21), IPVS-LRSG (SEQ ID NO: 22), HVLN-LRST (SEQ ID NO: 23), DPES-IRSE (SEQ ID NO: 24), DPLE-FKSH (SEQ ID NO: 25), RPIP-ITAS (SEQ ID NO: 26), RVLG-LKAH (SEQ ID NO: 27), KVLN-LTDN (SEQ ID NO: 28), PPEA-LRGI (SEQ ID NO: 29), IVAM-LRAP (SEQ ID NO: 30), TAAA-ITGA SEQ ID NO: 31), Ac-PLG-Hphe-OL (SEQ ID NO: 32), Suc-PLG-Hphe-YL (SEQ ID NO: 33), or Ac-POG-Hphe-L (SEQ ID NO: 34);
wherein
X is independently an amino acid residue;
Hy is a hydrophobic amino acid residue; and
G, A, V, L, I, M, F, P, S, T, Y, N, Q, D, E, K, R, H, B, and 0 are the one-letter abbreviations for specific amino acids, known to those of ordinary skill in the art.

The compounds of the disclosure may optionally contain a chelator (“C”). In certain embodiments of the compounds of the disclosure, the chelator is a surfactant capable of forming an echogenic substance-filled lipid sphere or microbubble. In certain other embodiments, the chelator is a bonding unit having a formula selected from 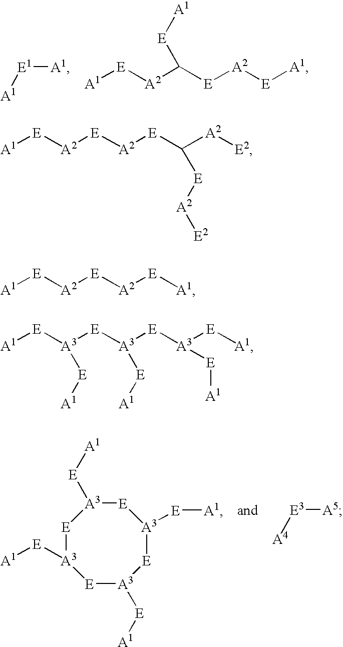
wherein
each A¹ is independently selected from —NR¹⁹R²⁰, —NHR²⁶, —SH, —S(Pg), —OH, —PR¹⁹R²⁰, —P(O)R²¹R²², a bond to said targeting moiety, and a bond to said linking group;
each A² is independently selected from N(R²⁶), N(R¹⁹), S, O, P(R¹⁹), and —OP(O)(R²¹)O—;
A³ is N;
A⁴ is selected from OH and OC(═O)C₁₋₂₀ alkyl;
A⁵ is OC(═O)C₁₋₂₀ alkyl;
each E is independently selected from C₁₋₁₆alkylene substituted with 0-3 R²³, C₆₋₁₀arylene substituted with 0-3 R²³, C₃₋₁₀cycloalkylene substituted with 0-3 R²³, heterocyclyl-C₁₋₁₀alkylene substituted with 0-3 R²³, C₆₋₁₀aryl-C₁₋₁₀alkylene substituted with 0-3 R²³, C₁₋₁₀alkyl-C₆₋₁₀arylene substituted with 0-3 R²³, and heterocyclylene substituted with 0-3 R²³;
E¹ is selected from a bond and E;
each E² is independently selected from C₁₋₁₆alkyl substituted with 0-3 R²³, C₆₋₁₀aryl substituted with 0-3 R²³, C₃₋₁₀cycloalkyl substituted with 0-3 R²³, heterocyclyl-C₁₋₁₀alkyl substituted with 0-3 R²³, C₆₋₁₀aryl-C₁₋₁₀alkyl substituted with 0-3 R²³, C₁₋₁₀alkyl-C₆₋₁₀aryl substituted with 0-3 R²³, and heterocyclyl substituted with 0-3 R²³;
E³ is C₁₋₁₀alkylene substituted with 1-3 R³²;
Pg is a thiol protecting group;
R¹⁹ and R²⁰ are each independently selected from a bond to the linking group, a bond to the targeting moiety, hydrogen, C₁₋₁₀alkyl substituted with 0-3 R²³, aryl substituted with 0-3 R²³, C₃₋₁₀cycloalkyl substituted with 0-3 R²³, heterocyclyl-C₁₋₁₀alkyl substituted with 0-3 R²³, C₆₋₁₀aryl-C₁₋₁₀alkyl substituted with 0-3 R²³, and heterocyclyl substituted with 0-3 R²³; I REMOVED THE POSSIBLITY OF R¹⁹ AND R²⁰ BEING ELECTRONS
R²¹ and R²² are each independently selected from a bond to the linking group, a bond to the targeting moiety, —OH, C₁₋₁₀alkyl substituted with 0-3 R²³, aryl substituted with 0-3 R²³, C₃₋₁₀cycloalkyl substituted with 0-3 R²³, heterocyclyl-C₁₋₁₀alkyl substituted with 0-3 R²³, C₆₋₁₀aryl-C₁₋₁₀alkyl substituted with 0-3 R²³, and heterocyclyl substituted with 0-3 R²³;
each R²³ is independently selected from a bond to the linking group, a bond to the targeting moiety, ═O, halo, trifluoromethyl, cyano, —CO₂R²⁴, —C(═O)R²⁴, —C(═O)N(R²⁴)₂, —CHO, —CH₂OR²⁴, —OC(═O)R²⁴, —OC(═O)OR²⁴, —OR²⁴, —OC(═O)N(R²⁴)₂, —NR²⁴C(═O)R²⁴, —NR²⁴C(—O)OR²⁴, —NR²⁴C(═O)N(R²⁴)₂, —NR²⁴SO₂N(R²⁴)₂, —NR²⁴SO₂R²⁴, —SO₃H, —SO₂R²⁴, —SR²⁴, —S(═O)R²⁴, —SO₂N(R²⁴)₂, —N(R²⁴)₂, —NHC(═S)NHR²⁴, ═NOR²⁴, NO₂, —C(—O)NHOR²⁴, —C(═O)NHNR²⁴R²⁴, —OCH₂CO₂H, 2-(1-morpholino)ethoxy, C₁₋₅alkyl, C₂₋₄alkenyl, C₃₋₆cycloalkyl, C₃₋₆cycloalkylmethyl, C₂₋₆alkoxyalkyl, aryl substituted with 0-2 R²⁴, and heterocyclyl;
each R²⁴ is independently selected from a bond to said linking group, a bond to said targeting moiety, hydrogen, C₁₋₆alkyl, phenyl, benzyl, and C₁₋₆ alkoxy; I'M REMOVING CYANO, NITRO, TRIFLUOROMETHYL, AND HALO SINCE THEY CAN'T EXIST ON MOST OF THE ABOVE COMPOUNDS
R²⁶ is a co-ordinate bond to a metal or a hydrazine protecting group;
each R³² selected from R³⁴, ═O, —CO₂R³³, —C(═O)R³³, —C(═O)N(R³³)₂, —CH₂OR³³, —OR³³, —N(R³³)₂, and C₂-C₄ alkenyl;
each R³³ is independently selected from R³⁴, hydrogen, C₁-C₆ alkyl, phenyl, benzyl, and trifluoromethyl; and
R³⁴ is a bond to said linking group;
wherein at least one of A¹, R¹⁹, R²⁰, R²¹, R²², R²³, R²⁴, and R³⁴ is a bond to said linking group or said targeting moiety; I ADDED R19, 20, 21, 22, 24, and 34 TO THIS PROVISO; IS THAT OK?

In an embodiment of the present disclosure, the chelant is of the formula: 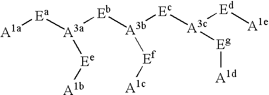
wherein
A^{1a} is a bond to said linking group;
A^{1b}, A^{1c}, A^{1d} and A^{1e} are each OH;
A^{3a}, A^{3b}, and A^{3c} are each N;
E^{a}, E^{b}, and E^{c} are C₂alkylene;
E^{d}, E^{e}, E^{f}, and E^{g} are C₂alkylene substituted with 0-1 R²³; and
R²³ is ═O.

In another embodiment of the present disclosure, the chelant is of the formula: 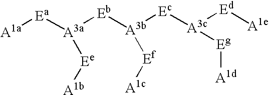
wherein
A^{1a}, A^{1b}, A^{1d} and A^{1e} are each OH;
A^{1c} is a bond to said linking group;
A^{3a}, A^{3b} and A^{3c} are each N;
E^{a}, E^{d}, E^{e}, E^{f}, and E^{g} are C₂alkylene substituted with 0-1 R²³;
E^{b} and E^{c} are C₂alkylene; and
R²³ is ═O.

In another embodiment of the present disclosure the chelant is of the formula: 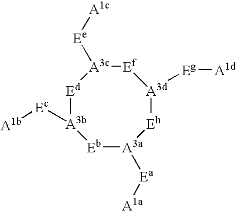
wherein:
A^{3a}, A^{3b}, A^{3c} and A^{3d} are each N;
A^{1a} is a bond to said linking group;
A^{1b}, A^{1c} and A^{1d} are each —OH;
E^{a}, E^{c}, E^{g} and E^{e} are each C₂alkylene substituted with 0-1 R²³;
E^{b}, E^{d}, E^{f} and E^{h} are each C₂alkylene; and
R²³ is ═O.

In another embodiment of the present disclosure, the chelant is of the formula: 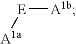
wherein
A^{1a} is —NHR²⁶;
A^{1b} is NHR¹⁹;
E is a bond;
R¹⁹ is heterocyclyl substituted with R²³, the heterocyclyl being selected from pyridine and pyrimidine;
R²⁶ is a co-ordinate bond to a metal or a hydrazine protecting group;
R²³ is selected from a bond to said linking group, C(═O)NHR²⁴ and C(═O)R²⁴; and
R²⁴ is a bond to said linking group.

In another embodiment of the present disclosure, the chelant is of the formula: 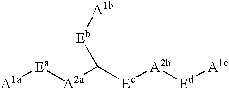
wherein
A^{1a} and A^{1c} are each —S(Pg);
A^{1b} is a bond to said linking group;
A^{2a} and A^{2b} are each —NH;
E^{a} and E^{d} are C₂alkylene substituted with 0-1 R²³;
E^{b} is C₁₋₃alkylene substituted with 0-1 R²³;
E^{c} is CH₂; and
R²³ is ═O;

In another embodiment of the present disclosure, the chelant is of the formula: 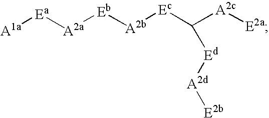
wherein:
A^{1a} is a bond to said linking group;
A^{2a} is NH;
A^{2b} is —OP(O)(R²¹)O—;
A^{2c} and A^{2d} are each O;
E^{a} is C₁ alkylene substituted by R²³;
E^{b} is C₂alkylene substituted with 0-1 R²³;
E^{c} and E^{d} are C₁alkylene;
E^{2a} and E^{2b} are each C₁₋₁₆alkyl substituted with 0-1 R²³;
R²¹ is —OH; and
R²³ is ═O.

One of the key features of the diagnostic agents of the disclosure is that once the MMP substrate domain has targeted the diagnostic agent to the vicinity of a target organ, compartment or region within the patient where there is MMP activity associated with a pathological disorder of interest, the diagnostic agent containing the diagnostic component becomes trapped, i.e., remains for a period of time suitable for imaging but typically is cleared from the body in a period of time that does not cause harm. The trapping of the diagnostic agents may be accomplished by the use of a masked trapping moiety. When the masked trapping moiety is “unmasked,” it permits the immobilization of the portion of the diagnostic agent containing the diagnostic component at the site of interest in the patient.

There are a number of mechanisms by which the unmasked trapping moiety may be trapped in the substance of interest. Suitable trapping mechanisms include, but are not limited to:
(1) trapping due to an increase in lipophilicity of the diagnostic agent containing an unmasked trapping moiety relative to the diagnostic agent containing a masked trapping moiety;
(2) trapping by lipid bilayer insertion of the diagnostic agent containing an unmasked trapping moiety;
(3) trapping by formation of covalent bond between the diagnostic agent containing an unmasked trapping moiety and the substance associated with a pathological disorder of interest; and
(4) trapping by cell transporter groups.

The trapping due to an increase in lipophilicity of the diagnostic agent containing an unmasked trapping moiety relative to the diagnostic agent containing a masked trapping moiety may be accomplished in a number of different ways, including, for example, incorporating lipophilic functionality or hydrophilic functionality in certain domains of the diagnostic agent.

In an embodiment of the present disclosure, the compounds incorporate lipophilic functionality in the portion of the diagnostic agent that contains the diagnostic component or domain. Once the MMP cleaves the MMP substrate, the fragment containing the diagnostic component or domain has a greater effective lipophilicity and thereby interacts through non-covalent association with a lipophilic substance of interest, such as the coronary plaque that contains high levels of oxidized lipoproteins in the soft, lipid-laden core, for example. In other embodiments, the unmasked trapping moiety itself comprises lipophilic functionality. The lipophilic functionality may be derived from a long chain alkyl group, long chain alkenyl group, long chain alkynyl group, cycloalkyl group, or a lipophilic residue of an amino acid. In one example the lipophilic functionality contains at least six carbon atoms. In another example the lipophilic functionality contains twelve carbon atoms, and in another example it contains eighteen carbon atoms. The long chain alkyl groups, long chain alkenyl groups, long chain alkynyl groups and cycloalkyl groups may be optionally substituted with aromatic rings. The long chain alkenyl groups and long chain alkynyl groups may optionally additional sites of unsaturation, including double or triple bonds or combinations thereof. In addition, the long chain alkyl groups, long chain alkenyl groups, long chain alkynyl groups, and cycloalkyl groups may optionally contain non-ionizable functional groups, such as, for example, ethers, thioethers, alcohols, aldehydes, ketones; and amines which are considered to be non-basic at physiological pH, such as pyridine and aniline. The lipophilic functionality may be derived from amino acids, such as, but not limited to, valine, norvaline, leucine, norleucine, isoleucine, phenylalanine, proline, homophenylalanine, tetrahydroisoquinoline-3-carboxylic acid, methionine, O-methylserine, and pyridylalanine.

In other embodiments, the matrix metalloproteinase substrate further comprises hydrophilic functionality. The hydrophilic functionality may be derived from polar amino acids, such as, for example, aspartic acid, glutamic acid, lysine, arginine, cysteic acid and ornithine; sugars, and polar polymers, such as, for example, polyalkylene glycols, linear polyamines and dendrimers. Alternatively, functionality may be added for the purpose of reducing the lipophilicity of the MMP substrate. Suitable functionality includes, but is not limited to, amines, alcohols, carboxylic acids, sulfonic acids, phosphonic acids and phosphonates. Once the MMP cleaves the MMP substrate, the fragment containing the diagnostic component or domain has a greater effective lipophilicity and thereby interacts through non-covalent association with a lipophilic substance of interest.

Examples 1 to 40 and 58 demonstrate trapping due to an increase in lipophilicity. Literature reports suggest that compounds of greater lipophilicity diffuse through tissue at a slower rate than compounds of lower lipophilicity. See, for example, Circ. Res., 2000, 879-884. In Examples 1 to 40 and 58, the diagnostic component is attached to the more lipophilic end of the MMP substrate molecule. Upon digestion by MMPs, polar amino acids are removed, resulting in an overall increase in lipophilicity.

Another trapping approach is lipid bilayer insertion of the unmasked trapping moiety of the diagnostic agent. In this trapping mechanism, a lipophilic group can be prevented from inserting itself into a lipid bilayer by attachment to an MMP substrate peptide. Removal of the peptide by MMPs and aminopeptidase N (APN) unmasks the trapping moiety, resulting in retention of the portion of the diagnostic agent containing the targeting moiety in the lipid bilayer material of interest. Aminopeptidases are reported to be present in coronary plaque, for example, at higher concentration than normal aorotic wall (Atheroschlerosis, 1971, 14, 169-180) and are found in most cells types, including macrophages (Adv. Exp. Med. Biol., 2000, 477, 1-24). Typically, the functional group (X, below) remaining on the lipid bilayer-inserting group is as small and as nonpolar as possible. Suitable examples include hydroxyalkanoic acids, hydroxyphenylalkanoic acids, pyridinium salts, aminophenylalkanoic acids, enamides and 4-aminopyridinium salts. A number of different chemicals may be used to mask the lipid bilayer inserting groups, where the remaining functional groups X are groups such as alcohols, phenols, and weakly basic amines. See, for example, J. Pharm. Sci., 1997, 86, 765-767; Advanced Drug Delivery Reviews, 1989, 3, 39-65. 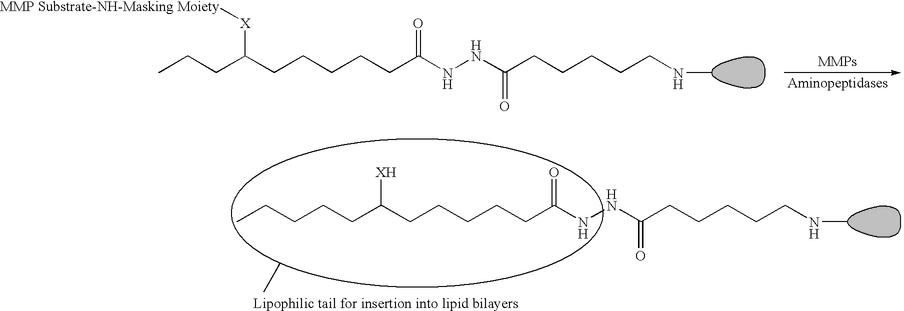
A. Hydroxyalkanoic Acids

Examples 19-23 demonstrate the insertion of hydroxyalkanoic acid into lipid bilayers. In experiments with live cell suspensions, cell association is observed (Example 47). A p-aminobenzyl alcohol is a self-immolative masking moiety for many of these compounds. Removal of the MMP substrate peptide produces an electron-donating amine that destabilizes the bond with the carbonate oxygen. The result is rapid elimination of p-aminobenzyl alcohol, carbon dioxide, and the hydroxyalkanoic acid. Example 24 is a model compound for determining that aminopeptidase will remove the last MMP substrate amino acid from the masking moiety. The group being unmasked in this example is a hydrazide. Example 25 uses the same spacer, but unmasks a hydroxyalkanoic acid. For an example of p-aminobenzyl alcohol as a mask (referred to therein as a prodrug), see Bioorg. Med. Chem. Lett., 2002, 12, 217-219. 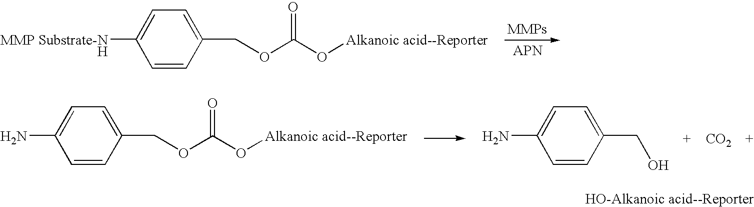
B. Hydroxyphenylalkanoic Acids

Example 26 shows that a hydroxyphenylalkanoic acid will associate with cells. Prophetic examples 51 and 52 illustrate the use of two self-immolative masking moieties that release phenols by a cyclization reaction as shown below. Removal of the MMP substrate peptide converts the non-nucleophilic amide into a nucleophilic amine, promoting the cyclization reaction. 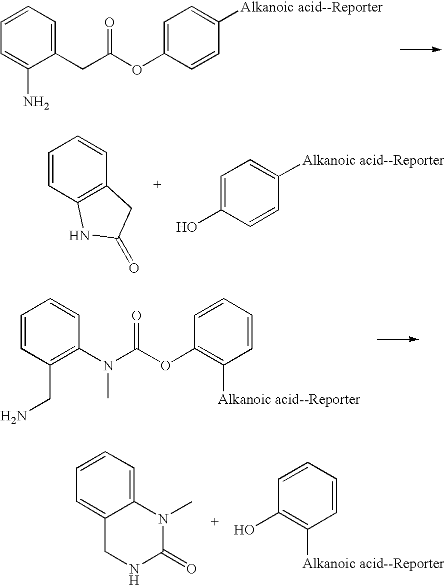
C. Pyridinium Salts (Example 53)

Quaternary ammonium salts produced from pyridines, anilines, and other amines may be used as leaving groups with prodrug linkers, such as the p-aminobenzyl group shown below. The concept is the same as described above for p-aminobenzyl alcohol. Electron donation by the unmasked amine destabilizes the benzyl-nitrogen bond, resulting in a rapid elimination of the tertiary amine (see, for example, J. Pharm. Sci., 1982, 71, 729-735). 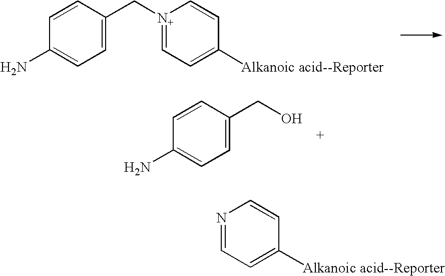
D. Aminophenylalkanoic Acids (Example 55)

Like the pyridine example above, an aniline will remain unprotonated at physiological pH and will therefore be tolerated by a lipid bilayer. Aminopeptidases in the target tissue will recognize the molecule as a substrate and remove the final amino acid, unmasking the aniline.

E. Enamides (Example 54)

Removal of the MMP substrate peptide will produce an enamine of a primary amine, which will then tautomerize to the imine and then hydrolyze to the ketone. The ketone is sufficiently non-polar to allow lipid bilayer insertion. 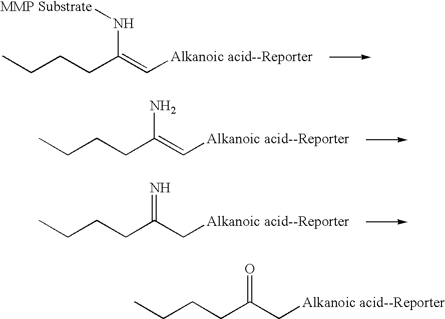
F. 4-Aminopyridinium Salts (Example 56)

MMP substrate may be removed by MMP and APN, resulting in electron donation into the ring to form the substituted 1H-pyridine-4-imine. This will then hydrolyze to form the 1H-pyridine-4-one.

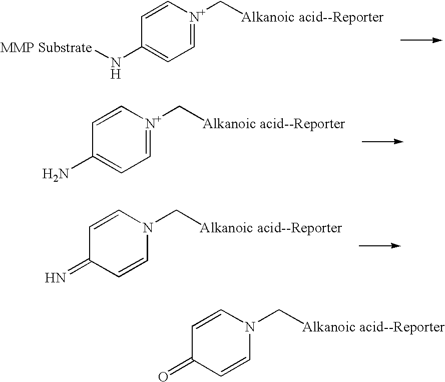

In certain embodiments, the unmasked trapping moiety is capable of forming a covalent bond with a substance associated with a pathological disorder. Suitable unmasked trapping moieties may form a Michael adduct, a hydrazone, a β-sulphone, a Schiff base, a disulfide, a cyclohexene, a cyclohexene derivative, or an oxime with a moiety in said substance. The Michael adduct may formed between a maleimide and an amine or thiol. The hydrazone may be formed between a hydrazine or hydrazide and an aldehyde or a ketone. The β-sulphone may be formed from the 1,4-addition of a nucleophile to a vinyl sulphone. The Schiff base may be formed from the condensation of an amine (aryl or aliphatic) with an aldehyde or ketone. The disulfide may be formed from the reaction of two thiol groups. The cyclohexene (or its derivative products) may be formed from the Diels-Alder condensation of a diene and a dienophile. The oxime may be formed from a ketone or aldehyde reacting with an O-alkoxy hydroxylamine. In other embodiments, functionality on the compounds of the disclosure may react and form a covalent bond with arginine residues in target proteins.

The diagnostic agent may be trapped by formation of stable hydrazones (Examples 6 to 18). The oxidation of LDL in plaque results in the formation of aldehydes. It is well known that aldehydes react with hydrazines and hydrazides to form stable hydrazones, as shown below. In these examples, the MMPs and aminopeptidases (e.g., APN) will remove the masking peptide to generate a free hydrazine or hydrazide, which will subsequently undergo a reaction with aldehydes to form stable hydrazones, trapping the reporter group in the plaque.

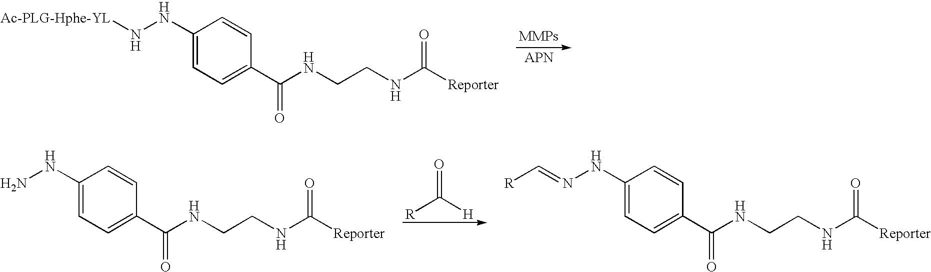

Examples 6 to 9 describe model compounds designed to verify that APN will remove the final amino acid of the MMP substrate sequence to unmask the reactive functionality. Examples 10 to 18 represent complete peptide-hydrazides. These were tested as substrates for MMPs.

The diagnostic agent may be trapped by reaction with arginine (Example 57) or any endogenous biological molecule. 1,2-Dicarbonyl compounds readily react with the guanidino side chain of arginine in proteins, and this reaction is the basis of methods to derivatize peptides and proteins. In Example 57, the dicarbonyl group is masked by the use of a vinyl ester. The linking group belongs to the trimethyl lock category (see J. Org. Chem., 1997, 62, 1363-1367).

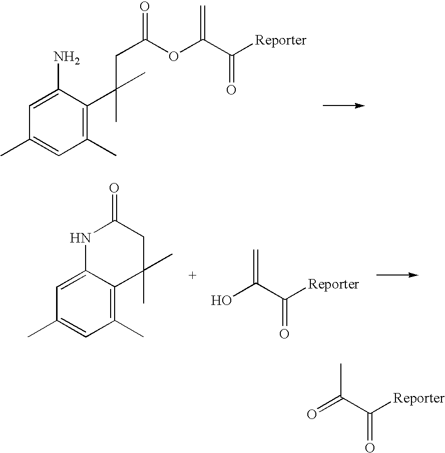

Another trapping mechanism involves trapping by cell transporter groups, such as described in Example 59. A number of small peptides have been shown to have the ability to cross cell membranes, and molecules normally impermeable to cell membranes can be transported into cells when conjugated to these peptides (see Bioconj. Chem., 2001, 12, 825-841). In Example 60, a reporter is conjugated to the C-terminus of a transporter peptide, while the MMP substrate peptide is conjugated off the lysine side chain, where it prevents entry into cells until removed by MMPs and APN.

Yet a further trapping mechanism is trapping by binding of ligands of soluble enzymatic proteins, such as MMPs, cathepsins, aminopeptidases, neprolysin, and the like, or non-enzymatic pretins, such as albumin. Suitable ligands include drugs, lipophilic or amphiphilic organic molecules, porphyrins, steroids, lipids, hormones, peptides, proteins, oligonucleotides (DNA, RNA, or chemically-modified versions thereof), antibodies (including monoclonal and genetically engineered versions and their fragments) or other biomolecules known to bind to at least one soluble enzymatic protein or non-enzymatic protein in the tissue containing the bioactivity to be imaged. In one embodiment, the binding of the ligands is irreversible to promote excretion from the patient after imaging. Suitable examples of soluble enzymatic proteins and soluble non-enzymatic proteins include those disclosed in US 2002/064476, the disclosure of which is incoporated herein in its entirety.

It should be understood that the compounds of this disclosure may be modified by appending appropriate chemical groups to enhance selective biological properties. Such modifications are known in the art and include those that increase biological penetration into a given biological compartment (e.g., blood, lymphatic system, central nervous system), increase oral availability, increase solubility to allow administration by injection, alter metabolism and alter rate of excretion.

It should also be understood that the compounds of this disclosure may adopt a variety of conformational and ionic forms in solution, in pharmaceutical compositions and in vivo. Although the depictions herein of specific compounds of this disclosure are of particular conformations and ionic forms, other conformations and ionic forms of those compounds are envisioned and embraced by those depictions.

Pharmaceutically acceptable carriers, adjuvants and vehicles that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, TRIS (tris(hydroxymethyl)amino-methane), partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropyle-ne-block polymers, polyethylene glycol and wool fat.

According to this disclosure, the pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

In some cases, depending on the dose and rate of injection, the binding sites on plasma proteins may become saturated with prodrug and activated agent. This leads to a decreased fraction of protein-bound agent and could compromise its half-life or tolerability as well as the effectiveness of the agent. In these circumstances, it is desirable to inject the prodrug agent in conjunction with a sterile albumin or plasma replacement solution. Alternatively, an apparatus/syringe can be used that contains the contrast agent and mixes it with blood drawn up into the syringe; this is then re-injected into the patient.

The compounds, diagnostic agents and pharmaceutical compositions of the present disclosure may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term “parenteral” as used herein includes subcutaneous, intravenous, intramuscular, intra-articular, intra-synovial, intrasternal, intrathecal, intrahepatic, intralesional and intracranial injection or infusion techniques.

When administered orally, the pharmaceutical compositions of this disclosure may be administered in any orally acceptable dosage form including, but not limited to, capsules, tablets, aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, when administered in the form of suppositories for rectal administration, the pharmaceutical compositions of this disclosure may be prepared by mixing the agent with a suitable non-irritating excipient that is solid at room temperature but liquid at rectal temperature and therefore will melt in the rectum to release the drug. Such materials include cocoa butter, beeswax and polyethylene glycols.

As noted before, the pharmaceutical compositions of this disclosure may also be administered topically, especially when the target of treatment includes areas or organs readily accessible by topical application, including the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, poly-oxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, typically, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

For administration by nasal aerosol or inhalation, the pharmaceutical compositions of this disclosure are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of active ingredient that may be combined with the carrier materials to produce a single dosage form will vary depending upon the host treated and the particular mode of administration. A typical preparation will contain from about 5% to about 95% active compound (w/w). Typically, such preparations contain from about 20% to about 80% active compound.

For intravenous and other types of administration, acceptable dose ranges range from about 0.001 to about 1.0 mmol/kg of body weight, with the typical dose of the active ingredient compound ranging from about 0.001 to about 0.5 mmol/kg of body weight. Even more typical is from about 0.01 to about 0.1 mmol/kg, and the most typical dose of the active ingredient compound is from about 0.02 and to about 0.05 mmol/kg.

As the skilled artisan will appreciate, lower or higher doses than those recited above may be required. Specific dosage regimens for any particular patient will depend upon a variety of factors, including the activity of the specific compound employed, the age, body weight, general health status, sex, diet, time of administration, rate of excretion, drug combination and the judgment of the treating physician.

It will be appreciated that the preferred pharmaceutical compositions are those comprising the preferred compounds and diagnostic agents of this disclosure.

Another aspect of the present disclosure is diagnostic kits for the preparation of diagnostic agents for detecting, imaging, and/or monitoring a pathological disorder associated with matrix metalloproteinase activity. Diagnostic kits of the present disclosure comprise one or more vials containing the sterile, non-pyrogenic, formulation comprising a predetermined amount of a reagent of the present disclosure, and optionally other components such as one or two ancillary ligands such as tricine and 3-[bis(3-sulfophenyl)phosphine]benzenesulfonic acid (TPPTS), reducing agents, transfer ligands, buffers, lyophilization aids, stabilization aids, solubilization aids and bacteriostats. The kits may also comprise a reducing agent, such as, for example, tin(II).

The inclusion of one or more optional components in the formulation will frequently improve the ease of synthesis of the diagnostic agent by the practicing end user, the ease of manufacturing the kit, the shelf-life of the kit, or the stability and shelf-life of the radiopharmaceutical. The inclusion of one or two ancillary ligands is required for diagnostic kits comprising reagent comprising a hydrazine or hydrazone bonding moiety. The one or more vials that contain all or part of the formulation can independently be in the form of a sterile solution or a lyophilized solid.

Another aspect of the present disclosure is diagnostic kits for the preparation of diagnostic agents for the diagnosis of cardiovascular disorders, infectious disease, inflammatory disease and cancer. Diagnostic kits of the present disclosure contain one or more vials containing the sterile, non-pyrogenic, formulation comprising a predetermined amount of the chelant described in this disclosure, a stabilizing coligand, a reducing agent, and optionally other components such as buffers, lyophilization aids, stabilization aids, solubilization aids and bacteriostats.

The inclusion of one or more optional components in the formulation will frequently improve the ease of synthesis of the diagnostic agent by practicing end user, the ease of manufacturing the kit, the shelf-life of the kit, or the stability and shelf-life of the radiopharmaceutical. The improvement achieved by the inclusion of an optional component in the formulation must be weighed against the added complexity of the formulation and added cost to manufacture the kit. The one or more vials that contain all or part of the formulation can independently be in the form of a sterile solution or a lyophilized solid.

Buffers useful in the preparation of diagnostic agents and kits thereof include but are not limited to phosphate, citrate, sulfosalicylate, and acetate. A more complete list can be found in the United States Pharmacopeia.

Lyophilization aids useful in the preparation of diagnostic agents and kits thereof include but are not limited to mannitol, lactose, sorbitol, dextran, Ficoll, and polyvinylpyrrolidine (PVP).

Stabilization aids useful in the preparation of of diagnostic agents and kits thereof include but are not limited to ascorbic acid, cysteine, monothioglycerol, sodium bisulfite, sodium metabisulfite, gentisic acid, and inositol.

Solubilization aids useful in the preparation of diagnostic agents and kits thereof include but are not limited to ethanol, glycerin, polyethylene glycol, propylene glycol, polyoxyethylene sorbitan monooleate, sorbitan monoloeate, polysorbates, poly(oxyethylene)-poly(oxypropylene)poly(oxyethylene) block copolymers (Pluronics) and lecithin. Typical solubilizing aids are polyethylene glycol, and Pluronics copolymers.

Bacteriostats useful in the preparation of of diagnostic agents and kits thereof include but are not limited to benzyl alcohol, benzalkonium chloride, chlorbutanol, and methyl, propyl or butyl paraben.

A component in a diagnostic kit can also serve more than one function. A reducing agent can also serve as a stabilization aid, a buffer can also serve as a transfer ligand, a lyophilization aid can also serve as a transfer, ancillary or coligand and so forth.

The predetermined amounts of each component in the formulation are determined by a variety of considerations that are in some cases specific for that component and in other cases dependent on the amount of another component or the presence and amount of an optional component. In general, the minimal amount of each component is used that will give the desired effect of the formulation. The desired effect of the formulation is that the practicing end user can synthesize the diagnostic agent and have a high degree of certainty that the diagnostic agent can be injected safely into a patient and will provide diagnostic information about the disease state of that patient.

The diagnostic kits of the present disclosure can also contain written instructions for the practicing end user to follow to synthesize the diagnostic agents. These instructions may be affixed to one or more of the vials or to the container in which the vial or vials are packaged for shipping or may be a separate insert, termed the package insert.

X-ray contrast agents, ultrasound contrast agents and metallopharmaceuticals for magnetic resonance imaging contrast agents are provided to the end user in their final form in a formulation contained typically in one vial, as either a lyophilized solid or an aqueous solution. The end user reconstitutes the lyophilized solid with water or saline and withdraws the patient dose or simply withdraws the dose from the aqueous solution formulation as provided.

These diagnostic agents, whether for gamma scintigraphy, positron emission tomography, MRI, ultrasound or x-ray image enhancement, are useful, inter alia, to detect and monitor changes in cardiovascular diseases over time. Since the degree of overexpression of MMPs is related to the degradation of cardiac or vascular tissue (JACC, 1999, 33: 835-842) it is possible to assess the severity and current activity of cardiovascular disease lesions (i.e. plaques) by quantitating the degree of localization of these imaging agents at the diseased sites of interest. Moreover, with these diagnostic agents it is possible to monitor changes in MMP activity associated with the institution of pharmaceutical therapies that slow the progression or cause a reversal of atheroschlerotic changes in the vascular system or a reversal of myocardial degradation associated with congestive heart failure. Therefore, it can be appreciated that the imaging of MMPs in the heart would be generally useful for detecting, localizing and monitoring the progression/regression of a variety of cardiac diseases that are associated with alterations in the MMP content of cardiac tissues.

The pathological disorders for which the methods of the disclosure are useful for detecting, imaging, and/or monitoring include cancer (especially in the degradation of extracellular matrix prior to metastases), atherosclerosis (especially in the degradation of the fibrous cap of atherosclerotic plaque leading to rupture, thrombosis, and myocardial infarction or unstable angina), rheumatoid arthritis and osteoarthritis (destruction of cartilage aggrecan and collagen), periodontal disease, inflammation, autoimmune disease, organ transplant rejection, ulcerations (corneal, epidermal, and gastric), scleroderma, epidermolysis bullosa, endometriosis, kidney disease, and bone disease. The compounds, diagnostic agents, compositions, kits and methods of the disclosure are particularly useful in the diagnosis of atherosclerosis, including coronary atherosclerosis and cerebrovascular atherosclerosis and cancerous tumors. The compounds, diagnostic agents, compositions, kits and methods of the disclosure are particularly useful in the diagnosis of patients at high risk for transient ischemic attacks or stroke or at high risk for acute cardiac ischemia, myocardial infarction or cardiac death.

The ultrasound contrast agents of the present disclosure comprise a plurality of matrix metalloproteinase substrate moieties attached to or incorporated into a microbubble of a biocompatible gas, a liquid carrier, and a surfactant microsphere, further comprising an optional linking moiety between the targeting moieties and the microbubble. In this context, the phrase “liquid carrier” means aqueous solution and the term “surfactant” means any amphiphilic material that produces a reduction in interfacial tension in a solution. A list of suitable surfactants for forming surfactant microspheres is disclosed in EP-A-0,727,225, herein incorporated by reference in its entirety. The phrase “surfactant microsphere” includes nanospheres, liposomes, vesicles and the like. The biocompatible gas may air, or a fluorocarbon, such as a C₃ perfluoroalkane, which provides the difference in echogenicity and thus the contrast in ultrasound imaging. The gas is encapsulated or contained in the microsphere to which is attached the biodirecting group, optionally via a linking group. The attachment can be covalent, ionic or by van der Waals forces. Specific examples of such contrast agents include lipid encapsulated perfluorocarbons with a plurality of MMP inhibiting compounds.

X-ray contrast agents of the present disclosure comprise one or more matrix metalloproteinase substrate targeting moieties attached to one or more X-ray absorbing or “heavy” atoms of atomic number 20 or greater, further comprising an optional linking moiety, between the targeting moieties and the X-ray absorbing atoms. The frequently used heavy atom in X-ray contrast agents is iodine. Recently, X-ray contrast agents comprising metal chelates (U.S. Pat. No. 5,417,959) and polychelates comprising a plurality of metal ions (U.S. Pat. No. 5,679,810) have been disclosed. More recently, multinuclear cluster complexes have been disclosed as X-ray contrast agents (U.S. Pat. No. 5,804,161, U.S. Pat. No. 5,458,869, U.S. Pat. No. 5,614,168, U.S. Pat. No. 5,482,699 and U.S. Pat. No. 5,932,190).

MRI diagnostic agents of the present disclosure comprise one or more matrix metalloproteinase substrate targeting moieties attached to one or more paramagnetic metal ions, further comprising an optional linking moiety between the targeting moieties and the paramagnetic metal ions. The paramagnetic metal ions are present in the form of metal complexes or metal oxide particles. U.S. Pat. No. 5,412,148, and U.S. Pat. No. 5,760,191 describe examples of chelators for paramagnetic metal ions for use in MRI contrast agents. U.S. Pat. No. 5,801,228, U.S. Pat. No. 5,567,411 and U.S. Pat. No. 5,281,704, describe examples of polychelants useful for complexing more than one paramagnetic metal ion for use in MRI contrast agents. U.S. Pat. No. 5,520,904 describes particulate compositions comprising paramagnetic metal ions for use as MRI contrast agents.

The diagnostic agents of the present disclosure can be synthesized by several approaches:
(1) One approach involves the synthesis of the targeting MMP substrate moiety, and direct attachment of one or more of the substrate moieties to one or more metal chelators or bonding moieties or to a paramagnetic metal ion or heavy atom containing solid particle, or to an echogenic gas microbubble.
(2) Another approach involves the attachment of the MMP substrate moiety to the linking group, which is then attached to one or more metal chelators or bonding moieties or to a paramagnetic metal ion or heavy atom containing solid particle, or to an echogenic gas microbubble.
(3) Another approach involves the synthesis of the moiety where the MMP substrate is attached to a linking group, by incorporating a residue bearing the linking group into the synthesis of the MMP substrate. The resulting moiety is then attached to one or more metal chelators or bonding moieties or to a paramagnetic metal ion or heavy atom containing solid particle, or to an echogenic gas microbubble.
(4) Another approach involves the synthesis of an MMP substrate bearing a fragment of the linking group, one or more of which are then attached to the remainder of the linking group and then to one or more metal chelators or bonding moieties, or to a paramagnetic metal ion or heavy atom containing solid particle, or to an echogenic gas microbubble.

The MMP substrate moieties optionally bearing a linking group, Ln, or a fragment of the linking group, may be synthesized using standard synthetic methods known to those skilled in the art.

Generally, peptides, polypeptides and peptidomimetics are elongated by deprotecting the alpha-amine of the C-terminal residue and coupling the next suitably protected amino acid through a peptide linkage using the methods described. This deprotection and coupling procedure is repeated until the desired sequence is obtained. This coupling can be performed with the constituent amino acids in a stepwise fashion, or condensation of fragments (two to several amino acids), or combination of both processes, or by solid phase peptide synthesis according to the method originally described in J. Am. Chem. Soc., 1963, 85, 2149-2154.

The peptides, polypeptides and peptidomimetics may also be synthesized using automated synthesizing equipment. In addition to the foregoing, procedures for peptide, polypeptide and peptidomimetic synthesis are described in Stewart and Young, Solid Phase Peptide Synthesis, 2nd ed, Pierce Chemical Co., Rockford, Ill. (1984); Gross, Meienhofer, Udenfriend, Eds., The Peptides: Analysis, Synthesis, Biology, Vol. 1, 2, 3, 5, and 9, Academic Press, New York, (1980-1987); Bodanszky, Peptide Chemistry: A Practical Textbook, Springer-Verlag, New York (1988); and Bodanszky et al., The Practice of Peptide Synthesis, Springer-Verlag, New York (1984).

The coupling between two amino acid derivatives, an amino acid and a peptide, polypeptide or peptidomimetic, two peptide, polypeptide or peptidomimetic fragments, or the cyclization of a peptide, polypeptide or peptidomimetic can be carried out using standard coupling procedures such as the azide method, mixed carbonic acid anhydride (isobutyl chloroformate) method, carbodiimide (dicyclohexylcarbodiimide, diisopropylcarbodiimide, or water-soluble carbodiimides) method, active ester (p-nitrophenyl ester, N-hydroxysuccinic imido ester) method, Woodward reagent K method, carbonyldiimidazole method, phosphorus reagents such as BOP-Cl, or oxidation-reduction method. Some of these methods (especially the carbodiimide) can be enhanced by the addition of 1-hydroxybenzotriazole. These coupling reactions may be performed in either solution (liquid phase) or solid phase.

The functional groups of the constituent amino acids or amino acid mimetics are typically protected during the coupling reactions to avoid undesired bonds being formed. The protecting groups that can be used are listed in Greene, Protective Groups in Organic Synthesis, John Wiley & Sons, New York (1981) and The Peptides: Analysis, Synthesis, Biology, Vol. 3, Academic Press, New York (1981).

The α-carboxyl group of the C-terminal residue may be protected by an ester that can be cleaved to give the carboxylic acid. These protecting groups include:
(1) alkyl esters such as methyl and t-butyl;
(2) aryl esters such as benzyl and substituted benzyl, or
(3) esters that can be cleaved by mild base treatment or mild reductive means such as trichloroethyl and phenacyl esters.

In the solid phase case, the C-terminal amino acid is attached to an insoluble carrier (usually polystyrene). These insoluble carriers contain a group that will react with the carboxyl group to form a bond which is stable to the elongation conditions but readily cleaved later. Examples include: oxime resin (DeGrado and Kaiser (1980) J. Org. Chem. 45, 1295-1300) chloro or bromomethyl resin, hydroxymethyl resin, and aminomethyl resin. Many of these resins are commercially available with the desired C-terminal amino acid already incorporated.

The α-amino group of each amino acid is typically protected. Any protecting group known in the art may be used. Examples of these are:
(1) acyl types such as formyl, trifluoroacetyl, phthalyl, and p-toluenesulfonyl;
(2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc);
(3) aliphatic carbamate types such as tert-butyloxycarbonyl (Boc), ethoxycarbonyl, diisopropylmethoxycarbonyl, and allyloxycarbonyl;
(4) cyclic alkyl carbamate types such as cyclopentyloxycarbonyl and adamantyloxycarbonyl;
(5) alkyl types such as triphenylmethyl and benzyl;
(6) trialkylsilane such as trimethylsilane; and
(7) thiol containing types such as phenylthiocarbonyl and dithiasuccinoyl.

Typical alpha-amino protecting groups are either Boc or Fmoc. Many amino acid or amino acid mimetic derivatives suitably protected for peptide synthesis are commercially available.

The α-amino protecting group is cleaved prior to the coupling of the next amino acid. When the Boc group is used, the methods of choice are trifluoroacetic acid, neat or in dichloromethane, or HCl in dioxane. The resulting ammonium salt is then neutralized either prior to the coupling or in situ with basic solutions such as aqueous buffers, or tertiary amines in dichloromethane or dimethylformamide. When the Fmoc group is used, the reagents of choice are piperidine or substituted piperidines in dimethylformamide, but any secondary amine or aqueous basic solutions can be used. The deprotection is carried out at a temperature between 0° C. and room temperature.

Any of the amino acids or amino acid mimetics bearing side chain functionalities are typically protected during the preparation of the peptide using any of the above-identified groups. Those skilled in the art will appreciate that the selection and use of appropriate protecting groups for these side chain functionalities will depend upon the amino acid or amino acid mimetic and presence of other protecting groups in the peptide, polypeptide or peptidomimetic. The selection of such a protecting group is important in that it must not be removed during the deprotection and coupling of the α-amino group.

For example, when Boc is chosen for the α-amine protection the following protecting groups are acceptable: p-toluenesulfonyl (tosyl) moieties and nitro for arginine; benzyloxycarbonyl, substituted benzyloxycarbonyls, tosyl or trifluoroacetyl for lysine; benzyl or alkyl esters such as cyclopentyl for glutamic and aspartic acids; benzyl ethers for serine and threonine; benzyl ethers, substituted benzyl ethers or 2-bromobenzyloxycarbonyl for tyrosine; p-methylbenzyl, p-methoxybenzyl, acetamidomethyl, benzyl, or t-butylsulfonyl for cysteine; and the indole of tryptophan can either be left unprotected or protected with a formyl group.

When Fmoc is chosen for the α-amine protection usually tert-butyl based protecting groups are acceptable. For instance, Boc can be used for lysine, tert-butyl ether for serine, threonine and tyrosine, and tert-butyl ester for glutamic and aspartic acids.

Once the elongation of the peptide, polypeptide or peptidomimetic, or the elongation and cyclization of a cyclic peptide or peptidomimetic is completed all of the protecting groups are removed. For the liquid phase synthesis the protecting groups are removed in whatever manner as dictated by the choice of protecting groups. These procedures are well known to those skilled in the art.

When a solid phase synthesis is used to synthesize a cyclic peptide or peptidomimetic, the peptide or peptidomimetic should be removed from the resin without simultaneously removing protecting groups from functional groups that might interfere with the cyclization process. Thus, if the peptide or peptidomimetic is to be cyclized in solution, the cleavage conditions need to be chosen such that a free α-carboxylate and a free α-amino group are generated without simultaneously removing other protecting groups. Alternatively, the peptide or peptidomimetic may be removed from the resin by hydrazinolysis, and then coupled by the azide method. Another very convenient method involves the synthesis of peptides or peptidomimetics on an oxime resin, followed by intramolecular nucleophilic displacement from the resin, which generates a cyclic peptide or peptidomimetic (Tetrahedron Letters, 1990, 43, 6121-6124). When the oxime resin is employed, the Boc protection scheme is generally chosen. Then, the preferred method for removing side chain protecting groups generally involves treatment with anhydrous HF containing additives such as dimethyl sulfide, anisole, thioanisole, or p-cresol at 0° C. The cleavage of the peptide or peptidomimetic can also be accomplished by other acid reagents such as trifluoromethanesulfonic acid/trifluoroacetic acid mixtures.

Unusual amino acids used in this disclosure can be synthesized by standard methods familiar to those skilled in the art (The Peptides: Analysis, Synthesis, Biology, Vol. 5, pp. 342-449, Academic Press, New York (1981)). N-Alkyl amino acids can be prepared using procedures described previously (Cheung et al., Can. J. Chem., 1977, 55, 906; Freidinger et al., J. Org. Chem., 1982, 48, 77).

The attachment of linking groups to the MMP substrate; chelators or bonding units to the substrates or to the linking groups; and substrates bearing a fragment of the linking group to the remainder of the linking group, in combination forming the moiety, MMP substrate-linking group, and then to the chelator may all be performed by standard techniques. These include, but are not limited to, amidation, esterification, alkylation, and the formation of ureas or thioureas. Procedures for performing these attachments can be found in Brinkley, M., Bioconjugate Chemistry, 1992, 3, 1.

A number of methods can be used to attach the MMP substrates to paramagnetic metal ion or heavy atom containing solid particles by one skilled in the art of the surface modification of solid particles. In general, the targeting moiety or the combination of targeting moiety and linking group is attached to a coupling group that react with a constituent of the surface of the solid particle. The coupling groups can be any of a number of silanes which react with surface hydroxyl groups on the solid particle surface, as described in U.S. Pat. No. 6,254,852, and can also include polyphosphonates, polycarboxylates, polyphosphates or mixtures thereof which couple with the surface of the solid particles, as described in U.S. Pat. No. 5,520,904.

A number of reaction schemes can be used to attach the MMP substrates, S, to the surfactant microsphere, X3. These are illustrated in following reaction schemes where F represents a surfactant moiety that forms the surfactant microsphere.

Acylation Reaction:
F—C(═O)—Y+S—NH₂ or S—OH→F—C(═O)—NH—S or F—C(═O)—O—S
where Y is a leaving group or active ester
   Disulfide Coupling:
   F—SH+S—SH→F—S—S—S
   Sulfonamide Coupling:
   F—S(═O)₂—Y+S—NH₂→F—S(═O)₂—NH—S
   Reductive Amidation:
   F—CHO+S—NH₂→F—NH—S
   In these reaction schemes, the substituents F and S can be reversed as well.

The linking group Ln can serve several roles. First it provides a spacing group between the metal chelator or bonding moiety, Ch, the paramagnetic metal ion or heavy atom containing solid particle, X2, and the surfactant microsphere, X3, and the one or more of the MMP substrates, S, so as to minimize the possibility that the moieties Ch-X, Ch-X1, X2, and X3, will interfere with the interaction of the recognition sequences of S with MMPs associated with cardiovascular pathologies. The necessity of incorporating a linking group in a reagent is dependent on the identity of S, Ch-X, Ch-X1, X2, and X3. If Ch-X, Ch-X1, X2, and X3, cannot be attached to S without substantially diminishing its ability to inhibit MMPs, then a linking group is used. A linking group also provides a means of independently attaching multiple substrates to one group that is attached to Ch-X, Ch-X1, X2, or X3.

The linking group also provides a means of incorporating a pharmacokinetic modifier into the diagnostic agents of the present disclosure. The pharmacokinetic modifier serves to direct the biodistibution of the injected pharmaceutical other than by the interaction of the targeting moieties with the MMPs expressed in the cardiovascular pathologies. A wide variety of functional groups can serve as pharmacokinetic modifiers, including, but not limited to, carbohydrates, polyalkylene glycols, peptides or other polyamino acids, and cyclodextrins. The modifiers can be used to enhance or decrease hydrophilicity and to enhance or decrease the rate of blood clearance. The modifiers may also be used to direct the route of elimination of the pharmaceuticals. Preferred pharmacokinetic modifiers are those that result in moderate to fast blood clearance and enhanced renal excretion.

The metal chelator or bonding moiety is selected to form stable complexes with the metal ion chosen for the particular application. Chelators or bonding moieties for diagnostic radiopharmaceuticals are selected to form stable complexes with the radioisotopes that have imageable gamma ray or positron emissions, such as ^{99m}Tc, ⁹⁵Tc, ¹¹¹In, ⁶²Cu, ⁶⁰Cu, ⁶⁴Cu, ⁶⁷Ga, ⁶⁸Ga, ⁸⁶Y.

Chelators for technetium, copper and gallium isotopes are selected from diaminedithiols, monoamine-monoamidedithiols, triamide-monothiols, monoamine-diamide-monothiols, diaminedioximes, and hydrazines. The chelators are generally tetradentate with donor atoms selected from nitrogen, oxygen and sulfur. Typical reagents are comprised of chelators having amine nitrogen and thiol sulfur donor atoms and hydrazine bonding units. The thiol sulfur atoms and the hydrazines may bear a protecting group which can be displaced either prior to using the reagent to synthesize a radiopharmaceutical or more often in situ during the synthesis of the radiopharmaceutical.

Exemplary thiol protecting groups include those listed in Greene and Wuts, Protective Groups in Organic Synthesis, John Wiley & Sons, New York (1991). Any thiol protecting group known in the art may be used. Examples of thiol protecting groups include, but are not limited to, the following: acetamidomethyl, benzamidomethyl, 1-ethoxyethyl, benzoyl, and triphenylmethyl.

Exemplary protecting groups for hydrazine bonding units are hydrazones which can be aldehyde or ketone hydrazones having substituents selected from hydrogen, alkyl, aryl and heterocycle. Examples of hydrazones are described in U.S. Pat. No. 5,750,088.

The hydrazine bonding unit when bound to a metal radionuclide is termed a hydrazido, or diazenido group and serves as the point of attachment of the radionuclide to the remainder of the radiopharmaceutical. A diazenido group can be either terminal (only one atom of the group is bound to the radionuclide) or chelating. In order to have a chelating diazenido group at least one other atom of the group must also be bound to the radionuclide. The atoms bound to the metal are termed donor atoms.

Chelators for ¹¹¹In and ⁸⁶Y are selected from cyclic and acyclic polyaminocarboxylates such as DTPA, DOTA, DO3A, 2-benzyl-DOTA, alpha-(2-phenethyl) 1,4,7,10-tetraazazcyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyphenyl)-2,2′:6′,2″-terpyridine. Procedures for synthesizing these chelators that are not commercially available can be found in J. Chem. Soc. Perkin Trans., 1992, 1, 1175; Bioconjugate Chem., 1991, 2, 187; J. Nucl. Med., 1990, 31, 473; U.S. Pat. No. 5,064,956, and U.S. Pat. No. 4,859,777.

The coordination sphere of metal ion includes all the ligands or groups bound to the metal. For a transition metal radionuclide to be stable it typically has a coordination number (number of donor atoms) comprised of an integer greater than or equal to 4 and less than or equal to 8; that is there are 4 to 8 atoms bound to the metal and it is said to have a complete coordination sphere. The requisite coordination number for a stable radionuclide complex is determined by the identity of the radionuclide, its oxidation state, and the type of donor atoms. If the chelator or bonding unit does not provide all of the atoms necessary to stabilize the metal radionuclide by completing its coordination sphere, the coordination sphere is completed by donor atoms from other ligands, termed ancillary or co-ligands, which can also be either terminal or chelating.

A large number of ligands can serve as ancillary or co-ligands, the choice of which is determined by a variety of considerations such as the ease of synthesis of the radiopharmaceutical, the chemical and physical properties of the ancillary ligand, the rate of formation, the yield, and the number of isomeric forms of the resulting radiopharmaceuticals, the ability to administer said ancillary or co-ligand to a patient without adverse physiological consequences to said patient, and the compatibility of the ligand in a lyophilized kit formulation. The charge and lipophilicity of the ancillary ligand will effect the charge and lipophilicity of the radiopharmaceuticals. For example, the use of 4,5-dihydroxy-1,3-benzene disulfonate results in radiopharmaceuticals with an additional two anionic groups because the sulfonate groups will be anionic under physiological conditions. The use of N-alkyl substituted 3,4-hydroxypyridinones results in radiopharmaceuticals with varying degrees of lipophilicity depending on the size of the alkyl substituents.

Preferred technetium radiopharmaceuticals of the present disclosure are comprised of a hydrazido or diazenido bonding unit and an ancillary ligand, A_{L1}, or a bonding unit and two types of ancillary ligands A_{L1} and A_{L2}, or a tetradentate chelator comprised of two nitrogen and two sulfur atoms. Ancillary ligands A_{L1} are comprised of two or more hard donor atoms such as oxygen and amine nitrogen (sp³ hybridized). The donor atoms occupy at least two of the sites in the coordination sphere of the radionuclide metal; the ancillary ligand A_{L1} serves as one of the three ligands in the ternary ligand system. Examples of ancillary ligands A_{L1} include but are not limited to dioxygen ligands and functionalized aminocarboxylates. A large number of such ligands are available from commercial sources.

Ancillary dioxygen ligands include ligands that coordinate to the metal ion through at least two oxygen donor atoms. Examples include but are not limited to: glucoheptonate, gluconate, 2-hydroxyisobutyrate, lactate, tartrate, mannitol, glucarate, maltol, Kojic acid, 2,2-bis(hydroxymethyl)propionic acid, 4,5-dihydroxy-1,3-benzene disulfonate, or substituted or unsubstituted 1,2- or 3,4-hydroxypyridinones. (The names for the ligands in these examples refer to either the protonated or non-protonated forms of the ligands.)

Functionalized aminocarboxylates include ligands that have a combination of amine nitrogen and oxygen donor atoms. Examples include but are not limited to: iminodiacetic acid, 2,3-diaminopropionic acid, nitrilotriacetic acid, N,N′-ethylenediamine diacetic acid, N,N,N′-ethylenediamine triacetic acid, hydroxyethylethylenediamine triacetic acid, and N,N′-ethylenediamine bis-hydroxyphenylglycine. (The names for the ligands in these examples refer to either the protonated or non-protonated forms of the ligands.)

A series of functionalized aminocarboxylates are disclosed in U.S. Pat. No. 5,350,837 that result in improved rates of formation of technetium labeled hydrazino modified proteins. We have determined that certain of these aminocarboxylates result in improved yields of the radiopharmaceuticals of the present disclosure. The preferred ancillary ligands A_{L1} include functionalized aminocarboxylates that are derivatives of glycine; the most preferred is tricine (tris(hydroxymethyl)methylglycine).

The most preferred technetium diagnostic agent of the present disclosure comprised a hydrazido or diazenido bonding unit and two types of ancillary ligand designated A_{L1} and A_{L2}, or a diaminedithiol chelator. The second type of ancillary ligands A_{L2} comprise one or more soft donor atoms selected from phosphine phosphorus, arsine arsenic, imine nitrogen (sp² hybridized), sulfur (sp² hybridized) and carbon (sp hybridized); atoms which have p-acid character. Ligands A_{L2} can be monodentate, bidentate or tridentate; the denticity is defined by the number of donor atoms in the ligand. One of the two donor atoms in a bidentate ligand and one of the three donor atoms in a tridentate ligand must be a soft donor atom. U.S. Pat. No. 5,744,120 and U.S. Pat. No. 5,739,789 disclose radiopharmaceuticals comprising one or more ancillary or co-ligands A_{L2} that are more stable compared to radiopharmaceuticals that do not comprise one or more ancillary ligands, A_{L2}; that is, they have a minimal number of isomeric forms, the relative ratios of which do not change significantly with time, and that remain substantially intact upon dilution.

The ligands A_{L2} that comprise phosphine or arsine donor atoms are trisubstituted phosphines, trisubstituted arsines, tetrasubstituted diphosphines and tetrasubstituted diarsines. The ligands A_{L2} that comprise imine nitrogen are unsaturated or aromatic nitrogen-containing, 5 or 6-membered heterocycles. The ligands that comprise sulfur (sp² hybridized) donor atoms are thiocarbonyls, and comprise the moiety C═S. The ligands comprising carbon (sp hybridized) donor atoms are isonitriles, comprising the moiety CNR, where R is an organic radical. A large number of such ligands are available from commercial sources. Isonitriles can be synthesized as described in U.S. Pat. No. 4,452,774 and U.S. Pat. No. 4,988,827.

Preferred ancillary ligands A_{L2} are trisubstituted phosphines and unsaturated or aromatic 5 or 6 membered heterocycles. The most preferred ancillary ligands A_{L2} are trisubstituted phosphines and unsaturated 5-membered heterocycles.

The ancillary ligands A_{L2} may be substituted with alkyl, aryl, alkoxy, heterocyclyl, arylalkyl, alkylaryl and arylalkylaryl groups and may or may not bear functional groups comprising heteroatoms such as oxygen, nitrogen, phosphorus or sulfur. Examples of such functional groups include but are not limited to: hydroxyl, carboxyl, carboxamide, nitro, ether, ketone, amino, ammonium, sulfonate, sulfonamide, phosphonate, and phosphonamide. The functional groups may be chosen to alter the lipophilicity and water solubility of the ligands that may affect the biological properties of the radiopharmaceuticals, such as altering the distribution into non-target tissues, cells or fluids, and the mechanism and rate of elimination from the body.

Chelators for magnetic resonance imaging contrast agents are selected to form stable complexes with paramagnetic metal ions, such as Gd(III), Dy(III), Fe(III), and Mn(II), are selected from cyclic and acyclic polyaminocarboxylates such as DTPA, DOTA, DO3A, 2-benzyl-DOTA, alpha-(2-phenethyl)1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyphenyl)-2,2′:6′,2″-terpyridine.

There are two key features of the diagnostic agents of the present disclosure that determine their efficacy: MMP selectivity and the rate of clearance from the blood. Preferred diagnostic agents of the present disclosure comprise targeting moieties that exhibit selectivity for MMP-1, MMP-2, MMP-3, MMP-9, or MMP-14 alone or in combination over the other MMPs. Most preferred are MMP substrates that exhibit selectivity for MMP-2, MMP-9, or MMP-14 alone or in combination over the other MMPs.

The rate of clearance from the blood is of particular importance for cardiac imaging procedures, since the cardiac blood pool is large compared to the disease foci that one desires to image. For an effective cardiac imaging agent, the target to background ratios (disease foci-to-blood and disease foci-to-muscle) are typically greater or equal to about 1.5, typically greater or equal to about 2.0, and more typically even greater. Preferred pharmaceuticals of the present disclosure have blood clearance rates that result in less than about 10% i.d./g at 2 hours post-injection, measured in a mouse model, or less than about 0.5% i.d./g at 2 hours post-injection, measured in a dog model. Most preferred diagnostic agents of the present disclosure have blood clearance rates that result in less than about 3% i.d./g at 2 hours post-injection, measured in a mouse model, or less than about 0.05% i.d./g at 2 hours post-injection, measured in a dog model.

The diagnostic agents of the disclosure containing technetium further comprising hydrazido or diazenido bonding units can be easily prepared by admixing a salt of a radionuclide, a reagent of the present disclosure, an ancillary ligand A_{L1}, an ancillary ligand A_{L2}, and a reducing agent, in an aqueous solution at temperatures from about 0° C. to about 100° C. The diagnostic agents of the disclosure containing technetium comprising a tetradentate chelator having two nitrogen and two sulfur atoms can be easily prepared by admixing a salt of a radionuclide, a reagent of the present disclosure, and a reducing agent, in an aqueous solution at temperatures from about 0° C. to about 100° C.

When the bonding unit in the reagent of the present disclosure is present as a hydrazone group, then it first typically converted to a hydrazine, which may or may not be protonated, prior to complexation with the metal radionuclide. The conversion of the hydrazone group to the hydrazine can occur either prior to reaction with the radionuclide, in which case the radionuclide and the ancillary or co-ligand or ligands are combined not with the reagent but with a hydrolyzed form of the reagent bearing the chelator or bonding unit, or in the presence of the radionuclide in which case the reagent itself is combined with the radionuclide and the ancillary or co-ligand or ligands. In the latter case, the pH of the reaction mixture is usually neutral or acidic.

Alternatively, the diagnostic agents of the present disclosure comprising hydrazido or diazenido bonding unit may be prepared by first admixing a salt of a radionuclide, an ancillary ligand A_{L1}, and a reducing agent in an aqueous solution at temperatures from about 0° C. to about 100° C. to form an intermediate radionuclide complex with the ancillary ligand A_{L1} then adding a reagent of the present disclosure and an ancillary ligand A_{L2} and reacting further at temperatures from about 0° C. to about 100° C.

Alternatively, the diagnostic agents of the present disclosure comprising a hydrazido or diazenido bonding unit may be prepared by first admixing a salt of a radionuclide, an ancillary ligand A_{L1}, a reagent of the present disclosure, and a reducing agent in an aqueous solution at temperatures from about 0° C. to about 100° C. to form an intermediate radionuclide complex, and then adding an ancillary ligand A_{L2} and reacting further at temperatures about 0° C. to about 100° C.

The technetium radionuclides are typically in the chemical form of pertechnetate or perrhenate and a pharmaceutically acceptable cation. The pertechnetate salt form is typically sodium pertechnetate such as obtained from commercial ^{99m}Tc generators. The amount of pertechnetate used to prepare the radiopharmaceuticals of the present disclosure can range from about 0.1 mCi to about 1 Ci, or more typically from about 1 to about 200 mCi.

The amount of the reagent of the present disclosure used to prepare the technetium diagnostic agent of the present disclosure may range from about 0.01 μg to about 10 mg, or more typically from about 0.5 μg to about 200 μg. The amount used will be dictated by the amounts of the other reactants and the identity of the radiopharmaceuticals of the present disclosure to be prepared.

The amounts of the ancillary ligands A_{L1} used may range from about 0.1 mg to about 1 g, or more typically from about 1 mg to about 100 mg. The exact amount for a particular radiopharmaceutical is a function of identity of the radiopharmaceuticals of the present disclosure to be prepared, the procedure used and the amounts and identities of the other reactants. Too large an amount of A_{L1} will result in the formation of by-products comprised of technetium labeled A_{L1} without a biologically active molecule or by-products comprised of technetium labeled biologically active molecules with the ancillary ligand A_{L1} but without the ancillary ligand A_{L2}. Too small an amount of A_{L1} will result in other by-products such as technetium labeled biologically active molecules with the ancillary ligand A_{L2} but without the ancillary ligand A_{L1}, or reduced hydrolyzed technetium, or technetium colloid.

The amounts of the ancillary ligands A_{L2} used may range from about 0.001 mg to about 1 g, or more typically from about 0.01 mg to about 10 mg. The exact amount for a particular radiopharmaceutical is a function of the identity of the radiopharmaceuticals of the present disclosure to be prepared, the procedure used and the amounts and identities of the other reactants. Too large an amount of A_{L2} will result in the formation of by-products comprised of technetium labeled A_{L2} without a biologically active molecule or by-products comprised of technetium labeled biologically active molecules with the ancillary ligand A_{L2} but without the ancillary ligand A_{L1}.

In another embodiment of the current disclosure, a scintigraphic image of a radiolabeled MMP substrate-containing diagonistic agent would be acquired at the same time as a scintigraphic image of a radiolabeled cardiac perfusion imaging agent. This simultaneous dual isotope imaging would be done by utilizing radioisotopes of the MMP substrate and perfusion imaging agents that had spectrally separable gamma emission energies. For example, a ^{99m}Tc cardiac perfusion imaging agent (such as ^{99m}Tc-Sestamibi) or T1201 (as Thallous Chloride), and an ¹¹¹In-labeled MM substrate compound would be imaged simultaneously with a standard gamma camera. This is possible because the ^{99m}Tc gamma energy of about 140 KeV or the T1201 gamma energy of about 80 KeV are easily separable from the ¹¹¹In gamma energies of about 160 KeV and 250 KeV. This simultaneous imaging of cardiac perfusion and extracellular matrix degradation (as evidenced by localization of the diagnostic agent containing MMP substrate) is extremely useful for improved anatomic assessment of the location of diagnostic agent distribution in the heart based on the comparison to the perfusion distribution seen on the ^{99m}Tc-Sestamibi or T1201 image. In addition, the simultaneous imaging of perfusion and extracellular matrix degradation allows a more complete assessment of the underlying cardiac disease, both in terms of blood flow alterations and biochemical changes, in a single imaging session on a patient.

The simultaneous dual-isotope imaging of cardiac perfusion and extracellular matrix degradation allows the localization of sites of vulnerable plaque and cardiac perfusion to be visualized during one imaging session. In addition, the simultaneous imaging of tissue changes associated with congestive heart failure (from the diagnostic agent containing the MMP substrate) and coronary artery disease (from the perfusion imaging agent) is extremely useful in characterizing the underlying causes of congestive heart failure.

The simultaneous imaging of different radioisotopically-labeled radiopharmaceuticals in patients has been reported. For example, Antunes, et al., Am J. Cardiol., 1992, 70, 426-431, have demonstrated that it is possible to image myocardial infarction with an ¹¹¹In-antimyosin antibody along with the imaging of cardiac perfusion with T1201. However, the dual isotope imaging of the present disclosure is new, because it is the first reported approach to the simultaneous, dual isotope imaging of a radiolabeled diagnostic agent containing the MMP substrate and a cardiac perfusion imaging compound. The combination of the scintigraphic imaging using diagnostic agent containing the MMP substrate scintigraphic imaging with perfusion imaging provides the imaging physician with an extraordinary amount of clinical information regarding ischemic coronary artery disease or congestive heart failure in one imaging session.

Suitable reducing agents for the synthesis of the diagnostic agent of the present disclosure include stannous salts, dithionite or bisulfite salts, borohydride salts, ascorbic acid, cysteine, phosphines, and cuprous or ferrous salts and formamidinesulfinic acid, wherein the salts are of any pharmaceutically acceptable form. A specific reducing agent is a stannous salt. Other reducing agents are described in U.S. Pat. No. 5,662,882. The amount of a reducing agent used can range from about 0.001 mg to about 10 mg, or more typically from about 0.005 mg to about 1 mg.

The indium, copper, gallium, and yttrium diagnostic agents of the present disclosure can be easily prepared by admixing a salt of a radionuclide and a reagent of the present disclosure, in an aqueous solution at temperatures from about 0° C. to about 100° C. These radionuclides are typically obtained as a dilute aqueous solution in a mineral acid, such as hydrochloric, nitric or sulfuric acid. The radionuclides are combined with from one to about one thousand equivalents of the reagents of the present disclosure dissolved in aqueous solution. A buffer is typically used to maintain the pH of the reaction mixture from about 3 to about 10.

The gadolinium, dysprosium, iron and manganese diagnostic agents of the present disclosure can be easily prepared by admixing a salt of the paramagnetic metal ion and a reagent of the present disclosure, in an aqueous solution at temperatures from about 0° C. to about 100° C. These paramagnetic metal ions are typically obtained as a dilute aqueous solution in a mineral acid, such as hydrochloric, nitric or sulfuric acid. The paramagnetic metal ions are combined with from one to about one thousand equivalents of the reagents of the present disclosure dissolved in aqueous solution. A buffer is typically used to maintain the pH of the reaction mixture from about 3 to about 10.

The total time of preparation will vary depending on the identity of the metal ion, the identities and amounts of the reactants and the procedure used for the preparation. The preparations may be complete, resulting in greater than about 80% yield of the radiopharmaceutical, in about 1 minute or may require more time. If higher purity metallopharmaceuticals are needed or desired, the products can be purified by any of a number of techniques well known to those skilled in the art such as liquid chromatography, solid phase extraction, solvent extraction, dialysis or ultrafiltration.

The diagnostic radiopharmaceuticals are administered by intravenous injection, usually in saline solution, at a dose of about 1 to about 100 mCi per 70 kg body weight, or typically at a dose of about 5 to about 50 mCi. Imaging is performed using known procedures.

The diagnostic agents of the disclosure containing a magnetic resonance imaging contrast component may be used in a similar manner as other MRI agents as described in U.S. Pat. No. 5,155,215; U.S. Pat. No. 5,087,440; Magn. Reson. Med., 1986, 3, 808; Radiology, 1988, 166, 835; and Radiology, 1988, 166, 693. Generally, sterile aqueous solutions of the contrast agents are administered to a patient intravenously in dosages ranging from about 0.01 to about 1.0 mmoles per kg body weight.

For use as X-ray contrast agents, the diagnostic agents of the present disclosure should generally have a heavy atom concentration of about 1 mM to about M, typically about 0.1 M to about 2 M. Dosages, administered by intravenous injection, will typically range from about 0.5 mmol/kg to about 1.5 mmol/kg, typically about 0.8 mmol/kg to about 1.2 mmol/kg. Imaging is performed using known techniques, typically X-ray computed tomography.

The diagnostic agents of the disclosure containing ultrasound contrast components are administered by intravenous injection in an amount of about 10 to about 30 μL of the echogenic gas per kg body weight or by infusion at a rate of about 3 μL/kg/min. Imaging may be performed using known techniques of sonography.

Other features of the disclosure will become apparent in the course of the following descriptions of exemplary embodiments which are given for illustration of the disclosure and are not intended to be limiting thereof. The present disclosure will now be illustrated by reference to the following specific, non-limiting examples. Those skilled in the art of organic synthesis may be aware of still other synthetic routes to the disclosure compounds. The reagents and intermediates used herein are either commercially available or prepared according to standard literature procedures, unless otherwise described.

### EXAMPLE 1

### Synthesis of (1S)-1-[(2S)-2-((2S)-2-{(2S)-2-[(2S)-2-(2-{(2S)-2-[((2S)-1-{6-[(6-Hydrazino(3-pyridyl))carbonylamino]hexanoyl}pyrrolidin-2-yl)carbonylamino]-4-methylpentanoylamino}acetylamino)-4-phenylbutanoylamino]-5-aminopentanoylamino}-4-methylpentanoylamino)-4-carboxybutanoylamino]propane-1,3-dicarboxylic Acid Trifluoroacetic Acid Salt

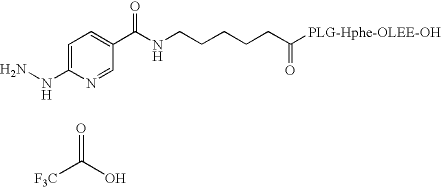

### Part A—Preparation of Fmoc-Ahx-PLG-Hphe-OLEE-Wang Resin

Fmoc-Glu(Ot-Bu)-Wang resin (2.000 g, substitution level=0.9 mmol/g) was placed in a 50 ml Advanced ChemTech reaction vessel. The resin was swollen by washing with N,N-dimethylformamide (2×20 mL), and the following steps were performed: (Step 1) The Fmoc group was removed using 20% piperidine in N,N-dimethylformamide (20 mL) for 30 minutes. (Step 2) The resin was washed thoroughly (20 mL volumes) with N,N-dimethylformamide (3×), dichloromethane (3×), methanol (3×), dichloromethane (3×), N,N-dimethylformamide (3×). (Step 3) Fmoc-Glu(Ot-Bu)-OH (3.064 g, 7.2 mmol), HOBt (1.102 g, 7.2 mmol), HBTU (2.731 g, 7.2 mmol) in 10 mL of N,N-dimethylformamide and 3 mL of diisopropylethylamine were added to the resin and the reaction was allowed to proceed for 4 hours (Step 4) The resin was washed thoroughly (20 ml volumes) with N,N-dimethylformamide (3×), dichloromethane (3×), methanol (3×), dichloromethane (3×), N,N-dimethylformamide (3×). (Step 5) The coupling reaction was found to be more than 95% complete as assessed by the semi-quantitative ninhydrin assay and quantitative picric assay or fulvene-piperidine assay. Steps 1-5 were repeated until the sequence G-Hphe-OLEE had been attained. Coupling of the remaining amino acids required double coupling in 40% DMSO in N,N-dimethylformamide in order to achieve high coupling yields.

### Part B—Preparation of Hynic-Ahx-PLG-Hphe-OLEE-OH

Half of the peptide-resin prepared in Part A, above, was treated with 20% piperidine in N,N-dimethylformamide (20 mL) for 30 minutes. The resin was washed thoroughly (20 mL volumes) with N,N-dimethylformamide (3×), dichloromethane (3×), methanol (3×), dichloromethane (3×), N,N-dimethylformamide (3×). Boc-Hynic-OH (0.912 g, 3.6 mmol), HOBt (0.551 g, 3.6 mmol), HBTU (1.366 g, 3.6 mmol) in 10 mL of N,N-dimethylformamide and 3 ml of diisopropylethylamine were added and the reaction was allowed to proceed for 4 hours. The resin was washed thoroughly (20 mL volumes) with N,N-dimethylformamide (3×), dichloromethane (3×), methanol (3×), dichloromethane (3×), N,N-dimethylformamide (3×). The coupling reaction was found to be complete as assessed by the semi-quantitative ninhydrin assay and quantitative picric assay or fulvene-piperidine assay.

Half of the above resin was stirred with 9.00 mL of trifluoroacetic acid, 0.236 mL of H₂O and 0.236 mL of TIS for 2 hours. The resin was removed by filtering through a sintered glass funnel and washed thoroughly with trifluoroacetic acid (2×2 mL). The filtrate was concentrated to 2 mL and diluted with ether (10 mL). The resulting precipitate was collected by filtration, washed with ether (3×5 mL) and dried to give the title compound as a colorless solid (0.673 g). Purification was accomplished by reversed-phase HPLC with a Phenomenex Luna C18(2) column (41.2×250 mm) and a 0.50%/minute gradient of 18 to 36% acetonitrile containing 0.1% trifluoroacetic acid at a flow rate of 80 mL/min, followed by purification on a Phenomenex Jupiter C18 column (21.2×250 mm) using a 0.67%/minute gradient of 18 to 36% acetonitrile containing 0.1 M NH₄OAc (pH 7) at a flow rate of 20 mL/min. Lyophilization of the product fraction gave the title compound as a colorless solid (0.040 g, overall yield 7.5%, HPLC purity 100%). MS: m/e 591.0 [2M+H] (100%), 1180.9 [M+H] (20%); FT-MS: Calculated for C56H85N13O15 [M+2H]: 590.8217, Found: 590.8214. Chiral analysis for L-leucine: 99.8%.

### EXAMPLE 2

### Synthesis of 1-(2-{2-[2-(2-{2-[2-({1-[6-(2-{2-[(6-{[(1E)-1-Aza-2-(2-sulfophenyl)vinyl]amino}(3-pyridyl))carbonylamino](2R)-3-phenylpropanoylamino}-(2R)-3-phenylpropanoylamino)hexanoyl](2S)pyrrolidin-2-yl}carbonylamino)(2S)-4-methylpentanoylamino]acetylamino}(2S)-4-phenylbutanoylamino)(2S)-5-aminopentanoylamino](2S)-4-methylpentanoylamino}(2S)-4-carboxybutanoylamino)(1S)propane-1,3-dicarboxylic Acid Trifluoroacetic Acid Salt

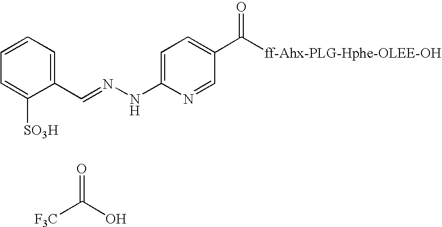

The peptide-resin from Example 1, Part A (0.500 g, substitution level=0.45 mmol/g) was placed in a 50 mL reaction vessel. The resin was swollen by washing with N,N-dimethylformamide (2×20 mL), and the following steps were performed: (Step 1) The Fmoc group was removed using 20% piperidine in N,N-dimethylformamide (20 mL) for 30 minutes. (Step 2) The resin was washed thoroughly (20 mL volumes) with N,N-dimethylformamide (3×), dichloromethane (3×), methanol (3×), dichloromethane (3×), N,N-dimethylformamide (3×). (Step 3) Fmoc-f-OH (0.349 g, 0.9 mmol), HOBt (0.138 g, 0.9 mmol), HBTU (0.341 g, 0.9 mmol) in 10 mL of 40:60 DMSO:N,N-dimethylformamide and 3 mL of diisopropylethylamine were added to the resin and the reaction was allowed to proceed for 10 hours. (Step 4) The resin was washed thoroughly (20 mL volumes) with N,N-dimethylformamide (3×), dichloromethane (3×), methanol (3×), dichloromethane (3×), N,N-dimethylformamide (3×). (Step 5) Fmoc-f-OH (0.349 g, 0.9 mmol), HOBt (0.138 g, 0.9 mmol), HBTU (0.341 g, 0.9 mmol) in 10 ml of 40% DMSO in N,N-dimethylformamide and 3 ml of diisopropylethylamine were added to the resin and the reaction allowed to proceed for 4 hours. (Step 6) The resin was washed thoroughly (20 mL volumes) with N,N-dimethylformamide (3×), dichloromethane (3×), methanol (3×), dichloromethane (3×), N,N-dimethylformamide (3×). (Step 7) The coupling reaction was found to be complete as assessed by the semi-quantitative ninhydrin assay and quantitative picric assay or fulvene-piperidine assay. Steps 1-7 were repeated for the addition of the second D-phenylalanine.

The resin was treated with 20% piperidine in N,N-dimethylformamide (20 mL) for 30 minutes, and washed thoroughly (20 mL volumes) with N,N-dimethylformamide (3×), dichloromethane (3×), methanol (3×), dichloromethane (3×), N,N-dimethylformamide (3×). Sodium 2-[(1E)-2-aza-2-({5-[(2,5-dioxopyrrolidinyl)oxycarbonyl](2-pyridyl)}amino)vinyl]benzenesulfonate (0.396 g, 0.9 mmol) and HOAt (0.122 g, 0.9 mmol) in 10 ml of 40:60 DMSO:N,N-dimethylformamide and 3 mL of diisopropylethylamine were added to the resin and the reaction was allowed to proceed for 18 hours. The resin was washed thoroughly (20 mL volumes) with N,N-dimethylformamide (3×), dichloromethane (3×), methanol (3×), dichloromethane (3×), N,N-dimethylformamide (3×). The above coupling procedure was repeated three more times until the reaction was determined to be complete as assessed by LC/MS of a small portion of cleaved peptide. During the last coupling, chaotropic salt KSCN (0.776 g, 0.4 M in 20 ml solution) was added to the coupling solution as a catalyst.

Half of the above resin was stirred with 2 mL of 95% trifluoroacetic acid, 2.5% H₂O and 2.5% TIS for 2 hours. The resin was removed by filtration through a sintered glass funnel and washed thoroughly with trifluoroacetic acid (2×2 mL). The filtrate was concentrated to 2 mL and diluted with ether (10 mL). The resulting precipitate were collected by filtration, washed with ether (3×5 mL) and dried to give the title compound as a colorless solid (0.126 g). Purification was accomplished by using reversed-phase HPLC using a Phenomenex Jupiter C18 column (41.2×250 mm) and a 0.83%/minute gradient of 22.5 to 45% acetonitrile containing 0.1 M NH₄OAc (pH 7) at a flow rate of 80 mL/min, followed by purification on a Phenomenex Jupiter C18 column (21.2×250 mm) and a 0.17%/minute gradient of 31.5 to 36% acetonitrile containing 0.1% trifluoroacetic acid at a flow rate of 20 mL/min. Lyophilization of the product fraction gave the title compound as a colorless solid (8.0 mg, overall yield 4.4%, HPLC purity 100%). MS: m/e 822.0 [2M+H] (100%), 1643.6 [M+H] (70%); FT-MS: Calculated for C81H107N15O20S [M+2H]: 821.8842, Found: 821.8831.

### EXAMPLE 3

### Synthesis of Synthesis of 1-(2-{2-[2-(2-{2-[2-({1-[6-(2-[2-{2-[(6-{[(1E)-1-Aza-2-(2-sulfophenyl)vinyl]amino}(3-pyridyl))carbonylamino](2R)-3-phenylpropanoylamino}(2R)-3-phenylpropanoylamino](2R)-3-phenylpropanoylamino)hexanoyl](2S)pyrrolidin-2-yl}carbonylamino)(2S)-4-methylpentanoylamino]acetylamino}(2S)-4-phenylbutanoylamino)(2S)-5-aminopentanoylamino](2S)-4-methylpentanoylamino}(2S)-4-carboxybutanoylamino)(1S)propane-1,3-dicarboxylic Acid Trifluoroacetic Acid Salt

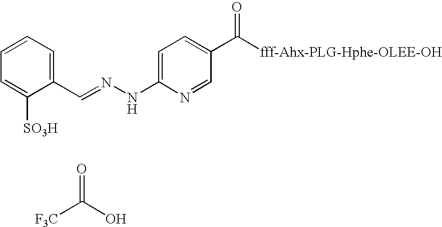

The HPLC purification of Example 2, above, also produced the tri-D-phenylalanine peptide. Lyophilization of the product fraction gave the title compound as a colorless solid (3.0 mg, overall yield 1.4%, HPLC purity 100%). MS: m/e 895.7 [2M+H] (100%), 1790.7 [M+H] (30%); FT-MS: Calculated for C90H116N16O21S [M+2H]: 895.4184, Found: 895.4172.

### EXAMPLE 4

### Synthesis of (1S)-1-[(2S)-2-((2S)-2-{(2S)-2-[(2S)-2-(2-{(2S)-2-[((2S)-1-{6-[(7-Methoxy-2-oxo(2H-chromen-3-yl))carbonylamino]hexanoyl}pyrrolidin-2-yl)carbonylamino]-4-methylpentanoylamino}acetylamino)-4-phenylbutanoylamino]-5-aminopentanoylamino}-4-methylpentanoylamino)-4-carboxybutanoylamino]propane-1,3-dicarboxylic Acid

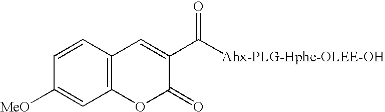

The peptide-resin of Example 1, Part A (0.2 g, substitution level=0.45 mmol/g) was placed in a 50 mL reaction vessel. The resin was swollen by washing with N,N-dimethylformamide (2×20 mL), and the Fmoc group was removed using 20% piperidine in N,N-dimethylformamide (20 mL) for 30 minutes. The resin was washed thoroughly (20 mL volumes) with N,N-dimethylformamide (3×), dichloromethane (3×), methanol (3×), dichloromethane (3×), N,N-dimethylformamide (3×). 7-Methoxycoumarin-3-carboxylix acid (0.04 g, 0.18 mmol), HOBt (0.028 g, 0.18 mmol), and HBTU (0.069 g, 0.18 mmol) in 10 mL of 40:60 DMSO:N,N-dimethylformamide, and 3 mL of diisopropylethylamine were added to the resin and the reaction was allowed to proceed for 3 hours. The resin was washed thoroughly (20 mL volumes) with N,N-dimethylformamide (3×), dichloromethane (3×), methanol (3×), dichloromethane (3×), N,N-dimethylformamide (3×). The above coupling procedure was repeated two more times until the reaction was determined to be complete as assessed by the semi-quantitative ninhydrin assay and quantitative picric assay or fulvene-piperidine assay.

The above resin was stirred with 2 mL of 95% trifluoroacetic acid, 2.5% H₂O and 2.5% TIS for 1.5 hours. The resin was removed by filtration through a sintered glass funnel and washed thoroughly with trifluoroacetic acid (2×2 mL). The filtrate was concentrated to 2 mL and diluted with ether (10 mL). The resulting precipitate was collected by filtration, washed with ether (3×5 ml) and dried to give the title compound as an oil (0.145 g). Purification was accomplished by reversed-phase HPLC using a Phenomenex Jupiter C18 column (21.2×250 mm) and a 1%/minute gradient of 18 to 45% acetonitrile containing 0.1 M NH₄OAc (pH 7) at a flow rate of 20 mL/min. Lyophilization of the product fraction gave the title compound as a colorless solid (0.011 g, overall yield 10%, HPLC purity 100%). MS: m/e 624.5 [2M+H] (60%), 1247.6 [M+H] (100%); FT-MS: Calculated for C61H86N10O18 [M+2H]: 624.3134, Found: 624.3127.

### EXAMPLE 5

### Synthesis of 4-(N-{6-[(6-{[(1E)-1-Aza-2-(2-sulfophenyl)vinyl]amino}(3-pyridyl))carbonylamino]hexyl}carbamoyl)(4S)-4-[(2S)-2-((2S)-2-{(2S)-2-[(2S)-2-(2-{(2S)-2-[((2S)-1-acetylpyrrolidin-2-yl)carbonylamino]-4-methylpentanoylamino}-acetylamino)-4-phenylbutanoylamino]-5-aminopentanoylamino}-4-methylpentanoylamino)-4-carboxybutanoylamino]butanoic Acid Bis-Ammonium Salt

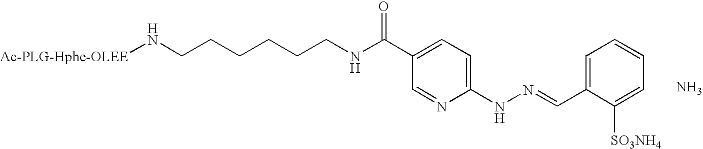

### Part A—Preparation of Ac-PLG-Hphe-OLEE-hexamethylene-NH-Trityl Resin

1,6-Diaminohexane trityl resin (2.000 g, substitution level=0.81 mmol/g) was placed in a 50 mL Advanced ChemTech reaction vessel. The following steps were performed: (Step 1) The resin was washed thoroughly (20 mL volumes) with dichloromethane (3×) and N,N-dimethylformamide (3×). (Step 2) Fmoc-Glu(t-Bu)-OH (2.76 g, 6.5 mmol), HOBt (0.99 g, 6.5 mmol), and HBTU (2.46 g, 6.5 mmol) in N,N-dimethylformamide (15 mL) and diisopropylethylamine (3 mL) were added to the resin and the reaction was allowed to proceed for 4 hours. (Step 3) The resin was washed thoroughly (20 mL volumes) with N,N-dimethylformamide (3×), dichloromethane (3×), methanol (3×), dichloromethane (3×), and N,N-dimethylformamide (3×). (Step 4) 20% Piperidine in N,N-dimethylformamide (20 mL) was added to the resin and allowed to react for 30 minutes. (Step 5) The resin was washed thoroughly (20 mL volumes) with N,N-dimethylformamide (3×), dichloromethane (3×), methanol (3×), dichloromethane (3×), and N,N-dimethylformamide (3×). (Step 6) Analysis of the resin by the Fulvene-Piperidine assay indicated a loading factor of 0.33 mmol/g. Steps 2-6 were repeated until the desired amino acid sequence was attained. All coupling steps proceeded in quantitative yield. Double coupling was required with Fmoc-Orn(Ot-Bu)-OH. The resin was treated with a solution of acetic anhydride (0.666 mL, 6.6 mmol) and diisopropylethylamine (1.4 mL, 7.92 mmol) in N,N-dimethylformamide (20 mL) for 2.0 hours, washed thoroughly (20 mL volumes) with N,N-dimethylformamide (3×), dichloromethane (3×), methanol (3×), and dichloromethane (3×), and dried under vacuum.

### Part B—Preparation of Ac-PLG-Hphe-OLEE-Hexamethylene-NH₂

The peptide-resin from part A (1.0 g) was placed in a 30 mL fritted glass funnel and washed with dichloromethane (2×25 mL). The peptide-resin was treated with a solution of 5:1:94 trifluoroacetic acid:Et3SiH:dichloromethane (10 mL) for 2 minutes. The solution was filtered, by the application of pressure, directly into a solution of 10% pyridine in methanol (2 mL). The cleavage step was repeated five times. The combined filtrates were concentrated to remove dichloromethane and methanol, providing a colorless oily solid. Trituration with water (40 mL) gave a colorless dry solid, which was collected by filtration. This crude product was purified by HPLC on a Phenomenex Jupiter C18 column (21.2×250 mm) using a 0.9%/minute gradient of 31.5 to 67.5% acetonitrile containing 100 mM ammonium acetate at a flow rate of 20 mL/min. The main product peak eluting at 28.5 minutes was lyophilized to give the title compound as a colorless solid (61.3 mg, 19.6%; HPLC purity, 100%). MS: m/e 537.0 [(M−Boc-2(t-Bu)+2H](100%), 565.2 [(M−Boc-(t-Bu))+2H](45%), 593.2 [(M−Boc)+2H](30%), 654.2 [(M+Na)+2H](65%), 1285.2 [M+H](95%), 1307.1 [M+Na](25%).

### Part C—Preparation of 4-(N-{6-[(6-{[(1E)-1-Aza-2-(2-sulfophenyl)vinyl]amino}(3-pyridyl))carbonylamino]hexyl}carbamoyl)(4S)-4-[(2S)-2-((2S)-2-{(2S)-2-[(2S)-2-(2-{(2S)-2-[((2S)-1-acetylpyrrolidin-2-yl)carbonylamino]-4-methylpentanoylamino}-acetylamino)-4-phenylbutanoylamino]-5-aminopentanoylamino}-4-methylpentanoylamino)-4-carboxybutanoylamino]butanoic Acid Bis-Ammonium Salt

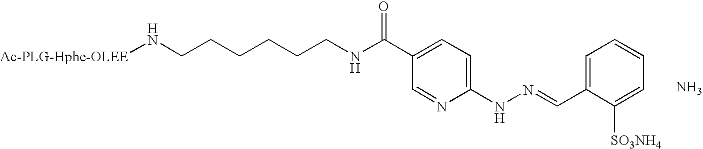

A solution of the product of Part B (20.2 mg, 0.0157 mmol) and diisopropylethylamine (20 μL, 0.0785 mmol) in N,N-dimethylformamide (7 mL) was treated with HOAt (2.15 mg, 0.0157 mmol) and sodium 2-[(1E)-2-aza-2-({5-[(2,5-dioxopyrrolidinyl)oxycarbonyl](2-pyridyl)}amino)vinyl]benzenesulfonate (6.9 mg, 0.0157 mmol). The resulting solution was stirred under nitrogen at ambient temperature. At 5 hours, additional HOAt (2.15 mg, 0.0157 mmol) and sodium 2-[(1E)-2-aza-2-({5-[(2,5-dioxopyrrolidinyl)oxycarbonyl](2-pyridyl)}amino) vinyl]benzenesulfonate (6.9 mg, 0.0157 mmol) were added to the reaction vessel. After stirring a total of 30 hours, N,N-dimethylformamide was removed under reduced pressure to give a green oil, which was triturated with ether (4×2 mL) to yield a powdery green solid. This solid was dissolved in 97:3 trifluoroacetic acid/Et₃SiH and stirred under nitrogen at 40° C. for 30 minutes. The solution was concentrated and the resulting oil was purified by HPLC on a Phenomenex Jupiter C18 column (21.2×250 mm) using a 1.12%/minute gradient of 5.85 to 50.85% acetonitrile containing 100 mM ammonium acetate at a flow rate of 20 mL/min. The main product peak eluting at 29.0 minute was lyophilized to give 12.1 mg (56.0%) of the desired compound as a colorless solid with 99.2% purity by HPLC. MS: m/e 688.8 [M+2H](100%), 1375.8 [M+H](30%); High Resolution MS: Calculated for C65H95N14O17S [M+H]: 1375.6715, Found: 1375.6704.

### EXAMPLE 6

### Synthesis of 2-{(1E)-2-[(5-{N-[2-({4-[((2S)-2-Amino-4-methylpentanoylamino)amino]phenyl}carbonylamino)ethyl]carbamoyl}(2-pyridyl))amino]-2-azavinyl}benzenesulfonic Acid

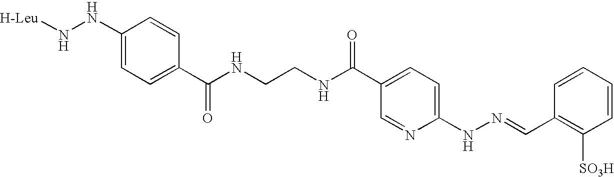

### Part A—Preparation of (4-{[(tert-Butoxy)carbonylamino]amino}phenyl)-N-{2-[(phenylmethoxy)carbonylamino]ethyl}carboxamide

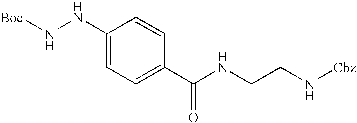

4-[2-(tert-Butoxycarbonyl)hydrazino]benzoic acid (Schwartz, D. A., et al.; Bioconj. Chem., 1991, 2, 333-336) (1.8 g, 7.29 mmol) and diisopropylethylamine (2.0 mL, 11.5 mmol) were dissolved in N,N-dimethylformamide (8 mL) and stirred under nitrogen at room temperature. The solution was treated with PyBroP (3.4 g, 7.29 mmol) and benzyl N-(2-aminoethyl)-carbamate hydrochloride (1.68 g, 7.29 mmol). Additional PyBroP (0.34 g, 0.729 mmol) and benzyl N-(2-aminoethyl)carbamate hydrochloride (0.17 g, 0.729 mmol) were added to the reaction solution at 2 hours. At 6 hours, additional PyBroP (0.68 g, 1.46 mmol) and benzyl N-(2-aminoethyl)carbamate hydrochloride (0.34 g, 1.46 mmol) were added. The solution was stirred a total of 8 hours and was concentrated under vacuum to give a dark amber oil. Crude product was crystallized (ether) to give 2.08 g (66.8%) of the title compound as a colorless solid in 100% purity by LC/MS. MS: m/e 429.3 [M+H](100%).

### Part B—Preparation of 2-[(1E)-2-({5-[N-(2-{[4-({(2S)-2-[(tert-Butoxy)carbonylamino]-4-methylpentanoylamino}amino)phenyl]carbonylamino}ethyl)carbamoyl](2-pyridyl)}amino)-2-azavinyl]benzenesulfonic Acid

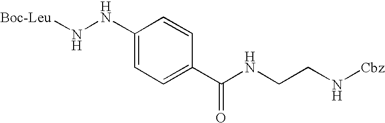

The product of Part A (405.9 mg, 0.95 mmol) was dissolved in 1:1 trifluoroacetic acid/dichloromethane (10 mL) and allowed to react for 10 minutes under nitrogen at ambient temperature. The solution was concentrated to a golden oil, and taken up in N,N-dimethylformamide (3 mL). This solution was added to a solution of Boc-Leucine hydrate (550 mg, 2.19 mmol, NovaBiochem), HBTU (664 mg, 1.75 mmol) and diisopropylethylamine (1.78 mL, 10.22 mmol) in N,N-dimethylformamide, and stirred for 30 minutes at ambient temperature. The N,N-dimethylformamide was removed under vacuum and the resulting amber oil was purified by HPLC on a Phenomenex Jupiter column (41.4×250 mm) using a 0.66%/minute gradient of 29.7 to 49.5% acetonitrile containing 0.1% trifluoroacetic acid at a flow rate of 80 mL/min. The main product peak eluting at 23.0 minutes was lyophilized to give 334.2 mg (62.1%) of the title compound as a colorless solid with 100% purity by HPLC. MS: m/e 442.5 [M+H-Boc](15%); 486.6 [M+H-(t-Bu)](60%); 542.5 [M+H](23%); 1084.1 [2M+H](100%); 1106.1 [2M+Na](25%).

### Part C—Preparation of (2S)-N-({4-[N-(2-Aminoethyl)carbamoyl]phenyl}amino)-2-[(tert-butoxy)carbonylamino]-4-methylpentanamide

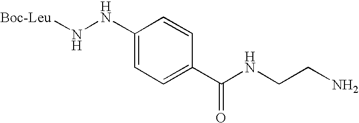

The product of Part B (291.2 mg, 0.538 mmol) was hydrogenolyzed in ethanol (25 mL) over 20% Pd/C (60 mg) at 60 psi for 20 hours. The catalyst was removed by filtration through Celite® and the filtrate was concentrated to give an oily solid. This oil was taken up in 1:1 acetonitrile:water (30 mL) and lyophilized to give the title compound as a colorless flaky solid 231.6 mg (105.7% y) in 87.9% purity by HPLC. MS: m/e 352.5 [M+H-(t-Bu)](42%); 408.6 [M+H](100%); 815.8 [2M+H](25%).

### Part D—Preparation of 2-{(1E)-2-[(5-{N-[2-({4-[((2S)-2-Amino-4-methylpentanoylamino)-amino]phenyl}carbonylamino)ethyl]carbamoyl}(2-pyridyl))amino]-2-azavinyl}benzenesulfonic Acid

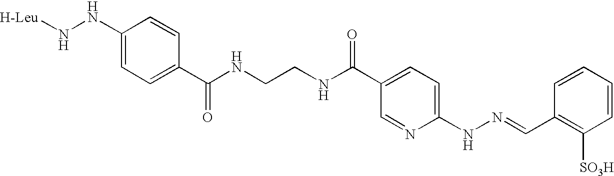

A solution of the product of part C (50.0 mg, 0.123 mmol), Sodium 2-[(1E)-2-aza-2-({5-[(2,5-dioxopyrrolidinyl)oxycarbonyl](2-pyridyl)}amino)vinyl]-benzenesulfonate (54.2 mg, 0.123 mmol), HOAt (16.9 mg, 0.123 mmol) and diisopropylethylamine (120 μL, 0.615 mmol) in N,N-dimethylformamide (5 mL) was stirred under nitrogen at ambient temperature for 3 hours. The N,N-dimethylformamide was removed under vacuum to give an amber oil, which was triturated with 0.1M HCl (2×5 mL) and washed with water (3×5 mL) to give a yellow/brown solid. This solid was dissolved in 1:1 trifluoroacetic acid/dichloromethane (7 mL) and allowed to react for 10 minutes under nitrogen at ambient temperature. The solution was concentrated under reduced pressure and the resulting amber oil was purified by HPLC on a Phenomenex Jupiter C18 column (21.2×250 mm) using a 0.675%/minute gradient of 0 to 27% acetonitrile containing 0.1% trifluoroacetic acid at a flow rate of 20 mL/min. The main product peak eluting at 28.5 minutes was lyophilized to give 57.4 mg (72.0%) of the title compound as a colorless solid with 100% purity by HPLC. ¹H NMR (DMSO d-6): δ 10.33 (s, 1H), 9.17 (broad s, 1H), 8.72-8.02 (m, 7H), 7.83-7.69 (m, 3H), 7.43-7.31 (m, 2H), 7.22 (d, J=9.0 Hz, 1H), 6.76 (d, J=8.8 Hz, 2H), 3.82 (s, 1H), 3.42 (s, 4H), 1.74-1.50 (m, 3H), 1.01-0.73 (m, 6H); MS: m/e 611.6 [M+H](100%); 1222.1 [2M+H](20%); High Resolution MS: Calculated for C31H45N4O10S [M+H]: 611.2395, Found: 611.2386.

### EXAMPLE 7

### Synthesis of 2-[2-({5-[N-((2S)-2-Amino-4-methylpentanoylamino)carbamoyl](2-pyridyl)}amino)(1Z)-2-azavinyl]benzenesulfonic Acid

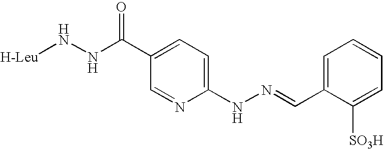

### Part A—Preparation of 2-{(1Z)-2-Aza-2-[(5-{N-[(tert-butoxy)carbonylamino]-carbamoyl}(2-pyridyl))amino]vinyl}benzenesulfonic Acid

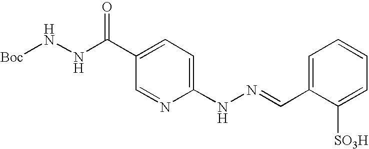

A solution of t-butyl carbazate (0.30 g, 2.27 mmol) and diisopropylethylamine (1.9 mL 11.35 mmol) in N,N-dimethylformamide (5 mL) was treated with HOAt (0.31 g, 2.27 mmol) and sodium 2-[(1E)-2-aza-2-({5-[(2,5-dioxopyrrolidinyl)oxycarbonyl](2-pyridyl)}amino)vinyl]benzene sulfonate (1.00 g, 2.27 mmol), and stirred under nitrogen at ambient temperature. At 27 hours, an additional (0.454 mmol) of t-butyl carbazate was added at 27 hours, and again at 45 hours. At 70 hours, N,N-dimethylformamide was removed by vacuum to give an amber oil, which was dissolved in 1:1 acetonitrile/water and lyophilized to give a sticky yellow solid. This solid was triturated with 0.1M HCl (2×25 mL), washed with water (3×15 mL) and dried under vacuum over calcium sulfate to give 0.961 g (97%) of desired product in 87.8% purity by HPLC. MS: m/e 436.5 [M+H](100%), 871.7 [2M+H](100%), 1307.0 [3M+H](30%).

### Part B—Preparation of 2-(2-{[5-(N-{(2S)-2-[(tert-Butoxy)carbonylamino]-4-methylpentanoylamino}carbamoyl)(2-pyridyl)]amino}(1Z)-2-azavinyl)benzenesulfonic Acid

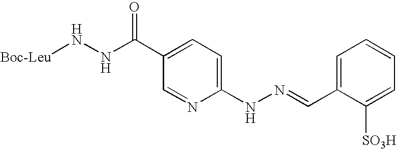

Product from part A, above (900 mg, 2.07 mmol) was dissolved in 1:1 trifluoroacetic acid/dichloromethane (15 mL) and allowed to react for 10 minutes at ambient temperatures. The solution was concentrated under reduced pressure to produce a golden oil, which was taken up in N,N-dimethylformamide (7 mL). This solution was added to a solution of Boc-leucine hydrate (770 mg, 3.1 mmol, NovaBiochem), HBTU (940 mg, 2.47 mmol) and diisopropylethylamine (4.3 mL, 25 mmol) in N,N-dimethylformamide, and stirred for 30 minutes at ambient temperatures. The N,N-dimethylformamide was removed under vacuum and the resulting amber oil was triturated with 0.1M HCl (2×20 mL), washed with water (3×20 mL) and dried under vacuum over calcium sulfate to give 1.25 g (111%) of desired product in 76.43% purity by HPLC. MS: m/e 449.5 [M+H-Boc](100%), 493.5 [M+H-(t-Bu)](35%), 1097.9 [2M+H](45%).

### Part C—Preparation of 2-[2-({5-[N-((2S)-2-Amino-4-methylpentanoylamino)carbamoyl](2-pyridyl)}amino)(1Z)-2-azavinyl]benzenesulfonic Acid

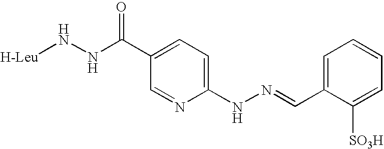

Product from part B, above (100 mg, 0.182 mmol) was dissolved in 1:1 trifluoroacetic acid/dichloromethane (6 mL) and allowed to react for 10 minutes. The solvent was removed under reduced pressure and the resulting amber oil was purified by HPLC on a Phenomenex Jupiter column (21.4×250 mm) using a 0.45%/minute gradient of 4.5 to 18% acetonitrile containing 0.1% trifluoroacetic acid at a flow rate of 80 mL/min. The main product peak eluting at 23.0 minutes was lyophilized to give 30.4 mg (37.5%) of the title compound as a colorless solid with 100% purity by HPLC. ¹H NMR (DMSO d-6): δ 10.53 (s, 1H), 9.14 (s, 1H), 8.62 (s, 1H), 8.34-8.00 (m, 4H), 7.79 (d, J=7.62 Hz, 1H), 7.46-7.30 (m, 2H), 7.27 (d, J=8.94 Hz, 1H), 3.86 (s, 1H), 1.89-1.54 (m, 3H), 1.02-0.82 (m, 6H); MS: m/e 336.3 [M+H-Leu](20%); 449.4 [M+H](100%); High Resolution MS: Calculated for C19H24N6O5S [M+H]: 449.1602, Found: 449.1586.

### EXAMPLE 8

### Synthesis of 2-[(1E)-2-({5-[N-({4-[N-((2S)-2-Amino-4-methylpentanoylamino)carbamoyl]phenyl}methyl)carbamoyl](2-pyridyl)}amino)-2-azavinyl]benzenesulfonic Acid

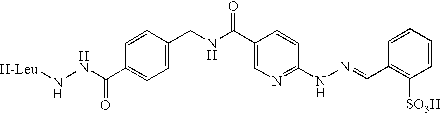 Part A—Preparation of N-[(tert-Butoxy)carbonylamino](4-{[(fluoren-9-ylmethoxy)carbonylamino]methyl}phenyl)carboxamide

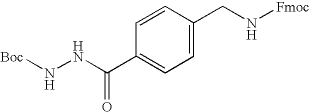

A solution of Fmoc-Amb-OH (2.50 g, 6.7 mmol), HOBt (1.11 g, 7.3 mmol), HBTU (2.77 g, 7.3 mmol) and diisopropylethylamine (3 mL, 17.2 mmol) in anhydrous N,N-dimethylformamide (10 mL) was stirred at ambient temperatures under nitrogen for 20 minutes, and treated with t-butyl carbazate (0.74 g, 5.6 mmol). After an additional 2 hours, the reaction was diluted with ethyl acetate (50 mL), washed consecutively with 0.1 N HCl (3×30 mL), 0.1 N NaOH (30 mL), water (30 mL), dried over MgSO₄ and evaporated to dryness. The resulting yellow solid was recrystallized from ethyl acetate/hexanes to give the title compound as a colorless solid (2.37 g, 87%). ¹H NMR (CDCl₃): δ 8.15 (bs, 1H), 7.79-7.51 (m, 6H), 7.45-7.20 (m, 6H), 6.85 (bs, 1H), 5.18 (s, 1H), 4.57-4.45 (m, 2H), 4.45-4.12 (m, 2H), 1.49 (s, 9H); ¹³C NMR (CDCl₃): δ 166.8, 156.6, 155.8, 143.8, 143.2, 141.4, 130.6, 127.8, 127.7, 127.5, 127.0, 124.9, 120.0, 82.5, 66.8, 47.3, 44.6, 28.1; MS: m/e 388.5 [M-Boc+H]; High Resolution MS: Calculated for C23H21N3O3 [M-Boc+H]: 388.1656, Found: 388.1643.

### Part B—Preparation of [4-(Aminomethyl)phenyl]-N-[(tert-butoxy)carbonylamino]-carboxamide

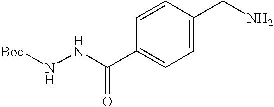

The product of Part A (0.80 g, 1.6 mmol) was treated with 2 mL of 20% piperidine in N,N-dimethylformamide at room temperature under nitrogen for 20 minutes. The N,N-dimethylformamide was removed under vacuum and the residue was chromatographed on silica gel, eluting consecutively with 9:1 CHCl₃/methanol, 8:1 CHCl₃/methanol, 4:1 CHCl₃/methanol, and 100% methanol to give the title compound as a colorless viscous oil (0.32 g, 74%). MS: m/e 166.3 [M-Boc+H].

### Part C—Preparation of 2-{(1E)-2-Aza-2-[(5-{N-[(4-{N-[(tert-butoxy)carbonylamino]carbamoyl}phenyl)methyl]carbamoyl}(2-pyridyl))amino]vinyl}benzenesulfonic Acid

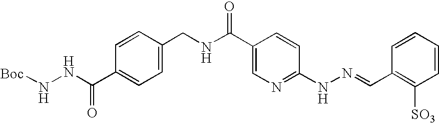

A solution of the product of Part B (0.309 g, 1.2 mmol), sodium 2-[(1E)-2-aza-2-({5-[(2,5-dioxopyrrolidinyl)oxycarbonyl](2-pyridyl)}amino)vinyl]benzenesulfonate (0.513 g, 1.2 mmol), HOAt (0.159 g, 1.2 mmol), and diisopropylethylamine (0.3 mL, 1.7 mmol) in anhydrous N,N-dimethylformamide (2 mL) was stirred at room temperature under nitrogen for 18 hours. The reaction was diluted with 10 mL of 0.1 N HCl. The resulting solid was collected by filtration, washed with 0.1 N HCl followed by water (3×10 mL), and dried to give the title compound as a colorless solid (0.625 g, 95%, HPLC purity>95%). MS: m/e 469.1 [M+H].

### Part D—Preparation of Sodium 2-((1E)-2-{[5-(N-{[4-(N-Aminocarbamoyl)phenyl]-methyl}carbamoyl)(2-pyridyl)]amino}-2-azavinyl)benzenesulfonate

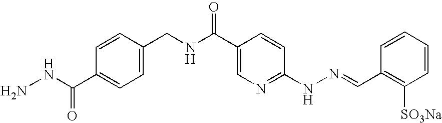

The product of Part C (0.22 g, 0.4 mmol) was treated with 6 mL of 50% trifluoroacetic acid in dichloromethane for 10 minutes at ambient temperatures under nitrogen. The solvents were removed under vacuum to give a colorless solid. The resulting solid was purified by HPLC on a Phenomenex Luna C18(2) column (41.4×250 mm) using a 1%/minute gradient of 9 to 36% acetonitrile containing 0.1M NaOAc (pH 7) at a flow rate of 80 mL/min. The main product peak eluting at 15 minutes was desalted on a Phenomenex Luna C18(2) column (41.4×250 mm) by diluting with water to an acetonitrile concentration of 5.4% and pumping onto the column. The column was eluted isocratically with 5.4% acetonitrile for 10 minutes at a flow rate of 80 mL/min, followed by a 2.2%/minute gradient of 5.4 to 45% acetonitrile at a flow rate of 80 mL/min. The main product peak eluting at 15 minutes was lyophilized to give the title compound as a colorless solid (0.14 g, 78%). MS: m/e 469.1 [M+H].

### Part E—Preparation of 2-((1E)-2-{[5-(N-{[4-(N-{(2S)-2-[(tert-Butoxy)carbonylamino]-4-methylpentanoylamino}carbamoyl)phenyl]methyl}carbamoyl)(2-pyridyl)]amino}-2-azavinyl)benzenesulfonic Acid

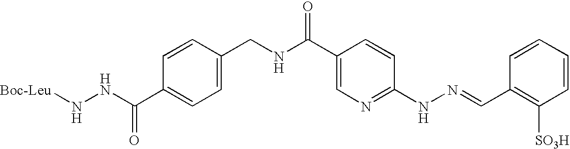

A solution of Boc-Leu-OH (0.130 g, 0.5 mmol), HOBt (0.078 g, 0.5 mmol), HBTU (0.190 g, 0.5 mmol) and diisopropylethylamine (0.149 mL, 0.5 mmol) in anhydrous N,N-dimethylformamide (2 mL) was stirred at ambient temperatures under nitrogen for 20 minutes, and treated with product of part D (0.200 g, 0.4 mmol). The solution was stirred for 4 hours at ambient temperatures and diluted with 0.1 N HCl (15 mL). The resulting precipitate was collected by filtration, washed consecutively with 0.1 N HCl (2×10 mL) and water (3×15 mL), and dried to give the title compound as a colorless solid (0.11 g, 38%). ¹H NMR (CD3CN:DMSO-d₆, 2:1): δ 13.04 (bs, 1H), 10.12 (s, 1H), 9.71 (s, 1H), 9.41 (s, 1H), 9.09 (s, 1H), 8.52 (s, 1H), 8.36 (d, J=9.0 Hz, 1H), 8.28 (d, J=6.9 Hz, 1H), 7.89-7.87 (m, 1H), 7.84 (d, J=8.13 Hz, 2H), 7.49-7.44 (m, 2H), 7.42 (d, J=8.13 Hz, 2H), 7.20 (d, J=8.90 Hz, 1H), 6.45 (d, J=8.90 Hz, 1H), 4.56 (d, J=5.8 Hz, 2H), 4.14 (q, J=7.9 Hz, 1H), 1.68-1.73 (m, 1H), 1.52 (t, J=7.3 Hz, 2H), 1.39 (s, 9H), 0.97-0.86 (m, 6H); ¹³C NMR (CD3CN:DMSO-d₆, 2:1): δ 173.3, 166.6, 156.5, 148.6, 144.2, 132.5, 130.9, 129.9, 128.7, 128.3, 127.9, 127.4, 122.1, 79.4, 52.7, 43.7, 42.2, 28.8, 25.3, 23.5, 22.2; MS: m/e 582.2 [M-Boc+H].

### Part F—Preparation of 2-[(1E)-2-({5-[N-({4-[N-((2S)-2-Amino-4-methylpentanoylamino)-carbamoyl]phenyl}methyl)carbamoyl](2-pyridyl)}amino)-2-azavinyl]benzenesulfonic Acid

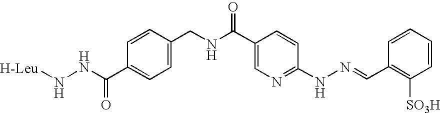

The product of Part E (0.11 g, 0.2 mmol) was treated with 8 mL of 50% trifluoroacetic acid in dichloromethane at ambient temperatures under nitrogen for 10 minutes. The solution was concentrated and the resulting colorless viscous oil was purified by HPLC on a Phenomenex Jupiter C18 column (21.2×250 mm) using a 1.2%/minute gradient of 9 to 45% acetonitrile containing 0.1% trifluoroacetic acid at a flow rate of 20 mL/min. The main product peak eluting at 12.9 minutes was lyophilized to give the title compound as a colorless solid (51 mg, yield 57%, HPLC purity 100%). ¹H NMR (DMSO-d₆): δ 10.56 (s, 1H), 10.53 (s, 1H), 9.20 (bs, 2H), 8.61 (s, 1H), 8.40-8.06 (m, 5H), 7.86 (d, J=8.2 Hz, 2H), 7.80 (d, J=6.7 Hz 1H), 7.46 (d, J=8.2 Hz, 2H), 7.44-7.34 (m, 2H), 7.25 (d, J=9.1 Hz, 1H), 4.55 (d, J=8.6 Hz, 2H), 1.85-1.77 (m, 1H), 1.72-1.63 (m, 1H), 1.63-1.52 (m, 1H), 0.94 (q, J=6.0 Hz, 6H); MS: m/e 582.6 [M+H]; High Resolution MS: Calculated for C27H31N7O6S [M+H]: 582.2129, Found: 582.2146.

### EXAMPLE 9

### Synthesis of N-((2S)-2-Amino-4-methylpentanoylamino)-6-[(7-methoxy-2-oxo(2H-chromen-3-yl))carbonylamino]hexanamide

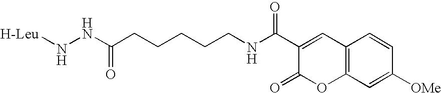 Part A—Preparation of (2S)-N-[(tert-Butoxy)carbonylamino]-2-[(fluoren-9-ylmethoxy)carbonylamino]-4-methylpentanamide

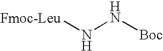

A solution of Fmoc-Leu-OH (0.50 g, 1.4 mmol) and diisopropylethylamine (0.62 mL, 3.5 mmol) in anhydrous THF (10 mL) was treated with isobutyl chloroformate (0.18 mL, 1.5 mmol) and stirred at 0° C. under nitrogen for 15 minutes. A solution of t-butyl carbazate (0.19 g, 1.4 mmol) in anhydrous THF (5 mL) was added and the reaction was stirred at ambient temperature under nitrogen for 16 hours. The reaction was diluted with ethyl acetate (25 mL), washed consecutively with 0.1 N HCl (25 mL), saturated NaHCO₃ (25 mL), 0.1 N NaOH (2×25 mL), water (25 mL), and brine (25 mL), dried (MgSO4), and concentrated to give the title compound as a colorless viscous oil (0.44 g, 66%, HPLC purity 100%). ¹H NMR (CD3CN): δ 8.17 Is, 1H), 7.85 (d, J=7.51 Hz, 2H), 7.72-7.65 (m, 2H), 7.43 (t, J=7.51 Hz, 2H), 7.39-7.32 (m, 2H), 6.93 (s, 1H), 5.90 (d, J=7.8 Hz, 1H), 4.41-4.21 (m, 3H), 4.17-4.06 (m, 1H), 1.74-1.63 (m, 1H), 1.59-1.50 (m, 2H), 1.42 (s 9H), 1.00-0.81 (m, 6H); ¹³C NMR (CD3CN): δ 173.3, 157.2, 156.3, 145.3, 145.2, 142.3, 129.0, 128.2, 81.4, 67.4, 53.2, 48.2, 41.9, 28.5, 25.5, 23.4, 21.9; MS: m/e 468.1 [M+H].

### Part B—Preparation of (2S)-N-Amino-2-[(fluoren-9-ylmethoxy)carbonylamino]-4-methylpentanamide Trifluoroacetic Acid Salt

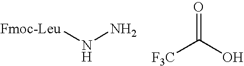

The product of Part A (0.44 g, 0.9 mmol) was treated with 10 mL of 50% trifluoroacetic acid in dichloromethane at room temperature under nitrogen for 10 minutes. The solution was concentrated to give the title compound as a pale yellow viscous oil (0.47 g, yield 138%, HPLC purity 100%). ¹H NMR (CD3CN): δ 7.84 (d, J=7.51 Hz, 2H), 7.68 (t, J=6.93 Hz, 2H), 7.43 (t, J=7.51 Hz, 2H), 7.38-7.31 (m, 2H), 5.96 (s, 1H), 5.78 (bs, 2H), 1.76-1.49 (m, 3H), 1.02-0.79 (m, 6H); MS: m/e 368.3 [M+H].

### Part C—Preparation of N-{(2S)-2-[(Fluoren-9-ylmethoxy)carbonylamino]-4-methylpentanoylamino}-6-[(tert-butoxy)carbonylamino]hexanamide

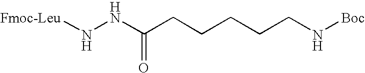

A solution of Boc-Ahx-OH (0.15 g, 0.6 mmol), HOBt (0.11 g, 0.7 mmol), HBTU (0.27 g, 0.7 mmol) and diisopropylethylamine (3 mL, 17.2 mmol) in anhydrous N,N-dimethylformamide (10 mL) was stirred at ambient temperatures under nitrogen for 15 minutes, and treated with the product of Part B (0.2 g, 0.5 mmol). The reaction was stirred for 1 hour, diluted with ethyl acetate (15 mL), washed consecutively with 0.1 N HCl (15 mL), 0.1 N NaOH (2×15 mL), water (15 mL), and brine (15 mL), dried (MgSO4), and concentrated to give the title compound as a colorless solid (0.28 g, 87%). MS: m/e 481.4 [M-Boc+H].

### Part D—Preparation of N-{(2S)-2-[(Fluoren-9-ylmethoxy)carbonylamino]-4-methylpentanoylamino}-6-aminohexanamide Trifluoroacetic Acid Salt

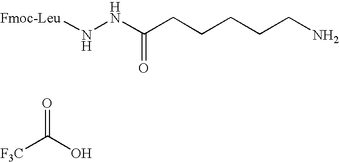

The product of Part C (0.28 g, 0.5 mmol) was treated with 12 mL of 50% trifluoroacetic acid in dichloromethane for 10 minutes at ambient temperatures under nitrogen. The solution was concentrated under reduced pressure and the residue was titurated with ether (3 mL) to give a colorless solid (0.26 g, 113%).%). ¹H NMR (CD3CN): δ 8.76-8.49 (m, 1H), 7.85 (d, J=7.5 Hz, 2H), 7.73-7.64 (m, 2H), 7.43 (t, J=7.5 Hz, 2H), 7.38-7.33 (m, 2H), 7.05 (bs, 2H), 4.41-4.12 (m, 4H), 2.96 (t, J=7.0 Hz, 2H), 2.22 (t, J=6.8 Hz, 2H), 1.76-1.35 (m, 11H), 1.00-0.81 (m, 6H); MS: m/e 481.4 [M+H].

### Part E—Preparation of N-{(2S)-2-[(Fluoren-9-ylmethoxy)carbonylamino]-4-methylpentanoylamino}-6-[(7-methoxy-2-oxo(2H-chromen-3-yl))carbonyl amino]hexanamide

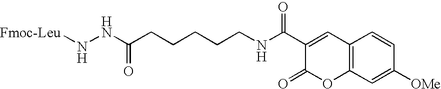

A solution of 7-methoxycoumarin-3-carboxylic acid (0.022 g, 0.1 mmol), HOBt (0.015 g, 0.1 mmol), HBTU (0.038 g, 0.1 mmol) and diisopropylethylamine (0.03 mL, 0.2 mmol) in anhydrous N,N-dimethylformamide (0.5 mL) was stirred at room temperature under nitrogen for 10 minutes, and treated with the product of Part D (0.040 g, 0.08 mmol). The solution was stirred for 4 hours at ambient temperatures and concentrated under reduced pressure. The resulting residue was washed with CH₂Cl₂ (3 mL) and THF (3 mL), and dried to give the title compound as a yellowish solid (0.031 g, 55%). MS: m/e 683.7 [M+H].

### Part F—Preparation of N-((2S)-2-Amino-4-methylpentanoylamino)-6-[(7-methoxy-2-oxo(2H-chromen-3-yl))carbonylamino]hexanamide Trifluoroacetic Acid Salt

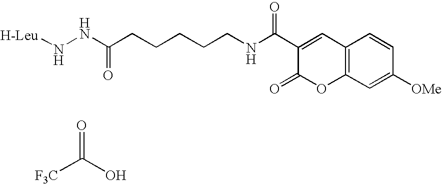

The product of Part E (0.020 g, 0.03 mmol) was treated with 1 mL of 20% piperidine in N,N-dimethylformamide at room temperature under nitrogen for 20 minutes. The N,N-dimethylformamide was removed under vacuum, and the residue was purified by HPLC on a Phenomenex Jupiter C18 column (21.2×250 mm) using a 1.35%/minute gradient of 4.5 to 45% acetonitrile containing 0.1% trifluoroacetic acid at a flow rate of 20 mL/min. The main product peak eluting at 23.4 minutes was lyophilized to give the title compound as a colorless solid (0.012 g, 89%). ¹H NMR (CDCl3): δ 10.32-9.55 (m, 1H), 8.95 (s, 1H), 8.83 (s, 1H), 8.25 (bs, 1H), 7.65-7.58 (m, 1H), 6.97-6.81 (m, 2H), 4.34 (s, 1H), 3.89 (s, 3H), 3.86-3.30 (m, 5H), 2.34 (s, 1H), 1.88-1.53 (m, 7H), 1.45-1.35 (m, 2H), 1.00-0.78 (m, 6H); MS: m/e 461.5 [M+H]; High Resolution MS: Calculated for C23H32N4O6 [M+H]: 461.2395, Found: 461.2391.

### EXAMPLE 10

### Synthesis of Ammonium 2-[(1E)-2-({5-[N-({4-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)carbamoyl]phenyl}methyl)carbamoyl](2-pyridyl)}amino)-2-azavinyl]benzenesulfonate

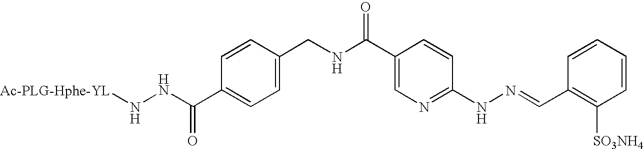

### Part A—Preparation of Fmoc-PLG-Hphe-Y(t-Bu)-L-HMPB-BHA Resin

HMPB-BHA resin (5.00 g, substitution level=0.61 mmol/g) was placed in a 100 mL Advanced ChemTech reaction vessel, and swollen by washing with N,N-dimethylformamide (2×40 mL). Fmoc-Leu-OH (3.23 g, 9.15 mmol) in N,N-dimethylformamide (35 mL) was added and the resin was mixed at room temperature for 15 minutes. Pyridine (1.09 g, 13.73 mmol) and 2,6-dichlorobenzoyl chloride (1.92 g, 9.15 mmol) were added and the mixture was gently shaken for 20 hours. The resin was washed thoroughly (40 mL volumes) with N,N-dimethylformamide (3×), dichloromethane (3×), methanol (3×), dichloromethane (3×), and N,N-dimethylformamide (3×). The remaining hydroxyl groups of the resin were capped by reacting with benzoyl chloride (1.5 mL) and pyridine (1.5 mL) in dichloromethane (40 mL) for 2 hours. The substitution level was determined to be 0.4 mmol/g by quantitative fulvene-piperidine assay.

The following steps were performed: (Step 1) The Fmoc group was removed using 20% piperidine in N,N-dimethylformamide for 30 minutes. (Step 2) The resin was washed thoroughly (40 mL volumes) with N,N-dimethylformamide (3×), dichloromethane (3×), methanol (3×), dichloromethane (3×), and N,N-dimethylformamide (3×). (Step 3) Fmoc-Tyr(Ot-Bu)-OH (3.68 g, 8 mmol), HOBt (1.22 g, 8 mmol), and HBTU (3.03 g, 8 mmol) in 10 mL of N,N-dimethylformamide and 3 mL of diisopropylethylamine were added to the resin and the reaction was allowed to proceed for 8 hours. (Step 4) The resin was washed thoroughly (40 mL volumes) with N,N-dimethylformamide (3×), dichloromethane (3×), methanol (3×), dichloromethane (3×), N,N-dimethylformamide (3×). (Step 5) Fmoc-Tyr(Ot-Bu)-OH (3.68 g, 8 mmol), HOBt (1.22 g, 8 mmol), HBTU (3.03 g, 8 mmol) in 10 mL of N,N-dimethylformamide and 3 mL of diisopropylethylamine were added to the resin and the reaction allowed to proceed for 4 hours. (Step 6) The resin was washed thoroughly (40 mL volumes) with N,N-dimethylformamide (3×), dichloromethane (3×), methanol (3×), dichloromethane (3×), and N,N-dimethylformamide (3×). (Step 7) The coupling reaction was found to be complete as assessed by the semi-quantitative ninhydrin assay and quantitative picric assay or fulvene-piperidine assay. Steps 1-7 were repeated until the sequence Fmoc-PLG-Hphe-Y(t-Bu)-L had been attained.

### Part B—Preparation of Ac-PLG-Hphe-Y(t-Bu)-L-OH

The product of Part A (1 g, substitution level=0.4 mmol/g), was placed in a 50 mL Advanced ChemTech reaction vessel, and swollen by washing with N,N-dimethylformamide (2×20 mL). The Fmoc group was removed using 20% piperidine in N,N-dimethylformamide (20 mL) for 30 minutes. The resin was washed thoroughly (20 mL volumes) with N,N-dimethylformamide (3×), dichloromethane (3×), methanol (3×), dichloromethane (3×), and N,N-dimethylformamide (3×). Acetic anhydride (0.38 mL, 4 mmol), and diisopropylethylamine (0.84 mL, 4 mmol) were added, and the resin was mixed for 18 hours. The reaction was found to be complete as assessed by LC/MS of a small portion of cleaved peptide.

The peptide-resin was placed in a sintered glass funnel and treated with 1% trifluoroacetic acid in dichloromethane (10 mL). After 2 minutes, the solution was filtered, by the application of pressure, directly into a solution of 10% pyridine in methanol (2 mL). The cleavage step was repeated nine times. The combined filtrates were evaporated to 5% of their volume, diluted with water (15 mL), and cooled in an ice-water bath. The resulting precipitate was collected by filtration in a sintered glass funnel, washed with water, and dried under vacuum. Purification was accomplished by HPLC on a Phenomenex Jupiter C18 column (41.2×250 mm) using a 1.2%/minute gradient of 45 to 81% acetonitrile containing 0.1% trifluoroacetic acid to give the title compound as a colorless solid (0.103 g, overall yield 31%, HPLC purity 100%). MS: m/e 821.8 [M+H] (100%); FT-MS: Calculated for C44H64N6O9 [M+H]: 821.4808, Found: 821.4792.

### Part C—Preparation of Ammonium 2-[(1E)-2-({5-[N-({4-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-[4-(tert-butoxy)phenyl]propanoylamino}-4-methylpentanoylamino)carbamoyl]phenyl}-methyl)carbamoyl](2-pyridyl)}amino)-2-azavinyl]benzenesulfonate

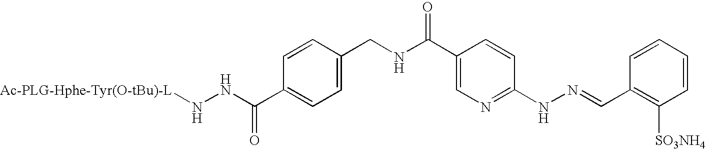

A solution of the product of Part B, above, (5.0 mg, 0.006 mmol), and the product of Example 8, Part D (2.9 mg, 0.006 mmol), were dissolved in N,N-dimethylformamide (60 μL) and made basic with collidine (0.8 μL, 0.006 mmol). The solution was treated with HOAt (1.7 mg, 0.012 mmol) and DIC (2.0 μL, 0.012 mmol), and stirred at room temperature under nitrogen for 18 hours. The N,N-dimethylformamide was removed under reduced pressure and the residue was purified by HPLC on a Phenomenex Luna C18(2) column (21.2×250 mm) using a 1.12%/minute gradient of 36 to 58.5% acetonitrile containing 0.1M NH4OAc (pH 7) at a flow rate of 20 mL/min. The main product peak eluting at 12.3 minutes was lyophilized to give the title compound as a colorless solid (3.9 mg, 51%, HPLC purity 100%). MS: m/e 1272.4 [M+H]. Chiral analysis for L-Leucine: 99.6%.

### Part D—Preparation of Ammonium 2-[(1E)-2-({5-[N-({4-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)carbamoyl]-phenyl}methyl)carbamoyl](2-pyridyl)}amino)-2-azavinyl]benzenesulfonate

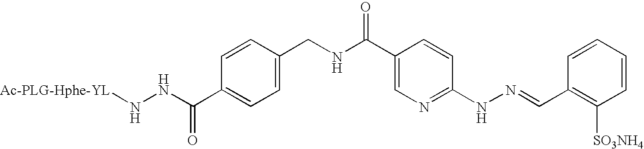

The product of Part C (6.9 mg, 0.005 mmol) was dissolved in 95:2.5:2.5 trifluoroacetic acid:anisole:water (2 mL) was stirred at room temperature under nitrogen for 10 minutes. The solution was concentrated under vacuum, and the resulting residue was purified by HPLC on a Phenomenex Luna C18(2) column (21.2×250 mm) using a 0.9%/minute gradient of 22.5 to 45% acetonitrile containing 0.05M NH4OAc (pH 7) at a flow rate of 20 mL/min. The main product peak eluting at 21.9 minutes was lyophilized to give the title compound as a colorless solid (1 mg, 15%, HPLC purity 100%). MS: m/e 1215.3 [M+H]; High Resolution MS: Calculated for C61H74N12O13S [M+H]: 1215.5292, Found: 1215.5285. Chiral analysis for L-Leucine: 99.8%.

### EXAMPLE 11

### Synthesis of Ammonium 2-((1E)-2-{[5-(N-{[4-(N-{(2S)-2-[(2S)-2-(2-{(2S)-2-[((2S)-1-Acetylpyrrolidin-2-yl)carbonylamino]-5-aminopentanoylamino}acetylamino)-4-phenylbutanoylamino]-4-methylpentanoylamino}carbamoyl)phenyl]-methyl}carbamoyl)(2-pyridyl)]amino}-2-azavinyl)benzenesulfonate

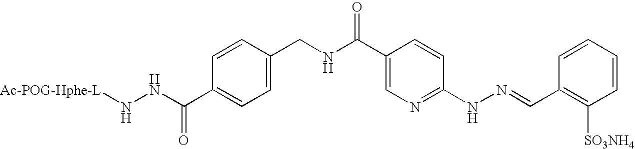

### Part A—Preparation of 2-((1E)-2-{[5-(N-{[4-(N-{(2S)-2-[(2S)-2-(2-{(2S)-2-[((2S)-1-Acetylpyrrolidin-2-yl)carbonylamino]-5-[(tert-butoxy)carbonylamino]pentanoylamino}-acetylamino)-4-phenylbutanoylamino]-4-methylpentanoylamino}carbamoyl)-phenyl]methyl}carbamoyl)(2-pyridyl)]amino}-2-azavinyl)benzenesulfonic Acid

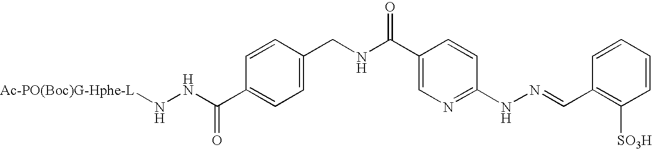

A solution of the product of Example 14, Part B (20.0 mg, 0.028 mmol), the product of Example 8, Part D (13.3 mg, 0.028 mmol), and HOAt (7.7 mg, 0.057 mmol) in DMSO (150 μL) were treated with collidine (3.4 μL, 0.028 mmol) and DIC (8.9 μL, 0.057 mmol), and stirred at room temperature under nitrogen. After 2 hours, additional product of Example 8, Part D (2 mg, 0.004 mmol) and collidine (7.6 μL, 0.063 mmol) were added. The reaction was stirred for an additional 18 hours, and purified by HPLC on a Phenomenex Luna C18(2) column (21.2×250 mm) using a 0.45%/minute gradient of 31.5 to 45% acetonitrile containing 0.1% trifluoroacetic acid at a flow rate of 20 mL/min. The main product peak eluting at 18.2 minutes was lyophilized to give the title compound as a colorless solid (9 mg, 27%, HPLC purity, 100%). MS: m/e 1153.4 [M+H].

### Part B—Preparation of Ammonium 2-((1E)-2-{[5-(N-{[4-(N-{(2S)-2-[(2S)-2-(2-{(2S)-2-[((2S)-1-Acetylpyrrolidin-2-yl)carbonylamino]-5-aminopentanoylamino}acetylamino)-4-phenylbutanoylamino]-4-methylpentanoylamino}carbamoyl)phenyl]-methyl}carbamoyl)(2-pyridyl)]amino}-2-azavinyl)benzenesulfonate

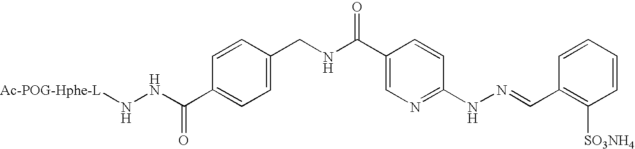

A solution of the product of Part A (9 mg, 0.008 mmol) in 95:2.5:2.5 trifluoroacetic acid:anisole:water (6.0 mL) was stirred at room temperature under nitrogen for 10 minutes. The solution was concentrated and the resulting residue was purified by HPLC on a Phenomenex Jupiter C18 column (21.2×250 mm) using a 0.9%/minute gradient of 9 to 36% acetonitrile containing 0.1M NH4OAc (pH 7) at a flow rate of 20 mL/min. The main product peak eluting at 29.5 minutes was lyophilized to give the title compound as a colorless solid (5.8 mg, 71%, HPLC purity, 100%). MS: m/e 1052.4 [M+H]; High Resolution MS: Calculated for C51H64N12O11S [M+H]: 1053.4611, Found: 1053.4592; Chiral analysis for L-leucine: 99.8%.

### EXAMPLE 12

### Synthesis of 3-(N-{2-[2-(N-{1-[N-({N-[1-(N-{1-[N-(1-{N-[(4-{[(6-{[(1E)-1-Aza-2-(2-sulfophenyl)vinyl]amino}(3-pyridyl))carbonylamino]methyl}phenyl)carbonylamino]-carbamoyl}(1S)-3-methylbutyl)carbamoyl](1S)-2-(4-hydroxyphenyl)ethyl}carbamoyl)(1S)-3-phenylpropyl]carbamoyl}methyl)carbamoyl](1S)-3-methylbutyl}-carbamoyl)(2S)pyrrolidinyl]-2-oxoethyl}acetylamino)propanoic Acid

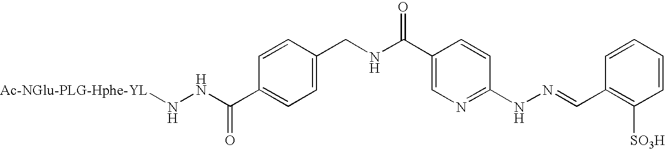

### Part A—Preparation of Fmoc-NGlu(Boc)-PLG-Hphe-Y(Ot-Bu)-L-HMPB-BHA Resin

The peptide-resin of Example 10, Part A (1 g, substitution level=0.4 mmol/g) was placed in a 50 mL Advanced ChemTech reaction vessel, and swollen by washing with N,N-dimethylformamide (2×20 mL). Fmoc group was removed using 20% piperidine in N,N-dimethylformamide (20 mL) for 30 minutes. The resin was washed thoroughly (20 mL volumes) with N,N-dimethylformamide (3×), dichloromethane (3×), methanol (3×), dichloromethane (3×), and N,N-dimethylformamide (3×). The resin was treated with Fmoc-NGlu(Boc)-OH (Simon, R. J. et al. Proc. Nat. Acad. Sci.: USA 1992, 89, 9367-9371) (0.51 g, 1.2 mmol), HOBt (0.18 g, 1.2 mmol), HBTU (0.46 g, 1.2 mmol), and diisopropylethylamine (0.68 mL, 4 mmol), and mixed for 10 hours. The coupling reaction was found to be complete as assessed by LC/MS of small portion cleaved peptide.

### Part B—Preparation of Ac-NGlu(Ot-Bu)-PLG-Hphe-Y(t-Bu)-L-ONH4

To peptide-resin of Part A was treated with 20% piperidine in N,N-dimethylformamide (20 mL) for 30 minutes. The resin was washed thoroughly (20 mL volumes) with N,N-dimethylformamide (3×), dichloromethane (3×), methanol (3×), dichloromethane (3×), and N,N-dimethylformamide (3×). Acetic anhydride (0.38 mL, 4 mmol), and diisopropylethylamine (0.84 mL, 4 mmol) were added and the resin was mixed for 18 hours. The coupling reaction was found to be complete as assessed by LC/MS of a small portion of cleaved peptide.

The peptide-resin was placed in a sintered glass funnel and treated with 1% trifluoroacetic acid in dichloromethane (10 mL). After 2 minutes, the solution was filtered, by the application of pressure, directly into a solution of 10% pyridine in methanol (2 mL). The cleavage step was repeated three times. The combined filtrates were concentrated and the resulting residue was purified by HPLC on a Phenomenex Luna C18(2) column (41.4×250 mm) using a 0.9%/minute gradient of 36 to 63% acetonitrile containing 0.1M NH4OAc (pH 7) to give the title compound as a colorless solid (0.12 g, overall yield 30%, HPLC purity 100%). MS: m/e 1006.5 [M+H] (100%).

### Part C—Preparation of 2-((1E)-2-{[5-(N-{[4-(N-{(2S)-2-[(2S)-2-((2S)-2-{2-[(2S)-2-({(2S)-1-[2-(N-{2-[(tert-Butyl)oxycarbonyl]ethyl}acetylamino)acetyl]pyrrolidin-2-yl}carbonylamino)-4-methylpentanoylamino]acetylamino}-4-phenylbutanoylamino)-3-[4-(tert-butoxy)phenyl]propanoylamino]-4-methylpentanoylamino}carbamoyl)phenyl]methyl}carbamoyl)(2-pyridyl)]amino}-2-azavinyl)benzenesulfonic Acid

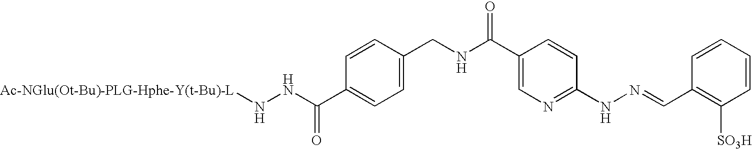

A solution of the product of part B, above (20.0 mg, 0.02 mmol), and the product of Example 8, Part D (9.3 mg, 0.02 mmol) in DMSO (100 μL) was treated with HOAt (5.4 mg, 0.04 mmol), collidine (2.6 μL, 0.02 mmol), and DIC (6.2 μL, 0.04 mmol), and stirred at room temperature under nitrogen for 3 hours. Additional product from Example 8, Part D (2 mg, 0.004 mmol) and collidine (2.6 mL, 0.02 mmol) were added and the reaction was stirred for another 2 hours. The solution was purified by HPLC on a Phenomenex Jupiter C18 column (21.2×250 mm) using a 1%/minute gradient of 40.5 to 63% acetonitrile containing 0.1% trifluoroacetic acid at a flow rate of 20 mL/min. The main product peak eluting at 22.4 minutes was lyophilized to give the title compound as a colorless solid (0.16 g, 57%, HPLC purity 100%). MS: m/e 1456.5 [M+H].

### Part D—Preparation of 3-(N-{2-[2-(N-{1-[N-({N-[1-(N-{1-[N-(1-{N-[(4-{[(6-{[(1E)-1-Aza-2-(2-sulfophenyl)vinyl]amino}(3-pyridyl))carbonylamino]-methyl}phenyl)carbonylamino]carbamoyl}(1S)-3-methylbutyl)carbamoyl](1S)-2-(4-hydroxyphenyl)ethyl}carbamoyl)(1S)-3-phenylpropyl]carbamoyl}methyl)carbamoyl]-(1S)-3-methylbutyl}carbamoyl)(2S)pyrrolidinyl]-2-oxoethyl}acetylamino)propanoic Acid

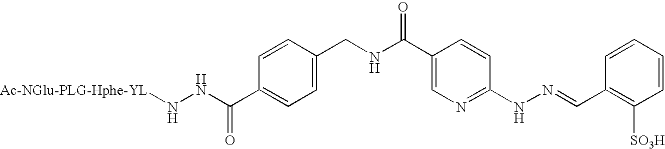

The product of Part A was dissolved in 95:2.5:2.5 trifluoroacetic acid:anisole:water (3 mL) was stirred at room temperature under nitrogen for 10 minutes. The solution was concentrated under reduced pressure, and the resulting residue was purified by HPLC on a Phenomenex Jupiter C18 column (21.2×250 mm) using a 1%/minute gradient of 9 to 36% acetonitrile containing 0.1% trifluoroacetic acid at a flow rate of 20 mL/min. The main product peak eluting at 28.2 minutes was lyophilized to give the title compound as a colorless solid (2.6 mg, 57%, HPLC purity, 100%). MS: m/e 1344.4 [M+H]; High Resolution MS: Calculated for C66H81N13O16S [M+H]: 1344.5718, Found: 1344.5706; Chiral analysis for L-Leucine: 99.2%.

### EXAMPLE 13

### Synthesis of 2-((1E)-2-{[5-(N-{5-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino) carbamoyl]pentyl}carbamoyl)(2-pyridyl)]amino}-2-azavinyl)benzenesulfonic Acid

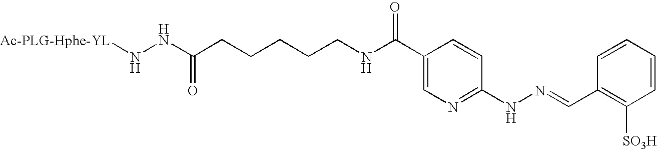

### Part A—Preparation of N-[(tert-Butoxy)carbonylamino]-6-[(fluoren-9-ylmethoxy)carbonylamino]hexanamide

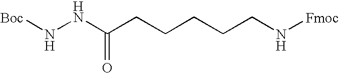

A solution of Fmoc-6-Ahx-OH (3.00 g, 8.5 mmol), HOBt (1.41 g, 9.2 mmol), HBTU (3.49 g, 9.2 mmol) and diisopropylethylamine (3.45 mL, 19.9 mmol) in anhydrous N,N-dimethylformamide (15 mL) was stirred at ambient temperatures under nitrogen for 20 minutes, and treated with t-butyl carbazate (0.93 g, 7.0 mmol) and diisopropylethylamine (1 mL, 5.8 mmol). The solution was stirred for 5 hours, diluted with ethyl acetate (15 mL), washed consecutively with 0.1 N HCl (3×15 mL), water (25 mL), and brine (30 mL), dried (Mugs4), and concentrated to give a yellow oil. The oil was purified by flash chromatography over silica gel, eluting with 95:5 CH₂Cl₂:methanol to give the title compound as a colorless solid (2.51 g, 71%, HPLC purity, 100%). ¹H NMR (CDCl₃): δ 7.75 (d, J=7.5 Hz, 2H), 7.58 (d, J=7.5 Hz, 2H), 7.39 (t, J=7.5 Hz, 2H), 7.37-7.28 (m, 3H), 6.48 (s, 1H), 4.95 (s, 1H), 4.39 (d, J=6.7 Hz, 2H), 4.21 (t, J=6.7 Hz, 1H), 3.17 (s, 2H), 2.21 (t, J=7.2 Hz, 2H), 1.82-1.59 (m, 4H), 1.45 (s, 9H), 1.40-1.32 (m, 2H); ¹³C NMR (CDCl₃): δ 172.4, 156.5, 155.5, 144.0, 141.3, 127.6, 127.0, 125.0, 119.9, 81.9, 66.5, 47.3, 40.7, 33.8, 29.5, 28.1, 25.9, 24.6; MS: m/e 368.3 [M-Boc+H]; High Resolution MS: Calculated for C26H33N3O5 [M+H]: 468.2493, Found: 468.2485.

### Part B—Preparation of 6-Amino-N-[(tert-butoxy)carbonylamino]hexanamide

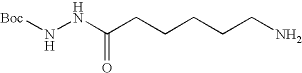

The product of Part A (1.44 g, 3.1 mmol) was treated with 20% piperidine in N,N-dimethylformamide (4.0 mL) at room temperature under nitrogen for 20 minutes. The solution was concentrated under reduced pressure and the resulting solid was purified by flash chromatography over silica gel, eluting consecutively with methanol, 100:3 methanol:TEA, and 100:6 methanol:TEA, to give the title compound as a colorless solid (0.79 g, 104%). ¹H NMR (CDCl₃): δ 4.12 (bs, 2H), 2.80-2.68 (m, 2H), 2.24 (t, J=7.3 Hz, 2H), 1.72-1.60 (m, 2H), 1.58-1.46 (m, 2H), 1.45 (s, 9H), 1.43-1.33 (m 2H); MS: m/e 246.3 [M+H].

### Part C—Preparation of Sodium 2-[(1E)-2-Aza-2-({5-[N-(5-{N-[(tert-butoxy)carbonylamino]carbamoyl}pentyl)carbamoyl](2-pyridyl)}amino)vinyl]benzenesulfonate

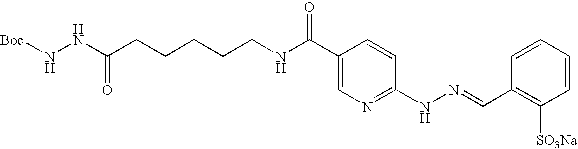

A solution of the product of Part B (0.72 g, 2.9 mmol), sodium 2-[(1E)-2-aza-2-({5-[(2,5-dioxopyrrolidinyl)oxycarbonyl](2-pyridyl)}amino)vinyl]benzenesulfonate (1.29 g, 2.9 mmol), HOAt (0.40 g, 2.9 mmol), and diisopropylethylamine (1.02 mL, 5.9 mmol) in anhydrous N,N-dimethylformamide (10 mL) was stirred at room temperature under nitrogen. After 2 hours, additional sodium 2-[(1E)-2-aza-2-({5-[(2,5-dioxopyrrolidinyl)oxycarbonyl](2-pyridyl)}amino)vinylbenzene sulfonate (0.27 g, 0.6 mmol) and diisopropylethylamine (0.1 mL, 0.6 mmol) were added and the reaction was stirred for overnight. The reaction mixture was filtered and the filtrate was concentrated. The resulting residue was purified by flash chromatography over silica gel, eluting with 85:15 CH₂Cl₂/methanol, to give the title compound as a colorless solid (0.81 g, yield 50%, HPLC purity, >95%). ¹H NMR (DMSO-d₆): δ 11.32 (s, 1H), 9.45 (s, 1H), 9.01 (s, 1H), 8.63 (s, 1H), 8.59 (d, J=2.1 Hz, 1H), 8.34-8.23 (m, 1H), 8.08-7.97 (m, 2H), 7.78 (dd, J=1.4, 7.5 Hz, 1H), 7.40-7.18 (m, 3H), 3.28-3.17 (m, 2H), 2.07 (t, J=7.2 Hz, 2H), 1.60-1.45 (m, 4H), 1.45-1.21 (m, 11H); MS: m/e 449.2 [M-Boc+H].

### Part D—Preparation of 2-{(1E)-2-[(5-{N-[5-(N-Aminocarbamoyl)pentyl]carbamoyl}(2-pyridyl))amino]-2-azavinyl}benzenesulfonic Acid

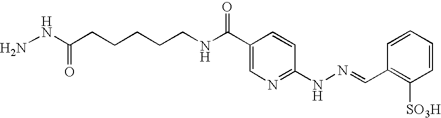

The product of Part C (0.37 g, 0.7 mmol) was treated with 50% trifluoroacetic acid in dichloromethane (5 mL) for 10 minutes at room temperature under nitrogen. The solution was concentrated under reduced pressure and the residue was purified by HPLC on a Phenomenex Jupiter C18 column (41.4×250 mm) using a 0.9%/minute gradient of 0 to 27% acetonitrile containing 0.1% trifluoroacetic acid at a flow rate of 80 mL/min. The main product peak eluting at 18.9 minutes was lyophilized to give the title compound as a colorless solid (0.24 g, 80%). ¹H NMR (DMSO-d₆): δ 10.75 (s, 1H), 9.22 (s, 1H), 8.64-8.54 (m, 1H), 8.53 (d, J=1.8 Hz, 1H), 8.29-8.11 (m, 2H), 7.80 (dd, J=1.9, 7.0 Hz, 1H), 7.47-7.32 (m, 2H), 7.23 (d, J=9.1 Hz, 1H), 4.50 (bs, 3H), 3.26 (q, J=6.4 Hz, 2H), 2.23 (t, J=7.3 Hz, 2H), 1.66-1.45 (m, 4H), 1.40-1.22 (m, 2H); MS: m/e 449.1 [M+H].

### Part E—Preparation of 2-((1E)-2-{[5-(N-{5-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-[4-(tert-butoxy)phenyl]propanoylamino}-4-methylpentanoylamino)carbamoyl]pentyl}carbamoyl)(2-pyridyl)]amino}-2-azavinyl)benzenesulfonic Acid

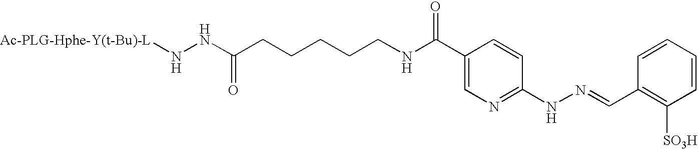

A solution of the product of Example 10, Part B (20.0 mg, 0.024 mmol), the product of Example 13, Part D (10.9 mg, 0.024 mmol), and HOAt (6.6 mg, 0.048 mmol) in anhydrous N,N-dimethylformamide (100 μL) was treated with collidine (11.2 μL, 0.084 mmol) and DIC (7.6 μL, 0.048 mmol), and stirred at room temperature under nitrogen. Additional product of Example 13, Part D was added at 2 hours (3 mg, 0.007 mmol) and at 5 hours (8 mg, 0.018 mmol). The reaction was stirred an additional 18 hours and concentrated under reduced pressure. The resulting residue was purified by HPLC on a Phenomenex Luna column (21.2×250 mm) using a 0.67%/minute gradient of 36 to 54% acetonitrile containing 0.1% trifluoroacetic acid at a flow rate of 20 mL/min. The main product peak eluting at 21.7 minutes was lyophilized to give the title compound as a colorless solid (11 mg, 36%, HPLC purity, 100%). MS: m/e 1251.6 [M+H].

### Part F—Preparation of 2-((1E)-2-{[5-(N-{5-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)carbamoyl]pentyl}carbamoyl)(2-pyridyl)]amino}-2-azavinyl)benzenesulfonic Acid

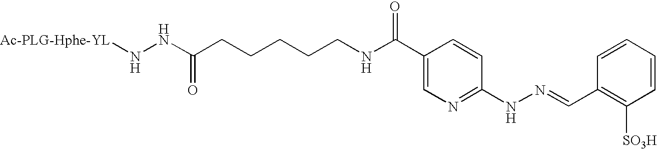

A solution of the product of Part E (11 mg, 0.009 mmol) in 95:2.5:2.5 trifluoroacetic acid:anisole:water (2 mL) was stirred at room temperature under nitrogen for 10 minutes. The solution was concentrated under reduced pressure and the resulting residue was purified by HPLC on a Phenomenex Luna C18(2) column (21.2×250 mm) using a 0.5%/minute gradient of 31.5 to 45% acetonitrile containing 0.1% trifluoroacetic acid at a flow rate of 20 mL/min. The main product peak eluting at 15.4 minutes was lyophilized to give the title compound as a colorless solid (3 mg, 29%, HPLC purity, 100%). MS: m/e 1195.5 [M+H]; High Resolution MS: Calculated for C59H78N12O13S [M+H]: 1195.5605, Found: 1195.5579. Chiral analysis for L-leucine: 99.8%.

### EXAMPLE 14

### Synthesis of Ammonium 2-((1E)-2-{[5-(N-{(2S)-2-[(2S)-2-(2-{(2S)-2-[((2S)-1-Acetylpyrrolidin-2-yl)carbonylamino]-5-aminopentanoylamino}acetylamino)-4-phenylbutanoylamino]-4-methylpentanoylamino}carbamoyl)(2-pyridyl)]amino}-2-azavinyl)benzenesulfonate

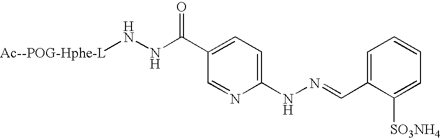

### Part A—Preparation of Fmoc-PO(Boc)G-Hphe-L-HMPB-BHA Resin

HMPB-BHA resin (2.000 g, substitution level=0.68 mmol/g) was placed in a 200 mL Advanced ChemTech reaction vessel and swollen by washing with N,N-dimethylformamide (2×50 mL). A solution of Fmoc-Leu-OH (3.60 g, 10.2 mmol) in N,N-dimethylformamide (40 mL) was added to the vessel and the mixture was gently agitated for 15 minutes. 2,6-Dichlorobenzoyl chloride (1.5 mL, 10.9 mmol) and pyridine (1.23 mL, 15.3 mmol) in N,N-dimethylformamide (10 mL) were added and the mixture was shaken under nitrogen at ambient temperature for 15 hours. The resin was washed (50 mL volumes) with N,N-dimethylformamide (3×), dichloromethane (3×), methanol (1×), dichloromethane (3×), and N,N-dimethylformamide (3×). A solution of benzoyl chloride (2.5 mL, 21.0 mmol) and pyridine (2.5 mL, 30.6 equiv) in N,N-dimethylformamide (50 mL) was added to the resin and the vessel was shaken under nitrogen for 10 hours and washed (50 mL volumes) with N,N-dimethylformamide (3×), dichloromethane (3×), methanol (1×), and dichloromethane (3×). Fulvene-Piperidine assay performed on dry sample of resin showed a loading of 0.450 mmol/g.

The following steps were performed: (Step 1) The Fmoc group was removed using 20% piperidine in N,N-dimethylformamide (50 mL) for 30 minutes. (Step 2) The resin was washed (50 ml volumes) with N,N-dimethylformamide (3×), dichloromethane (3×), methanol (3×), dichloromethane (3×), and N,N-dimethylformamide (3×). (Step 3) Fmoc-Hphe-OH (3.01 g, 7.5 mmol), HOBt (1.15 g, 7.5 mmol), and HBTU (2.84 g, 7.5 mmol) in 50 ml of N,N-dimethylformamide and 2 ml of diisopropylethylamine were added to the resin and the reaction was allowed to proceed for 5 hours. (Step 4) The resin was washed as in step 2. (Step 5) Repeat steps 3 and 4. (Step 6) Reaction completeness was monitored by qualitative Kaiser test. Steps 1-6 were repeated until the desired sequence had been attained.

### Part B—Preparation of Ac-PO(Boc)G-Hphe-L-OH

The product from Part A (1.5 g) was placed in a 100 mL Advanced ChemTech reaction vessel and swollen by washing with N,N-dimethylformamide (2×20 mL). The peptide-resin was treated with 20% piperidine in N,N-dimethylformamide (30 mL) for 30 minutes, followed by washing (30 ml volumes) with N,N-dimethylformamide (3×), dichloromethane (3×), methanol (3×), dichloromethane (3×), and N,N-dimethylformamide (3×). The resin was treated with acetic anhydride (0.63 mL, 6.75 mmol) and diisopropylethylamine (1.4 mL, 8.1 mmol) in N,N-dimethylformamide (30 mL), followed by washing (30 ml volumes) with N,N-dimethylformamide (3×), dichloromethane (3×), methanol (3×), and dichloromethane (3×), and drying under vacuum. The peptide-resin was placed in a sintered glass funnel and treated with a solution of 1% trifluoroacetic acid in dichloromethane (12 mL). After 2 minutes the solution was filtered, by the application of nitrogen pressure, directly into a flask containing 1:9 pyridine/methanol (2 μL). The cleavage procedure was repeated ten (10) times. The combined filtrates were concentrated to an oily solid. This crude product was purified by HPLC on a Phenomenex Jupiter C18 column (21.2×250 mm) using a 0.9%/minute gradient of 18 to 45% acetonitrile containing 0.1% trifluoroacetic acid at a flow rate of 20 L/min. The main product peak eluting at 28.5 minutes was lyophilized to give 313.1 mg (66.0%) of the title compound as a colorless solid with 100% purity by HPLC. MS: m/e 603.7 [M+H-Boc](100%), 703.8 [M+H](95%), 1428.4 [2M+Na].

### Part C—Preparation of Sodium 2-((1Z)-2-{[5-(N-Aminocarbamoyl)(2-pyridyl)]amino}-2-azavinyl)benzenesulfonate

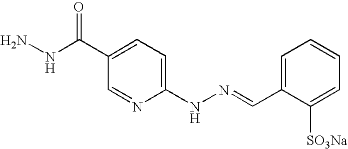

The product of Example 7, Part A (150 mg, 0.344 mmol) was dissolved in 1:1 trifluoroacetic acid:dichloromethane (8 mL) and stirred for 10 minutes under nitrogen gas at ambient temperature. The solution was concentrated under reduced pressure to give a golden oil which was purified by HPLC on a Phenomenex Luna C18(2) column (21.2×250 mm) using a 1.08%/minute gradient of 4.5 to 31.5% acetonitrile containing 50 mM ammonium acetate at a flow rate of 20 mL/min. The product fractions were lyophilized to a colorless solid which was repurified by HPLC on a Phenomenex Luna C18(2) column (21.2×250) using a 1%/minute gradient of 0 to 30% acetonitrile containing 100 mM sodium acetate. The main product peak was desalted on a Phenomenex Luna C18(2) column (21.2×250 mm) by diluting with water to an acetonitrile concentration of 4% and pumping onto the column. The column was eluted isocratically with 4% acetonitrile for 15 minutes at 20 mL/min, followed by a 2.3%/minute gradient of 4 to 50% acetonitrile at a flow rate of 20 mL/min. The main product fraction was lyophilized to give the title compound as a colorless solid (86.3 g, 59.0%) in 98.6% purity by HPLC. MS: m/e 336.1 [M+H](100%), 671.1 [2M+H](75%), 1006.3 [3M+H](15%).

### Part D—Preparation of 2-((1E)-2-{[5-(N-{(2S)-2-[(2S)-2-(2-{(2S)-2-[((2S)-1-Acetylpyrrolidin-2-yl)carbonylamino]-5-[(tert-butoxy)carbonylamino]-pentanoylamino}acetylamino)-4-phenylbutanoylamino]-4-methylpentanoylamino}-carbamoyl)(2-pyridyl)]amino}-2-azavinyl)benzenesulfonic Acid

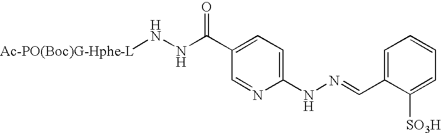

A solution of the product of Part B (20.0 mg, 0.0285 mmol), the product from Part C (9.5 mg, 0.0285 mmol), and HOAt (3.9 mg, 0.0285 mmol) in DMSO (150 μL) was treated with collidine (16 μL, 0.114 mmol) and DIC (4.5 μL, 0.0285 mmol), and stirred under nitrogen at room temperature. After 24 hours, the reaction solution was treated with additional product of Part C (4.8 mg, 0.0143 mmol), DIC (2.3 μL, 0.0143 mmol) and collidine (12 μL, 0.0855 mmol). At 44 hours, the reaction was purified by HPLC on a Phenomenex Jupiter C18 column (21.2×250 mm) using a 1.29%/minute gradient of 13.5 to 52.2% acetonitrile containing 0.1% trifluoroacetic acid at a flow rate of 20 mL/min. The main product peak eluting from 23 to 26.5 minutes was lyophilized to give the title compound (19.6 mg, 68.0%) as a colorless solid with 100% purity by HPLC. MS: m/e 460.9 [M-Boc+2H](30%), 920.4 [M+H-Boc](10%), 1020.4 [M+H](100%).

### Part E—Preparation of Ammonium 2-((1E)-2-{[5-(N-{(2S)-2-[(2S)-2-(2-{(2S)-2-[((2S)-1-Acetylpyrrolidin-2-yl)carbonylamino]-5-aminopentanoylamino}acetylamino)-4-phenylbutanoylamino]-4-methylpentanoylamino}carbamoyl)(2-pyridyl)]amino}-2-azavinyl)benzenesulfonate

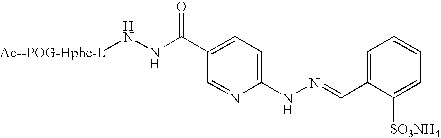

The product from Part D (19.0 mg, 0.0186 mmol) was dissolved in 1:1 trifluoroacetic acid:dichloromethane (5 mL) and stirred under nitrogen at ambient temperature for 10 minutes. The solution was concentrated under reduced pressure and the resulting solid was purified by HPLC on a Phenomenex Jupiter C18 column (21.2×250 mm) using a 0.45%/minute gradient of 18 to 36% acetonitrile containing 100 mM ammonium acetate at a flow rate of 20 mL/min. The main product peak eluting at 27 minutes was lyophilized to give 10.9 mg (60.0%) of the title compound as a colorless solid with 100% purity by HPLC. MS: m/e 460.7 [M+2H] (100%); 920.3 [M+H] (90%); High Resolution MS: Calculated for C43H58N11O10S [M+H]: 920.4083, Found: 920.4063; Chiral analysis for L-leucine: 99.9%.

### EXAMPLE 15

### Synthesis of 3-[N-(2-{2-[N-(1-{N-[(N-{1-[N-(1-{N-[(6-{[(1E)-1-Aza-2-(2-sulfophenyl)vinyl]amino}(3-pyridyl))carbonylamino]carbamoyl}(1S)-3-methylbutyl)carbamoyl](1S)-3-phenylpropyl}carbamoyl)methyl]carbamoyl}(1S)-4-aminobutyl)carbamoyl](2S)pyrrolidinyl}-2-oxoethyl)acetylamino]propanoic Acid Ammonium Salt

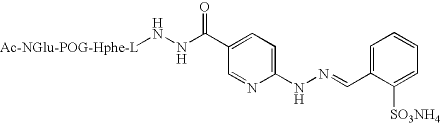

### Part A—Preparation of Ac-NGlu(O-t-Bu)-PO(Boc)G-Hphe-L-OH

The product of Example 14, Part A (1.00 g, substitution level=0.5 mmol/g) was placed in a 200 ml Advanced ChemTech reaction vessel and swollen by washing with N,N-dimethylformamide (2×50 mL). The following steps were performed: (Step 1) The Fmoc group was removed using 20% piperidine in N,N-dimethylformamide (50 mL) for 30 minutes. (Step 2) The resin was washed (50 mL volumes) with N,N-dimethylformamide (3×), dichloromethane (3×), methanol (3×), dichloromethane (3×), and N,N-dimethylformamide (3×). (Step 3) Fmoc-NGlu(Ot-Bu)-OH (0.64 g, 1.5 mmol), HOBt (0.23 g, 1.5 mmol), and HBTU (0.57 g, 1.5 mmol) in N,N-dimethylformamide (60 mL) and diisopropylethylamine (1 mL) were added to the resin and the reaction allowed to proceed for 10 hours followed by washing as in step 2. (Step 4) The Fmoc group was removed using 20% piperidine in N,N-dimethylformamide (50 m]L) for 30 minutes, followed by washing as in step 2. (Step 5) The resin was treated with acetic anhydride (0.3 mL, 5 mmol) and diisopropylethylamine (0.81 mL, 6 mmol) in N,N-dimethylformamide (60 mL) and the mixture was shaken under nitrogen for 18 hours. The resin was washed (50 mL volumes) with N,N-dimethylformamide (3×), dichloromethane (3×), methanol (1×), and dichloromethane (3×), and dried under vacuum.

The peptide-resin was placed in a sintered glass funnel and treated with 1% trifluoroacetic acid in dichloromethane (12 mL) for 2 minutes. The solution was filtered, by application of nitrogen pressure, directly into a flask containing 1:9 pyridine:methanol (2 mL). The cleavage procedure was repeated ten (10) times. The combined filtrates were concentrated to an oily solid. This crude product was purified by HPLC on a Phenomenex Jupiter C18 column (41.4×250 mm) using a 0.9%/minute gradient of 31.5 to 58.5% acetonitrile containing 0.1% trifluoroacetic acid at a flow rate of 80 mL/min. The main product peak eluting at 20.3 minutes was lyophilized to give 165.3 mg (37.1%) of the title compound as a colorless solid with 93.7% purity by HPLC. MS: m/e 788.4 [M+H-Boc](85%), 888.5 [M+H](100%).

### Part B—Preparation Ammonium of 2-{(1E)-2-[(5-{N-[(2S)-2-((2S)-2-{2-[(2S)-2-({(2S)-1-[2-(N-{2-[(tert-Butyl)oxycarbonyl]ethyl}acetylamino)acetyl]pyrrolidin-2-yl}carbonylamino)-5-[(tert-butoxy)carbonylamino]pentanoylamino]acetylamino}-4-phenylbutanoylamino)-4-methylpentanoylamino]carbamoyl}(2-pyridyl))amino]-2-azavinyl}benzenesulfonate

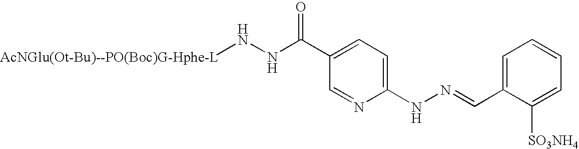

A solution of the product of Part A (15.0 mg, 0.0169 mmol), the product of Example 14, Part C (5.67 mg, 0.0169 mmol), and HOAt (2.32 mg, 0.0169 mmol) in DMSO (150 μL) was treated with collidine (9 μL, 0.0676 mmol) and DIC (2.65 μL, 0.0169 mmol) and allowed to stir under nitrogen at room temperature. After 4 hours, additional product of Example 14, Part C (2.85 mg, 0.0084 mmol), DIC (1.33 μL, 0.0084 mmol) and collidine (4.5 μL, 0.0338 mmol) were added. The reaction was stirred an additional 16 hours, and purified by HPLC on a Phenomenex Jupiter C18 column (21.2×250 mm) using a 0.52%/minute gradient of 33.8 to 49.5% acetonitrile containing 100 mM ammonium acetate at a flow rate of 20 mL/min. The main product peak eluting from 17 to 22.5 minutes. was lyophilized to give 10.6 mg (52.0%) of the title compound as a colorless solid with 100% purity by HPLC. MS: m/e 525.4 [(M−Boc-(t-Bu))+2H](90%), 1205.4 [M+H](100%), Chiral analysis for L-leucine: 95.4%.

### Part C—Preparation of 3-[N-(2-{2-[N-(1-{N-[(N-{1-[N-(1-{N-[(6-{[(1 E)-1-Aza-2-(2-sulfophenyl)vinyl]amino}(3-pyridyl))carbonylamino]carbamoyl}(1S)-3-methylbutyl)carbamoyl](1 S)-3-phenylpropyl}carbamoyl)methyl]carbamoyl}(1S)-4-aminobutyl)carbamoyl](2S)pyrrolidinyl}-2-oxoethyl)acetylamino]propahoic Acid Ammonium Salt

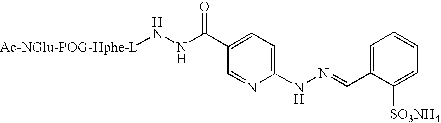

The product of Part B (9.6 mg, 0.008 mmol) was dissolved in 38:1:1 trifluoroacetic acid/Anisole/Water (4 mL) and stirred under nitrogen at ambient temperature for 10 minutes. The solution was concentrated and the resulting solid was purified by HPLC on a Phenomenex Jupiter C18 column (21.2×250 mm) using a 0.45%/minute gradient of 18 to 36% acetonitrile containing 100 mM ammonium acetate at a flow rate of 20 mL/min. The main product peak eluting at 20 minutes. was lyophilized to give 5.6 mg (66.7%) of the title compound as a colorless solid with 100% purity by HPLC. MS: m/e 525.3 [M+2H] (40%); 1049.4 [M+H] (100%); High Resolution MS: Calculated for C43H58N11O10S [M+H]: 1049.4509 Found: 1049.4512; Chiral analysis for L-leucine: 99.5%.

### EXAMPLE 16

### Synthesis of Amino 2-[(1E)-2-({5-[N-((2S)-2-{(2S)-2-[(2S)-2-(2-{(2S)-2-[((2S)-1-Acetylpyrrolidin-2-yl)carbonylamino]-4methylpentanoylamino}acetylamino)-4-phenylbutanoylamino]-3-(4-hydroxyphenyl)propanoylamino}-4-methylpentanoylamino)carbamoyl](2-pyridyl)}amino)-2-azavinyl]benzenesulfonate

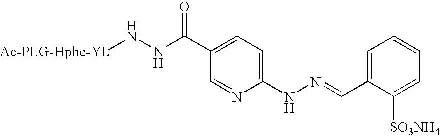

### Part A—Preparation of 2-[(1E)-2-({5-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-[4-(tert-butoxy)phenyl]propanoylamino}-4-methylpentanoylamino)carbamoyl](2-pyridyl)}amino)-2-azavinyl]benzenesulfonic Acid

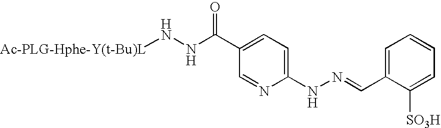

A solution of the product of Example 10, Part B (15.0 mg, 0.0183 mmol), the product of Example 14, Part C (6.12 mg, 0.0183 mmol), and HOAt (2.51 mg, 0.0183 mmol) in DMSO (150 μL) was treated with collidine (9.7 μL, 0.0732 mmol) and DIC (2.87 μL, 0.0183 mmol), and stirred under nitrogen at room temperature. After 1.5 hours, the reaction mixture was treated with additional product of Example 14, Part C (3.0 mg, 0.0092 mmol), DIC (1.45 μL, 0.0092 mmol), and collidine (4.9 μL, 0.0366 mmol). The reaction was stirred a total of 22 hours and purified by HPLC on a Phenomenex Luna C18(2) column (21.2×250 mm) using a 0.9%/minute gradient of 36 to 63% acetonitrile containing 0.1% trifluoroacetic acid at a flow rate of 20 mL/min. The main product peak eluting at 23.7 minutes was lyophilized to give 11.3 mg (54.3%) of the title compound as a colorless solid with 100% purity by HPLC. MS: m/e 1138.5 [M+H](100%); Chiral analysis for L-leucine: 98.7%.

### Part B—Deprotection

The product of Part A (9.6 mg, 0.0084 mmol) was dissolved in 38:1:1 trifluoroacetic acid:Anisole:water (4 mL) and stirred under nitrogen at ambient temperature for 15 minutes. The solution was concentrated under reduced pressure and the resulting solid was purified by HPLC on a Phenomenex Jupiter C, 18 column (21.2×250 mm) using a 0.0.9%/minute gradient of 22.5 to 49.5% acetonitrile containing 100 mM ammonium acetate at a flow rate of 20 mL/min. The main product peak eluting at 21.5 minutes was lyophilized to give 3.1 mg (34.2%) of the title compound as a colorless solid with 100% purity by HPLC. MS: m/e 541.7 [M+2H] (25%); 1082.5 [M+H] (100%); High Resolution MS: Calculated for C53H68N11O12S [M+H]: 1082.4764. Found: 1082.4762.

### EXAMPLE 17

### Synthesis of Ammonium 2-[(1E)-2-({5-[N-({4-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-5-aminopentanoylamino}-4-methylpentanoylamino)carbamoyl]phenyl}methyl)carbamoyl](2-pyridyl)}amino)-2-azavinyl]benzenesulfonate

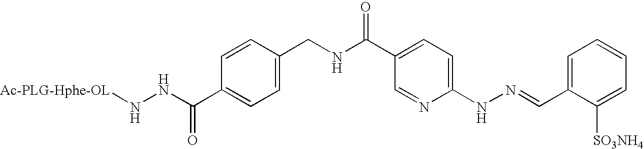

### Part A—Preparation of Fmoc-PLG-Hphe-O(Boc)L-HMPB-BHA Resin

HMPB-BHA resin (8.000 g, substitution level=0.68 mmol/g) was placed in a 200 mL Advanced ChemTech reaction vessel and swollen by washing with N,N-dimethylformamide (2×45 mL). A solution of Fmoc-Leu-OH (5.77 g, 16.32 mmol) in N,N-dimethylformamide (45 mL) was added to the vessel and the mixture was shaken for 15 minutes. 2,6-Dichlorobenzoyl chloride (2.5 mL, 16.32 mmol) and pyridine (2.0 mL, 24.5 mmol) in N,N-dimethylformamide (45 mL) were added and the mixture was shaken under nitrogen at ambient temperature for 18 hours. The resin was washed (90 mL volumes) with N,N-dimethylformamide (3×), dichloromethane (3×), methanol (1×), dichloromethane (3×) and N,N-dimethylformamide (3×). A solution of benzoyl chloride (3.0 mL, 26 mmol) and pyridine (3.0 mL, 36.7 mmol) in N,N-dimethylformamide (90 mL) was added to the resin and the vessel was shaken under nitrogen for 3 hours and washed (90 mL volumes) with N,N-dimethylformamide (3×), dichloromethane (3×), methanol (1×) and dichloromethane (3×). Fulvene-Piperidine assay performed on dry sample of resin showed a loading of 0.340 mmol/g.

The following steps were performed: (Step 1) The Fmoc group was removed using 20% piperidine in N,N-dimethylformamide (90 mL) for 30 minutes. (Step 2) The resin was washed (90 ml volumes) with N,N-dimethylformamide (3×), dichloromethane (3×), methanol (3×), dichloromethane (3×), and N,N-dimethylformamide (3×). (Step 3) Fmoc-Orn(Boc)-OH (3.71 g, 8.16 mmol), HOBt (1.25 g, 8.16 mmol), and HBTU (3.10 g, 8.16 mmol) in 90 mL of N,N-dimethylformamide and 2 ml of diisopropylethylamine were added to the resin and the reaction was allowed to proceed for 5 hours. (Step 4) The resin was washed as in step 2. (Step 5) Fmoc-Orn(Boc)-OH (3.71 g, 8.16 mmol) and PyBroP (3.8 g, 8.16 mmol) in 90 ml of N,N-dimethylformamide and 2 mL of diisopropylethylamine were added to the resin and the reaction was allowed to proceed for 5 hours. (Step 7) The resin was washed (90 mL volumes) with N,N-dimethylformamide (3×), dichloromethane (3×), methanol (3×), and dichloromethane (3×). (Step 6) Reaction completeness monitored by Fulvene-Piperidine assay. Steps 1 to 7 were repeated until the desired sequence was attained. Coupling yields were >95%.

### Part B—Preparation of Ac-PLG-Hphe-O(Boc)L-OH

The peptide-resin of Part A (2.5 g) was placed in a 100 mL Advanced ChemTech reaction vessel and swollen by washing with N,N-dimethylformamide (2×30 mL). The resin was treated with 20% piperidine in N,N-dimethylformamide (30 mL) for 30 minutes to remove Fmoc protecting group, followed by washing (30 ml volumes) with N,N-dimethylformamide (3×), dichloromethane (3×), methanol (3×), dichloromethane (3×), and N,N-dimethylformamide (3×). Acetic anhydride (0.78 mL, 4.2 mmol), diisopropylethylamine (0.88 mL, 5.0 mmol), and N,N-dimethylformamide (30 mL) were added and the mixture was gently agitated for 2 hours. The peptide-resin was washed (30 mL volumes) with N,N-dimethylformamide (3×), dichloromethane (3×), methanol (3×), and dichloromethane (3×), and dried under vacuum. The peptide-resin was placed in a sintered glass funnel and treated with 1% trifluoroacetic acid in dichloromethane (12 mL) for 2 minutes. The solution was filtered, by application of nitrogen pressure, directly into a flask containing 1:9 pyridine:methanol (2 mL). The cleavage procedure was repeated ten (10) times. The combined filtrates were concentrated to give a colorless oily solid. This crude product triturated with water (2×25 mL) and dried under reduced pressure to give a dry solid. This solid was purified by HPLC on a Phenomenex Luna C18(2) column (21.2×250 mm) using a 0.9%/minute gradient of 22.5 to 58.5% acetonitrile containing 0.1% trifluoroacetic acid at a flow rate of 20 mL/min. The main product peak eluting at 28.5 minutes was lyophilized to give 68.4 mg (9.3%) of the title compound as a colorless solid with 100% purity by HPLC. MS: m/e 716.6 [M+H-Boc](90%), 816.7 [M+H](100%).

### Part C—Preparation of 2-[(1E)-2-({5-[N-({4-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-5-[(tert-butoxy)carbonylamino]pentanoylamino}-4-methylpentanoylamino)carbamoyl]phenyl}methyl)carbamoyl](2-pyridyl)}amino)-2-azavinyl]benzenesulfonic Acid

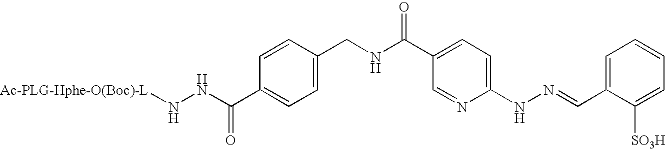

A solution of the product of Part B (15.0 mg, 0.0184 mmol), the product of Example 8, Part D (8.62 mg, 0.0184 mmol), and HOAt (2.52 mg, 0.0184 mmol) in DMSO (150 μL) was treated with collidine (9.7 μL, 0.0736 mmol) and DIC (2.88 μL, 0.0184 mmol), and stirred under nitrogen at room temperature. After 5 hours, the reaction solution was treated with additional product of Example 8, Part D (2.16 mg, 0.0046 mmol), DIC (0.72 μL, 0.0046 mmol), and collidine (2.5 μL, 0.0184 mmol) and stirred an additional 15 hours. The reaction was purified by HPLC on a P C18henomenex Luna column (21.2×250 mm) using a 0.9%/minute gradient of 27 to 54% acetonitrile containing 0.1% trifluoroacetic acid at a flow rate of 20 mL/min. The main product peak eluting at 24.9 minutes was lyophilized to give 14.1 mg (60.0%) of the desired compound as a colorless solid with 100% purity by HPLC. MS: m/e 583.9 [M-Boc+2H](100%), 1166.5 [M+H-Boc](20%), 1266.5 [M+H](100%); Chiral analysis for L-leucine: 98.9%.

### Part D—Preparation of Ammonium 2-[(1E)-2-({5-[N-({4-[N-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-5-aminopentanoylamino}-4-methylpentanoylamino)carbamoyl]phenyl}methyl)carbamoyl](2-pyridyl)}amino)-2-azavinyl]benzenesulfonate

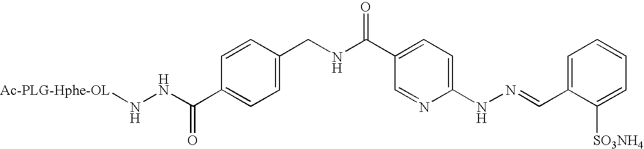

The product of Part C (13.0 mg, 0.0103 mmol) was dissolved in 1:1 trifluoroacetic acid:dichloromethane (3 mL) and stirred under nitrogen at ambient temperatures for 10 minutes. The solution was concentrated under reduced pressure and the resulting solid was purified by HPLC on a Phenomenex Jupiter C18 column (21.2×250 mm) using a 0.45%/minute gradient of 22.5 to 36% acetonitrile containing 100 mM ammonium acetate at a flow rate of 20 mL/min. The main product peak eluting at 28.0 minutes was lyophilized to give 10.9 mg (60.0%) of the title compound as a colorless solid with 100% purity by HPLC. MS: m/e 584.0 [M+2H] (55%); 1166.5 [M+H] (100%); High Resolution MS: Calculated for C57H76N13O12S [M+H]: 1166.5451, Found: 1166.5456; Chiral analysis for L-leucine: 99.9%.

### EXAMPLE 18

### Synthesis of Ammonium 2-((1E)-2-{[5-(N-{[4-(N-{2-[2-(2-{2-[2-({1-[(2R)-2-(Acetylamino)-3-(aminooxysulfonyl)propanoyl](2S)pyrrolidin-2-yl}carbonylamino)(2S)-4-methylpentanoylamino]acetylamino}(2S)-4-phenylbutanoylamino)(2S)-3-(4-hydroxyphenyl)propanoylamino](2S)-4-methylpentanoylamino}carbamoyl)phenyl]methyl}carbamoyl)(2-pyridyl)]amino}-2-azavinyl)benzenesulfonate

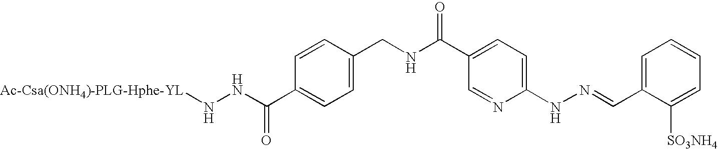

### Part A—Preparation of Ac-Csa-PLG-Hphe-Y(t-Bu)L-OH

The peptide-resin from Example 10, Part A (500 mg, substitution level=0.4 mmol/g) was placed in a 50 mL Advanced ChemTech reaction vessel and swollen by washing with N,N-dimethylformamide (2×20 mL). The following steps were performed: (Step 1) The Fmoc group was removed using 20% piperidine in N,N-dimethylformamide (20 mL) for 30 minutes. (Step 2) The resin was washed (20 mL volumes) with N,N-dimethylformamide (3×), dichloromethane (3×), methanol (3×), dichloromethane (3×), and N,N-dimethylformamide (3×). (Step 3) Fmoc-Csa-OH (Hubbuch, A.; Danho, W.; Zahn, H. Liebigs Ann. Chem. 1979, 776-783) (240 mg, 0.60 mmol), HOBt (90 mg, 0.60 mmol), and HBTU (230 mg, 0.60 mmol) in N,N-dimethylformamide (20 mL) and diisopropylethylamine (1 mL) were added to the resin and the mixture was gently agitated for 5 hours followed by washing as in step 2. (Step 4) Step 3 was repeated. (Step 5) The Fmoc group was removed using 20% piperidine in N,N-dimethylformamide (20 mL) for 30 minutes, followed by washing as in step 2. (Step 5) The peptide-resin was treated with acetic anhydride (0.35 mL 4 mmol) and diisopropylethylamine (0.87 mL, 5 mmol) in N,N-dimethylformamide (20 mL) and the mixture was shaken under nitrogen for 18 hours. The resin was washed (20 mL volumes) with N,N-dimethylformamide (3×), dichloromethane (3×), methanol (1×), and dichloromethane (3×), and dried under vacuum.

The peptide-resin was placed in a sintered glass funnel and treated with 1% trifluoroacetic acid in dichloromethane (10 mL) for 2 minutes. The solution was filtered, by application of nitrogen pressure, directly into a flask containing 1:9 pyridine:methanol (2 mL). The cleavage procedure was repeated ten (10) times. The combined filtrates were concentrated to give a colorless oily solid. This crude product was purified by HPLC on a Phenomenex Jupiter C18 column (41.4×250 mm) using a 0.66%/minute gradient of 26.1 to 45.9% acetonitrile containing 0.1% trifluoroacetic acid at a flow rate of 80 mL/min. The main product peak eluting from 24 to 28 minutes was lyophilized to give 67.3 mg of a 51:49 mixture of the title compound and peptide having lost the t-butyl group from tyrosine. Total yield for these two products was 17.0%. MS (protected): m/e 972.5 [M+H](100%); MS (deprotected): m/e 916.3 [M+H](100%).

### Part B—Conjugation Reaction

A solution of the product of Part A (15.0 mg, 0.0154 mmol), the product of Example 8, Part D (7.3 mg, 0.0154 mmol), and HOAt (2.15 mg, 0.0154 mmol) in DMSO (150 μL) was treated with collidine (7.2 μL, 0.0543 mmol) and DIC (2.50 μL, 0.0154 mmol), and stirred under nitrogen at room temperature. After 3 hours, the reaction solution was treated with additional product of Example 8, Part D (1.83 mg, 0.0039 mmol), DIC (0.54 μL, 0.0039 mmol), and collidine (1.8 μL, 0.0154 mmol), and stirred an additional 17 hours. The reaction was purified by HPLC on a Phenomenex Luna C18(2) column (21.2×250 mm) using a 0.9%/minute gradient of 18 to 54% acetonitrile containing 0.1% trifluoroacetic acid at a flow rate of 20 mL/min. The conjugate of the protected product eluted at 29.5 minutes and was lyophilized to give a colorless solid (5.0 mg). The title compound eluted at 19.0 minutes and was lyophilized to give a colorless solid that was purified further by HPLC on a Phenomenex Jupiter C18 column (21.2×250 mm) using a 0.9%/minute gradient of 18 to 54% acetonitrile containing 100 mM ammonium acetate at a flow rate of 20 mL/min. The main product peak eluting at 21.0 minutes. was lyophilized to give 7.1 mg (64.5% corrected for the protected conjugate) of the title compound as a colorless solid with 100% purity by HPLC. MS: m/e 1367.4 [M+H] (100%); High Resolution MS: Calculated for C64H80N13O17S2 [M+H]: 1366.5215, Found: 1366.5208; Chiral analysis for L-leucine: 99.9%.

### EXAMPLE 19

### Synthesis of 2-((1E)-2-{[5-(N-{5-[N-(Acetylamino)carbamoyl]pentyl}carbamoyl)(2-pyridyl)]amino}-2-azavinyl)benzenesulfonic Acid

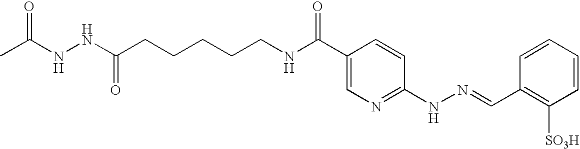

A solution of acetic anhydride (10.9 μL, 0.12 mmol), the product of Experiment 13, Part D (52 mg, 0.12 mmol), and HOAt (30.8 mg, 0.23 mmol) in anhydrous N,N-dimethylformamide (0.2 mL) was treated with diisopropylethylamine (100 μL, 0.57 mmol) and DIC (35.5 μL, 0.24 mmol), and stirred at room temperature under nitrogen for 3 hours. The solution was concentrated and the resulting residue was purified by HPLC on a Phenomenex Luna C18(2) column (21.2×250 mm) using a 0.9%/minute gradient of 0 to 27% acetonitrile containing 0.1% trifluoroacetic acid at a flow rate of 20 mL/min. The main product peak eluting at 23 minutes was lyophilized to give the title compound as a colorless solid (36 mg, 63%, HPLC purity 100%). ¹H NMR (DMSO-d₆): δ 9.72-9.60 (m, 2H), 9.32 (s, 1H), 8.66 (s, 1H), 8.50-8.43 (m, 1H), 8.42-8.19 (m, 2H), 7.85-7.73 (m, 1H), 7.53-7.36 (m, 2H), 7.20 (d, J=9.3 Hz, 1H), 3.13-3.32 (m, 2H), 2.12 (t, J=7.2 Hz, 2H), 1.83 (s, 1H), 1.63-1.42 (m, 4H), 1.41-1.22 (m, 2H); MS: m/e 491.2 [M+H]; High Resolution MS: Calculated for C21H26N6O6S [M+H]: 491.1707, Found: 491.1702.

### EXAMPLE 20

### Synthesis of 2-((1E)-2-Aza-2-{[5-(N-{5-[N-(12-hydroxydodecanoylamino)carbamoyl]pentyl}carbamoyl)(2-pyridyl)]amino}vinyl)benzenesulfonic Acid

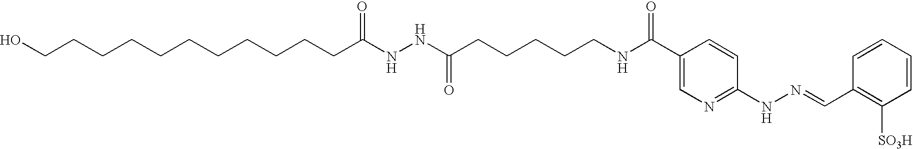

A solution of 12-hydroxydodecanoic acid (25 mg, 0.12 mmol), the product of Experiment 13, Part D (52 mg, 0.12 mmol), and HOAt (30.8 mg, 0.23 mmol) in anhydrous N,N-dimethylformamide (0.2 mL) was treated with diisopropylethylamine (100 μL, 0.57 mmol) and DIC (35.5 μL, 0.24 mmol), and stirred at room temperature under nitrogen for 3 hours. Additional product of Experiment 13, Part D (8 mg, 0.02 mmol) was added and the reaction was stirred for another 3 hours. The reaction was purified by HPLC on a Phenomenex Luna C18(2) column (21.2×250 mm) using a 0.9%/minute gradient of 18 to 45% acetonitrile containing 0.1% trifluoroacetic acid at a flow rate of 20 mL/min. The main product peak eluting at 21 minutes was lyophilized to give the title compound as a colorless solid (29 mg, 39%, HPLC purity 100%). ¹H NMR (DMSO-d₆): δ 9.63 (s, 2H), 9.30 (s, 1H), 8.64 (s, 1H), 8.50-8.44 (m, 1H), 8.40-8.18 (m, 2H), 7.88-7.75 (m, 1H), 7.52-7.46 (m, 2H), 7.20 (d, J=9.2 Hz, 1H), 3.36 (t, J=6.4 Hz, 2H), 3.31-3.18 (m, 2H), 2.17-2.00 (m, 4H), 1.62-1.18 (m, 24H); MS: m/e 647.4 [M+H]; High Resolution MS: Calculated for C31H46N6O7S [M+H]: 647.3221, Found: 647.3217.

### EXAMPLE 21

### Synthesis of 2-((1E)-2-Aza-2-{[5-(N-{5-[N-(dodecanoylamino)carbamoyl]pentyl}-carbamoyl)(2-pyridyl)]amino}vinyl)benzenesulfonic Acid

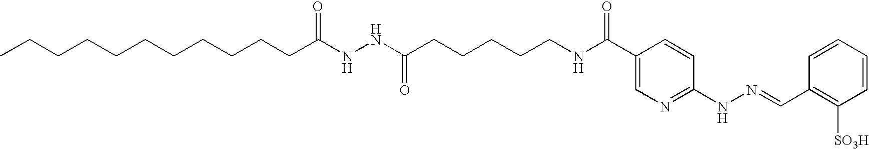

A solution of lauric acid (23.2 mg, 0.12 mmol), the product of Experiment 13, Part D (52 mg, 0.12 mmol), and HOAt (30.8 mg, 0.23 mmol) in anhydrous N,N-dimethylformamide (0.2 mL) was treated with diisopropylethylamine (100 μL, 0.57 mmol) and DIC (35.5 μL, 0.24 mmol), and stirred at room temperature under nitrogen for 2 hours. The solution was concentrated under reduced pressure and purified by HPLC on a Phenomenex Luna C18(2) column (21.2×250 mm) using a 0.6%/minute gradient of 31.5 to 49.5% acetonitrile containing 0.1% trifluoroacetic acid at a flow rate of 20 mL/min. The main product peak eluting at 31.1 minutes was lyophilized to give the title compound as a colorless solid (34 mg, 47%, HPLC purity 100%). ¹H NMR (DMSO-d₆): δ 9.63 (s, 2H), 9.30 (s, 1H), 8.64 (s, 1H), 8.50-8.43 (m, 1H), 8.40-8.18 (m, 2H), 7.85-7.75 (m, 1H), 7.50-7.36 (m, 2H), 7.20 (d, J=9.2 Hz, 1H), 3.31-3.18 (m, 2H), 2.18-2.00 (m, 4H), 1.62-1.39 (m, 6H), 1.39-1.11 (m, 18H), 0.90-0.78 (m, 3H); MS: m/e 631.3 [M+H]. High Resolution MS: Calculated for C31H46N6O6S [M+H]: 631.3272, Found: 631.3272.

### EXAMPLE 22

### Synthesis of 2-[(1E)-2-Aza-2-({5-[N-(5-hydroxydodecanoylamino)carbamoyl](2-pyridyl)}amino)vinyl]benzenesulfonic Acid

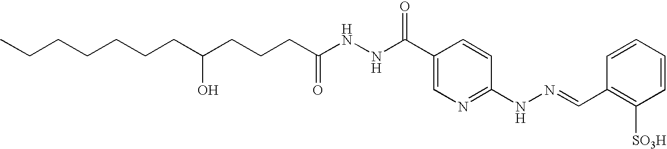

A solution of 6-dedocanolactone (7.9 mg, 0.04 mmol) and the product of Example 14, Part C (20 mg, 0.06 mmol) in anhydrous N,N-dimethylformamide (0.2 mL) was treated with sodium 2-ethylhexanoate (16.5 mg, 0.1 mmol) and stirred at room temperature under nitrogen for 18 hours followed by heating at 50° C. for 48 hours. The solution was concentrated and the residue was purified by HPLC on a Phenomenex Luna C18(2) column (21.2×250 mm) using a 1.35%/minute gradient of 18 to 45% acetonitrile containing 0.1% trifluoroacetic acid at a flow rate of 20 mL/min. The main product peak eluting at 19.2 minutes was lyophilized to give the title compound as a colorless solid (1.2 mg, 7.0%, HPLC purity 100%). MS: m/e 534.3 [M+H]; High Resolution MS: Calculated for C25H35N5O6S [M+H]: 534.2381, Found: 534.2375.

### EXAMPLE 23

### Synthesis of 2-{(1E)-2-Aza-2-[(5-{N-[2-(8-hydroxydodecanoylamino)ethyl]carbamoyl}(2-pyridyl))amino]vinyl}benzenesulfonic Acid

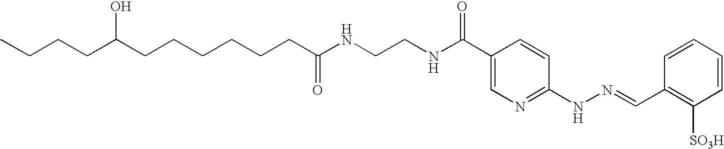

### Part A—Preparation of Ethyl 7-(Chlorocarbonyl)heptanoate

A solution of ethyl hydrogen seburate (5.0 g, 24.7 mmol) in anhydrous dichloromethane (15 mL) containing 5 drops of N,N-dimethylformamide was treated with oxalyl chloride (2.16 mL, 24.7 mmol), and stirred at room temperature under nitrogen for 3 hours. The solvents were removed under reduced pressure to afford a colorless oil (5.49 g, 101%). IR (deposit from CH₂Cl₂ solution onto a NaCl plate, cm⁻¹): 1797.4 (C═O), 1730.9 (C═O); ¹H NMR (CDCl₃): δ 4.11 (q, J=7.1 Hz, 2H), 2.87 (t, J=7.3 Hz, 2H), 2.28 (t, J=7.5 Hz, 2H), 1.73-1.67 (m, 2H), 1.67-1.57 (m, 2H), 1.38-1.30 (m, 4H), 1.24 (t, J=7.1 Hz, 3H); ¹³C NMR (CDCl₃): δ 173.7, 173.6, 60.2, 47.0, 34.2, 28.5, 28.0, 24.7, 24.5, 14.2.

### Part B—Preparation of Ethyl 8-Oxododecanoate

A solution of anhydrous Zinc chloride (0.69 g, 5.1 mmol) in anhydrous ether (10 mL) was treated with butylmagnesium chloride (2.53 mL, 2.0 M solution in ether, 5.1 mmol) dropwise at −78° C. The temperature was increased to 0° C. and the reaction mixture was treated with product of part A (1.23 g, 5.6 mmol) in anhydrous THF (10 mL) followed by Pd(PPh₃)₄ (0.057 g, 0.05 mmol). The resulting mixture was stirred at 0° C. for 30 minutes, then at room temperature for 1.5 hours. The reaction was quenched by the addition of 1N HCl (2 mL) and extracted with hexanes (2×20 mL). The combined organic layers were washed with saturated NaHCO₃ (30 mL), dried (MgSO₄), and concentrated. The resulting residue was chromatographed on silica gel, eluting with 1:3 ethyl acetate/Hexanes to give the title compound as a pale yellow oil (1.06 g, 96%). IR (deposit from CH₂Cl₂ solution onto a NaCl plate, cm⁻¹): 1737.5 (C═O), 1704.3 (C═O); ¹H NMR (CDCl₃): δ4.10 (q, J=7.1 Hz, 2H), 2.37 (t, J=7.5 Hz, 4H), 2.26 (t, J=7.5 Hz, 2H), 1.63-1.50 (m, 6H), 1.31-1.26 (m, 6H), 1.24 (t, J=7.1 Hz, 3H), 0.89 (t, J=7.5 Hz, 3H); ¹³C NMR (CDCl₃): δ 211.4, 173.7, 60.2, 42.6, 42.5, 34.3, 28.9, 28.8, 26.0, 24.8, 23.6, 22.4, 14.2, 13.8; MS: m/e 279.1 [M+Na].

### Part C—Preparation of 8-Oxododecanoic Acid

A solution of the product of Part B (0.50 g, 2.1 mmol) in THF (7 mL) and water (2 mL) was treated with 3N LiOH (7.06 mL, 20.1 mmol), and stirred rapidly at room temperature under nitrogen for 18 hours. The THF was removed and the resulting mixture was acidified with 37% HCl (2.5 mL) to pH 4 and extracted with CH₂Cl₂ (20 mL). The organic layer was washed with saturated NaHCO₃ (20 mL), dried (MgSO₄), and concentrated to give the title compound as a colorless solid (0.32 g, 72%). ¹H NMR (DMSO-d₆): δ 2.42-2.33 (m, 4H), 2.08-2.03 (m, 2H), 1.47-1.39 (m, 6H), 1.28-1.14 (m, 6H), 0.85 (t, J=7.4 Hz; 3H); ¹³C NMR (DMSO-d₆): δ 210.5, 174.7, 41.7, 41.5, 34.2, 28.4, 28.3, 25.4, 24.6, 23.1, 21.7, 13.7; MS: m/e 197.3 [M-H₂O+H].

### Part D—Preparation of 8-Hydroxydodecanoic Acid

A solution of the product of Part C (0.15 g, 0.7 mmol) in ethanol (3 mL) was treated with NaBH₄ (0.013 g, 0.3 mmol) at 0° C. under nitrogen for 10 minutes. Additional NaBH₄ (0.052 g, 1.2 mmol) was added and the reaction was stirred for 1.5 hours. The reaction was quenched with 1N HCl (10 mL). The ethanol was removed under reduced pressure and the resulting solution was extracted with CH₂Cl₂ (3×10 mL). The combined organic layers were dried (MgSO₄) and concentrated to give the title compound as a colorless solid (0.118 g, 78%). ¹H NMR (DMSO-d₆): δ 11.95 (s, 1H), 4.19 (s, 1H), 2.18 (t, J=7.4 Hz, 2H), 1.52-1.47 (m, 2H), 1.35-1.20 (m, 14H), 0.86 (t, J=7.0 Hz, 3H); ¹³C NMR (DMSO-d₆): δ 174.4, 69.4, 37.1, 36.9, 33.6, 28.9, 28.6, 27.5, 25.1, 24.4, 22.3, 14.0; MS: m/e 181.4 [M−H₂O+H].

### Part E—Preparation of 2-((1E)-2-Aza-2-{[5-(N-{2-[(tert-butoxy)carbonylamino]-ethyl}carbamoyl)(2-pyridyl)]amino}vinyl)benzenesulfonic Acid

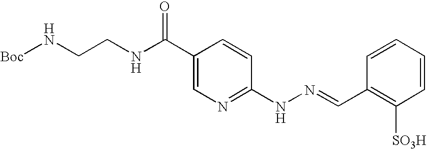

A solution of sodium 2-[(1E)-2-aza-2-({5-[(2,5-dioxopyrrolidinyl)oxycarbonyl](2-pyridyl)}amino)vinyl]benzenesulfonate (5.50 g, 12.5 mmol) and HOAt (1.70 g, 12.5 mmol) in N,N-dimethylformamide (8 mL) was treated with N-Boc-ethylenediamine (2.00 g, 12.5 mmol) and diisopropylethylamine (4.38 mL, 25.0 mmol), and the resulting solution was stirred at room temperature under nitrogen for 4 hours. The N,N-dimethylformamide was removed under reduced pressure and the resulting residue was chromatographed on silica gel, eluting with methanol to give the title compound as a pale yellow solid (3.48 g, 120%). MS: m/e 464.1 [M+H].

### Part F—Preparation of 2-[(1E)-2-({5-[N-(2-Aminoethyl)carbamoyl](2-pyridyl)}amino)-2-azavinyl]benzenesulfonic Acid

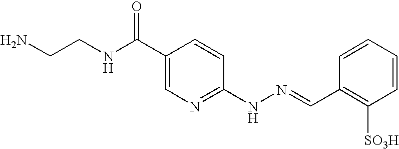

The product of Part E (2.8 g, 6.0 mmol) was dissolved in 50:50 trifluoroacetic acid:dichloromethane (10 mL) and stirred at room temperature under nitrogen for 10 minutes. The solution was concentrated and the resulting residue was purified by HPLC on Phenomenex Luna C18(2) column (41.4×250 mm) using a 0.9%/minute gradient of 0 to 18% acetonitrile containing 0.1% trifluoroacetic acid at a flow rate of 80 mL/min. The main product peaks eluting around 17.0 minutes were combined and lyophilized to give the title compound as a colorless solid (1.39 g, yield 64%, HPLC purity: 100%). ¹H NMR (DMSO-d₆): δ 9.18 (s, 1H), 8.68-8.52 (m, 2H), 8.28-8.05 (m, 2H), 7.91-7.65 (m, 4H), 7.50-7.32 (m, 2H), 7.27 (d, J=9.0 Hz, 1H), 3.62-3.45 (m, 2H), 3.15-2.94 (m, 2H); MS: m/e 364.1 [M+H]. High Resolution MS: Calculated for C15H17N5O4S [M+H]: 364.1074, Found: 364.1078.

### Part G—Preparation of 2-{(1E)-2-Aza-2-[(5-{N-[2-(8-hydroxydodecanoylamino)ethyl]carbamoyl}(2-pyridyl))amino]vinyl}benzenesulfonic Acid

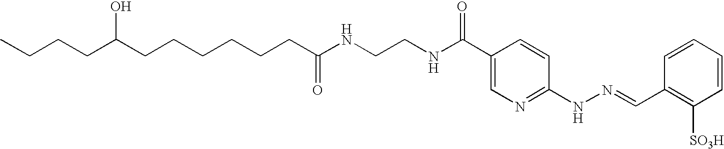

A solution of the product of Part F (0.025 g, 0.07 mmol), the product of Part D (0.015 g, 0.07 mmol), diisopropylethylamine (23 μL, 0.14 mmol), and HOAt (19 mg, 0.14 mmol) in anhydrous N,N-dimethylformamide (1.5 mL) was treated with DIC (21 μL, 0.14 mmol) and diisopropylethylamine (21 μL, 0.13 mmol) and the reaction was stirred at room temperature under nitrogen for 18 hours. The solution was concentrated under reduced pressure and the resulting residue was purified by HPLC on a Phenomenex Luna C18(2) column (21.2×250 mm) using a 0.9%/minute gradient of 18 to 41.4% acetonitrile containing 0.1% trifluoroacetic acid at a flow rate of 20 mL/min. The main product peak eluting at 21 minutes was lyophilized to give the title compound as a colorless solid (22.7 mg, yield 58%, HPLC purity 100%). MS: m/e 562.3 [M+H]; High Resolution MS: Calculated for C27H39N6O6S [M+H]: 562.2694, Found: 562.2681.

### EXAMPLE 24

### Synthesis of 2-((1E)-2-{[5-(N-{5-[N-({[4-((2S)-2-Smino-4-methylpentanoylamino)phenyl]methoxy}carbonylamino)carbamoyl]pentyl}carbamoyl)(2-pyridyl)]amino}-2-azavinyl)benzenesulfonic Acid

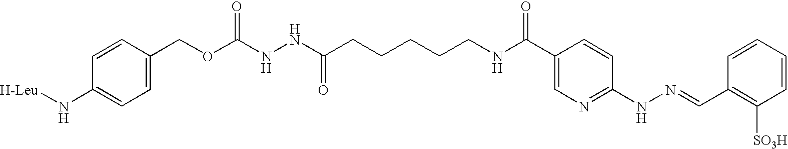

### Part A—Preparation of (2S)-2-[(tert-Butoxy)carbonylamino]-N-[4-(hydroxymethyl)phenyl]-4-methylpentanamide

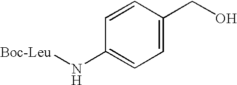

A solution of Boc-Leu-OH (2.02 g, 8.1 mmol), PABA (1.00 g, 8.1 mmol), and EEDQ (2.21 g, 8.9 mmol) in 1:1 toluene:ethanol (20 mL) was stirred at room temperature under nitrogen for 4 hours. The solution was concentrated under reduced pressure and the resulting residue was chromatographed on silica gel, eluting consecutively with 1:4 ethyl acetate:hexanes, 1:2 ethyl acetate:hexanes, and 1:1 ethyl acetate:hexanes to give the title compound as a colorless solid (2.62 g, 96%). ¹H NMR (CDCl₃): δ 8.46 (s, 1H), 7.49 (d, J=8.3 Hz, 2H), 7.28 (d, J=8.3 Hz, 2H), 4.98 (s, 1H), 4.64 (s, 2H), 4.27 (s, 1H), 1.83-1.73 (m, 2H), 1.70 (s, 1H), 1.62-1.55 (m, 1H), 1.47 (s, 9H), 1.030.93 (m, 6H); MS: m/e 237.3 [M-Boc+H]; High Resolution MS: Calculated for C18H28N2O4 [M+H]: 337.2122, Found: 337.2118.

### Part B—Preparation of (4-{(2S)-2-[(tert-Butoxy)carbonylamino]-4-methylpentanoylamino}phenyl)methyl(4-nitrophenoxy)formate

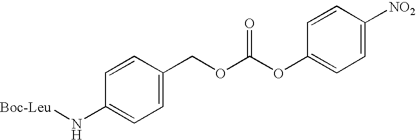

A solution of the product of Part A (1.00 g, 3.0 mmol) and 4-nitrophenyl chloroformate (0.6 g, 3.0 mmol) in anhydrous dichloromethane (10 mL) was cooled to 0° C., treated with pyridine (0.4 mL, 4.9 mmol) and stirred at ambient temperatures under nitrogen for 2 hours. The solution was diluted with CH₂Cl₂ (30 mL), washed with water (50 mL) and brine (50 mL), dried over MgSO₄, and concentrated under reduced pressure. The resulting residue was purified by flash chromatography on silica gel, eluting with 3:1 ethyl acetate/Hexanes to give the title compound as a colorless crystalline solid (1.02 g, 68%). ¹H NMR (CDCl₃): δ 8.48 (s, 1H), 8.30-8.26 (m, 2H), 7.57 (d, J=8.4 Hz, 2H), 7.42-7.36 (m, 4H), 5.25 (s, 2H), 4.92 (s, 1H), 4.24 (s, 1H), 1.85-1.70 (m, 2H), 1.62-1.53 (m, 1H), 1.48 (s, 9H), 1.02-0.95 (m, 6H); ¹³C NMR (CDCl₃): δ 170.9, 155.5, 152.4, 145.4, 138.6, 129.8, 129.7, 12.5.3, 121.8, 119.9, 80.8, 70.7, 53.8, 40.2, 28.3, 24.8, 22.9, 21.9; MS: m/e 524.3 [M+Na]; High Resolution MS: Calculated for C18H28N2O4 [M+H]: 502.2184, Found: 502.2183.

### Part C—Preparation of 2-((1E)-2-{[5-(N-{5-[N-({[4-((2S)-2-amino-4-methylpentanoylamino)phenyl]methoxy}carbonylamino)carbamoyl]-pentyl}carbamoyl)(2-pyridyl)]amino}-2-azavinyl)benzenesulfonic Acid

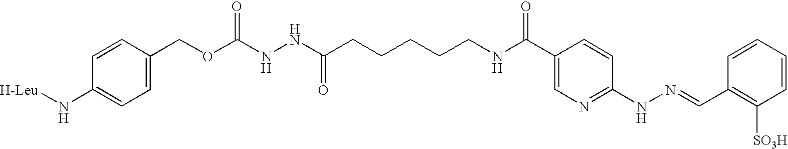

A solution of the product of Part B (105 mg, 0.2 mmol) and the product of Example 13, Part D (50 mg, 0.11 mmol) in anhydrous N,N-dimethylformamide (1 mL) was treated with TEA (17 μL, 0.12 mmol) and stirred at room temperature under nitrogen for 2 days. The solution was concentrated under reduced pressure and the resulting yellow viscous oil was dissolved in 50:50 trifluoroacetic acid:dichloromethane (4 mL) and stirred at room temperature under nitrogen for 10 minutes. The solution was concentrated and the resulting residue was purified by HPLC on a Phenomenex Luna C18(2) column (21.2×250 mm) using a 0.67%/minute gradient of 15 to 35% acetonitrile containing 0.1M NH₄OAc (pH 7) at a flow rate of 20 mL/min. The main product peak eluting at 23.2 minutes was lyophilized to give the title compound as a colorless solid (14 mg, yield 18%, HPLC purity 100%). ¹H NMR (DMSO-d₆): δ 11.30 (s, 1H), 10.43 (s, 1H), 9.60 (s, 1H), 9.05-9.00 (m, 2H), 8.59 (d, J=2.1 Hz, 1H), 8.30-8.25 (m, 1H), 8.05-7.98 (m, 2H), 7.78 (dd, J₁=7.7 Hz, J₂=1.3 Hz, 1H), 7.60 (d, J=8.1 Hz, 2H), 7.37-7.25 (m, 4H), 7.22 (d, J=8.8 Hz, 1H), 5.0 (s, 2H), 3.83 (t, J=7.0 Hz, 1H), 3.26-15 (m, 2H), 2.06-2.01 (m, 2H), 1.72-1.48 (m, 7H), 1.43-1.23 (m, 2H), 0.95-0.83 (m, 6H); ¹³C NMR (DMSO-d₆): δ 171.9, 171.8, 164.8, 158.5, 156.1, 147.8, 145.9, 137.8, 136.7, 132.2, 132.0, 128.7, 128.6, 127.5, 126.7, 125.1, 121.0, 119.3, 105.2, 65.5, 52.1, 40.7, 33.0, 28.9, 25.9, 24.7, 23.7, 22.7, 21.8, 21.0; MS: m/e 711.3 [M+H].

### EXAMPLE 25

### Synthesis of [4-((2S)-2-Amino-4-methylpentanoylamino)phenyl]methyl [11-(N-{2-[(tert-butoxy)carbonylamino]ethyl}carbamoyl)undecyloxy]formate

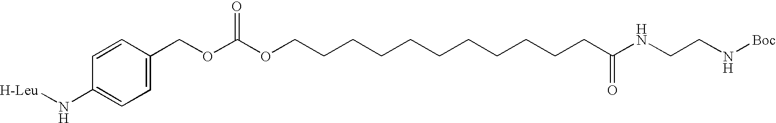

### Part A—Preparation of (2S)-2-[(Fluoren-9-ylmethoxy)carbonylamino]-N-[4-(hydroxymethyl)phenyl]-4-methylpentanamide

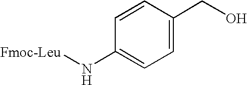

A solution of Fmoc-Leu-OH (2.0 g, 5.7 mmol), PABA (0.7 g, 5.7 mmol), and EEDQ (1.4 g, 6.3 mmol) in 1:1 toluene:ethanol (30 mL) was stirred at room temperature under nitrogen for 3 days. Additional PABA (0.14 g, 1.1 mmol) was added and the reaction was stirred for another 18 hours. Additional EEDQ (0.4 g, 1.9 mmol) was added and the reaction was stirred for another 2 hours, and concentrated. The resulting residue was dissolved in dichloromethane (20 mL), washed consecutively with 1N HCl (3×20 mL), saturated NaHCO₃ (3×20 mL), and brine (20 mL), dried (MgSO4), and concentrated. The resulting solid was purified by flash chromatography on silica gel, eluting with 50:1 dichloromethane:methanol to give the title compound as a colorless solid (2.03 g, 78%). ¹H NMR (DMSO-d₆): δ 9.96 (s, 1H), 7.89 (d, J=7.5 Hz, 2H), 7.74 (t, J=7.0 Hz, 2H), 7.63 (d, J=8.2 Hz, 1H), 7.55 (d, J=8.4 Hz, 2H), 7.44-7.38 (m, 2H), 7.34-7.29 (m, 2H), 7.23 (d, J=8.4 Hz, 2H), 5.08 (t, J=5.7 Hz, 1H), 4.43 (d, J=5.7 Hz, 2H), 4.30-4.19 (m, 4H), 1.73-1.64 (m, 1H), 1.63-1.56 (m, 1H), 1.49-1.44 (m, 1H), 0.96-0.73 (m, 6H); ¹³C NMR (DMSO-d₆): δ 171.3, 156.0, 143.9, 143.7, 140.7, 137.5, 137.4, 127.6, 127.0, 126.8, 125.3, 120.1, 119.0, 65.5, 62.5, 53.8, 46.7, 40.6, 24.3, 23.0, 21.4; MS: m/e 459.2 [M+H] (100%), 481.2 [M+Na] (60%).

### Part B—Preparation of (4-{(2S)-2-[(Fluoren-9-ylmethoxy)carbonylamino]-4-methylpentanoylamino}phenyl)methyl(4-nitrophenoxy)formate

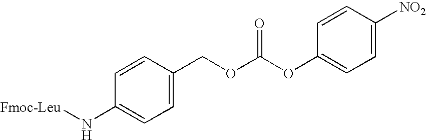

A solution of the product of Part A (0.50 g, 1.1 mmol) and 4-nitrophenyl chloroformate (0.66 g, 3.3 mmol) in anhydrous dichloromethane (15 mL) was treated with pyridine (0.73 mL, 8.9 mmol) and stirred at room temperature under nitrogen for 1.5 hours. The reaction mixture was filtered and the filtrate was concentrated. The resulting residue was purified by flash chromatography on silica gel, eluting with 1:3 EtOA:hexanes to give the title compound as a colorless crystalline solid (0.13 g, 19%). ¹H NMR (DMSO-d₆): δ 10.13 (s, 1H), 8.31 (d, J=9.1 Hz, 2H), 7.88 (d, J=7.3 Hz, 2H), 7.74 (t, J=7.0 Hz, 2H), 7.69-7.62 (m, 3H), 7.59-7.53 (m, 2H), 7.44-7.35 (m, 4H), 7.36-7.29 (m, 2H), 5.25 (s, 2H), 4.33-4.20 (m, 4H), 1.74-1.65 (m, 1H), 1.64-1.56 (m, 1H), 1.51-1.43 (m, 1H), 0.95-0.83 (m, 6H); ¹³C NMR (DMSO-d₆): δ 171.7, 156.0, 155.3, 151.9, 145.1, 143.8, 143.7, 140.7, 139.4, 129.4, 129.3, 127.6, 127.0, 126.2, 125.4, 125.3, 123.9, 122.6, 120.1, 119.2, 115.9, 70.2, 65.6, 53.8, 46.6, 40.5, 24.3, 23.0, 21.4; MS: m/e 624.2 [M+H].

### Part C—Preparation of N-{2-[(tert-Butoxy)carbonylamino]ethyl}-12-hydroxydodecanamide

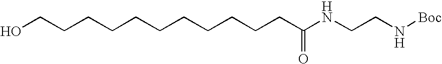

A solution of 12-hydroxydodecanoic acid (0.135 g, 0.6 mmol), N-Boc-ethylenediamine (0.100 g, 0.6 mmol), HOAt (0.170 g, 1.2 mmol), and diisopropylethylamine (0.22 mL, 1.2 mmol) in anhydrous N,N-dimethylformamide (1 mL) was treated with DIC (0.19 mL, 1.2 mmol) and the reaction was stirred at room temperature under nitrogen for 18 hours. The reaction was diluted with ethyl acetate (25 mL), washed consecutively with 1N HCl (25 mL), 0.5N NaOH (25 mL), and brine (25 mL), dried (MgSO₄), and concentrated. The resulting residue was purified by flash chromatography on silica gel, eluting with ethyl acetate to give the title compound as a colorless solid (0.237 g, contaminated with 1,3-diisopropylurea according to LC/MS [1]). MS: m/e 259.4 [M-Boc+H].

### Part D—Preparation of (4-{(2S)-2-[(Fluoren-9-ylmethoxy)carbonylamino]-4-methylpentanoylamino}phenyl)methyl [11-(N-{2-[(tert-butoxy)carbonylamino]-ethyl}carbamoyl)undecyloxy]formate

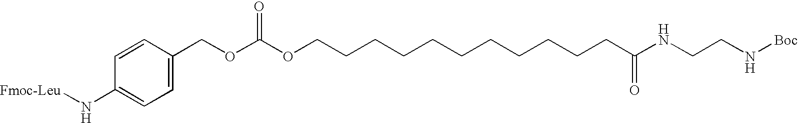

A solution of the product of Part B (50 mg, 0.08 mmol), the product of Part C (42 mg, 0.08 mmol), and DMAP (11 mg, 0.09 mmol) in anhydrous dichloromethane (3 mL) was stirred at room temperature under nitrogen for 28 hours. The solution was concentrated under reduced pressure and the resulting yellowish viscous oil was treated with 4 mL of 50% acetonitrile:water at room temperature under nitrogen for 10 minutes. The solvents were removed and the resulting residue was purified by HPLC on Phenomenex Luna C18(2) column (21.2×250 mm) using a 1.76%/minute gradient of 51.3 to 90% acetonitrile containing 0.1% formic acid at a flow rate of 20 mL/min. The main product peak eluting at 23.2 minutes was lyophilized to give the title compound as a colorless solid (22 mg, yield 33%, HPLC purity 100%). ¹H NMR (CDCl₃): δ 8.32 (bs, 1H), 7.75 (d, J=7.5 Hz, 2H), 7.58-7.53 (m, 2H), 7.53-7.47 (m, 2H), 7.37 (t, J=7.4 Hz, 2H), 7.33 (d, J=8.4 Hz, 2H), 7.28-7.25 (m, 2H), 6.15 (bs, 1H), 5.31 (bs, 1H), 5.10 (s, 2H), 4.95 (bs, 1H), 4.49-4.42 (m, 2H), 4.30 (bs, 1H), 4.20 (t, J=6.8 Hz, 1H), 4.13 (t, J=6.5 Hz, 2H), 3.39-3.27 (m, 2H), 3.26-3.21 (m, 2H), 2.14 (t, J=7.5 Hz, 2H), 1.81-1.53 (m, 7H), 1.42 (s, 9H), 1.36-1.30 (m, 2H), 1.30-1.19 (m, 12H), 1.00-0.90 (m, 6H); MS: m/e 843.5 [M+H]; High Resolution MS: Calculated for C48H66N4O9 [M+H]: 843.4903, Found: 843.4897.

### Part E—Preparation of [4-((2S)-2-Amino-4-methylpentanoylamino)phenyl]methyl [11-{2-[(tert-butoxy)carbonylamino]ethyl}carbamoyl)undecyloxy]formate

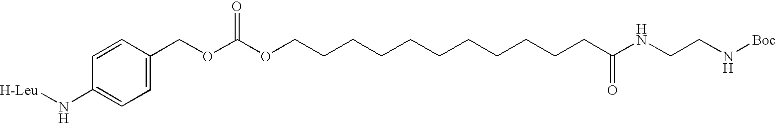

The product of Part D (7.0 mg, 0.008 mmol) was treated with 20% piperidine in N,N-dimethylformamide (1 mL) at room temperature under nitrogen for 5 minutes. The solution was concentrated under reduced pressure to give the title compound as a pale yellow solid. MS: m/e 621.5 [M+H](100%).

### EXAMPLE 26

### Synthesis of 2-((1E)-2-Aza-2-{[5-(N-{2-[8-(4-hydroxyphenyl)octanoylamino]ethyl}-carbamoyl)(2-pyridyl)]amino}vinyl)benzenesulfonic Acid

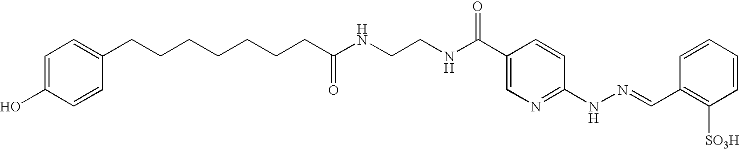

A solution of 8-(4-Hydroxyphenyl)octanoic acid (15.0 mg, 0.0635 mmol), the product of Example 23, Part F (23.1 mg, 0.0635 mmol), and HOAt (8.7 mg, 0.0635 mmol) in DMSO (200 μL) was treated with collidine (35 μL, 0.254 mmol) and DIC (9.9 μL, 0.0635 mmol), and allowed to stir under nitrogen at room temperature. After 21 hours, reaction mixture was treated with additional product of Example 23, Part F (11.6 mg, 0.0318 mmol), DIC (5.0 μL, 0.0318 mmol), and collidine (17.5 μL, 0.127 mmol). After 48 hours, the reaction mixture was treated with additional product of Example 23, Part F (5.8 mg, 0.0159 mmol), DIC (0.2.5 μL, 0.0159 mmol), and collidine (9 μL, 0.0635 mmol). After 58 hours, the reaction mixture was treated again with the product of Example 23, Part F (5.8 mg, 0.0159 mmol), DIC (0.2.5 μL, 0.0159 mmol), and collidine (9 μL, 0.0635 mmol). At a total reaction time of 63 hours, the reaction solution was purified by HPLC on a Phenomenex Luna column (21.2×250 mm) using a 1.12%/minute gradient of 0 to 56.2% acetonitrile containing 0.1% trifluoroacetic acid at a flow rate of 20 mL/min. The main product peak eluting at 36.2 minutes was lyophilized to give 16.3 mg (51.7%) of the desired compound as a colorless solid with 100% purity by HPLC. MS: m/e 582.2 [M+H](100%), 1163.3 [2M+H](35%).

### EXAMPLES 27 TO 44

### Synthesis of Complexes [99mTc(HYNIC-MMPsub)(tricine)(TPPTS)]

To a lead shielded lyophilized vial containing 4.84 mg TPPTS, 6.3 mg tricine, 40 mg mannitol, succinic acid buffer, pH 4.8, and 0.1% Pluronic F-64 surfactant, was added 1.1 mL sterile water for injection, 0.2 mL (20 μg) of the appropriate HYNIC-conjugated matrix metalloproteinase substrate (MMPsub) in deionized water or 50% aqueous ethanol, and 0.2 mL of 99 mTcO4- (50±5 mCi) in saline. The reconstituted kit was heated in a 95° C. water bath for 10 minutes, and was allowed to cool 5 minutes at room temperature. A sample of the reaction mixture was analyzed by HPLC. The RCP results are listed in the Table 1.

HPLC Method

Detector: INUS β-Ram, UV at 220 nm
Column: Zorbax Rx C18, 25 cm×4.6 mm
Guard: Zorbax C18
Temperature: Ambient
Flow: 1.0 mL/min
Solvent A: 25 mM ammonium acetate (no pH adjustment)

Solvent B: 100% Acetonitrile

| Gradient A | | | | | | |
|---|---|---|---|---|---|---|
| time (minutes) | 0 | 20 | 21 | 25 | 26 | 32 |
| % Solvent B | 10 | 40 | 60 | 60 | 10 | 10 |
| Gradient B | | | | | | |
| time (minutes) | 0 | 20 | 21 | 25 | 26 | 32 |
| % Solvent B | 5 | 15 | 60 | 60 | 5 | 5 |
| Gradient C | | | | | | |
| time (minutes) | 0 | 20 | 21 | 25 | 26 | 32 |
| % Solvent B | 0 | 20 | 60 | 60 | 0 | 0 |
| Gradient D | | | | | | |
| time (minutes) | 0 | 20 | 21 | 25 | 26 | 32 |
| % Solvent B | 30 | 50 | 70 | 70 | 30 | 30 |

TABLE 1
Analytical and Yield data for
[99mTc(HYNIC-MMPsub)(tricine)(TPPTS)] Complexes

| | HYNIC | HPLC | | RT |
|---|---|---|---|---|
| Example | Conjugate # | Gradient | % RCP | (minutes) |
| 27 | 1 | A | 95.7 | 11.7 |
| 28 | 2 | A | 97.2 | 15.1 |
| 29 | 3 | A | 84.1 | 14.2 |
| 30 | 5 | A | 79.6 | 12.7 |
| 31 | 8 | B | 76.1 | 12.8 |
| 32 | 10 | A | 93.7 | 18.3 |
| 33 | 11 | A | 94.5 | 14.4 |
| 34 | 12 | A | 89.8 | 14.2 |
| 35 | 13 | A | 96.8 | 16.9 |
| 36 | 14 | A | 94.9 | 13.8 |
| 37 | 15 | A | 94.4 | 11.9 |
| 38 | 16 | A | 95.2 | 16.6 |
| 39 | 17 | A | 91.2 | 16.9 |
| 40 | 19 | C | 99.3 | 9.8 |
| 41 | 20 | A | 90.8 | 12.8 |
| 42 | 21 | D | 87.4 | 8.9 |
| 43 | 22 | A | 91.1 | 14.6 |
| 44 | 26 | A | 97.8 | 12.8 |

### EXAMPLE 45

Kinetic Measurements of Hydrolysis of MMP Substrates

Part A—Activation and Active Site Titration of MMP-2 and MMP-9

Purified MMP-2 (10 μg) or MMP-9 (10 μg) were reconstituted in 100 μL of TCN buffer. Purified human MMP-9 was activated by incubation with 2 nM amino phenyl mercuric acetate (APMA) for 5.5 hours at 37° C. Pro-MMP-2 was activated by incubation with 2 nM APMA for 2 hours at 37° C. At the end of incubation 100 μl of 100% glycerol was added to active MMP-2 and active MMP-9 (final concentration 50% glycerol). Active MMP-2 and active MMP-9 were aliquoted and stored at −20° C.

Part B—Active Site Titration of MMP-2/MMP-9

The level of active protease was always quantified by active site titration studies prior to kinetic studies. The active site of MMP-9 and MMP-2 was titrated using the GM6001 dissolved in 100% DMSO at a stock concentration of 2.5 mM. Dilutions (1:2) of GM6001 were prepared in TCN buffer to give a final concentration of 5 nM to 0.04 nM GM6001 in the active site titration assay. Activated MMP-2 or activated MMP-9 (2 nM) was preincubated with increasing concentrations of GM60001 at 37° C. for 15 minutes in 96 well black microtiter plates. Fluorescent substrate I (Mca-P-L-G-L-Dpa-A-R-NH₂) (150 μL) in assay buffer (500 mM tricine/pH 7.5, 100 mM CaCl₂, 0.2% NaN₃) was added to the each well. The plate was shaken vigorously for 1 minute at room temperature and incubated at 27° C. for 1 hour. The reaction was stopped with 20 μL of 0.5 M EDTA. Plates were read on fluorescence spectrophotometer at excitation wavelength of 320 nm and emission wavelength of 395 nm. The concentration of the active enzyme was determined using the Morrison equation and Kaleidagraph software (Reading, Pa.).

Part C—Kinetic Measurements of Substrate Hydrolysis

The kinetic parameters of substrate hydrolysis were determined using a radio HPLC assay. The turnover of different substrates by active MMP-2 and active MMP-9 was determined using this assay. A stock solution of different test substrates (10 mM) was prepared in 100% DMSO. Stock solutions of the test substrates were diluted 1000 fold (10 nM) in buffer (50 mM Hepes/pH 7.5, 10 mM CaCl₂, 0.1% Brij) to give working stock solution. Working stock solution of the test substrate (15 μl) was added to buffer (120 μL) in a test tube and warmed at 37° C. for 2 minutes. To this solution 15 μL of working stock of active MMP-2 (final concentration 10 nM) or active MMP-9 was added (final concentration 2 nM). Finally, 4 μCi of radiolabeled test substrate was added and the solution was mixed and immediately 67.5 □L of the mix was transferred to HPLC vials containing 7.5 μl of 0.5M EDTA for t=0 minute measurement. The rest of the mix in the test tube was incubated at 37° C. for 60 minutes. At the 60 minute time point 67.5 μl of the mix was transferred to the HPLC vial containing 7.5 μL of 0.5 M EDTA for t=60 minute measurement. The radiolabeled substrates and products were separated by reversed phase HPLC on a Zorbax Rx-C18 column (4.6×250 mm) maintained at a column temperature of 25° C. with a 1 mL/min flow rate and 60 μL sample size. Mobile phase A (MPA) was 25 mM ammonium acetate and mobile phase B (MPB) was 100% acetonitrile. A step gradient of 2% MPB at 3 minutes, 40% MPB at 13 minutes, 80% MPB at 18 minutes was used for separation of products and substrate. The radiolabel was detected by a IN/US beta ram detector. The peak areas were integrated and the substrate peak area was used to determine rate constant k in the following equation:
k=(−ln(St/So))/t
where
St=Substrate peak at 60 minutes
So=Substrate peak at 0 minutes
T=3600 seconds.
   In this reaction substrate concentration is much lower than Km therefore
   Kcat/Km=k/[Et](M⁻¹S⁻¹)

The Kcat/Km values of various test substrates are presented in Table 2.

TABLE 2
Results from substrate hydrolysis assays

| | MMP2 | MMP9 | mouse MMP9 |
|---|---|---|---|
| Example | K_{cat}/Kₘ (M⁻¹s⁻¹) | K_{cat}/Kₘ (M⁻¹s⁻¹) | K_{cat}/Kₘ (M⁻¹s⁻¹) |
| 1 | 83,900 | 1670 | |
| 5 | 8025 | 1986 | |
| 3 | 11631 | 1742 | |
| 2 | 81562 | 6675 | |
| 1 | 42526 | 2978 | |
| 10 | 63172 | 189715.1454 | |
| 14 | 63685 | 4454 | 897 |
| 11 | 77740 | 22049 | 14352 |
| 12 | >100,000 | >100,000 | >100,000 |
| 16 | 63199 | >100,000 | >100,000 |
| 13 | >100,000 | >100,000 | >100,000 |
| 17 | 42684 | 57730 | 47964 |
| 15 | 265 | 613 | 571 |
| 18 | 19465 | 41623 | 30996 |

### EXAMPLE 46

Aminopeptidase N Cleavage of Test Substrates

Aminopeptidase N cleaves amino acids at the N-terminus of proteins and peptides attached to another amino acid. The final attachment in our test substrates consists of an amino acid linked to a hydrazide. The cleavage of this amino acid by aminopeptidases exposes the reactive hydrazide species. Our goal was to study the cleavage of amide bond between an amino acid and a hydrazide by aminopeptidase N. A stock solution of test substrates was prepared in 100% DMSO at a concentration of 25 mM. The stock soluton (6 μL) was added to buffer (50 mM Hepes/pH 7.5, 10 mM CaCl₂, 0.1% Brij) for a final concentration of 1 mM test substrate in the reaction. To this reaction mix 0.02 U of the enzyme (APN) was added, the solution was mixed, and immediately 67.5 μL of the mix was transferred to HPLC vials containing 33.2 μL of acetic acid for t=0 minute measurement. The rest of the mix in the test tube was incubated at 37° C. for 25 minutes. At the 25 minutes time point 67.5 μL of the mix was transferred to the HPLC vial containing 33.2 μl of acetic acid for t=25 minute measurement. The test substrates and products were separated by reversed phase HPLC and substrates on a Zorbax SB-C18 column (4.6×150 mm, 5 micron) using 0.1% trifluoroacetic acid/cetonitrile gradient method with UV detection. The peak areas were integrated and the substrate peak area was used to determine rate constant k in the following equation:
K={(% hydrolyzed/100)*[S]}/[E]*[time]
where
S=test substrate concentration in μmoles
E=aminopeptidase N concentration in units/ml
K=μmoles of substrate hydrolyzed/minute/unit enzyme

The rate of hydrolysis of the test substrates is shown in Table 3.

TABLE 3
Results from APN hydrolysis of test substrates

| Example | Rate of hydrolysis (min⁻¹, U⁻¹) |
|---|---|
| 8 | 0.6 μmoles |
| 9 | 0.62 μmoles |
| 7 | 1.2 μmoles |
| 6 | 0 μmoles |
| 24 | 0.52 μmoles |

### EXAMPLE 47

Lipid Bilayer Insertion

This assay was designed to study localization of test substrates in lipid bilayers of cells. THP-1 cell line a human monocytic cell line was used in this assay. THP-1 cells were washed with phosphate buffered saline (PBS) and 2×10⁶ cells were used for each reaction in a 150 μL reaction volume. Test substrates were added to these cell suspensions to give a final concentration of 0.15 mM in the reaction. The reactions were incubated at 37° C. for 1 hour. The test compound in the supernatant was analyzed by HPLC and quantified. The level of compound in the supernatant in the presence and absence of cells was determined and the following ratio was generated:
R=Level of compound in absence of cells/level of compound in the presence of cells

The ratio increases with increased binding to cells. A ratio of 1 denotes no binding to cells. The data for cell binding of various test compounds is shown in Table 4.

TABLE 4
Results from cell binding of test substrates

| Example | Ratio |
|---|---|
| 29 | >5 |
| 19 | 1.1 |
| 22 | 1.55 |

### EXAMPLE 48

### Synthesis of 2-{(1E)-2-[(5-{N-[2-(12-{[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)phenyl]methoxycarbonyloxy}dodecanoylamino)ethyl]-carbamoyl}(2-pyridyl))amino]-2-azavinyl}benzenesulfonic Acid

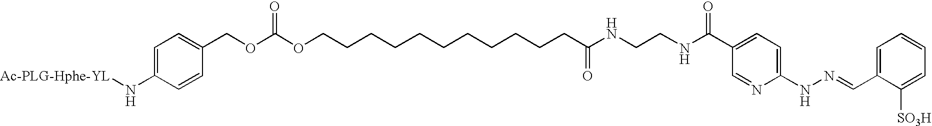

### Part A—Preparation of Ac-PLG-Hphe-Y(t-Bu)-OH

HMPB-BHA resin is placed in a peptide synthesis reaction vessel, and swollen by washing with N,N-dimethylformamide (2×). Fmoc-Tyr(t-Bu)-OH in N,N-dimethylformamide is added and the resin is mixed at room temperature for 15 minutes. Pyridine and 2,6-dichlorobenzoyl chloride are added and the mixture is gently shaken for 20 hours. The resin is then washed thoroughly with N,N-dimethylformamide (3×), dichloromethane (3×), methanol (3×), dichloromethane (3×), and N,N-dimethylformamide (3×). The remaining hydroxyl groups of the resin are capped by reacting with benzoyl chloride and pyridine in dichloromethane for 2 hours. The substitution level is determined by the quantitative fulvene-piperidine assay. The following steps are then performed: (Step 1) The Fmoc group is removed using 20% piperidine in N,N-dimethylformamide for 30 minutes. (Step 2) The resin is washed thoroughly with N,N-dimethylformamide (3×), dichloromethane (3×), methanol (3×), dichloromethane (3×), and N,N-dimethylformamide (3×). (Step 3) Fmoc-Hphe-OH, HOBt, and HBTU in N,N-dimethylformamide and diisopropylethylamine are added to the resin and the reaction is allowed to proceed for 8 hours. (Step 4) The resin is washed thoroughly with N,N-dimethylformamide (3×), dichloromethane (3×), methanol (3×), dichloromethane (3×), and N,N-dimethylformamide (3×). (Step 5) A double coupling is performed if the quantitative fulvene-piperidine assay shows the first coupling to be incomplete. (Step 6) The resin is washed thoroughly with N,N-dimethylformamide (3×), dichloromethane (3×), methanol (3×), dichloromethane (3×), and N,N-dimethylformamide (3×). Steps 1-6 are repeated until the sequence Fmoc-PLG-Hphe-Y(t-Bu)-OH is attained.

The peptide-resin is treated with 20% piperidine in N,N-dimethylformamide for 30 minutes, and washed thoroughly with N,N-dimethylformamide (3×), dichloromethane (3×), methanol (3×), dichloromethane (3×), and N,N-dimethylformamide (3×). Acetic anhydride, and diisopropylethylamine are added, and the resin is mixed until the capping reaction is found to be complete as assessed by LC/MS of a small portion of cleaved peptide. The peptide-resin is placed in a sintered glass funnel and treated with 1% trifluoroacetic acid in dichloromethane. After 2 minutes, the solution is filtered, by the application of pressure, directly into a solution of 10% pyridine in methanol. The cleavage step is repeated nine times. The combined filtrates are evaporated to 5% of their volume, diluted with water, and cooled in an ice-water bath. The resulting precipitate is collected by filtration in a sintered glass funnel, washed with water, and dried under vacuum. The resulting residue is purified by HPLC on a C18 column using a water:acetonitrile:0.1% trifluoroacetic acid gradient to give the title compound.

### Part B—Preparation of 11-(N-{2-[(tert-Butoxy)carbonylamino]ethyl}carbamoyl)undecyl {[4-((2S)-2-{(2S)-2-[(2S)-2-(2-{(2S)-2-[((2S)-1-acetylpyrrolidin-2-yl)carbonylamino]-4-methylpentanoylamino}acetylamino)-4-phenylbutanoylamino]-3-[4-(tert-butoxy)phenyl]propanoylamino}-4-methylpentanoylamino)phenyl]methoxy}formate

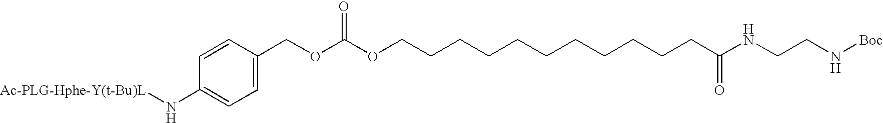

The product of Part A, above, the product of Example 25, Part E, HOAt, collidine, and DIC are dissolved in the minimal amount of DMSO and stirred at ambient temperatures under nitrogen for 24 hours. The solution is purified by HPLC on a C18 column using a water:acetonitrile:0.1% trifluoroacetic acid gradient. The product fraction is lyophilized to give the title compound.

### Part C—2-{(1E)-2-[(5-{N-[2-(12-{[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)phenyl]methoxycarbonyloxy}dodecanoylamino)ethyl]-carbamoyl}(2-pyridyl))amino]-2-azavinyl}benzenesulfonic Acid

The product of Part B is dissolved in 50:50 trifluoroacetic acid:dichloromethane and stirred at ambient temperatures under nitrogen for 60 minutes. The solution is concentrated under reduced pressure. The residue is dissolved in 1:1 toluene:ethanol, the pH is adjusted to 7 with diisopropylethylamine, and the solution is treated with 6-({(1E)-2-[2-(sodiooxysulfonyl)phenyl]-1-azavinyl}amino)pyridine-3-carboxylic acid (Bioconjugate Chem. 1999, 10, 808-814) and EEDQ. The reaction is allowed to proceed at ambient temperatures under nitrogen for 4 hours and concentrated under reduced pressure. The resulting residue is purified by HPLC on a C18 column using a water:acetonitrile:0.1% trifluoroacetic acid gradient. The product fraction is lyophilized to give the title compound.

### EXAMPLE 49

### Synthesis of 2-{(1E)-2-[(5-{N-[2-(8-{[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)phenyl]methoxycarbonyloxy}dodecanoylamino)ethyl]-carbamoyl}(2-pyridyl))amino]-2-azavinyl}benzenesulfonic Acid

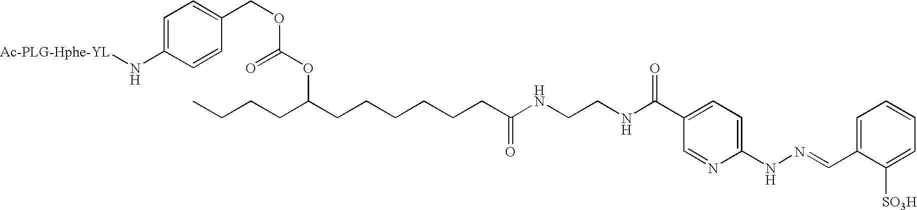

### Part A—Preparation of (2S)-N-({N-[(1S)-1-(N-{(1S)-1-[N-((1S)-1-{N-[4-(Hydroxymethyl)phenyl]carbamoyl}-3-methylbutyl)carbamoyl]-2-[4-(tert-butoxy)phenyl]ethyl}carbamoyl)-3-phenylpropyl]carbamoyl}methyl)-2-[((2S)-1-acetylpyrrolidin-2-yl)carbonylamino]-4-methylpentanamide

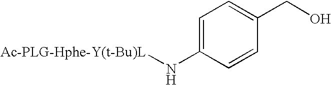

A solution of the product of Example 10, Part B, PABA, and EEDQ in 1:1 toluene:ethanol is stirred at room temperature under nitrogen for 3 days. Additional PABA is added if the reaction is incomplete, and the reaction is stirred for another 24 hours. The solution is concentrated under reduced pressure, and the resulting residue is purified by HPLC on a C18 column using a water:acetonitrile:0.1% trifluoroacetic acid gradient. The product fraction is lyophilized to give the title compound.

### Part B—Preparation of 4-Nitrophenyl {[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-[4-(tert-butoxy)phenyl]propanoylamino}-4-methylpentanoylamino)phenyl]methoxy}formate

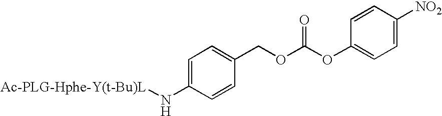

A solution of the product of Part A and 4-nitrophenyl chloroformate in anhydrous dichloromethane is cooled to 0° C., treated with pyridine and stirred at ambient temperatures under nitrogen for 2 hours. The solution is diluted with CH₂Cl₂, washed with water and brine, dried over MgSO₄, and concentrated under reduced pressure. The resulting residue is purified by flash chromatography on silica gel, eluting with ethyl acetate/Hexanes to give the title compound.

### Part C—Preparation of 2-((1E)-2-{[5-(N-{2-[8-({[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-[4-(tert-butoxy)phenyl]propanoylamino}-4-methylpentanoylamino)phenyl]methyl}oxycarbonyloxy)dodecanoylamino]ethyl}-carbamoyl)(2-pyridyl)]amino}-2-azavinyl)benzenesulfonic Acid

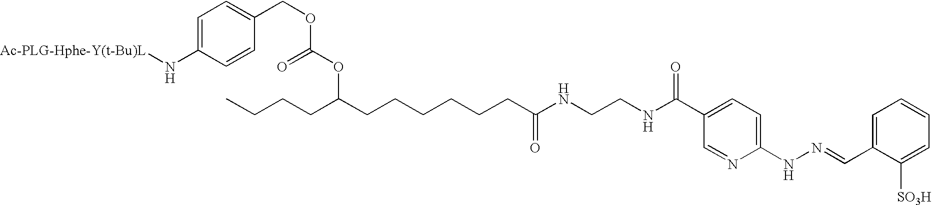

A solution of the product of Part B, above, the product of Example 23, and DMAP in anhydrous dichloromethane is stirred at room temperature under nitrogen until HPLC analysis determines the reaction is complete. The solution is concentrated under reduced pressure and the resulting residue is purified by reverse phase HPLC chromatography on a C18 column using a water:acetonitrile:0.1% trifluoroacetic acid gradient. The main product fraction is lyophilized to give the title compound.

### Part D—Final Deprotection

The product of Part C is dissolved in 50:50 trifluoroacetic acid:dichloromethane and stirred at ambient temperatures under nitrogen for 60 minutes. The solution is concentrated under reduced pressure and the resulting residue is purified by HPLC on a C18 column using a water:acetonitrile:0.1% trifluoroacetic acid gradient. The product fraction is lyophilized to give the title compound.

### EXAMPLE 50

### Synthesis of 2-{(1E)-2-[(5-{N-[2-(8-{[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-5-aminopentanoylamino}-4-methylpentanoylamino) phenyl]methoxycarbonyloxy}hexadec-15-enoylamino)ethyl]carbamoyl}(2-pyridyl))amino]-2-azavinyl}benzenesulfonic Acid

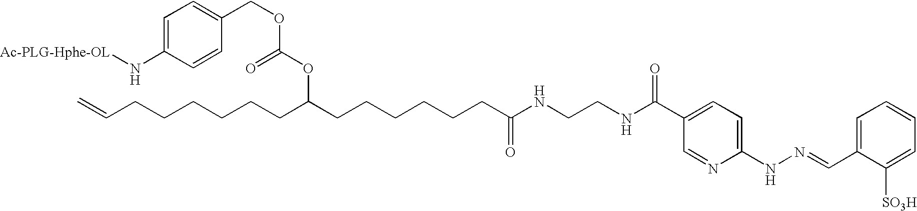

### Part A—Preparation of Ethyl 8-Oxohexadec-15-enoate

A solution of anhydrous Zinc chloride in anhydrous ether is treated with 7-octenylmagnesium bromide (prepared from 8-bromo-1-octene and magnesium in ether) dropwise at −78° C. The temperature is increased to 0° C. and the reaction mixture is treated with product of Example 23, part A in anhydrous THF followed by Pd(PPh₃)₄. The resulting mixture is stirred at 0° C. for 30 minutes, then at room temperature until complete by TLC or HPLC analysis. The reaction is quenched by the addition of 1N HCl and extracted with hexanes. The combined organic layers are washed with saturated NaHCO₃, dried (MgSO₄), and concentrated. The resulting residue is chromatographed on silica gel, eluting with ethyl acetate/Hexanes to give the title compound.

### Part B—Preparation of 8-Oxohexadec-15-enoic Acid

A mixture of the product of Part A in THF and water is treated with 3N LiOH and stirred rapidly at room temperature under nitrogen for 18 hours. The THF is removed and the resulting mixture is acidified with concentrated HCl to pH 4 and extracted with dichloromethane. The combined organic extracts are washed with saturated NaHCO₃, dried (MgSO₄), and concentrated to give the title compound, which is use in the next reaction without purification.

### Part C—Preparation of 8-Hydroxyhexadec-15-enoic Acid

A solution of the product of Part B in ethanol is treated with NaBH₄ at 0° C. under nitrogen until TLC or HPLC indicates the reaction is complete. Additional NaBH₄ is added if necessary. The reaction is quenched with 1N HCl. The ethanol is removed under reduced pressure and the resulting solution is extracted with CH₂Cl₂. The combined organic layers are dried (MgSO₄) and concentrated to give the title compound, which is used in the next reaction without purification.

### Part D—Preparation of 2-{(1E)-2-Aza-2-[(5-{N-[2-(8-hydroxyhexadec-15-enoylamino)ethyl]carbamoyl}(2-pyridyl))amino]vinyl}benzenesulfonic Acid

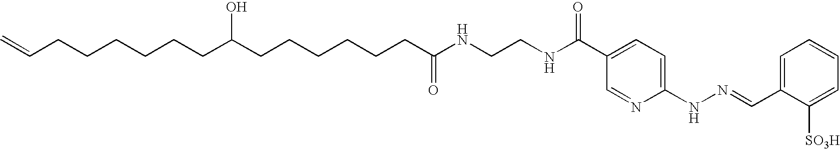

A solution of the product of Part C, above, the product of Experiment 23, Part F, diisopropylethylamine, and HOAt in anhydrous N,N-dimethylformamide is treated with DIC and the reaction is stirred at room temperature under nitrogen for 18 hours. The solution is concentrated under reduced pressure and the resulting residue is purified by HPLC on a C18 column using a water:acetonitrile:0.1% trifluoroacetic acid gradient. The main product peak is lyophilized to give the title compound.

### Part E—Preparation of (2S)-N-({N-[(1S)-1-(N-{(1S)-1-[N-((1S)-1-{N-[4-(Hydroxymethyl)phenyl]carbamoyl}-3-methylbutyl)carbamoyl]-4-[(tert-butoxy)carbonylamino]butyl}carbamoyl)-3-phenylpropyl]carbamoyl}methyl)-2-[((2S)-1-acetylpyrrolidin-2-yl)carbonylamino]-4-methylpentanamide

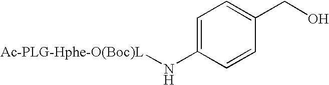

A solution of the product of Example 17, Part B, PABA, and EEDQ in 1:1 toluene:ethanol is stirred at room temperature under nitrogen for 3 days. Additional PABA is added if the reaction is incomplete, and the reaction is stirred for another 24 hours. The solution is concentrated under reduced pressure, and the resulting residue is purified by HPLC on a C18 column using a water:acetonitrile:0.1% trifluoroacetic acid gradient. The product fraction is lyophilized to give the title compound.

### Part F—Preparation of [4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-5-[(tert-butoxy)carbonylamino]pentanoylamino}-4-methylpentanoylamino)phenyl]methyl(4-Nitrophenoxy)formate

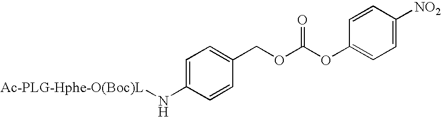

A solution of the product of Part E and 4-nitrophenyl chloroformate in anhydrous dichloromethane is cooled to 0° C., treated with pyridine and stirred at ambient temperatures under nitrogen for 2 hours. The solution is diluted with CH₂Cl₂, washed with water and brine, dried over MgSO₄, and concentrated under reduced pressure. The resulting residue is purified by HPLC on a C18 column using a water:acetonitrile:0.1% formic acid gradient. The product fraction is lyophilized to give the title compound.

### Part G—Preparation of 2-{(1E)-2-[(5-{N-[2-(8-{[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-5-[(tert-butoxy)carbonylamino]pentanoylamino)phenyl]methoxycarbonyloxy}hexadec-15-enoylamino)ethyl]carbamoyl}(2-pyridyl))amino]-2-azavinyl}benzenesulfonic Acid

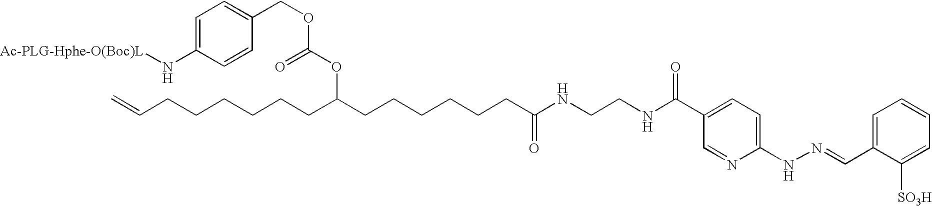

A solution of the products of Parts D and F, and DMAP in anhydrous dichloromethane is stirred at room temperature under nitrogen until HPLC analysis determines the reaction is complete. The solution is concentrated under reduced pressure and the resulting residue is purified by reverse phase HPLC chromatography on a C18 column using a water:acetonitrile:0.1% trifluoroacetic acid gradient. The main product fraction is lyophilized to give the title compound.

### Part H—Final Deprotection

The product of Part G is dissolved in 50:50 trifluoroacetic acid:dichloromethane and stirred at ambient temperatures under nitrogen for 10 minutes. The solution is concentrated under reduced pressure and the resulting residue is purified by HPLC on a C18 column using a water:acetonitrile:0.1% trifluoroacetic acid gradient. The product fraction is lyophilized to give the title compound.

### EXAMPLE 51

### Synthesis of 4-[(6-{[(1E)-1-Aza-2-(2-sulfophenyl)vinyl]amino}(3-pyridyl))carbonylamino](4S)-4-(N-{2-[8-(4-{2-[2-((2S)-2-{(2S)-2-[(2S)-2-(2-{(2S)-2-[((2S)-1-acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)-phenyl]acetyloxy}phenyl)octanoylamino]ethyl}carbamoyl)butanoic Acid

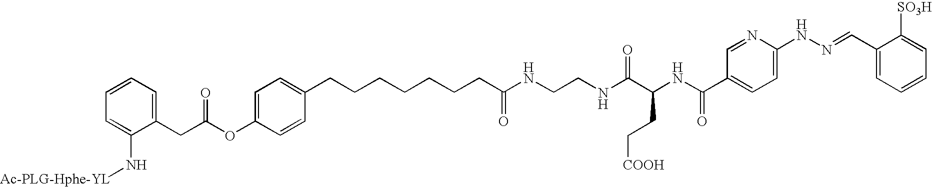 Part A—Preparation of (2S)-2-[(Fluoren-9-ylmethoxy)carbonylamino]-N-[2-(2-hydroxyethyl)phenyl]-4-methylpentanamide

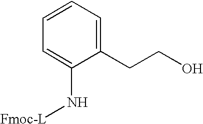

A solution of Fmoc-Leu-OH, 2-(4-aminophenyl)ethanol, and EEDQ in 1:1 toluene:ethanol is stirred at room temperature under nitrogen for 3 days. Additional 2-(4-aminophenyl)ethanol, and EEDQ are added if the reaction is incomplete, and the reaction is stirred for another 24 hours. The solution is concentrated under reduced pressure, and the resulting residue is taken up in dichloromethane, and washed consecutively with 0.1 N HCl, saturated NaHCO3, and saturated NaCl. The organic solution is dried (MgSO4) and concentrated, and the residue is purified by silica flash chromatography using a hexane:ethyl acetate mobile phase to give the title compound.

### Part B—Preparation of 2-(2-{(2S)-2-[(Fluoren-9-ylmethoxy)carbonylamino]-4-methylpentanoylamino}phenyl)acetic Acid

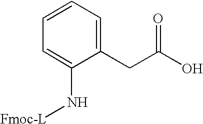

A solution of the product of Part A and pyridinium dichromate in N,N-dimethylformamide is stirred at ambient temperatures for 8 hours. The solution is diluted with 10 volumes of water and the precipitated product is extracted into ether. The combined ether extracts are washed consecutively with water and saturated NaCl, dried (MgSO4), and concentrated. The crude product is purified by recrystallization from ethanol to give the title compound.

### Part C—N-{2-[(tert-Butoxy)carbonylamino]ethyl}-8-(4-hydroxyphenyl)octanamide

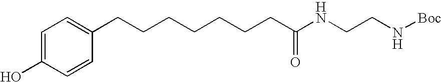

A solution of 8-(4-hydroxyphenyl)octanoic acid, N-Boc-ethylenediamine, and EEDQ in 1:1 toluene:ethanol is stirred at room temperature under nitrogen for 24 hours. The solution is concentrated under reduced pressure, and the resulting residue is taken up in dichloromethane, and washed consecutively with 0.1 N HCl, saturated NaHCO3, and saturated NaCl. The organic solution is dried (MgSO4) and concentrated, and the residue is purified by silica flash chromatography using a hexane:ethyl acetate mobile phase to give the title compound.

### Part D—Preparation of 4-[7-(N-{2-[(tert-Butoxy)carbonylamino]ethyl}carbamoyl)heptyl]phenyl 2-[2-(Methylamino)phenyl]acetate

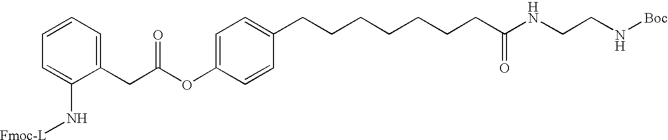

A solution of the product of Part B in anhydrous dichloromethane containing several drops of N,N-dimethylformamide is treated with one equivalent of oxalyl chloride and stirred at ambient temperatures for 3 hours. The solution is treated with the product of Part C and diisopropylethylamine, and stirred at ambient temperatures under nitrogen for 18 hours. The solution is washed consecutively with 0.1 N HCl, saturated NaHCO3, and saturated NaCl, dried (MgSO4), and concentrated. The residue is purified by flash chromatography on silica gel using a hexanes:ethyl acetate mobile phase to give the title compound.

### Part E—Preparation of tert-Butyl(4S)-4-{N-[2-(8-{4-[2-(2-{(2S)-2-[(Fluoren-9-ylmethoxy)carbonylamino]-4-methylpentanoylamino}phenyl)acetyloxy]phenyl}-octanoylamino)ethyl]carbamoyl}-4-[(phenylmethoxy)carbonylamino]butanoate

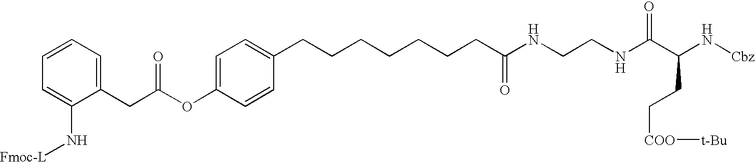

A solution of the product of Part E is dissolved in 50:50 trifluoroacetic acid:dichloromethane and stirred at ambient temperatures under nitrogen for 10 minutes. The solution is concentrated and the residue is taken up in anhydrous N,N-dimethylformamide and treated with diisopropylethylamine (to pH 8-9), and Cbz-Glu(t-Bu)-OSu. The solution is stirred at ambient temperatures for 18 hours and concentrated. The resulting residue is purified by HPLC on a C18 column using a water:acetonitrile:0.1% trifluoroacetic acid gradient. The product fraction is lyophilized to give the title compound.

### Part F—Preparation of tert-Butyl(4S)-4-(N-{2-[8-(4-{2-[2-((2S)-2-{(2S)-2-[(2S)-2-(2-{(2S)-2-[((2S)-1-Acetylpyrrolidin-2-yl)carbonylamino]-4-methylpentanoylamino}-acetylamino)-4-phenylbutanoylamino]-3-[4-(tert-butoxy)phenyl]propanoylamino}-4-methylpentanoylamino)phenyl]acetyloxy}phenyl)octanoylamino]ethyl}carbamoyl)-4-[(phenylmethoxy)carbonylamino]butanoate

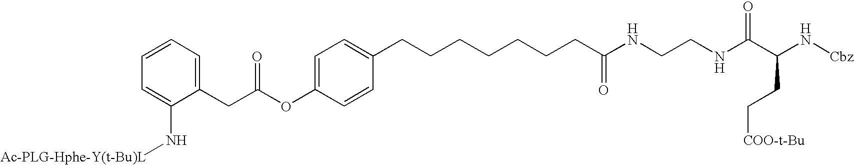

The product of Part E is dissolved in 20% piperidine in N,N-dimethylformamide and stirred at ambient temperatures for 10 minutes. The solution is concentrated under reduced pressure and dried thoroughly under high vacuum. The resulting residue is dissolved in a minimal amount of anhydrous DMSO along with the product of Example 48, Part A, and the solution is treated with HOAt, collidine, and DIC. The solution is stirred at ambient temperatures under nitrogen for 24 hours and purified by HPLC on a C18 column using a water:acetonitrile:0.1% trifluoroacetic acid gradient. The product fraction is lyophilized to give the title compound.

### Part G—Preparation of tert-Butyl-4-[(6-{[(1E)-1-aza-2-(2-sulfophenyl)vinyl]amino}(3-pyridyl))carbonylamino](4S)-4-(N-{2-[8-(4-{2-[2-((2S)-2-{(2S)-2-[(2S)-2-(2-{(2S)-2-[((2S)-1-acetylpyrrolidin-2-yl)carbonylamino]-4-methylpentanoylamino}acetylamino)-4-phenylbutanoylamino]-3-(4-[tert-butoxy]phenyl)propanoylamino}-4-methylpentanoylamino)phenyl]acetyloxy}phenyl)-octanoylamino]ethyl}-carbamoyl)butanoate

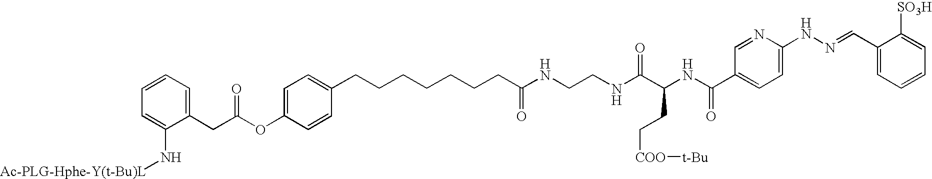

A solution of the product of Part F in ethanol is hydrogenated over 10% Pd/C at 60 psi until HPLC shows that the Cbz group is totally removed. The catalyst is removed by filtration thru Celite® and the filtrate is concentrated under reduced pressure. The residue is taken up in anhydrous N,N-dimethylformamide and treated with diisopropylethylamine, HOAt, and 2-[(1E)-2-aza-2-({5-[(2,5-dioxopyrrolidinyl)oxycarbonyl](2-pyridyl)}amino)vinyl]benzenesulfonate. The solution is stirred at ambient temperatures under nitrogen for 24 hours and concentrated under reduced pressure. The residue is purified by HPLC on a C18 column using a water:acetonitrile:0.1% trifluoroacetic acid gradient. The product fraction is lyophilized to give the title compound.

### Part H—Final Deprotection

The product of part G is dissolved in 95:2.5:2.5 trifluoroacetic acid:anisole:water (2 mL) and stirred at room temperature under nitrogen for 10 minutes. The solution is concentrated under reduced pressure and the resulting residue is purified by HPLC on a C18 column using a water:acetonitrile:0.1% trifluoroacetic acid gradient. The product fraction is lyophilized to give the title compound.

### EXAMPLE 52

### Synthesis of 2-[(1E)-2-({5-[N-(2-{8-[2-(N-{2-[((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-6-(dimethylamino)hexanoylamino}-4-methylpentanoylamino)methyl]phenyl}-N-methylcarbamoyloxy)-5-butylphenyl]octanoylamino}-ethyl)carbamoyl](2-pyridyl)}amino)-2-azavinyl]benzenesulfonic Acid

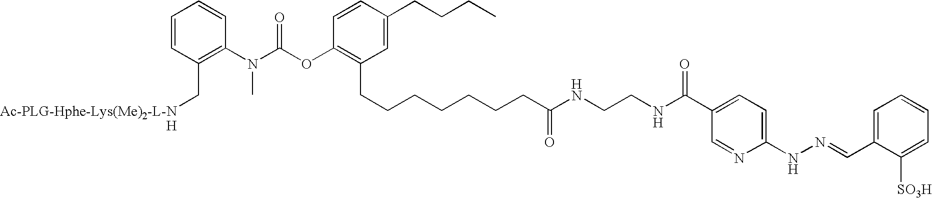

### Part A—Preparation of Ac-PLG-Hphe-K(Me2)-L-OH

The title compound is made using the procedure of Example 10, Parts A and B, by replacing Fmoc-Tyr(t-Bu)-OH with Fmoc-Lys(Me2) in the second coupling step. The crude peptide is purified by HPLC on a C18 column using a water:acetonitrile:0.1% trifluoroacetic acid gradient. The product fraction is lyophilized to give the title compound.

### Part B—Preparation of (2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-N-[(1S)-3-methyl-1-(N-{[2-(methylamino)phenyl]methyl}carbamoyl)butyl]-6-(dimethylamino)hexanamide

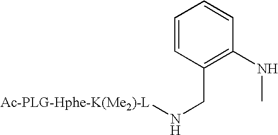

A solution of the product of Part A, N-methyl-2-aminomethylaniline (Coyne, W. E.; Cusic, J. W. J. Med. Chem. 1968, 11, 1208-1213), HBTU, and diisopropylethylamine in N,N-dimethylformamide is stirred at ambient temperatures under nitrogen for 18 hours. The solution is concentrated and the residue is purified by HPLC on a C18 column using a water:acetonitrile:10 mM NH4OAc gradient. The product fraction is lyophilized to give the title compound.

### Part C—Preparation of Ethyl 8-(5-Butyl-2-hydroxyphenyl)-8-oxooctanoate

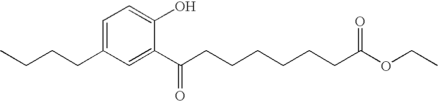

A solution of the product of Example 23, Part A, 4-butylphenol, and pyridine in dichloromethane is stirred at room temperature under nitrogen for 2 days. The solution is washed consecutively with 1.0 N HCl, saturated NaHCO3, and saturated NaCl, dried (MgSO4), and concentrated. The residue is dissolved in a minimum volume of 1,2-dichloroethane (DCE) and treated with aluminum chloride. The mixture is heated to reflux for 6 hours, cooled to room temperature, and poured onto ice. The layers are separated and the aqueous layer is extracted with dichloromethane. The combined dichloromethane and DCE layers are washed consecutively saturated NaHCO3 and saturated NaCl, dried (MgSO4), and concentrated. The residue is purified by flash chromatography on silica gel using a hexane:ethyl acetate mobile phase to give the title compound.

### Part D—Preparation of 8-(5-Butyl-2-hydroxyphenyl)octanoic Acid

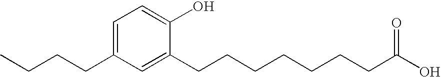

A solution of the product of Part C in aqueous ethanolic KOH is heated to reflux for 3 hours and concentrated to remove ethanol. The aqueous solution is washed with ether and acidified with concentrated HCl. The resulting precipitate is extracted into dichloromethane. The dichloromethane extracts are washed with water, dried (MgSO4), and concentrated. The residue is dissolved in diethylene glycol, and treated with 2 equivalents of hydrazine hydrate and 3 equivalents of KOH. The solution is heated to reflux for 1 hour, cooled, and diluted with water. The solution is made acidic with concentrated HCl, and the product is extracted into dichloromethane. The combined dichloromethane extracts are dried (MgSO4), and concentrated, and the residue is recrystallized to give the title compound.

### Part E—Preparation of N-{2-[(tert-Butoxy)carbonylamino]ethyl}-8-(5-butyl-2-hydroxyphenyl)octanamide

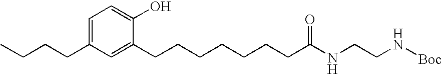

A solution of the product of Part D, N-Boc-ethylenediamine, and EEDQ in 1:1 toluene:ethanol is stirred at room temperature under nitrogen for 24 hours. The solution is concentrated under reduced pressure, and the resulting residue is taken up in dichloromethane, and washed consecutively with 0.1 N HCl, saturated NaHCO3, and saturated NaCl. The organic solution is dried (MgSO4) and concentrated, and the residue is purified by flash chromatography over silica gel using a hexane:ethyl acetate mobile phase to give the title compound.

### Part F—Preparation of 8-[2-(N-{2-[((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-6-(dimethylamino)hexanoylamino}-4-methylpentanoylamino)methyl]phenyl}-N-methylcarbamoyloxy)-5-butylphenyl]-N-{2-[(tert-butoxy)carbonylamino]ethyl}octanamide

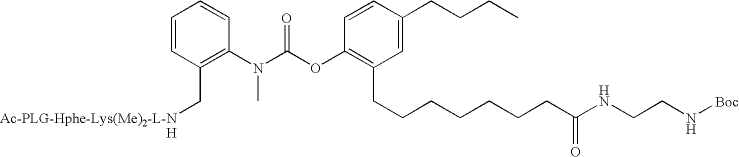

A solution of the product of Part E, pyridine, and triphosgene in dichloromethane is stirred at 0° C. for 30 minutes. The product of Part B is added and the solution is stirred at ambient temperatures for 18 hours. The solution is concentrated and the residue is purified by HPLC on a C18 column using a water:acetonitrile:0.1% trifluoroacetic acid gradient. The product fraction is lyophilized to give the title compound.

### Part G—Preparation of 2-[(1E)-2-({5-[N-(2-{8-[2-(N-{2-[((2S)-2-{(2S)-2-[(2S)-2-(2-{(2S)-2-[((2S)-1-Acetylpyrrolidin-2-yl)carbonylamino]-4-methylpentanoylamino}-acetylamino)-4-phenylbutanoylamino]-6-(dimethylamino)hexanoylamino}-4-methylpentanoylamino)methyl]phenyl}-N-methylcarbamoyloxy)-5butylphenyl]-octanoylamino}ethyl)carbamoyl](2-pyridyl)}amino)-2-azavinyl]benzenesulfonic Acid

The product of Part F is dissolved in 50:50 trifluoroacetic acid:dichloromethane and stirred at ambient temperatures under nitrogen for 10 minutes. The solution is concentrated, and the residue is dissolved in N,N-dimethylformamide, made basic with diisopropylethylamine and treated with sodium 2-[(1E)-2-aza-2-({5-[(2,5-dioxopyrrolidinyl)oxycarbonyl](2-pyridyl)}amino)vinyl]benzenesulfonate and HOAt. The solution is stirred at ambient temperatures under nitrogen for 18 hours and concentrated under vacuum. The residue is purified by HPLC on a C18 column using a water:acetonitrile:0.1% trifluoroacetic acid gradient. The product fraction is lyophilized to give the title compound.

### EXAMPLE 53

### Synthesis of 2-{(1E)-2-[(5-{N-[2-(10-{1-[(4-{(2S)-2-[(2S)-2-(2-{(2S)-2-[((2S)-1-{2-[N-(4-Aminobutyl)acetylamino]acetyl}pyrrolidin-2-yl)carbonylamino]-5-aminopentanoylamino}-acetylamino)-4-phenylbutanoylamino]-4-methylpentanoylamino}-phenyl)methyl](4-pyridinium)}undecanoylamino)-ethyl]carbamoyl}(2-pyridyl))amino]-2-azavinyl}benzenesulfonate Bis-Trifluoroacetate Salt

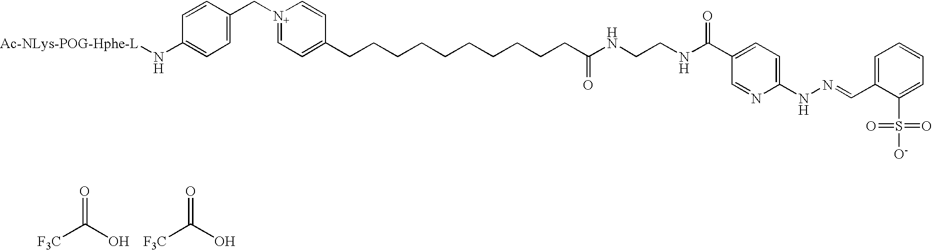

### Part A—Preparation of Methyl(10E)-11-(4-Pyridyl)undec-10-enoate

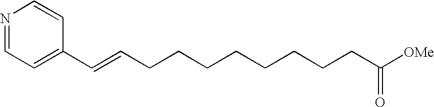

A solution of methyl 10-bromodecanoate and triphenyl phosphine in ethyl acetate is heated to reflux for 6 hours. The mixture is cooled and diluted with ether. The resulting precipitate of phosphonium salt is collected by filtration, washed with ether, and dried. In a separate flask anhydrous DMSO is treated with NaH and warmed at 60° C. under nitrogen to form the dimsyl sodium reagent. The phosphonium salt is added to the solution of dimsyl sodium and the solution is stirred at ambient temperatures for 3 hours. 4-Pyridinecarboxaldehyde is added and the solution is stirred at ambient temperatures for 18 hours. The solution is diluted with hexanes, washed with water, dried (MgSO4), and concentrated. The product is purified by flash chromatography over silica gel using a hexane:ethyl acetate mobile phase to give the title compound.

### Part B—Preparation of 11-(4-Pyridyl)undecaenoic Acid

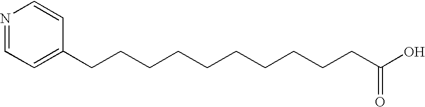

The product of Part A is dissolved in ethanol and hydrogenated over 10% Pd/C at 60 psi. The catalyst is removed by filtration through Celite® and the filtrate is concentrated under reduced pressure. The residue is dissolved in a slight excess of ethanolic KOH and heated to reflux for 24 hours. The solution is desalted by passing through an ion-exchange column made from IRC-50 resin. The eluant is concentrated under reduced pressure to give the title compound.

### Part C—Preparation of N-{2-[(tert-Butoxy)carbonylamino]ethyl}-11-(4-pyridyl)undecanamide

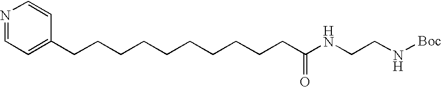

A solution of the product of Part B, N-Boc-ethylenediamine, and HBTU in anhydrous N,N-dimethylformamide is stirred at room temperature under nitrogen for 18 hours. The solution is concentrated under reduced pressure, and the resulting residue is taken up in dichloromethane, and washed consecutively with water, saturated NaHCO3, and saturated NaCl. The organic solution is dried (MgSO4) and concentrated, and the residue is purified by flash chromatography over silica gel using a hexane:ethyl acetate mobile phase to give the title compound.

### Part D—Preparation of 11-{1-[(4-{(2S)-2-[(Fluoren-9-ylmethoxy)carbonylamino]-4-methylpentanoylamino}phenyl)methyl](4-pyridinium)}-N-{2-[(tert-butoxy)carbonylamino]ethyl}undecanamide, Bromide

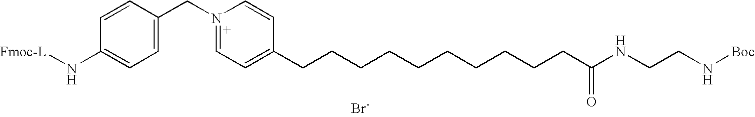

A solution of the product of Example 25, Part A, triphenylphosphine, and carbon tetrabromide in dichloromethane is stirred at ambient temperatures for 18 hours. The solution is concentrated to a small volume and filtered through alumina to remove triphenylphosphine oxide. The eluant is concentrated and the residue is taken up in anhydrous N,N-dimethylformamide, and treated with the product of Part C, above. The solution is stirred at ambient temperature for 18 hours and concentrated. The residue is purified by HPLC on a C18 column using a water:acetonitrile:0.1% formic acid gradient. The product fraction is lyophilized to give the title compound.

### Part E—Preparation of 2-{(1E)-2-[(5-{N-[2-(11-{1-[(4-{(2S)-2-[(Fluoren-9-ylmethoxy)carbonylamino]-4-methylpentanoylamino}phenyl)methyl](4-pyridinium)}undecanoylamino)ethyl]carbamoyl}(2-pyridyl))amino]-2-azavinyl}benzenesulfonate

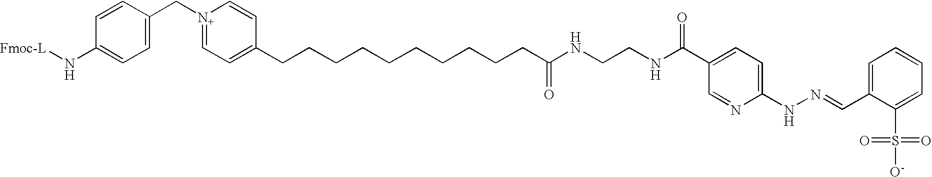

The product of Part D is dissolved in 50:50 trifluoroacetic acid:dichloromethane and stirred at room temperature under nitrogen for 10 minutes. The solution is concentrated and dried under vacuum. The residue is dissolved in anhydrous N,N-dimethylformamide and treated with diisopropylethylamine, HOAt, and 2-[(1E)-2-aza-2-({5-[(2,5-dioxopyrrolidinyl)oxycarbonyl](2-pyridyl)}amino)vinyl]benzene sulfonate. The solution is stirred at ambient temperatures under nitrogen for 24 hours and concentrated under reduced pressure. The residue is purified by HPLC on a C18 column using a water:acetonitrile:0.1% trifluoroacetic acid gradient. The product fraction is lyophilized to give the title compound.

### Part F—Preparation of Preparation of Ac-NLys(Boc)-PO(Boc)G-Hphe-OH

HMPB-BHA resin is placed in a peptide synthesis reaction vessel, and swollen by washing with N,N-dimethylformamide (2×). Fmoc-Hphe-OH in N,N-dimethylformamide is added and the resin is mixed at room temperature for 15 minutes. Pyridine and 2,6-dichlorobenzoyl chloride are added and the mixture is gently shaken for 20 hours. The resin is then washed thoroughly with N,N-dimethylformamide (3×), dichloromethane (3×), methanol (3×), dichloromethane (3×), and N,N-dimethylformamide (3×). The remaining hydroxyl groups of the resin are capped by reacting with benzoyl chloride and pyridine in dichloromethane for 2 hours. The substitution level is determined by the quantitative fulvene-piperidine assay. The following steps are then performed: (Step 1) The Fmoc group is removed using 20% piperidine in N,N-dimethylformamide for 30 minutes. (Step 2) The resin is washed thoroughly with N,N-dimethylformamide (3×), dichloromethane (3×), methanol (3×), dichloromethane (3×), and N,N-dimethylformamide (3×). (Step 3) Fmoc-Gly-OH, HOBt, and HBTU in N,N-dimethylformamide and diisopropylethylamine are added to the resin and the reaction is allowed to proceed for 8 hours. (Step 4) The resin is washed thoroughly with N,N-dimethylformamide (3×), dichloromethane (3×), methanol (3×), dichloromethane (3×), and N,N-dimethylformamide (3×). (Step 5) A double coupling is performed if the quantitative fulvene-piperidine assay shows the first coupling to be incomplete. (Step 6) The resin is washed thoroughly with N,N-dimethylformamide (3×), dichloromethane (3×), methanol (3×), dichloromethane (3×), and N,N-dimethylformamide (3×). Steps 1-6 are repeated until the sequence Fmoc-NLys(Boc)-PO(Boc)G-Hphe-OH is attained.

The peptide-resin is treated with 20% piperidine in N,N-dimethylformamide for 30 minutes, and washed thoroughly with N,N-dimethylformamide (3×), dichloromethane (3×), methanol (3×), dichloromethane (3×), and N,N-dimethylformamide (3×). Acetic anhydride, and diisopropylethylamine are added, and the resin is mixed until the capping reaction is found to be complete as assessed by LC/MS of a small portion of cleaved peptide. The peptide-resin is placed in a sintered glass funnel and treated with 1% trifluoroacetic acid in dichloromethane. After 2 minutes, the solution is filtered, by the application of pressure, directly into a solution of 10% pyridine in methanol. The cleavage step is repeated nine times. The combined filtrates are evaporated to 5% of their volume, diluted with water, and cooled in an ice-water bath. The resulting precipitate is collected by filtration in a sintered glass funnel, washed with water, and dried under vacuum. The resulting residue is purified by HPLC on a C18 column using a water:acetonitrile:0.1% trifluoroacetic acid gradient to give the title compound.

### Part G—Preparation of 2-[(1E)-2-({5-[N-(2-{10-[1-({4-[(2S)-2-((2S)-2-{2-[(2S)-2-({(2S)-1-[2-(N-{4-[(tert-butoxy)carbonylamino]butyl}acetylamino)acetyl]pyrrolidin-2-yl}carbonylamino)-5-[(tert-butoxy)carbonylamino]pentanoylamino]acetylamino}-4-phenylbutanoylamino)-4-methylpentanoylamino]phenyl}methyl)(4-pyridinium)]-decanoylamino}ethyl)carbamoyl](2-pyridyl)}amino)-2-azavinyl]benzenesulfonate

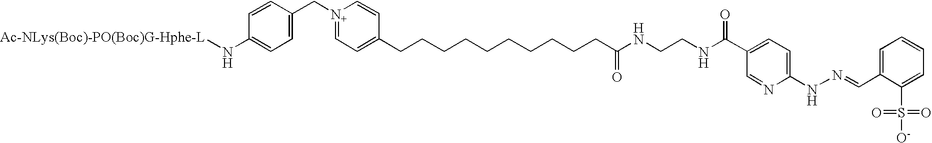

The product of Part E is dissolved in 20% piperidine in N,N-dimethylformamide and stirred at ambient temperatures for 10 minutes. The solution is concentrated under reduced pressure and dried thoroughly under high vacuum. The resulting residue is dissolved in a minimal amount of anhydrous DMSO along with the product of Part F and the solution is treated with HOAt, collidine, and DIC. The solution is stirred at ambient temperatures under nitrogen for 24 hours and concentrated under vacuum. The residue is purified by HPLC on a C18 column using a water:acetonitrile:0.1% trifluoroacetic acid gradient. The product fraction is lyophilized to give the title compound.

### Part H—Final Deprotection

A solution of the product of Part G in 50:50 trifluoroacetic acid:dichloromethane is stirred at ambient temperatures under nitrogen for 10 minutes and concentrated to dryness under high vacuum. The residue is purified by HPLC on a C18 column using a water:acetonitrile:0.1% trifluoroacetic acid gradient. The product fraction is lyophilized to give the title compound.

### EXAMPLE 54

### Synthesis of 2-{(1E)-2-[(5-{N-[2-(8-{(2S)-2-[(2S)-2-(2-{(2S)-2-[((2S)-1-{2-[N-(4-Aminobutyl)acetylamino]acetyl}pyrrolidin-2-yl)carbonylamino]-6-(amidinoamino)hexanoylamino}acetylamino)-4-phenylbutanoylamino]-4-methylpentanoylamino}(7Z)undec-7-enoylamino)ethyl]carbamoyl}(2-pyridyl))amino]-2-azavinyl}benzenesulfonic Acid Trifluoroacetate Salt

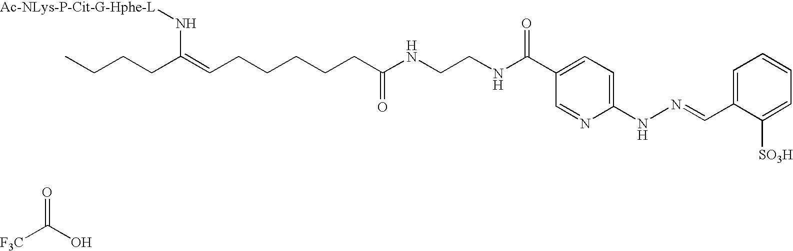

### Part A—Preparation of Ac-NLys(Boc)P-Cit-G-Hphe-OH

The title compound is prepared by the procedure described for Example 53, Part F, by replacing Fmoc-O(Boc)-OH with Fmoc-Cit-OH.

### Part B—Preparation of Ethyl(8Z)-9-Aza-8-butyl-12,12-dimethyl-12-silatridec-8-enoate

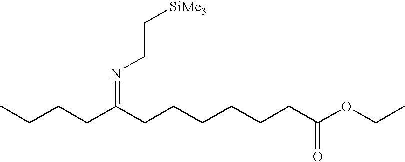

To a solution of the product of Example 23, Part B, 2-(trimethylsilyl)ethanamine (Sommer, L. H.; Rockett, J. J. Am. Chem. Soc. 1951, 73, 5130-5134), and a catalytic amount of p-toluenesulfonic acid in chloroform is added activated 4A molecular sieves. The reaction is allowed to stand at ambient temperatures under nitrogen for 2 days. The organic solution is decanted from the molecular sieves, washed consecutively with saturated NaHCO3, and saturated NaCl, dried (MgSO4), and concentrated to give the title compound, which is used directly in the next reaction.

### Part C—Preparation of Ethyl 8-{(2S)-2-[(tert-Butoxy)carbonylamino]-N-(3,3-dimethyl-3-silabutyl)-4-methylpentanoylamino}(7Z)dodec-7-enoate

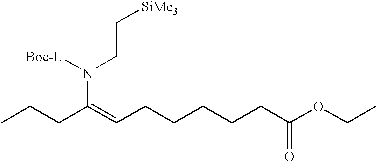

A solution of the product of Part B and Fmoc-leucine anhydride (Heimer, E. P.; Chang, C. D.; Lambros, T.; Meienhofer, J. Int. J. Peptide Protein Res. 1981, 18, 237) in pyridine is heated at reflux for 1 hour. The solution is concentrated and the residue is taken up in ethyl acetate and washed consecutively with 0.1 N HCl, saturated NaHCO3, and saturated NaCl, dried (MgSO4), and concentrated. The resulting residue is purified by flash chromatography over silica gel using a hexane:ethyl acetate mobile phase to give the title compound.

### Part D—Preparation of Ethyl 8-{(2S)-2-[(tert-Butoxy)carbonylamino]-4-methylpentanoylamino}(7Z)undec-7-enoate

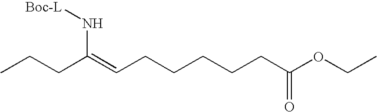

A solution of the product of part C in THF is treated with TBAF and stirred at ambient temperature under nitrogen for 2 hours. The solution is concentrated and the residue is taken up in ethyl acetate. The organic solution is washed consecutively with water and saturated NaCl, dried (MgSO4), and concentrated. The crude product is purified by flash chromatography over silica gel using a hexane:ethyl acetate mobile phase to give the title compound.

### Part E—Preparation of 2-{(1E)-2-[(5-{N-[2-(8-{(2S)-2-[(tert-Butoxy)carbonylamino]-4-methylpentanoylamino}(7Z)undec-7-enoylamino)ethyl]carbamoyl}(2-pyridyl))amino]-2-azavinyl}benzenesulfonic Acid

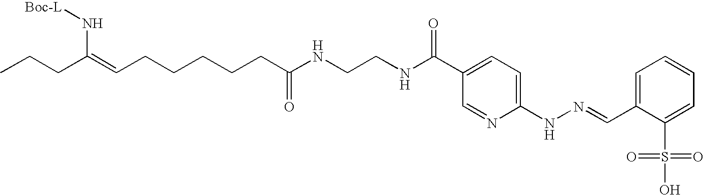

A solution of the product of Part D in THF and water is treated with 3N LiOH and stirred rapidly at room temperature under nitrogen until the ester hydrolysis is determined to be complete by TLC. The THF is removed and the resulting mixture is carefully acidified with HCl to pH 4 and extracted with dichloromethane. The organic extracts are washed with water, dried (MgSO4), and concentrated. The residue is purified by flash chromatography over silica gel using a hexane:ethyl acetate mobile phase. The resulting product is dissolved in anhydrous N,N-dimethylformamide along with the product of Example 23, Part F. The solution is made basic with diisopropylethylamine and treated with HBTU and HOAt. The reaction is sturred at ambient temperatures under nitrogen for 6 hours and concentrated under reduced pressure. The resulting residue is purified by HPLC on a C18 column using a water:acetonitrile:0.1% trifluoroacetic acid gradient. The product fraction is lyophilized to give the title compound.

### Part F—Preparation of 2-[(1E)-2-({5-[N-(2-{8-[(2S)-2-((2S)-2-{2-[(2S)-2-({(2S)-1-[2-(N-{4-[(tert-Butoxy)carbonylamino]butyl}acetylamino)acetyl]pyrrolidin-2-yl}carbonylamino)-6-(amidinoamino)hexanoylamino]acetylamino}-4-phenylbutanoylamino)-4-methylpentanoylamino](7Z)undec-7-enoylamino}ethyl)carbamoyl](2-pyridyl)}amino)-2-azavinyl]benzenesulfonic Acid

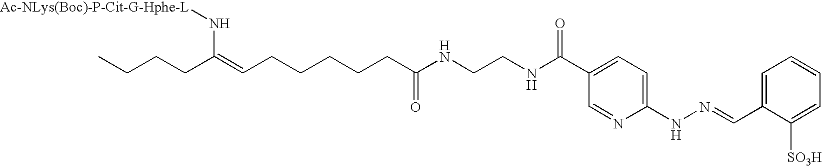

The product of Part E is dissolved in 50:50 trifluoroacetic acid:dichloromethane and stirred at room temperature under nitrogen for 10 minutes. The solution is concentrated and dried under high vacuum. A solution of the residue, the product of Part A, above, HBTU, HOAt, and diisopropylethylamine in anhydrous N,N-dimethylformamide is stirred at room temperature under nitrogen for 24 hours. The solution is concentrated and the residue is purified by HPLC on a C18 column using a water:acetonitrile:0.1% trifluoroacetic acid gradient. The product fraction is lyophilized to give the title compound.

### Part G—Final Deprotection

A solution of the product of Part G in 50:50 trifluoroacetic acid:dichloromethane is stirred at ambient temperatures under nitrogen for 10 minutes and concentrated to dryness under high vacuum. The residue is purified by HPLC on a C18 column using a water:acetonitrile:0.1% trifluoroacetic acid gradient. The product fraction is lyophilized to give the title compound.

### EXAMPLE 55

### Synthesis of 2-((1E)-2-{[5-(N-{2-[11-(4-{(2S)-2-[(2S)-2-(2-{(2S)-2-[((2S)-1-{2-[N-(4-Aminobutyl)acetylamino]acetyl}pyrrolidin-2-yl)carbonylamino]-6-(amidinoamino)hexanoylamino}acetylamino)-4-phenylbutanoylamino]-4-methylpentanoylamino}phenyl)undecanoylamino]ethyl}carbamoyl)(2-pyridyl)]amino}-2-azavinyl)benzenesulfonic Acid Trifluoroacetate Salt

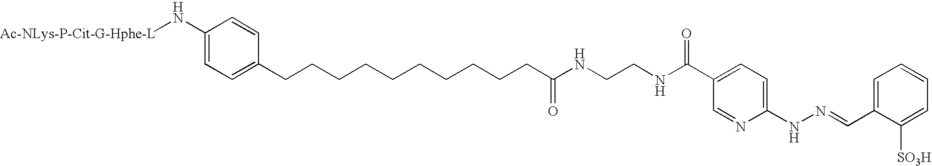

### Part A—Preparation of Methyl(10E)-11-[4-(2,2,2-Trifluoroacetylamino)phenyl]undec-10-enoate

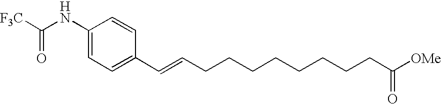

A solution of methyl 10-bromodecanoate and triphenyl phosphine in ethyl acetate is heated to reflux for 6 hours. The mixture is cooled and diluted with ether. The resulting precipitate of phosphonium salt is collected by filtration, washed with ether, and dried. In a separate flask anhydrous DMSO is treated with NaH and warmed at 60° C. under nitrogen to form the dimsyl sodium reagent. The phosphonium salt is added to the solution of dimsyl sodium and the solution is stirred at ambient temperatures for 3 hours. 4-(Trifluoroacetamido)benzaldehyde (Bonar-Law, R. P. J. Org. Chem. 1996, 61, 3623-3634) is added and the solution is stirred at ambient temperatures for 18 hours. The solution is diluted with hexanes, washed with water, dried (MgSO4), and concentrated. The product is purified by flash chromatography over silica gel using a hexane:ethyl acetate mobile phase to give the title compound.

### Part B—Preparation of 11-(4-Aminophenyl)undecanoic Acid

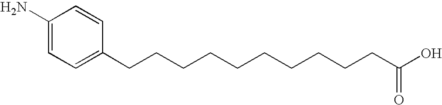

The product of Part A is dissolved in ethanol and hydrogenated over 10% Pd/C at 60 psi. The catalyst is removed by filtration through Celite® and the filtrate is concentrated under reduced pressure. The residue is dissolved in a slight excess of ethanolic KOH and heated to reflux for 24 hours. The solution is desalted by passing through an ion-exchange column made from IRC-50 resin. The eluant is concentrated under reduced pressure to give the title compound.

### Part C—Preparation of 2-((1E)-2-{[5-(N-{2-[11-(4-Aminophenyl)undecanoyl amino]ethyl}carbamoyl)(2-pyridyl)]amino}-2-azavinyl)benzenesulfonic Acid

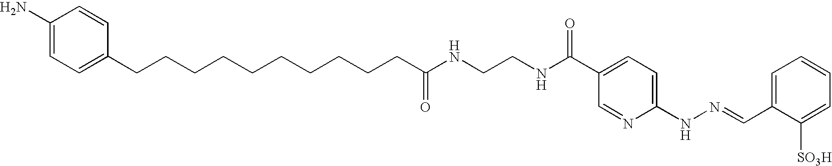

A solution of the product of Part B, the product of Example 23, Part F, and HBTU in anhydrous N,N-dimethylformamide is stirred at room temperature under nitrogen for 18 hours. The solution is concentrated under reduced pressure, and the resulting residue is taken up in dichloromethane, and washed consecutively with water, saturated NaHCO3, and saturated NaCl. The organic solution is dried (MgSO4) and concentrated, and the residue is purified by flash chromatography over silica gel using a hexane:ethyl acetate mobile phase to give the title compound.

### Part D—Preparation of 2-((1E)-2-{[5-(N-{2-[11-(4-{(2S)-2-[(Fluoren-9-ylmethoxy)carbonyl amino]-4-methylpentanoylamino}phenyl)undecanoylamino]-ethyl}carbamoyl)(2-pyridyl)]amino}-2-azavinyl)benzenesulfonic Acid

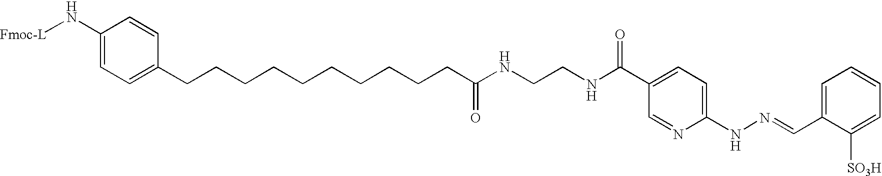

The product of Part C, Fmoc-Leu-OH, Part E, HOAt, collidine, and DIC are dissolved in the minimal amount of DMSO and stirred at ambient temperatures under nitrogen for 24 hours. The solution is purified by HPLC on a C18 column using a water:acetonitrile:0.1% trifluoroacetic acid gradient. The product fraction is lyophilized to give the title compound.

### Part E—Preparation of 2-{(1E)-2-[(5-{N-[2-(11-{4-[(2S)-2-((2S)-2-{2-[(2S)-2-({(2S)-1-[2-(N-{4-[(tert-Butoxy)carbonylamino]butyl}acetylamino)acetyl]pyrrolidin-2-yl}carbonylamino)-6-(amidinoamino)hexanoylamino]acetylamino}-4-phenylbutanoylamino)-4-methylpentanoylamino]phenyl}undecanoylamino)ethyl]-carbamoyl}(2-pyridyl))amino]-2-azavinyl}benzenesulfonic Acid

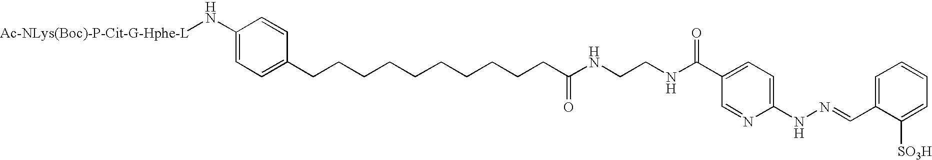

The product of Part D is dissolved in 20% piperidine in N,N-dimethylformamide and stirred at ambient temperatures for 10 minutes. The solution is concentrated under reduced pressure and dried thoroughly under high vacuum. The resulting residue is dissolved in a minimal amount of anhydrous DMSO along with the product of Example 54, Part A, and the solution is treated with HOAt, collidine, and DIC. The solution is stirred at ambient temperatures under nitrogen for 24 hours and concentrated under vacuum. The residue is purified by HPLC on a C18 column using a water:acetonitrile:0.1% trifluoroacetic acid gradient. The product fraction is lyophilized to give the title compound.

### Part F—Final Deprotection

A solution of the product of Part E in 50:50 trifluoroacetic acid:dichloromethane is stirred at ambient temperatures under nitrogen for 10 minutes and concentrated to dryness under high vacuum. The residue is purified by HPLC on a C18 column using a water:acetonitrile:0.1% trifluoroacetic acid gradient. The product fraction is lyophilized to give the title compound.

### EXAMPLE 56

### Synthesis of 2-[(1E)-2-({5-[N-(2-{12-[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino) pyridinium]dodecanoylamino}ethyl)carbamoyl](2-pyridyl)}amino)-2-azavinyl]benzenesulfonate

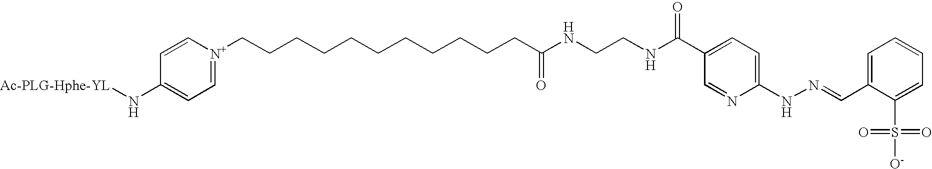

### Part A—Preparation of N-{2-[(tert-Butoxy)carbonylamino]ethyl}-12-bromododecanamide

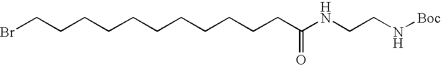

A solution of 12-bromododecanoic acid, N-Boc-ethylenediamine, HBTU, and 2,6-di-t-butylpyridine in anhydrous N,N-dimethylformamide is stirred at room temperature under nitrogen for 6 hours. The solution is concentrated under reduced pressure and the residue is taken up in ethyl acetate. The organic solution is washed consecutively with 1.0 N HCl, saturated NaHCO3, and saturated NaCl, dried (MgSO4), and concentrated. The resulting residue is purified by flash chromatography over silica gel using a hexane:ethyl acetate mobile phase to give the title compound.

### Part B—Preparation of (2S)-2-[(Fluoren-9-ylmethoxy)carbonylamino]-4-methyl-N-(4-pyridyl)pentanamide

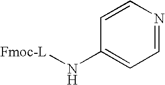

A solution of Fmoc-Leu-OH, 4-aminopyridine, HOAt, collidine, and DIC in the minimal amount of DMSO are stirred at ambient temperatures under nitrogen for 24 hours. The solution is purified by flash chromatography over silica gel to give the title compound.

### Part C—Preparation of (2S)-N-[(N-{(1S)-1-[N-((1S)-1-{N-[(1S)-3-Methyl-1-(N-(4-pyridyl)carbamoyl)butyl]carbamoyl}-2-[4-(tert-butoxy)phenyl]ethyl)carbamoyl]-3-phenylpropyl}carbamoyl)methyl]-2-[((2S)-1-acetylpyrrolidin-2-yl)carbonylamino]-4-methylpentanamide

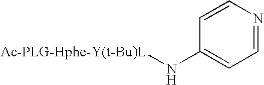

The product of Part B is dissolved in 20% piperidine in N,N-dimethylformamide and stirred at ambient temperatures for 10 minutes. The solution is concentrated under reduced pressure and dried thoroughly under high vacuum. The resulting residue is dissolved in a minimal amount of anhydrous DMSO along with the product of Example 48, Part A, and the solution is treated with HOAt, collidine, and DIC. The solution is stirred at ambient temperatures under nitrogen for 24 hours and concentrated under vacuum. The residue is purified by HPLC on a C18 column using a water:acetonitrile:50 mM NH4OAc gradient. The product fraction is lyophilized to give the title compound.

### Part D—Preparation of 12-[4-((2S)-2-{(2S)-2-[(2S)-2-(2-{(2S)-2-[((2S)-1-acetylpyrrolidin-2-yl)carbonylamino]-4-methylpentanoylamino}acetylamino)-4-phenylbutanoylamino]-3-[4-(tert-butoxy)phenyl]propanoylamino}-4-methylpentanoylamino)-pyridyl]-N-{2-[(tert-butoxy)carbonylamino]ethyl}dodecanamide, Bromide

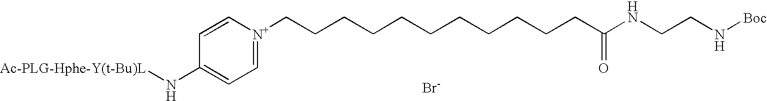

The products of Parts A and C are dissolved in anhydrous N,N-dimethylformamide, stirred at ambient temperature for 18 hours, and concentrated. The residue is purified by HPLC on a C18 column using a water:acetonitrile:0.1% formic acid gradient. The product fraction is lyophilized to give the title compound.

### Part E—Preparation of 2-[(1E)-2-({5-[N-(2-{12-[4-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)pyridinium]dodecanoylamino}ethyl)carbamoyl](2-pyridyl)}-amino)-2-azavinyl]benzenesulfonate

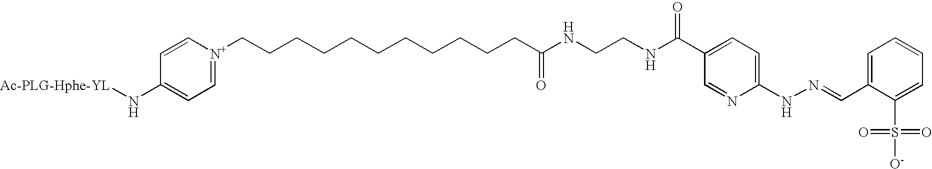

The product of Part D is dissolved in 95:2.5:2.5 trifluoroacetic acid:Et3SiH:water and heated with stirring at 60° C. under nitrogen for 30 minutes. The solution is concentrated under reduced pressure. The residue is dissolved in 1:1 toluene:ethanol, the pH is adjusted to 7 with diisopropylethylamine, and the solution is treated with 6-({(1E)-2-[2-(sodiooxysulfonyl)phenyl]-1-azavinyl}amino)pyridine-3-carboxylic acid (Bioconjugate Chem. 1999, 10, 808-814) and EEDQ. The reaction is allowed to proceed at ambient temperatures under nitrogen for 4 hours and concentrated under reduced pressure. The resulting residue is purified by HPLC on a C18 column using a water:acetonitrile:0.1% trifluoroacetic acid gradient. The product fraction is lyophilized to give the title compound.

### EXAMPLE 57

### Synthesis of 2-[(1E)-2-({5-[N-(2-{2-[4-(2-{3-[2-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-(4-hydroxyphenyl)propanoylamino}-4-methylpentanoylamino)-4,6-dimethylphenyl]-3-methylbutanoyloxy}prop-2-enoyl)phenyl]acetylamino}-ethyl)carbamoyl](2-pyridyl)}amino)-2-azavinyl]benzenesulfonic Acid

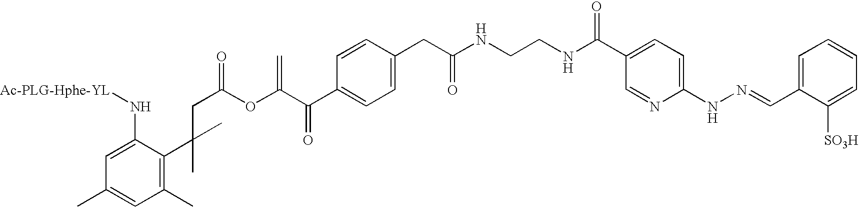 Part A—Preparation of 3-(2-Amino-4,6-dimethylphenyl)-3-methylbutan-1-ol

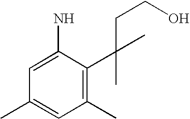

A solution of 3,5-dimethylaniline, 3,3-dimethylacryloyl chloride, and TEA in dichloromethane is stirred at room temperature for 2 hours. The solution is washed consecutively with water, saturated NaHCO3, and saturated NaCl, dried (MgSO4), and concentrated. The residue is purified by flash chromatography over silica gel using a hexane:ethyl acetate mobile phase. This purified intermediate is dissolved in anhydrous THF and treated with lithium aluminum hydride. The reaction is stirred under nitrogen at ambient temperatures for 2 hours and quenched by the addition of a saturated solution of ammonium chloride. The precipitated inorganic salts are removed by filtration through Celite®. The filtrate is concentrated and the residue is purified by flash chromatography over silica gel using a hexane:ethyl acetate mobile phase to give the title compound.

### Part B—Preparation of (2S)-2-[(Fluoren-9-ylmethoxy)carbonylamino]-N-[2-(3-hydroxy-1,1-dimethylpropyl)-3,5-dimethylphenyl]-4-methylpentanamide

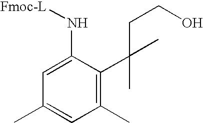

A solution of Fmoc-Leu-OH, the product of Part A, and EEDQ in 1:1 toluene:ethanol is stirred at room temperature under nitrogen for 3 days. Additional 2-(4-aminophenyl)ethanol, and EEDQ are added if the reaction is incomplete, and the reaction is stirred for another 24 hours. The solution is concentrated under reduced pressure, and the resulting residue is taken up in dichloromethane, and washed consecutively with 0.1 N HCl, saturated NaHCO3, and saturated NaCl. The organic solution is dried (MgSO4) and concentrated, and the residue is purified by silica flash chromatography using a hexane:ethyl acetate mobile phase to give the title compound.

### Part C—Preparation of (2S)-N-({N-[(1S)-1-(N-{(1S)-1-[N-((1S)-1-{N-[2-(3-Hydroxy-1,1-dimethylpropyl)-3,5-dimethylphenyl]carbamoyl}-3methylbutyl)carbamoyl]-2-[4-(3,3-dimethyl-3-silabutoxy)phenyl]ethyl}carbamoyl)-3-phenylpropyl]carbamoyl}methyl)-2-[((2S)-1-acetylpyrrolidin-2-yl)carbonylamino]-4-methylpentanamide

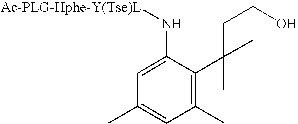

The product of Part B is dissolved in 20% piperidine in N,N-dimethylformamide and stirred at ambient temperatures for 10 minutes. The solution is concentrated under reduced pressure and dried thoroughly under high vacuum. The resulting residue is dissolved in a minimal amount of anhydrous DMSO along with Ac-PLG-Hphe-Y(Tse)-OH (prepared according to the procedure of Example 48, Part A by replacing Fmoc-Tyr(t-Bu)-OH with Fmoc-Tyr(Tse)-OH), and the solution is treated with HOAt, collidine, and DIC. The solution is stirred at ambient temperatures under nitrogen for 24 hours and purified by HPLC on a C18 column using a water:acetonitrile:0.1% trifluoroacetic acid gradient. The product fraction is lyophilized to give the title compound.

### Part D—Preparation of 3-[2-((2S)-2-{(2S)-2-[(2S)-2-(2-{(2S)-2-[((2S)-1-Acetylpyrrolidin-2-yl)carbonylamino]-4-methylpentanoylamino}acetylamino)-4phenylbutanoylamino]-3-[4-(3,3-dimethyl-3-silabutoxy)phenyl]propanoylamino}-4-methylpentanoylamino)-4,6-dimethylphenyl]-3-methylbutanoic Acid

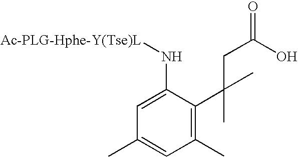

A solution of the product of Part D, TEMPO, and BAIB in 50:50 acetonitrile:water is stirred at 0° C. for 6 hours and concentrated. The iodobenzene by-product is removed azeotropically by dissolving the residue in 50:50 i-PrOH:water and concentrating. The cruce product is purified by HPLC on a C18 column using a water:acetonitrile:0.1% trifluoroacetic acid gradient. The product fraction is lyophilized to give the title compound.

### Part E—Preparation of 1-Methylvinyl 3-[2-((2S)-2-{(2S)-2-[(2S)-2-(2-{(2S)-2-[((2S)-1-Acetylpyrrolidin-2-yl)carbonylamino]-4-methylpentanoylamino}acetylamino)-4-phenylbutanoylamino]-3-[4-(3,3-dimethyl-3-silabutoxy)phenyl]propanoylamino}-4-methylpentanoylamino)-4,6-dimethylphenyl]-3-methylbutanoate

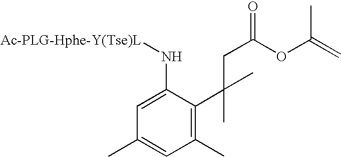

A solution of the product of Part D, vinyl acetate, mercuric acetate, and concentrated sulfuric acid is heated at reflux for 3 hours. Sodium acetate is added to neutralize the acid, and the mixture is concentrated to dryness. The residue is purified by HPLC on a C18 column using a water:acetonitrile gradient. The product fraction is lyophilized to give the title compound.

### Part F—Preparation of N-{2-[(Fluoren-9-ylmethoxy)carbonylamino]ethyl}-2-[4-(2-oxopropanoyl)phenyl]acetamide

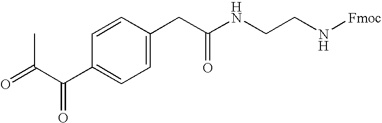

A solution of 2-[4-(2-oxopropanoyl)phenyl]acetic acid (McPherson, D. W.; Umbricht, G.; Knapp, F. F., Jr. J. Labelled Compounds Radiopharm. 1990, 28, 877-899), N-(2-aminoethyl)(fluoren-9-ylmethoxy)carboxamide, HBTU, and diisopropylethylamine in anhydrous N,N-dimethylformamide is stirred at ambient temperatures for 6 hours and concentrated under reduced pressure. The residue is dissolved in ethyl acetate and washed consecutively with 1.0 N HCl, saturated NaHCO3, and saturated NaCl, dried (MgSO4), and concentrated. The residue is purified by flash chromatography over silica gel using a hexane:ethyl acetate mobile phase to give the title compound.

### Part G—Preparation of 2-{4-[(N-{2-[(Fluoren-9-ylmethoxy)carbonylamino]ethyl}-carbamoyl)methyl]phenyl}-1-methylene-2-oxoethyl 3-[2-((2S)-2-{(2S)-2-[(2S)-2-(2-{(2S)-2-[((2S)-1-acetylpyrrolidin-2-yl)carbonylamino]-4-methylpentanoylamino}-acetylamino)-4-phenylbutanoylamino]-3-[4-(3,3-dimethyl-3-silabutoxy)phenyl]-propanoylamino}-4-methylpentanoylamino)-4,6-dimethylphenyl]-3-methylbutanoate

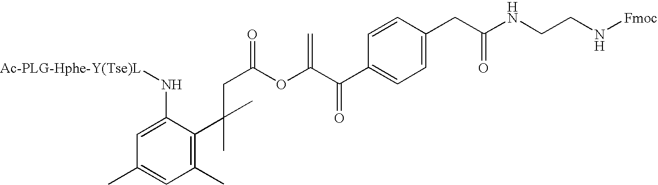

A solution of the products of Parts E and F and p-TsOH in CHCl₃ is heated at reflux for 18 hours. The solution is washed consecutively with 1.0 N HCl, saturated NaHCO3, and saturated NaCl, dried (MgSO4), and concentrated to dryness. The residue is purified by HPLC on a C18 column using a water:acetonitrile gradient. The product fraction is lyophilized to give the title compound.

### Part H—Preparation of 2-[(1E)-2-({5-[N-(2-{2-[4-(2-{3-[2-((2S)-2-{(2S)-2-[(2S)-2-(2-{(2S)-2-[((2S)-1-Acetylpyrrolidin-2-yl)carbonylamino]-4-methylpentanoylamino}-acetylamino)-4-phenylbutanoylamino]-3-(4-(3,3-dimethyl-3-silabutoxy)phenyl)propanoylamino}-4-methylpentanoylamino)-4,6-dimethylphenyl]-3-methylbutanoyloxy}prop-2-enoyl)phenyl]acetylamino}ethyl)carbamoyl](2-pyridyl)}amino)-2-azavinyl]benzenesulfonic Acid

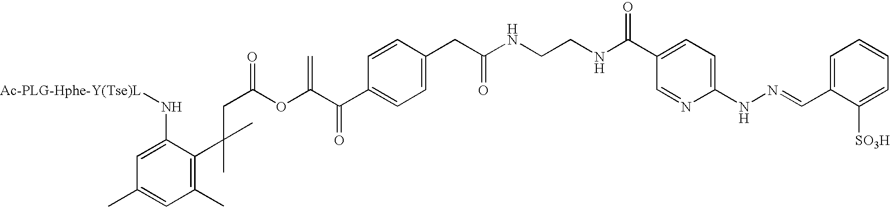

The product of Part G is dissolved in 20% piperidine in N,N-dimethylformamide and stirred at ambient temperatures for 10 minutes. The solution is concentrated under reduced pressure and dried thoroughly under high vacuum. The residue is taken up in anhydrous N,N-dimethylformamide and treated with diisopropylethylamine, HOAt, and 2-[(1E)-2-aza-2-({5-[(2,5-dioxopyrrolidinyl)oxycarbonyl]-(2-pyridyl)}amino)vinyl]benzenesulfonate. The solution is stirred at ambient temperatures under nitrogen for 24 hours and concentrated under reduced pressure. The residue is purified by HPLC on a C18 column using a water:acetonitrile:0.1% trifluoroacetic acid gradient. The product fraction is lyophilized to give the title compound.

### Part I—Final Deprotection

A solution of the product of part H in THF is treated with TBAF and stirred at ambient temperature under nitrogen for 2 hours. The solution is concentrated and the resulting residue is purified by HPLC on a C18 column using a water:acetonitrile:0.1% trifluoroacetic acid gradient. The product fraction is lyophilized to give the title compound.

### EXAMPLE 58

### Synthesis of the 4-[((4,4,4-Triphenylbutyl) {[N-(4,4,4-triphenylbutyl)carbamoyl]methyl}-amino)methyl]benzoic Acid Conjugate of Peptide H-D-Tic-D-Tic-PLG-Hphe-OLEE-OH

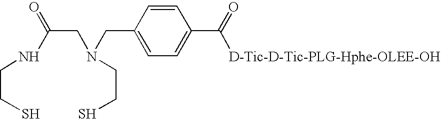

### Part A—Preparation of Fmoc-D-Tic-D-Tic-Ahx-PLG-Hphe-O(Boc)LE(t-Bu)E(t-Bu)-Wang Resin

The peptide-resin from Example 1, Part A is placed in a 50 mL reaction vessel, swollen by washing with N,N-dimethylformamide, and the following steps are performed: (Step 1) The Fmoc group is removed using 20% piperidine in N,N-dimethylformamide for 30 minutes. (Step 2) The resin is washed thoroughly with N,N-dimethylformamide (3×), dichloromethane (3×), methanol (3×), dichloromethane (3×), and N,N-dimethylformamide (3×). (Step 3) Fmoc-D-Tic-OH, HOBt, and HBTU in 40:60 DMSO:N,N-dimethylformamide and diisopropylethylamine is added to the resin and the reaction is allowed to proceed for 10 hours. (Step 4) The resin is washed thoroughly with N,N-dimethylformamide (3×), dichloromethane (3×), methanol (3×), dichloromethane (3×), and N,N-dimethylformamide (3×). (Step 5) Fmoc-D-Tic-OH, HOBt, and HBTU in 10 ml of 40% DMSO in N,N-dimethylformamide and diisopropylethylamine is added to the resin and the reaction allowed to proceed for 4 hours. (Step 6) The resin is washed thoroughly with N,N-dimethylformamide (3×), dichloromethane (3×), methanol (3×), dichloromethane (3×), and N,N-dimethylformamide (3×). (Step 7) The coupling reaction is found to be complete as assessed by the semi-quantitative ninhydrin assay and quantitative picric assay or fulvene-piperidine assay. Steps 1-7 were repeated for the addition of the second D-Tic.

### Part B—4-[((4,4,4-Triphenylbutyl) {[N-(4,4,4-triphenylbutyl)carbamoyl]methyl}-amino)methyl]benzoic Acid Conjugate with Fmoc-D-Tic-D-Tic-Ahx-PLG-Hphe-O(Boc)LE(t-Bu)E(t-Bu)-Wang Resin

The peptide-resin of Part A is treated with 20% piperidine in N,N-dimethylformamide for 30 minutes, and washed thoroughly with N,N-dimethylformamide (3×), dichloromethane (3×), methanol (3×), dichloromethane (3×), and N,N-dimethylformamide (3×). 2,5-Dioxopyrrolidinyl 4-[((4,4,4-triphenylbutyl) {[N-(4,4,4-triphenylbutyl)carbamoyl]methyl}amino)methyl]benzoate (Harris, T. D.; Rajopadhye, M.; Damphousse, P. R.; Glowacka, D.; Yu, K.; Bourque, J. P.; Barrett, J. A.; Damphousse, D. J.; Heminway, S. J.; Lazewatsky, J.; Mazaika, T.; Carroll, T. R. Bioorg. Med. Chem. Lett. 1996, 6, 1741-1746), and HOAt in 40:60 DMSO:N,N-dimethylformamide and diisopropylethylamine is added to the resin and the reaction is allowed to proceed for 18 hours. The resin is washed thoroughly with N,N-dimethylformamide (3×), dichloromethane (3×), methanol (3×), dichloromethane (3×), and N,N-dimethylformamide (3×). The above coupling procedure is repeated until the reaction is determined to be complete as assessed by LC/MS of a small portion of cleaved peptide.

### Part C—Cleavage and Final Deprotection

The peptide-resin of Part B is stirred with 95:2.5:25.5 trifluoroacetic acid:H₂O:TIS for 2 hours. The resin is removed by filtration through a sintered glass funnel and washed thoroughly with trifluoroacetic acid. The filtrate is concentrated to a small volume and diluted with ether. The resulting precipitate is collected by filtration, washed with ether and purified by HPLC on a C18 column using a water:acetonitrile:0.1% trifluoroacetic acid gradient. The product fraction is lyophilized to give the title compound.

### EXAMPLE 59

### Synthesis of the HYNIC Conjugate of Ac-RRRR-K[Ac-PLG-Hphe-YL]-RRRR-OH

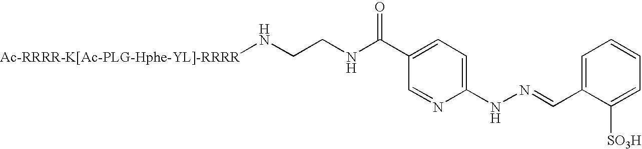

### Part A—Preparation of Ac-D-Arg(Pbf)-D-Arg(Pbf)-D-Arg(Pbf)-D-Arg(Pbf)-k(Teoc)-D-Arg(Pbf)-D-Arg(Pbf)-D-Arg(Pbf)-D-Arg(Pbf)-HMBP-BHA Resin

HMPB-BHA resin is placed in a peptide synthesis reaction vessel, and swollen by washing with N,N-dimethylformamide (2×). Fmoc-D-Arg(Pbf)-OH in N,N-dimethylformamide is added and the resin is mixed at room temperature for 15 minutes. Pyridine and 2,6-dichlorobenzoyl chloride are added and the mixture is gently shaken for 20 hours. The resin is washed thoroughly with N,N-dimethylformamide (3×), dichloromethane (3×), methanol (3×), dichloromethane (3×), and N,N-dimethylformamide (3×). The remaining hydroxyl groups of the resin are capped by reacting with benzoyl chloride and pyridine in dichloromethane for 2 hours. The substitution level is determined by the quantitative fulvene-piperidine assay. The following steps are then performed: (Step 1) The Fmoc group is removed using 20% piperidine in N,N-dimethylformamide for 30 minutes. (Step 2) The resin is washed thoroughly with N,N-dimethylformamide (3×), dichloromethane (3×), methanol (3×), dichloromethane (3×), and N,N-dimethylformamide (3×). (Step 3) Fmoc-Hphe-OH, HOBt, and HBTU in N,N-dimethylformamide and diisopropylethylamine are added to the resin and the reaction is allowed to proceed for 8 hours. (Step 4) The resin is washed thoroughly with N,N-dimethylformamide (3×), dichloromethane (3×), methanol (3×), dichloromethane (3×), and N,N-dimethylformamide (3×). (Step 5) A double coupling is performed if the quantitative fulvene-piperidine assay shows the first coupling to be incomplete. (Step 6) The resin is washed thoroughly with N,N-dimethylformamide (3×), dichloromethane (3×), methanol (3×), dichloromethane (3×), and N,N-dimethylformamide (3×). Steps 3-6 are repeated until the sequence Fmoc-D-Arg(Pbf)-D-Arg(Pbf)-D-Arg(Pbf)-D-Arg(Pbf)-k(Teoc)-D-Arg(Pbf)-D-Arg(Pbf)-D-Arg(Pbf)-D-Arg(Pbf)-HMPB-BHA resin is attained. The peptide-resin is treated with 20% piperidine in N,N-dimethylformamide for 30 minutes, and washed thoroughly with N,N-dimethylformamide (3×), dichloromethane (3×), methanol (3×), dichloromethane (3×), and N,N-dimethylformamide (3×). Acetic anhydride, and diisopropylethylamine are added, and the resin is mixed until the capping reaction is found to be complete as assessed by LC/MS of a small portion of cleaved peptide. The resin is washed thoroughly with N,N-dimethylformamide (3×), dichloromethane (3×), methanol (3×), dichloromethane (3×), and methanol (3×) and dried.

### Part B—Preparation of Ac-D-Arg(Pbf)-D-Arg(Pbf)-D-Arg(Pbf)-D-Arg(Pbf)-K[Ac-PLG-Hphe-Y(t-Bu)-L]-D-Arg(Pbf)-D-Arg(Pbf)-D-Arg(Pbf)-D-Arg(Pbf)-HMBP-BHA Resin

The peptide-resin from Part A is placed in a peptide synthesis reaction vessel, and swollen by washing with N,N-dimethylformamide (2×). The resin is treated with a solution of TBAF in N,N-dimethylformamide and the mixture is gently shaken for 18 hours. The following steps are then performed: (Step 1) The resin is washed thoroughly with N,N-dimethylformamide (3×), dichloromethane (3×), methanol (3×), dichloromethane (3×), and N,N-dimethylformamide (3×). (Step 2) Fmoc-Leu-OH, HOBt, and HBTU in N,N-dimethylformamide and diisopropylethylamine are added to the resin and the reaction is allowed to proceed for 8 hours. (Step 3) The resin is washed thoroughly with N,N-dimethylformamide (3×), dichloromethane (3×), methanol (3×), dichloromethane (3×), and N,N-dimethylformamide (3×). (Step 4) A double coupling is performed if the quantitative fulvene-piperidine assay shows the first coupling to be incomplete. (Step 5) The resin is washed thoroughly with N,N-dimethylformamide (3×), dichloromethane (3×), methanol (3×), dichloromethane (3×), and N,N-dimethylformamide (3×). (Step 6) The Fmoc group is removed using 20% piperidine in N,N-dimethylformamide for 30 minutes. Steps 1-6 are repeated until the sequence Fmoc-PLG-Hphe-Y(t-Bu)-L has been added to the lysine side chain. Acetic anhydride, and diisopropylethylamine are added, and the resin is mixed until the capping reaction is found to be complete as assessed by LC/MS of a small portion of cleaved peptide. The resin is washed thoroughly with N,N-dimethylformamide (3×), dichloromethane (3×), methanol (3×), dichloromethane (3×), and methanol (3×) and dried.

### Part C—Preparation of Ac-D-Arg(Pbf)-D-Arg(Pbf)-D-Arg(Pbf)-D-Arg(Pbf)-k[Ac-PLG-Hphe-Y(t-Bu)-L]-D-Arg(Pbf)-D-Arg(Pbf)-D-Arg(Pbf)-D-Arg(Pbf)-OH

The peptide-resin is placed in a sintered glass funnel and treated with 1% trifluoroacetic acid in dichloromethane. After 2 minutes, the solution is filtered, by the application of pressure, directly into a solution of 10% pyridine in methanol. The cleavage step is repeated nine times. The combined filtrates are evaporated to 5% of their volume, diluted with water, and cooled in an ice-water bath. The resulting precipitate is collected by filtration in a sintered glass funnel, washed with water, and dried under vacuum. The resulting residue is purified by HPLC on a C18 column using a water:acetonitrile:0.1% trifluoroacetic acid gradient to give the title compound.

### Part D—Preparation of the Hynic Conjugate of Ac-D-Arg(Pbf)-D-Arg(Pbf)-D-Arg(Pbf)-D-Arg(Pbf)-k[Ac-PLG-Hphe-Y(t-Bu)-L]-D-Arg(Pbf)-D-Arg(Pbf)-D-Arg(Pbf)-D-Arg(Pbf)-OH

A solution of the product of Part C, the product of Experiment 23, Part F, diisopropylethylamine, and HOAt in anhydrous N,N-dimethylformamide is treated with HBTU and stirred at ambient temperatures under nitrogen for 48 hours. The solution is concentrated and the resulting residue is purified by HPLC on a C18 column using a water:acetonitrile:0.1% trifluoroacetic acid gradient. The product fraction is lyophilized to give the title compound.

### Part E—Final Deprotection

The product of Part D is dissolved in 95:2.5:2.5 trifluoroacetic acid:Et3SiH:water and heated with stirring at 60° C. under nitrogen for 30 minutes. The solution is concentrated under reduced pressure and the resulting residue is purified by HPLC on a C18 column using a water:acetonitrile:0.1% trifluoroacetic acid gradient. The product fraction is lyophilized to give the title compound.

### EXAMPLE 61

### Synthesis of N-{(2S)-2-[(2S)-2-(2-{(2S)-2-[((2S)-1-Acetylpyrrolidin-2-yl)carbonylamino]-N-(4-aminobutyl)-4-methylpentanoylamino}acetylamino)-4-methylpentanoylamino]-4-methylpentanoylamino}-6-(acetylamino)hexanamide Trifluoroacetic Acid Salt

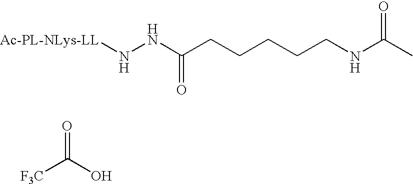

### Part A—Preparation of Fmoc-PL-NLys(Boc)-LL-HMPB-BHA Resin

HMPB-BHA resin (8.000 g, substitution level=0.68 mmol/g) was placed in a 200 mL Advanced ChemTech reaction vessel and swollen by washing with N,N-dimethylformamide (2×45 mL). A solution of Fmoc-Leu-OH (5.77 g, 16.32 mmol) in N,N-dimethylformamide (45 mL) was added to the vessel and the mixture was shaken for 15 min. 2,6-Dichlorobenzoyl chloride (2.5 mL, 16.32 mmol) and pyridine (2.0 mL, 24.5 mmol) in N,N-dimethylformamide (45 mL) were added and the mixture was shaken under nitrogen at ambient temperature for 18 h. The resin was washed (90 mL volumes) with N,N-dimethylformamide (3×), dichloromethane (3×), methanol (1×), dichloromethane (3×) and N,N-dimethylformamide (3×). A solution of benzoyl chloride (3.0 mL, 26 mmol) and pyridine (3.0 mL, 36.7 mmol) in N,N-dimethylformamide (90 mL) was added to the resin and the vessel was shaken under nitrogen for 3 h and washed (90 mL volumes) with N,N-dimethylformamide (3×), dichloromethane (3×), methanol (1×) and dichloromethane (3×). Fulvene-Piperidine assay performed on dry sample of resin showed a loading of 0.340 mmol/g.

The following steps were performed: (Step 1) The Fmoc group was removed using 20% piperidine in N,N-dimethylformamide (90 mL) for 30 min. (Step 2) The resin was washed (90 ml volumes) with N,N-dimethylformamide (3×), dichloromethane (3×), methanol (3×), dichloromethane (3×), and N,N-dimethylformamide (3×). (Step 3) Fmoc-Leu-OH (2.88 g, 8.16 mmol), HOBt (1.25 g, 8.16 mmol), and HBTU (3.10 g, 8.16 mmol) in 90 mL of N,N-dimethylformamide and 2 ml of diisopropylethylamine were added to the resin and the reaction was allowed to proceed for 5 h. (Step 4) The resin was washed as in step 2. (Step 5) Fmoc-Leu-OH (2.88 g, 8.16 mmol) and PyBroP (3.8 g, 8.16 mmol) in 90 ml of N,N-dimethylformamide and 2 mL of diisopropylethylamine were added to the resin and the reaction was allowed to proceed for 5 h. (Step 7) The resin was washed (90 mL volumes) with N,N-dimethylformamide (3×), dichloromethane (3×), methanol (3×), and dichloromethane (3×). (Step 6) Reaction completeness monitored by Fulvene-Piperidine assay. Steps 1-7 were repeated until the desired sequence was attained. Coupling yields were >95%.

### Part B—Preparation of Ac-PL-NLys(Boc)-LL-OH

The peptide-resin of Part A (2.5 g) was placed in a 100 mL Advanced ChemTech reaction vessel and swollen by washing with N,N-dimethylformamide (2×30 mL). The resin was treated with 20% piperidine in N,N-dimethylformamide (30 μL) for 30 minutes to remove Fmoc protecting group, followed by washing (30 ml volumes) with N,N-dimethylformamide (3×), dichloromethane (3×), methanol (3×), dichloromethane (3×), and N,N-dimethylformamide (3×). Acetic anhydride (0.78 mL, 4.2 mmol), diisopropylethylamine (0.88 mL, 5.0 mmol), and N,N-dimethylformamide (30 mL) were added and the mixture was gently agitated for 2 h. The peptide-resin was washed (30 mL volumes) with N,N-dimethylformamide (3×), dichloromethane (3×), methanol (3×), and dichloromethane (3×), and dried under vacuum. The peptide-resin was placed in a sintered glass funnel and treated with 1% trifluoroacetic acid in dichloromethane (12 mL) for 2 min. The solution was filtered, by application of nitrogen pressure, directly into a flask containing 1:9 pyridine:methanol (2 mL). The cleavage procedure was repeated ten (10) times. The combined filtrates were concentrated to give a colorless oily solid. This crude product triturated with water (2×25 mL) and dried under reduced pressure to give a dry solid. This solid was purified by HPLC on a Phenomenex Luna C18(2) column (21.2×250 mm) using a 0.9%/min gradient of 36 to 54% acetonitrile containing 0.1% trifluoroacetic acid at a flow rate of 20 mL/min. The main product peak eluting at 14.4 min was lyophilized to give 63.6 mg (63%) of the title compound as a colorless solid with 100% purity by HPLC. MS: m/e 725.4 [M+H](70%), 625.3 [M+H-Boc](100%).

### Part C—Preparation of N-Amino-6-[(fluoren-9-ylmethoxy)carbonylamino]hexanamide Trifluoroacetic Acid Salt

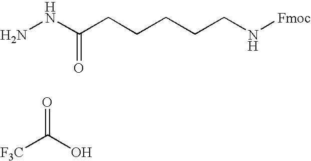

The product of Example 13, Part A (3.00 g, 6.44 mmol) was treated with 20 mL of 50% trifluoroacetic acid in dichloromethane for 30 min at ambient temperatures under nitrogen. The solution was concentrated under reduced pressure to give a pale yellow oil. The oil was dissolved in 30:70 acetonitrile:water (40 mL) and lyophilized to give an off-white solid (2.30 g, 74%) ¹H NMR (CDCl₃): δ 10.36 (s, 1H), 7.89 (d, J=7.3 Hz, 2H), 7.67 (d, J=7.7 Hz, 2H), 7.41 (t, J=7.7 Hz, 2H), 7.33 (t, J=7.3 Hz, 2H), 7.25 (t, J=6.0 Hz, 1H), 4.30 (d, J=6.6 Hz, 2H), 4.20 (t, J=6.6 Hz, 1H), 2.96 (q, J=6.0 Hz, 2H), 2.158 (t, J=7.5 Hz, 2H), 1.51 (pen, J=7.8 Hz, 2H), 1.39 (pen, J=7.8 Hz, 2H), 1.26 (m, 2H); MS: m/e 368.2 [M+H](100%).

### Part D—Preparation of N-((2S)-2-{(2S)-2-[2-((2S)-2-[((2S)-1-Acetylpyrrolidin-2-yl)carbonylamino]-N-{4-[(tert-butoxy)carbonylamino]butyl}-4-methylpentanoylamino)acetylamino]-4-methylpentanoylamino}-4-methylpentanoylamino)-6-aminohexanamide Trifluoroacetic Acid Salt

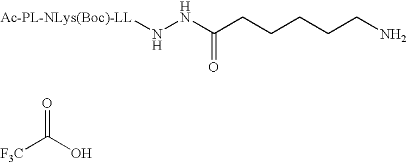

A solution of the peptide from Part B (31.0 mg, 0.043 mmol) and HOAt (5.8 mg, 0.043 mmol) in N,N-dimethylformamide (1 mL) was made basic with collidine (28.3 μL, 0.214 mmol). The solution was treated with DIC (13.2 μL, 0.086 mmol), and stirred at room temperature under nitrogen for 15 min. The product of Part C (31.4 mg, 0.086 mmol) was added, and the reaction was stirred at room temperature. Additional product of Part C (31.4 mg, 0.086 mmol) and DIC (13.2 μL, 0.086 mmol) were added after 18 h. After three days, the reaction was completed and the solvent was removed under reduced pressure to give crude title compound as a yellow oil.

The above oil was dissolved in 20% piperidine/N,N-dimethylformamide (0.25 mL) was stirred at room temperature under nitrogen for 15 min. The solution was concentrated under vacuum, and the resulting residue was purified by HPLC on a Phenomenex Luna C18(2) column (21.2×250 mm) using a 0.9%/min gradient of 18 to 45% acetonitrile containing 0.1% trifluoroacetic acid (pH 2) at a flow rate of 20 mL/min. The main product peak eluting at 24.0 min was lyophilized to give the title compound as a colorless solid (14.3 mg, 39%, HPLC purity 100%). MS: m/e 852.6 [M+H](100%).

### Part E—Preparation of N-{(2S)-2-[(2S)-2-(2-{(2S)-2-[((2S)-1-Acetylpyrrolidin-2-yl)carbonylamino]-N-(4-aminobutyl)-4-methylpentanoylamino}acetylamino)-4-methylpentanoylamino]-4-methylpentanoylamino}-6-(acetylamino)hexanamide Trifluoroacetic Acid Salt

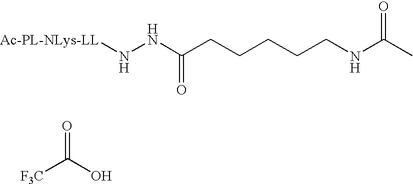

The product of Part D (4.4 mg, 0.005 mmol) in 0.5 mL of N,N-dimethylformamide was treated with acetic anhydride (2.4 μL, 0.026 mmol) and diisopropylethylamine (4.5 μL, 0.026 mmol). The solution was stirred at room temperature under nitrogen for 5 min, and the solvents were evaporated under reduced pressure. The resulting residue was dissolved in 50:50 trifluoroacetic acid:water (1 mL) and stirred at room temperature under nitrogen for 20 min. The solution was concentrated under vacuum, and the resulting residue was purified by HPLC on a Phenomenex Luna C18(2) column (21.2×250 mm) using a 0.9%/min gradient of 13.5 to 31.5% acetonitrile containing 0.1% trifluoroacetic acid (pH 2) at a flow rate of 20 mL/min. The main product peak eluting at 18.5 min was lyophilized to give the title compound as a colorless solid (3.2 mg, 83%, HPLC purity 100%). MS: m/e 794.5 [M+H](100%), 397.8 [M+2H](80%); High Resolution MS: Calcd for C₃₉H₇₁N₉O₈ [M+H]: 794.5498, Found: 794.5491. Chiral analysis for L-Leucine: 99.8%.

### EXAMPLE 62

### Synthesis of (2S)-N-{(1S)-1-[N-((1S)-1-{N-[6-(Acetylamino)hexanoylamino]carbamoyl}-3-methylbutyl)carbamoyl]-2-(4-hydroxyphenyl)ethyl}-2-(2-{(2S)-2-[((2S)-1-acetylpyrrolidin-2-yl)carbonylamino]-4-methylpentanoylamino}acetylamino)hept-6-enamide

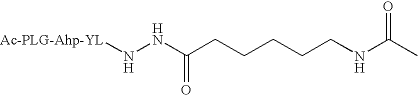

### Part A—Preparation of Fmoc-PLG-Ahp-YL-HMPB-BHA Resin

HMPB-BHA resin (8.000 g, substitution level=0.68 mmol/g) was placed in a 200 mL Advanced ChemTech reaction vessel and swollen by washing with N,N-dimethylformamide (2×45 mL). A solution of Fmoc-Leu-OH (5.77 g, 16.32 mmol) in N,N-dimethylformamide (45 mL) was added to the vessel and the mixture was shaken for 15 min. 2,6-Dichlorobenzoyl chloride (2.5 mL, 16.32 mmol) and pyridine (2.0 mL, 24.5 mmol) in N,N-dimethylformamide (45 mL) were added and the mixture was shaken under nitrogen at ambient temperature for 18 h. The resin was washed (90 mL volumes) with N,N-dimethylformamide (3×), dichloromethane (3×), methanol (1×), dichloromethane (3×) and N,N-dimethylformamide (3×). A solution of benzoyl chloride (3.0 mL, 26 mmol) and pyridine (3.0 mL, 36.7 mmol) in N,N-dimethylformamide (90 mL) was added to the resin and the vessel was shaken under nitrogen for 3 h and washed (90 mL volumes) with N,N-dimethylformamide (3×), dichloromethane (3×), methanol (1×) and dichloromethane (3×). Fulvene-Piperidine assay performed on dry sample of resin showed a loading of 0.340 mmol/g.

The following steps were performed: (Step 1) The Fmoc group was removed using 20% piperidine in N,N-dimethylformamide (90 mL) for 30 min. (Step 2) The resin was washed (90 ml volumes) with N,N-dimethylformamide (3×), dichloromethane (3×), methanol (3×), dichloromethane (3×), and N,N-dimethylformamide (3×). (Step 3) Fmoc-Tyr(O-tBu)-OH (3.75 g, 8.16 mmol), HOBt (1.25 g, 8.16 mmol), and HBTU (3.10 g, 8.16 mmol) in 90 mL of N,N-dimethylformamide and 2 ml of diisopropylethylamine were added to the resin and the reaction was allowed to proceed for 5 h. (Step 4) The resin was washed as in step 2. (Step 5) Fmoc-Tyr(O-tBu)-OH (3.75 g, 8.16 mmol) and PyBroP (3.8 g, 8.16 mmol) in 90 ml of N,N-dimethylformamide and 2 mL of diisopropylethylamine were added to the resin and the reaction was allowed to proceed for 5 h. (Step 7) The resin was washed (90 mL volumes) with N,N-dimethylformamide (3×), dichloromethane (3×), methanol (3×), and dichloromethane (3×). (Step 6) Reaction completeness monitored by Fulvene-Piperidine assay. Steps 1-7 were repeated until the desired sequence was attained. Coupling yields were >95%.

### Part B—Preparation of Ac-PLG-Ahp-Y(O-tBu)L-OH

The peptide-resin of Part A (2.5 g) was placed in a 100 mL Advanced ChemTech reaction vessel and swollen by washing with N,N-dimethylformamide (2×30 mL). The resin was treated with 20% piperidine in N,N-dimethylformamide (30 mL) for 30 minutes to remove Fmoc protecting group, followed by washing (30 ml volumes) with N,N-dimethylformamide (3×), dichloromethane (3×), methanol (3×), dichloromethane (3×), and N,N-dimethylformamide (3×). Acetic anhydride (0.78 mL, 4.2 mmol), diisopropylethylamine (0.88 mL, 5.0 mmol), and N,N-dimethylformamide (30 mL) were added and the mixture was gently agitated for 2 h. The peptide-resin was washed (30 mL volumes) with N,N-dimethylformamide (3×), dichloromethane (3×), methanol (3×), and dichloromethane (3×), and dried under vacuum. The peptide-resin was placed in a sintered glass funnel and treated with 1% trifluoroacetic acid in dichloromethane (12 mL) for 2 min. The solution was filtered, by application of nitrogen pressure, directly into a flask containing 1:9 pyridine:methanol (2 mL). The cleavage procedure was repeated ten (10) times. The combined filtrates were concentrated to give a colorless oily solid. This crude product triturated with water (2×25 mL) and dried under reduced pressure to give a dry solid. This solid was purified by HPLC on a Phenomenex Luna C18(2) column (21.2×250 mm) using a 1.0%/min gradient of 40 to 65% acetonitrile containing 0.1% trifluoroacetic acid at a flow rate of 20 mL/min. The main product peak eluting at 21.4 min was lyophilized to give 84.6 mg (77%) of the title compound as a colorless solid with 100% purity by HPLC. MS: m/e 785.5 [M+H](100%); High Resolution MS: Calcd for C₄₁H₆₄N₆O₉ [M+H]: 785.4807, Found: 785.4806.

### Part C—Preparation of (2S)-N-[(1S)-1-(N-{(1S)-1-[N-(6-Aminohexanoylamino)carbamoyl]-3-methylbutyl}carbamoyl)-2-[4-(tert-butoxy)phenyl]ethyl]-2-(2-{(2S)-2-[((2S)-1-acetylpyrrolidin-2-yl)carbonylamino]-4-methylpentanoylamino}-acetylamino)hept-6-enamide Trifluoroacetic Acid Salt

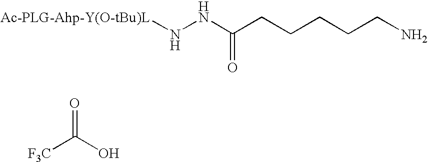

A solution of the product of Part B (52.1 mg, 0.066 mmol) and HOAt (9.0 mg, 0.066 mmol) in N,N-dimethylformamide (1 mL) was made basic with collidine (43.9 μL, 0.332 mmol). The solution was treated with DIC (20.6 μL, 0.133 mmol), and stirred at room temperature under nitrogen for 15 min. The product of Example 61, Part C (48.8 mg, 0.133 mmol) was added and the reaction was stirred at room temperature. Additional product of Example 61, Part C (48.8 mg, 0.133 mmol) and DIC (41.2 μL, 0.265 mmol) were added after 18 h. The reaction was complete in three day, and the solvent was removed under reduced pressure to give a yellow oil.

The above oil was dissolved in TAEA (0.25 mL, 1.659 mmol) was stirred at room temperature under nitrogen for 30 min. The solution was concentrated under vacuum, and the resulting residue was purified by HPLC on a Phenomenex Luna C18(2) column (21.2×250 mm) using a 0.9%/min gradient of 31.5 to 49.5% acetonitrile containing 0.1% trifluoroacetic acid (pH 2) at a flow rate of 20 mL/min. The main product peak eluting at 25.6 min was lyophilized to give the title compound as a colorless solid (38.3 mg, 63%, HPLC purity 100%). MS: m/e 912.6 [M+H](100%); High Resolution MS: Calcd for C₄₇H₇₇N₉O₉ [M+H]:912.5917, Found: 912.5913.

### Part D—Preparation of (2S)-N-{(1S)-1-[N-((1S)-1-{N-[6-(Acetylamino)hexanoylamino]carbamoyl}3-methylbutyl)carbamoyl]-2-(4-hydroxyphenyl)ethyl}-2-(2-{(2S)-2-[((2S)-1-acetylpyrrolidin-2-yl)carbonylamino]-4-methylpentanoylamino}acetylamino)hept-6-enamide

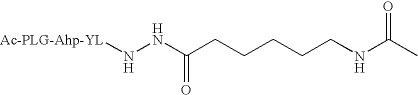

The product of Part C (9.1 mg, 0.010 mmol) in 0.5 mL of N,N-dimethylformamide was treated with Ac₂O (4.7 μL, 0.050 mmol) and diisopropylethylamine (8.7 μL, 0.050 mmol). The solution was stirred at room temperature under nitrogen for 5 min and the solvents were evaporated under reduced pressure. The resulting residue was dissolved in 95:2.5:2.5 trifluoroacetic acid:anisole:water (1 mL) and stirred at room temperature under nitrogen for 20 min. The solution was concentrated under vacuum, and the resulting residue was purified by HPLC on a Phenomenex Luna C18(2) column (21.2×250 mm) using a 0.9%/min gradient of 22.5 to 45% acetonitrile containing 0.1% trifluoroacetic acid (pH 2) at a flow rate of 20 mL/min. The main product peak eluting at 18.5 min was lyophilized to give the title compound as a colorless solid (8.5 mg, 94%, HPLC purity 100%). ¹H NMR (DMSO-d₆): δ 9.78-9.76 (m, 1H), 9.70-9.69 (m, 1H), 9.12 (bs, 1H), 7.99-7.89 (m, 3H), 7.80-7.70 (m, 2H), 7.01 (d, J=8.3 Hz, 2H), 6.62 (d, J=8.3 Hz), 5.77-5.70 (m, 1H), 4.98 (d, J=17.1 Hz, 1H), 4.92 (d, J=10.2 Hz, 1H), 4.44-4.35 (m, 3H), 4.28-4.20 (m, 2H), 3.78-3.64 (m, 2H), 3.57-3.51 (m, 1H), 2.99 (q, J=6.5 Hz, 2H), 2.89-2.86 (m, 1H), 2.67-2.62 (m, 1H), 2.09 (t, J=7.4 Hz, 2H), 2.03-1.73 (m, 13H), 1.66-1.21 (m, 17H), 0.89-0.81 (m, 12H); MS: m/e 898.5 [M+H] (90%), 449.4 [M+2H] (100%); Chiral analysis for L-Leucine: 99.8%.

### EXAMPLE 63

### N-[(1E)-8-(Acetylamino)oct-1-enyl](2S)-2-amino-4-methylpentanamide, Formic Acid Salt

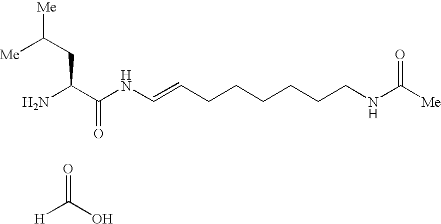

### Part A—Preparation of 8-Iodooct-1-yne

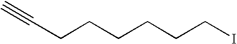

PPh₃ (13.7 g, 52.4 mmol) and imidazole (3.57 g, 52.4 mmol) were dissolved in CH₂Cl₂ (100 mL) and treated with 12 (13.3 g, 52.4 mmol) in one portion. To this solution was transferred oct-7-yn-1-ol (4.40 g, 34.9 mmol) as a solution in CH₂Cl₂ (50 mL) via cannula over 5 min at 22° C. After stirring 2 h, the mixture was diluted with pentane (450 mL) and the resulting precipitate removed by filtration through a fritted funnel. The filtrate was concentrated in vacuo and the trituration process repeated. The resulting pale yellow oil was purified by chromatography on silica (100% pentane; R_{f}=0.4 in pentane) to afford a colorless oil (7.01 g, 29.7 mmol; 85.1%). ¹H NMR (CDCl₃, 600 MHz): δ 3.20 (2H, t, J=6.6 Hz), 2.21 (2H, td, J=6.6, 2.4 Hz), 1.95 (1H, t, J=2.4 Hz), 1.85 (2H, quin, J=7.2 Hz), 1.55 (2H, m), 1.43 (4H, m). ³C NMR (CDCl₃, 150 MHz): δ 84.6, 68.5, 33.6, 30.2, 28.4, 27.8, 18.5, 7.2.

### Part B—Preparation of (1E)-1,8-Diiodooct-1-ene

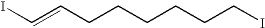

The product of part A (4.32 g, 18.3 mmol) was transferred via cannula as a solution in CH₂Cl₂ (20 m]L) to a solution of Cp₂ZrHCl (11.8 g, 45.8 mmol) in CH₂Cl₂ (80 mL) at 22° C. The now yellow solution was stirred 2.5 h before a saturated solution of 12 in CH₂Cl₂ was added, dropwise using an addition funnel, until the purple color persisted (˜100 mL). The mixture was then poured into pentane (500 mL) and the resulting precipitate removed by filtration through a fritted funnel. The filtrate was then washed with a saturated solution of Na₂S₂O₃ (3×200 mL), H₂O (100 mL) and saturated NaCl (200 mL). The organic layer was then dried over Na₂SO₄, filtered and concentrated in vacuo to afford a yellow oil. Purification by chromatography on silica (100% pentane; R_{f}=0.6 in pentane) afforded a colorless oil (4.27 g, 11.7 mmol; 64.1%). ¹H NMR (CDCl₃, 600 MHz): δ 6.49 (1H, dt, J=14.4, 7.2 Hz), 5.84 (1H, dt, J=14.4, 1.5 Hz), 3.17 (2H, t, J=6.9 Hz), 2.05 (2H, qd, J=7.2, 1.8 Hz), 1.81 (2H, m), 1.37 (4H, m), 1.31 (2H, m). ¹³C NMR (CDCl₃, 150 MHz): δ 146.4, 74.6, 35.6, 33.3, 30.2, 28.1, 27.8, 7.0.

### Part C—Preparation of (1E)-8-Azido-1-iodooct-1-ene

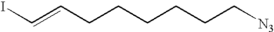

The product of part B (2.17 g, 5.96 mmol) was transferred as a solution in N,N-dimethylformamide (30 mL) to solid NaN₃ (657 mg, 10.1 mmol) at 22° C. The resulting homogeneous solution was stirred 1 h then diluted with a saturated solution of NaCl (150 mL). The resulting mixture was then transferred to a separatory funnel and washed with pentane (3×50 mL). The combined organic washes were dried over Na₂SO₄, filtered and concentrated in vacuo. Purification by chromatography on silica (100% pentane; R_{f}=0.3 in pentane) affored a colorless oil (1.30 g, 4.66 mmol; 78.1%). ¹H NMR (CDCl₃, 600 MHz): δ 6.49 (1H, dt, J=14.2, 7.1 Hz), 5.98 (1H, dt, J=14.4, 1.5 Hz), 3.25 (2H, t, J=6.9 Hz), 2.05 (2H, qd, J=7.4, 1.5 Hz), 1.59 (2H, m), 1.42-1.29 (6H, m). ¹³C NMR (CDCl₃, 150 MHz): δ 146.4, 74.5, 51.4, 35.9, 28.7, 28.4, 28.2, 26.4.

### Part D—Preparation of N-((1E)-8-Azidooct-1-enyl)(2S)-2-amino-4-methylpentanamide

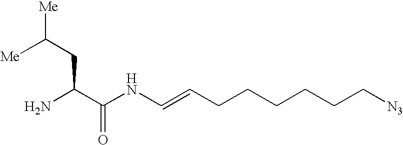

A 5 mL conical flask was charged with the product of part C (279 mg, 1.00 mmol), N,N′-dimethylethylenediamine (11 μL, 0.10 mmol; 10 mol %) and anhydrous THF (1.00 mL) and set aside. Copper (I) iodide (0.95×10¹ mg, 0.050 mmol; 5 mol %), leucine amide (2.60×10² mg, 2.00 mmol) and Cs₂CO₃ (489 mg, 1.50 mmol) were massed into an oven-dried 25 mL Schlenk tube. This vessel was then evacuated and back-filled with dry nitrogen three times. Using a gas-tight syringe, the previously prepared solution of vinyl iodide was then transferred to this flask through the side arm; an additional 1.00 mL THF was used to quantitate the transfer. The flask was sealed then immersed in a preheated oil bath and maintained for 16 h at 70° C. After cooling to 22° C. the resulting suspension was diluted with ethyl acetate (1 mL) and placed directly atop a previously prepared silica gel column. Elution with 9:1 CH₂Cl₂/methanol (R_{f}=0.4 in 9:1 CH₂Cl₂/methanol) afforded, after concentration, a pale yellow oil (244 mg, 0.867 mmol; 86.7%). ¹H NMR (C₆D₆, 600 MHz): δ 8.77 (1H, brd, J=10.2 Hz), 7.12 (1H, ddt, J=14.3, 11.1, 1.4 Hz), 4.94 (1H, dt, J=14.3, 7.2 Hz), 3.05 (1H, dd, J=9.6, 4.3 Hz), 2.67 (2H, t, J=7.0 Hz), 1.86 (2H, qd, J=7.2, 1.4 Hz), 1.72 (1H, ddd, J=13.8, 9.3, 4.4 Hz), 1.40 (1H, m), 1.16 (4H, m), 1.04 (1H, ddd, J=13.8, 9.6, 5.2 Hz), 1.00 (5H, m), 0.79 (3H, d, J=6.6 Hz), 0.72 (3H, d, J=6.6 Hz). ¹³C NMR (C₆D₆, 150 MHz): δ 171.8, 123.4, 111.9, 53.3, 51.2, 44.2, 30.1, 29.9, 28.9, 28.7, 26.7, 24.9, 23.4, 21.4. MS (ESI): m/z 304.4 (4, M+Na), 282.4 (100, M+H).

### Part E—Preparation of N-((1E)-8-Azidooct-1-enyl)(2S)-4-methyl-2-(prop-2-enyloxycarbonylamino)pentanamide

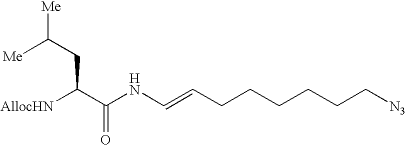

A solution of the product of part D (111 mg, 0.394 mmol) in THF (3.00 mL) was treated with i-Pr₂NEt (75 μL, 0.43 mmol) then cooled to 0° C. Allyl chloroformate (44 μL, 0.41 mmol) was then added and the solution stirred 1 h at 0° C. The resulting solution was then warmed to 22° C. and concentrated in vacuo. The crude oil thus obtained was purified by chromatography on silica (60:31:9 pentane/diethyl ether/methanol; R_{f}=0.4 in 60:31:9 pentane/diethyl ether/methanol) to afford a colorless oil (142 mg, 0.389 mmol; 98.5%). ¹H NMR (C₆D₆, 600 MHz): δ 8.08 (1H, brd, J=8.5 Hz), 7.03 (1H, dd, J=14.2, 10.5 Hz), 5.72 (1H, ddt, J=17.0, 10.7, 5.5 Hz), 5.37 (1H, d, J=7.8 Hz), 5.12 (1H, dq, J=17.2, 1.6 Hz), 5.03 (1H, dt, J=14.2, 7.1 Hz), 4.97 (1H, dq, J=10.5, 1.4 Hz), 4.46 (2H, ABqdt, J_{AB}=13.4 Hz, J_{d}=5.6 Hz, Jₜ=1.4 Hz), 4.34-4.30 (1H, m), 2.68 (2H, t, J=6.9 Hz), 1.83 (2H, brq, J=7.3 Hz), 1.61-1.56 (2H, m), 1.45-1.40 (1H, m), 1.21-1.12 (4H, m), 1.07-0.99 (4H, m), 0.84 (3H, d, J=5.8 Hz), 0.80 (3H, d, J=6.4 Hz). ¹³C NMR (C₆D₆, 150 MHz): δ 169.5, 156.8, 133.1, 123.2, 117.5, 113.5, 66.0, 53.8, 51.2, 41.2, 30.0(2), 28.8, 28.7, 26.7, 24.8, 23.0, 21.9. MS (ESI): m/z 388.3 (61, M+Na), 366.3 (100, M+H).

### Part F—Preparation of N-((1E)-8-Aminooct-1-enyl)(2S)-4-methyl-2-(prop-2-enyloxycarbonylamino)pentanamide, Formic Acid Salt

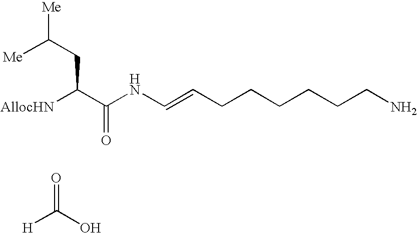

A solution of the product of part E (123 mg, 0.337 mmol) in THF (5.00 mL) was treated with PPh₃ (221 mg, 0.843 mmol) at 22° C. After complete dissolution, H₂O (182 μL, 10.1 mmol) was added and the solution stirred 1 h at 22° C. followed by 1 h at 70° C. With complete hydrolysis of the iminophosphorane, all volatiles were removed in vacuo and the residue purified by HPLC on a Phenomenex Luna C18 column (21.2×250 mm) using a 1.5%/minute gradient of 10 to 40% acetonitrile containing 0.1% HCO₂H at a flow rate of 20 mL/min. The main product peak eluting at 10 minutes was lyophilized to a white solid (45.0 mg, 0.117 mmol; 34.7%). ¹H NMR (C₆D₆, 600 MHz): δ 9.94 (1H, brd, J=10.0 Hz), 8.83 (1H, s), 7.36 (1H, d, J=8.6 Hz), 6.93 (1H, dd, J=14.3, 10.0 Hz), 5.76 (1H, ddt, J=17.1, 10.6, 5.4 Hz), 5.36 (1H, dt, J=14.3, 7.2 Hz), 5.18(1H, dq, J=17.2, 1.7 Hz), 4.96 (1H, dq, J=10.5, 1.6 Hz), 4.49-4.41 (3H, m), 2.62 (2H, dd, J=7.5, 7.4 Hz), 1.87 (2H, q, J=7.0 Hz), 1.78-1.73 (1H, m), 1.66 (1H, ddd, J=13.5, 10.1, 5.2 Hz), 1.57 (1H, ddd, J=13.5, 8.8, 5.1 Hz), 1.44 (2H, m), 1.19 (2H, m), 1.15-1.09 (5H, m), 0.87 (3H, d, J=6.6 Hz), 0.85 (3H, d, J=6.6 Hz). ¹³C NMR (C₆D₆, 150 MHz): δ 170.6, 166.7, 156.5, 133.9, 124.0, 116.9, 112.6, 65.0, 54.0, 41.8, 30.2(2) 29.9, 28.7, 26.6, 24.9, 23.3, 21.9. MS (ESI): m/z 362.3 (3, M+Na), 340.4 (100, M+H).

### Part G—Preparation of N-[(1E)-8-(Acetylamino)oct-1-enyl](2S)-2-amino-4-methylpentanamide, Formic Acid Salt

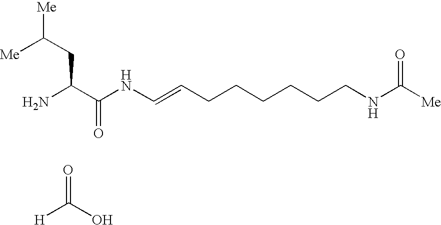

A solution of the product of part F (15.0 mg, 38.9 μmol) in N,N-dimethylformamide (3.00 mL) was treated with i-Pr₂NEt (27.0 μL, 155 μmol) followed by Ac₂O (11.0 μL, 117 μmol) at 22° C. The solution was stirred 0.5 h then diluted with H₂O (30 mL), transferred to a separatory funnel and washed with ethyl acetate (3×20 mL). The combined organic layers were washed with a saturated solution of NaHCO₃ (20 mL), H₂O (20 mL) and saturated NaCl (20 mL), then dried over Na₂SO₄, filtered and concentrated in vacuo. This material was used in the next step without further purification. MS (ESI): m/z 404.3 (22, M+Na), 382.4 (100, M+H).

The crude acetamide was redissolved in acetonitrile/H₂O (3.00 mL; 2:1 v/v) and treated with Pd(OAc)₂ (0.17 mg, 0.76 μmol; 2 mol %) followed by TPPTS (0.89 mg, 1.6 μmol; 4 mol %) and Et₂NH (10.0 μL, 97.3 μmol) at 22° C. Complete deprotection was observed in under 0.5 h. The solution was loaded directly onto a Phenomenex Luna C18 column (21.2×250 mm) using a 0.80%/minute gradient of 10 to 30% acetonitrile containing 0.1% HCO₂H at a flow rate of 20 mL/min. The main product peak eluting at 14 minutes was lyophilized to a white solid (8.0 mg, 23 μmol; 60% over two steps). ¹H NMR (C₆D₆, 600 MHz): δ 9.82 (1H, brd, J=9.9 Hz), 7.57 (1H, brs), 6.97 (1H, dd, J=14.2, 9.9 Hz), 5.33 (1H, dt, J=14.3, 7.2 Hz), 3.61 (1H, dd, J=8.5, 5.6 Hz), 3.20 (2H, td, J=7.1, 5.8 Hz), 1.92-1.88 (2H, m), 1.89 (3H, s), 1.77(1H, ddd, J=14.4, 6.5, 5.0 Hz), 1.67 (1H, ddd, J=13.7, 8.2, 5.6 Hz), 1.47-1.40 (3H, m), 1.25-1.15 (6H, m), 0.86 (3H, d, J=6.6 Hz), 0.84 (3H, d, J=6.5 Hz). ¹³C NMR (C₆D₆, 150 MHz): δ 172.0, 163.3, 123.7, 112.8, 53.5, 43.8, 42.1, 30.2, 30.1, 29.9, 28.9, 27.0, 24.8, 23.3, 23.1, 22.1. MS (ESI): m/z 298.4 (100, M+H), 284.4 (3).

### EXAMPLE 64

### N-[(1E)-5-(acetylamino)pent-1-enyl](2R)-2-amino-4-methylpentanamide, Formic Acid Salt

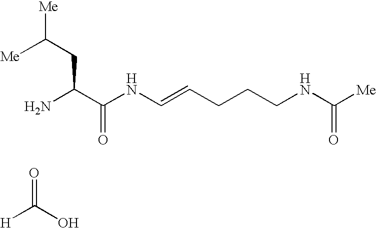 Part A—Preparation of N-((1E)-5-Azidopent-1-enyl)(2R)-2-amino-4-methylpentanamide

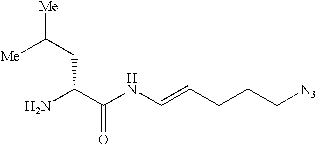

As described in part D of example 63, a 5 mL conical flask was charged with (1E)-5-azido-1-iodopent-1-ene (237 mg, 1.00 mmol), N,N′-dimethylethylenediamine (11 μL, 0.10 mmol; 10 mol %) and anhydrous THF (1.00 mL) and set aside. Copper (I) iodide (0.95×10¹ mg, 0.050 mmol; 5 mol %), leucine amide (2.60×10² mg, 2.00 mmol) and Cs₂CO₃ (489 mg, 1.50 mmol) were massed into an oven-dried 25 mL Schlenk tube. This vessel was then evacuated and back-filled with dry nitrogen three times. Using a gas-tight syringe, the previously prepared solution of vinyl iodide was then transferred to this flask through the side arm; an additional 1.00 mL THF was used to quantitate the transfer. The flask was sealed then immersed in a preheated oil bath and maintained for 16 h at 70° C. After cooling to 22° C. the resulting suspension was diluted with ethyl acetate (1 mL) and placed directly atop a previously prepared silica gel column. Elution with 9:1 CH₂Cl₂/methanol (R_{f}=0.3 in 9:1 CH₂Cl₂/methanol) afforded, after concentration, a pale yellow oil (2.10×10² mg, 0.877 mmol; 87.7%). ¹H NMR (C₆D₆), 600 MHz): δ 8.70 (1H, brd, J=9.0 Hz), 7.01 (1H, ddt, J=14.3, 11.1, 1.3 Hz), 4.69 (1H, dt, J=14.3, 7.2 Hz), 3.03 (1H, dd, J=9.7, 4.3 Hz), 2.63 (2H, t J=7.0 Hz), 1.73 (1H, ddd, J=13.7, 9.3, 4.3 Hz), 1.72-1.68 (2H, m), 1.43-1.36 (1H, m), 1.19 (2H, quin, J=7.1 Hz), 1.04 (1H, ddd, J=14.0, 9.6, 5.2 Hz), 0.80 (3H, d, J=6.6 Hz), 0.72 (3H, d, J=6.6 Hz). HRMS Calcd. for C₁₁H₂₂N₅O: 240.1824 (M+H). Found: 240.1819.

### Part B—Preparation of N-((1E)-5-Azidopent-1-enyl)(2R)-4-methyl-2-(prop-2-enyloxycarbonylamino)pentanamide

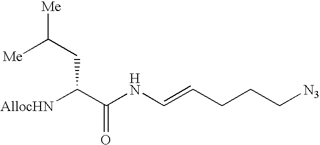

A solution of the product of part A (105 mg, 0.439 mmol) in THF (5.00 mL) was treated with i-Pr₂NEt (84.0 μL, 0.482 mmol) then cooled to 0° C. Allyl chloroformate (49.0 μL, 0.461 mmol) was then added and the solution stirred 0.5 h at 0° C. then warmed to 22° C. and stirred 0.75 h. The resulting solution was then concentrated in vacuo and directly purified by chromatography on silica (71:24:5 pentane/ethyl acetate/methanol; R_{f}=0.9 in 9:1 CH₂Cl₂/methanol) to afford a white solid (141 mg, 0.436 mmol; 99.4%). ¹H NMR (C₆D₆, 600 MHz): δ 7.36 (1H, brs), 6.86 (1H, ddt, J=14.3, 10.4, 1.4 Hz), 5.70 (1H, ddt, J=17.1, 10.5, 5.5 Hz), 5.09 (1H, dq, J=17.2, 1.6 Hz), 4.96 (1H, dq, J=10.5, 1.4 Hz), 4.75 (1H, brd, J=7.0 Hz), 4.64 (1H, dt, J=14.3, 7.2 Hz), 4.44 (2H, ABqdt, J_{AB}=13.4 Hz, J_{d}=5.6 Hz, Jₜ=1.4 Hz), 4.18-4.14 (1H, m), 2.60 (2H, t, J=6.9 Hz), 1.65-1.61 (2H, m), 1.56-1.46 (2H, m), 1.26 (1H, brs), 1.14 (2H, quin, J=7.2 Hz), 0.80 (3H, d, J=6.1 Hz), 0.74 (3H, d, J=6.5 Hz). MS (ESI): m/z 324.3 (10, M+H), 296.4 (100, M+H—N₂).

### Part C—Preparation of N-((1E)-5-Aminopent-1-enyl)(2R)-4-methyl-2-(prop-2-enyloxycarbonylamino)pentanamide, Formic Acid Salt

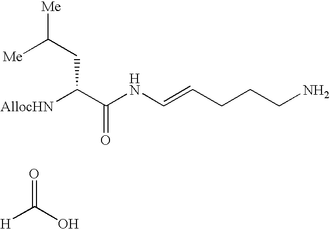

A solution of the product of part B (134 mg, 0.414 mmol) in THF (15.00 mL) was treated with PPh₃ (273 mg, 1.04 mmol) and H₂O (223 μL, 12.4 mmol) and stirred 1 h at 22° C. followed by 1 h at 70° C. With complete hydrolysis of the iminophosphorane, all volatiles were removed in vacuo and the residue purified by HPLC on a Phenomenex Luna C18 column (21.2×250 mm) using a 1.5%/minute gradient of 9 to 36% acetonitrile containing 0.1% HCO₂H at a flow rate of 20 mL/min. The main product peak eluting at 9 minutes was lyophilized to a white solid (69.0 mg, 0.201 mmol; 48.5%). ¹H NMR (DMSO-d₆, 600 MHz): δ9.91 (1H, brd, J=9.8 Hz), 8.50 (1H, s), 7.52 (1H, d, J=8.2 Hz), 6.66 (1H, dd, J=14.3, 10.0 Hz), 5.91 (1H, ddt, J=17.1, 10.6, 5.3 Hz), 5.30 (1H, dt, J=17.2, 1.4 Hz), 5.25 (1H, dt, J=14.2, 7.2 Hz), 5.17 (1H, brd, J=10.5 Hz), 4.51-4.45 (2H, m), 4.07 (1H, ddd, J=10.1, 8.2, 5.0 Hz), 2.72 (2H, dd, J=7.5, 7.3 Hz), 2.04 (2H, q, J=7.1 Hz), 1.66-1.62 (1H, m), 1.59 (2H, quin, J=7.3 Hz), 1.51 (1H, ddd, J=13.3, 10.4, 5.1 Hz), 1.39 (1H, ddd, J=13.6, 8.9, 4.9 Hz), 0.89 (3H, d, J=6.7 Hz), 0.87 (3H, d, J=6.6 Hz). HRMS Calcd. for C₁₅H₂₈N₃O₃ (M+H): 298.2131. Found: 298.2123.

### Part D—Preparation of N-[(1E)-5-(Acetylamino)pent-1-enyl](2R)-4-methyl-2-(prop-2-enyloxycarbonylamino)pentanamide

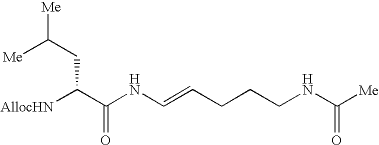

A solution of the product of part C (56.0 mg, 0.163 mmol) in N,N-dimethylformamide (4.00 mL) was treated with i-Pr₂NEt (142 μL, 0.815 mmol) followed by Ac₂O (77.0 μL, 0.815 mmol) at 22° C. The solution was stirred 0.5 h then diluted with H₂O and ethyl acetate (40 mL each), with transfer to a separatory funnel. The layers were separated and the aqueous layer washed with ethyl acetate (20 mL). The combined organic layers were washed with a saturated solution of NaHCO₃ (20 mL), H₂O (20 mL) and saturated NaCl (20 mL), then dried over Na₂SO₄, filtered and concentrated in vacuo to afford 45.0 mg of a pale yellow oil. This material was used in the next step without further purification. ¹H NMR (DMSO-d₆, 600 MHz): δ 9.75 (1H, d, J=9.9 Hz), 7.78 (1H, brs), 7.38 (1H, d, J=8.2 Hz), 6.57 (1H, dd, J=14.3, 9.9 Hz), 5.90 (1H, ddt, J=17.1, 10.6, 5.3 Hz), 5.28 (1H, dq, J=17.2, 1.6 Hz), 5.21 (1H, dt, J=14.3, 7.2 Hz), 5.17 (1H, dq, J=10.5, 1.3 Hz), 4.48-4.43 (2H, m), 4.00 (1H, ddd, J=10.1, 8.5, 5.0 Hz), 3.00 (2H, td, J=6.8, 6.0 Hz), 1.96 (2H, q, J=7.0 Hz), 1.78 (3H, s), 1.63-1.56 (1H, m), 1.47 (1H, ddd, J=13.6, 10.2, 5.1 Hz), 1.42 (2H, quin, J=7.2 Hz), 1.35 (1H, ddd, J=13.6, 8.8, 4.9 Hz), 0.87 (3H, d, J=6.6 Hz), 0.85 (3H, d, J=6.6 Hz). MS (ESI): m/z 362.4 (23.2, M+Na), 340.4 (100, M+H), 215.3 (6).

### Part E—Preparation of N-[(1E)-5-(Acetylamino)pent-1-enyl](2R)-2-amino-4-methylpentanamide, Formic Acid Salt

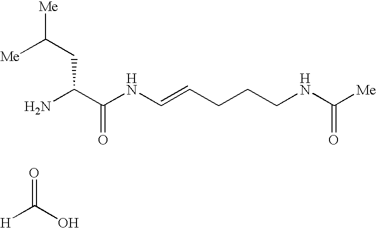

The crude acetamide from part D (45.0 mg, 0.133 mmol) was redissolved in acetonitrile/H₂O (3.00 mL; 2:1 v/v) and treated with Pd(OAc)₂ (0.60 mg, 2.7 μmol; 2 mol %) followed by TPPTS (3.0 mg, 5.3 μmol; 4 mol %) and Et₂NH (35.0 μL, 0.338 mmol) at 22° C. Complete deprotection was observed in under 0.5 h. The solution was loaded directly onto a Phenomenex Luna C18 column (21.2×250 mm) using a 0.86%/minute gradient of 5 to 35% acetonitrile containing 0.1% HCO₂H at a flow rate of 20 mL/min. The main product peak eluting at 17 minutes was lyophilized to a white solid (31.0 mg, 0.103 mmol; 63.1% over two steps). ¹H NMR (C₆D₆, 600 MHz): δ 9.99 (1H, brd, J=9.3 Hz), 8.21 (1H, s), 7.54 (1H, brs), 7.07 (1H, dd, J 14.1, 9.9 Hz), 5.39 (1H, dt, J=14.3, 7.3 Hz), 3.65 (1H, dd, J=8.3, 5.9 Hz), 3.25 (2H, td, J=6.6, 6.1 Hz), 2.04-1.99 (2H, m), 1.91 (3H, s), 1.82-1.78 (1H, m), 1.73 (1H, ddd, J=13.6, 8.1, 5.7 Hz), 1.55 (2H, quin, J=7.1 Hz), 1.50 (1H, ddd, J=13.5, 8.4, 5.8 Hz), 0.90 (3H, d, J=6.5 Hz), 0.88 (3H, d, J=6.5 Hz). ¹³C NMR (C₆D₆, 150 MHz): δ 171.2, 168.8, 162.9, 123.5, 111.6, 52.8, 43.0, 38.1, 29.8, 27.0, 24.2, 22.7, 22.5, 21.5. HRMS Calcd. for C₁₃H₂₆N₃O₂ (M+H): 256.2025. Found: 256.2016.

### EXAMPLES 65-147

### Synthesis of MMP Substrate-Hydrazide-Hynic Conjugates

The procedures used to prepare the Hynic conjugates of Examples 10-18 were used in the synthesis of the MMP substrate-hydrazide-Hynic conjugates of Examples 65-147. Yield and purity data is shown in Table 5, and mass spectrometry data are shown in Table 6.

TABLE 5

Yield and Purity Data for Examples 65-147

| | |
|---|---|
| Purity, | Chiral Purity |

| | | | |
|---|---|---|---|
| Ex. | Yield, | % | %/ |

| | | | |
|---|---|---|---|
| # | % | (HPLC) | Amino Acid |

| | | | | |
|---|---|---|---|---|
| 65 | NLys-PLG˜Hphe-YL-Ambh-Hynic | 43 | 100 | |
| 66 | Ac-P-Cit-G˜Hphe-L-Ahxh-Hynic | 29 | 100 | 97.4% L-Leu |
| 67 | Ac-PHG˜Hphe-L-Ahxh-Hynic | 37 | 96 | 94.7% L-Leu |
| 68 | NLys-NLys-PLG˜Hphe-YL-Ahxh-Hynic | 14 | 100 | 99.3% L-Leu |
| 69 | Ac-PRQ˜ITA-Ahxh-Hynic | 58 | 100 | |
| 70 | Ac-PRQ˜IT-Ahxh-Hynic | 40 | 93 | |
| 71 | Ac-PRR˜LTA-Ahxh-Hynic | 67 | 100 | 97.9% L-Ala |
| 72 | Ac-P-Cit-G˜Hphe-LA-Ahxh-Hynic | 36 | 100 | 99.3% L-Ala |
| 73 | Ac-PLG˜Hphe-Cit-L-Ahxh-Hynic | 75 | 97 | 99.9% L-Leu |
| 74 | Ac-PLG˜Hphe-OLR-Ahxh-Hynic | 73 | 100 | 95.0 %L-Arg |
| 75 | Ac-POG˜Hphe-LQ-Ahxh-Hynic | 44 | 100 | 93.8% L-Glu |
| 76 | Ac-PLG˜Hphe-YLA-Ahxh-Hynic | 26 | 98 | 96.9% L-Ala |
| 77 | Ac-PLG˜LL-Ahxh-Hynic | 35 | 100 | 92.8% L-Leu |
| 78 | Ac-PLG˜Hphe-RLA-Ahxh-Hynic | 54 | 100 | 83.9% L-Ala |
| 79 | Ac-PLG˜LYL-Ahxh-Hynic | 59 | 100 | 99.3% L-Leu |
| 80 | Ac-P-Cit-G˜Hphe-LT-Ahxh-Hynic | 3 | 98 | |
| 81 | Ac-PLG˜Hphe-RL-Ahxh-Hynic | 8 | 98 | |
| 82 | Ac-PLG˜Hphe-OLA-Ahxh-Hynic | 19 | 95 | |
| 83 | Ac-P-Cit-G-Hphe-LA-Hynic | 51 | 96 | 99.2% L-Ala |
| 84 | Ac-P-Cha-G˜Smc-HA-Ahxh-Hynic | 31 | 96 | 98.0% L-Ala |
| 85 | Ac-PLG˜LLA-Ahxh-Hynic | 45 | 98 | 85.3% L-Ala |
| 86 | Ac-POG˜Hphe-L-Nle-Ahxh-Hynic | 35 | 100 | 99.8% L-Nle |
| 87 | Ac-PLG˜Hphe-YLR-Ahxh-Hynic | 42 | 100 | 99.0% L-Arg |
| 88 | Ac-PLG˜LR-Ahxh-Hynic | 56 | 100 | 99.5% L-Arg |
| 89 | Ac-PLG˜LHL-Ahxh-Hynic | 61 | 100 | 99.9% L-Leu |
| 90 | Ac-POG˜Hphe-Smc-T-Ahxh-Hynic | 47 | 100 | 100% L-Thr |
| 91 | Ac-PRG˜LLT-Ahxh-Hynic | 98 | 100 | 100% L-Thr |
| 92 | Ac-PRG˜Hphe-LA-Ahxh-Hynic | 44 | 100 | 98.4% L-Ala |
| 93 | Ac-PLG˜LRA-Ahxh-Hynic | 56 | 100 | 96.3% L-Ala |
| 94 | Ac-P-Cit-G˜Hphe-LQ-Hynic | 36 | 100 | 99.5% L-Gln |
| 95 | Ac-POG˜Hphe-LA-Ahxh-Hynic | 38 | 100 | 98.9 % L-Ala |
| 96 | Ac-PLG˜LRL-Ahxh-Hynic | 64 | 100 | 99.7% L-Leu |
| 97 | Ac-PLG˜LYT-Ahxh-Hynic | 48 | 100 | 100% L-Thr |
| 98 | Ac-PLG˜LWA-Ahxh-Hynic | 72 | 100 | 89.8% L-Ala |
| 99 | Ac-PLG˜LOL-Ahxh-Hynic | 42 | 98 | 99.8% L-Leu |
| 100 | Ac-POG˜Hphe-LTR-Ahxh-Hynic | 55 | 97 | 89.1% L-Arg |
| 101 | Ac-POG˜LLA-Ahxh-Hynic | 53 | 100 | 90.7% L-Ala |
| 102 | Ac-PLG˜LL-Ambh-Hynic | 98 | 100 | 97.9% L-Leu |
| 103 | Ac-P-DArg-R˜LTA-Ahxh-Hynic | 8 | 98 | |
| 104 | Ac-P-NLys-R˜LTA-Ahxh-Hynic | 39 | 99 | |
| 105 | Ac-PLG˜Hphe-RLA-Ambh-Hynic | 97 | 100 | 98.0% L-Ala |
| 106 | Ac-P-Cit-G˜Aib-LA-Ahxh-Hynic | 40 | 99 | 96.5% L-Ala |
| 107 | H-DArg-P-Cit-G˜cLeu-LA-Ahxh-Hynic | 66 | 100 | 98.0% L-Ala |
| 108 | Ac-P-Cit-G˜Chg-LA-Ahxh-Hynic | 48 | 100 | |
| 109 | Ac-NLys-PLG˜LL-Ahxh-Hynic | 40 | 100 | 97.4% L-Leu |
| 110 | Ac-NLys-PLG˜Hphe-RLA-Ahxh-Hynic | 41 | 100 | 99.2% L-Ala |
| 111 | Ac-PLG˜LYA-Ahxh-Hynic | 83 | 97.6 | 84.7% L-Ala |
| 112 | Ac-PLG˜Hphe-RLT-Ahxh-Hynic | 53 | 98.3 | 100% L-Thr |
| 113 | Ac-PLG˜LAL-Ahxh-Hynic | 87 | 100 | 95.2% L-Leu |
| 114 | Ac-VRW˜LLA-Ahxh-Hynic | 28 | 100 | 99.8% L-Ala |
| 115 | Ac-VRW˜LTA-Ahxh-Hynic | 12 | 100 | 99.0% L-Ala |
| 116 | Ac-LRY˜Cha-TA-Ahxh-Hynic | 61 | 100 | 98.6% L-Ala |
| 117 | Ac-P-Cit-Cit˜LTA-Ahxh-Hynic | 66 | 93 | |
| 118 | Ac-Tic-Cit-G˜Hphe-SA-Ahxh-Hynic | 56 | 89 | |
| 119 | Ac-PRR˜Cha-TA-Ambh-Hynic | 4 | 100 | |
| 120 | Piv-PLG˜LYT-Ahxh-Hynic | 32 | 93.4 | 100% L-Thr |
| 121 | Suc-PLG˜LYT-Ahxh-Hynic | 41 | 100 | 100% L-Thr |
| 122 | Ac-P-Cit-G˜Tle-LA-Ahxh-Hynic | 62 | 100 | 99.5% L-Ala |
| 123 | Ac-PR-Cit˜LSA-Ahxh-Hynic | 59 | 99 | 98.8% L-Ala |
| 124 | H-γ-DGlu-PLG-LYT-Ahxh-Hynic | 11 | 92 | 100% L-Thr |
| 125 | Ac-Inp-Cit-G˜Hphe-LA-Ahxh-Hynic | 66 | 99 | 96.3% L-Ala |
| 126 | Ac-P-Cit-Aib˜Hphe-LA-Ahxh-Hynic | 59 | 99 | 98.4% L-Ala |
| 127 | H-NLys-PLG˜LYT-Ahxh-Hynic | 40 | 90 | 100% L-Thr |
| 128 | Ac-P-Cit-G˜Nle-LA-Ahxh-Hynic | 69 | 100 | 95.5% L-Ala |
| 129 | Ac-P-Cit-Hse˜Hphe-SA-Ahxh-Hynic | 71 | 100 | 99.5% L-Ala |
| 130 | Ac-P-Hcit-G˜Hphe-SA-Ahxh-Hynic | 39 | 100 | 100% L-Ala |
| 131 | Ac-Hpro-Cit-G˜Hphe-TA-Ahxh-Hynic | 52 | 100 | 100% L-Ala |
| 132 | Ac-P-O(Me)2-G˜Hphe-L-Nle-Ahxh-Hynic | 40 | 100 | 67.4% L-Nle |
| 133 | Ac-P-DLeu-G˜LL-Ahxh-Hynic | 36 | 100 | |
| 134 | Ac-P-Cit-G˜Igl-LA-Ahxh-Hynic | 36 | 98 | |
| 135 | Ac-PLG˜Hphe-KL-Ahxh-Hynic | 23 | 100 | |
| 136 | Ac-PLG˜Hphe-K(Me)2-L-Ahxh-Hynic | 70 | 100 | 86.0% L-Leu |
| 137 | Ac-P-NMeArg-R˜LTA-Ambh-Hynic | 5 | 100 | |
| 138 | Ac-P-Cit-G˜Abu-LA-Ahxh-Hynic | 50 | 100 | 97.4% L-Ala |
| 139 | Ac-PRG˜Hphe-Dab-A-Ahxh-Hynic | 50 | 100 | 92.8% L-Ala |
| 140 | Ac-DAla-PRG˜Ile-LA-Ahxh-Hynic | 64 | 100 | 48.2% L-Ala |
| 141 | Ac-DArg-P-Aib-G˜Hphe-LA-Ahxh-Hynic | 65 | 98 | 93.8% L-Ala |
| 142 | Ac-P-Cit-Abu˜LTA-Ahxh-Hynic | 63 | 96 | 97.6% L-Ala |
| 143 | Ac-P-Cit-G˜Hphe-Cit-L-Ahxh-Hynic | 46 | 98 | |
| 144 | Ac-PLG˜S(OBn)-LL-Ahxh-Hynic | 39 | 95 | |
| 145 | Ac-PL-DAla˜LL-Ahxh-Hynic | 18 | 100 | |
| 146 | Ac-PLG˜L-Cha-Ahxh-Hynic | 30 | 99 | |
| 147 | AC-P-Cit-G˜S(OBn)-LA-Ahxh-Hynic | 24 | 100 | |

TABLE 6
Mass Spectrometry Data for Examples 65-147

| | Low Resolution MS, Conjugate | | | |
|---|---|---|---|---|
| | Ion 1/Identity/ | Ion 2/Identity/ | High Resolution MS, Conjugate | |
| Ex. # | Intensity | Intensity | Calcd for CxHxNxOxSx [M + H]: | Found |
| 65 | 1301.6/M + H/40% | 651.3/M + 2H/100% | C65H84N14O13S [M + H]: 1301.6136 | 1301.6126 |
| 66 | 1076.4/M + H/100% | | | |
| 67 | 1056.4/M + H/100% | 528.7/M + 2H/75% | C50H65N13O11S [M + H]: 1056.4720 | 1056.4696 |
| 68 | 1410.6/M + H/30% | 705.9/M + 2H/95% | C68H100N16O14S [M + 2H]: 705.3736 | 705.3731 |
| 69 | 1157.3/M + H/60% | 579.2/M + 2H/100% | | |
| 70 | 1086.4/M + H/75% | 543.9/M + 2H/100% | | |
| 71 | 1185.4/M + H/25% | 593./M + 2H/100% | C51H80N18O13S [2M + H]: 593.3009 | 593.3004 |
| 72 | 1147.4/M + H/100% | 574.3/M + 2H/100% | | |
| 73 | 1189.4/M + H/100% | 595.3/M + 2H/20% | | |
| 74 | 1302.6/M + H/30% | 651.8/M + 2H/100% | | |
| 75 | 1161.4/M + H/100% | 581.8/M + 2H/50% | C54H76N14O13S [2M + H]: 581.2791 | 581.2789 |
| 76 | 1266.4/M + H/100% | 633.7/M + 2H/65% | C62H83N13O14S [2M + H]: 633.8024 | 633.803 |
| 77 | 984.5/M + H/100% | 536.2/20% | C46H69N11O11S {M + H]: 984.4971 | 984.4988 |
| 78 | 1259.6/M + H/90% | 630.5/M + 2H/100% | C59H86N16O13S [M + H]: 1259.6354 | 1259.6325 |
| 79 | 1147.5/M + H/100% | | C55H78N12O13S [M + H]: 1147.5605 | 1147.5627 |
| 80 | 1177.4/M + H/95% | 598.3/M + 2H/100% | | |
| 81 | 1188.4/M + H/95% | 594.8/M + 2H/100% | | |
| 82 | 1217.5/M + H/65% | 609.3/M + 2H/100% | | |
| 83 | 1034.0/M + H/70% | 517.3/M + 2H/100% | | |
| 84 | 1123.3/M + H/60% | 562.2/M + 2H/100% | C50H70N14O12S2 [M + H]: 1123.4812 | 1123.481 |
| 85 | 1055.4/M + H/100% | 607.3/20% | C49H74N12O12S [M + H]: 1055.5343 | 1055.5349 |
| 86 | 1146.4/M + H/100% | 573.8/M + 2H/40% | C55H79N13O12S [2M + H]: | 573.7926 |
| 87 | 1351.0/M + H/100% | 676.2/M + 2H/40% | C65H90N16O14S [2M + H]: 676.3244 | 676.3254 |
| 88 | 1027.5/M + H/75% | 514.3/M + 2H/100% | C46H70N14O11S [M + H]: 1027.5142 | 1027.5139 |
| 89 | 1121.6/M + H/93% | 561.3/M + 2H/100% | C52H76N14O12S [M + H]: 1121.5561 | 1121.5556 |
| 90 | 1138.3/M + H/45% | 569.8/M + 2H/100% | C51H71N13O12S2 [M + H]: 1138.4808 | 1138.4805 |
| 91 | 1128.4/M + H/100% | 564.9/M + 2H/45% | C50H77N15O13S [M + H]: 1128.5619 | 1128.5625 |
| 92 | 1146.4/M + H/100% | 573.7/M + 2H/50% | C53H75N15O12S [M + H]: 1146.5513 | 1146.5514 |
| 93 | 1098.4/M + H/85% | 549.8/M + 2H/100% | C49H75N15O12S [M + H]: 1098.5513 | 1098.5514 |
| 94 | 1204.4/M + H/100% | 602.8/M + 2H/30% | | |
| 95 | 1104.4/M + H/100% | 552.8/M + 2H/40% | | |
| 96 | 1140.4/M + H/100% | 570.9/M + 2H/95% | C52H81N15O12S [M + H]: 1140.5983 | 1140.5982 |
| 97 | 1135.5/M + H/100% | | C53H74N12O14S [M + H]: 1135.5241 | 1135.5243 |
| 98 | 1128.4/M + H/100% | | C54H73N13N12S [M + H]: 1128.5295 | 1128.5278 |
| 99 | 1098.4/M + H/60% | 549.9/M + 2H/100% | C51H79N13O12S [M + H]: 1098.5765 | 1098.5781 |
| 100 | 1290.6/M + H/50% | 645.8/M + 2H/100% | C59H87N17O14S [M + 2H]: 645.8242 | 645.8248 |
| 101 | 1056.4/M + H/100% | | C48H73N13O12S [M + H]: 1056.5295 | 1056.529 |
| 102 | 1004.4/M + H/100% | 536.4/45% | | |
| 103 | 1185.6/M + H/25% | 593.3/M + 2H/100% | | |
| 104 | 579.3/M + 2H/100% | 1157.4/M + 2H/20% | C51H80N16O13S [M + 2H]: 579.2978 | 579.2985 |
| 105 | 1279.5/M + H/100% | 640.5/M + 2H/40% | C61H82N16O13S [M + H]: 1279.6041 | 1279.604 |
| 106 | 1071.4/M + H/100% | 536.3/M + 2H/50% | | |
| 107 | 1211.5/M + H/25% | 606.4/M + 2H/100% | | |
| 108 | 1125.4/M + H/100% | 553.4/M + 2H/40% | | |
| 109 | 1112.6/M + H/100% | 556.8/M + 2H/65% | C52H81N13O12S [M + H]: 1112.5921 | 1112.592 |
| 110 | 1387.6/M + H/10% | 694.7/M + 2H/100% | C65H98N18O14S [M + 2H]: 694.3688 | 694.3719 |
| 111 | 1105.5/M + H/100% | 657.3/13% | C52H72N12O13S [M + H]: 1105.5135 | 1105.515 |
| 112 | 1289.6/M + H/100% | 645.5/M + 2H/75% | C60H88N16O14S [M + H]: 1289.6459 | 1289.642 |
| 113 | 1055.6/M + H/100% | 607.3/20% | C49H74N12O12S [M + H]: 1055.5343 | 1055.534 |
| 114 | 1229.6/M + H/100% | 615.3/M + 2H/80% | C58H84N16O12S [M + H]: 1229.6248 | 1229.626 |
| 115 | 1217.6/M + H/100% | 609.3/M + 2H/80% | C56H78N16O13S [M + H]: 1217.5884 | 1217.586 |
| 116 | 1248.4/M + H/100% | 624.9/M + 2H/60% | C58H85N15O14S [M + H]: 1248.6194 | 1248.622 |
| 117 | 1187.6/M + H/100% | 594.2/M + 2H/45% | | |
| 118 | 1183.5/M + H/100% | 592.2/M + 2H/85% | | |
| 119 | 1245.6/M + H/25% | 622.8/M + 2H/100% | | |
| 120 | 1177.5/M + H/100% | 589.8/M + 2H/40% | C56H80N12O14S [M + H]: 1177.5710 | 1177.572 |
| 121 | 1193.4/M + H/100% | 597.3/M + 2H/30% | C55H76N12O16S [M + H]: 1193.5296 | 1193.528 |
| 122 | 1099.4/M + H/100% | 550.3/M + 2H/75% | C49H74N14O13S [M + H]: 1099.5353 | 1099.535 |
| 123 | 1172.5/M + H/100% | 586.8/M + 2H/85% | C50H77N17O14S [M + H]: 1172.5629 | 1172.563 |
| 124 | 1222.4/M + H/100% | 611.8/M + 2H/100% | | |
| 125 | 1161.4/M + H/50% | 581.3/M + 2H/100% | | |
| 126 | 1175.5/M + H/95% | 588.4/M + 2H/100% | | |
| 127 | 1221.6/M + H/15% | 611.3/M + 2H/40% | C57H84N14O14S [M + 2H]: 611.3079 | 611.3085 |
| 128 | 1099.4/M + H/100% | 550.3/M + 2H/75% | | |
| 129 | 1165.4/M + H/100% | 583.3/M + 2H/70% | C52H72N14O15S [M + H]: 1165.5095 | 1165.511 |
| 130 | 1135.5/M + H/100% | 568.3/M + 2H/85% | C51H70N14O14S [M + H]: 1135.4989 | 1135.498 |
| 131 | 1149.4/M + H/100% | 575.3/M + 2H/60% | C52H72N14O14S [M + H]: 1149.5146 | 1149.517 |
| 132 | 1174.5/M + H/100% | 588.2/M + 2H/55% | C97H83N13O12S [M + H]: 1174.6077 | 1174.605 |
| 133 | 984.4/M + H/100% | 492.9/M + 2H/30% | C46H69N11O11S [M + H]: 984.4971 | 984.5009 |
| 134 | 1159.4/M + H/100% | 580.3/M + 2H/75% | C54H74N14O13S [M + H]: 1159.5353 | 1159.537 |
| 135 | 1160.5/M + H/100% | 581.0/M + 2H/85% | C56H81N13O12S [M + H]: 1160.5921 | 1160.588 |
| 136 | 1188.6/M + H/100% | | C58H85N13O12S [M + H]: 1188.6234 | 1188.626 |
| 137 | 1245.6/M + H/25% | 623.3/M + 2H/100% | | |
| 138 | 1071.4/M + H/100% | 536.3/M + 2H/70% | | |
| 139 | 1133.5/M + H/55% | 567.4/M + 2H/100% | | |
| 140 | 1170.4/M + H/100% | 585.8/M + 2H/90% | | |
| 141 | 1231.4/M + H/100% | 616.5/M + 2H/40% | | |
| 142 | 1115.3/M + H/100% | 558.4/M + 2H/50% | | |
| 143 | 1233.5/M + H/55% | 617.3/M + 2H/100% | | |
| 144 | 1161.4/M + H/100% | 581.3/M + 2H/30% | | |
| 145 | 998.2/M + H/100% | | | |
| 146 | 1024.4/M + H/100% | | | |
| 147 | 1163.5/M + H/100% | | | |

### Synthesis of Complexes [^{99m}Tc(HYNIC-MMPsub)(tricine)(TPPTS)]

The procedures described in Examples 27-44 were used to prepare these additional ^{99m}Tc complexes. Analytical and yield data for these complexes are shown in Table 7.

TABLE 7
Analytical and Yield data for
[^{99m}Tc(HYNIC-MMPsub)(tricine)(TPPTS)] Complexes.

| | Hynic | | RCP Purity, % | |
|---|---|---|---|---|
| Example # | Conjugate # | RT (min) | (HPLC) | HPLC Gradient |
| 148 | 65 | 13.7 | 80.0 | 20-40/20 min |
| 149 | 66 | 12.5 | 98.0 | 10-40/20 min |
| 150 | 67 | 13.1 | 74.0 | 10-40/20 min |
| 151 | 68 | 12.5 | 78.6 | 20-40/20 min |
| 152 | 69 | 11.8 | 90.0 | 0-40/20 min |
| 153 | 70 | 11.7 | 89.0 | 0-40/20 min |
| 154 | 71 | 11.8 | 86.9 | 0-40/20 min |
| 155 | 72 | 14.1 | 99.1 | 0-40/20 min |
| 156 | 73 | 15.9 | 96.7 | 10-40/20 min |
| 157 | 74 | 12.8 | 95.8 | 20-40/20 min |
| 158 | 75 | 13.2 | 88.7 | 20-40/20 min |
| 159 | 76 | 14.5 | 97.5 | 20-40/20 min |
| 160 | 77 | 11.1 | 97.0 | 20-40/20 min |
| 161 | 78 | 13.4 | 93.4 | 20-40/20 min |
| 162 | 79 | 12.6 | 100 | 20-40/20 min |
| 163 | 80 | 12.0 | 95.0 | 10-40/20 min |
| 164 | 81 | 13.4 | 100 | 20-40/20 min |
| 165 | 82 | 13.6 | 96.5 | 20-40/20 min |
| 166 | 83 | 13.7 | 96.1 | 10-40/20 min |
| 167 | 84 | 14.8 | 71.5 | 20-40/20 min |
| 168 | 85 | 16.1 | 97.0 | 20-40/20 min |
| 169 | 86 | 10.8 | 93.5 | 20-40/20 min |
| 170 | 87 | 13.4, 14.0 | 68.1 | 20-40/20 min |
| 171 | 88 | 12.2 | 100 | 10-40/20 min |
| 172 | 89 | 14.6 | 70.1 | 10-40/20 min |
| 173 | 90 | 11.3 | 95.1 | 20-40/20 min |
| 174 | 91 | 15.5 | 89.9 | 0-40/20 min |
| 175 | 92 | 14.8 | 97.6 | 10-40/20 min |
| 176 | 93 | 12.6 | 98.9 | 10-40/20 min |
| 177 | 94 | 13.8 | 100 | 10-40/20 min |
| 178 | 95 | 13.2 | 97.3 | 10-40/20 min |
| 179 | 96 | 15.0 | 92.4 | 10-40/20 min |
| 180 | 97 | 13.8 | 98.5 | 10-40/20 min |
| 181 | 98 | 15.6 | 98.4 | 10-40/20 min |
| 182 | 99 | 14.7 | 98.4 | 10-40/20 min |
| 183 | 100 | 14.0 | 96.9 | 10-40/20 min |
| 184 | 101 | 11.0 | 87.7 | 10-40/20 min |
| 185 | 102 | 12.5 | 97.1 | 20-40/20 min |
| 186 | 103 | 13.6 | 92.5 | 0-40/20 min |
| 187 | 104 | 12.9 | 83.9 | 0-40/20 min |
| 188 | 105 | 14.3 | 59.2 | 20-40/20 min |
| 189 | 106 | 14.3 | 87.2 | 0-40/20 min |
| 190 | 107 | 14.5 | 73.2 | 0-40/20 min |
| 191 | 108 | 15.8 | 80.6 | 0-40/20 min |
| 192 | 109 | 17.4 | 72.9 | 0-40/20 min |
| 193 | 110 | 19.1 | 82.0 | 0-40/20 min |
| 194 | 111 | 17.2 | 75.1 | 0-40/20 min |
| 195 | 112 | 19.9 | 100 | 0-40/20 min |
| 196 | 113 | 17.9 | 99.2 | 0-40/20 min |
| 197 | 114 | 21.1 | 94.9 | 0-40/20 min |
| 198 | 115 | 19.2 | 96.6 | 0-40/20 min |
| 199 | 116 | 13.2 | 90.3 | 0-40/20 min |
| 200 | 117 | 19.4 | 93.4 | 0-40/20 min |
| 201 | 118 | 17.3 | 100 | 0-40/20 min |
| 202 | 119 | 15.3 | 93.7 | 0-40/20 min |
| 203 | 120 | 19.5 | 100 | 0-40/20 min |
| 204 | 121 | 17.2 | 84.7 | 0-40/20 min |
| 205 | 122 | 13.3 | 96.0 | 0-40/20 min |
| 206 | 123 | 12.7 | 100 | 0-40/20 min |
| 207 | 124 | 15.6 | 92.0 | 0-40/20 min |
| 208 | 125 | 15.2 | 100 | 0-40/20 min |
| 209 | 126 | 18.0 | 99.0 | 0-40/20 min |
| 210 | 127 | 16.2 | 99.0 | 0-40/20 min |
| 211 | 128 | 15.3 | 98.7 | 0-40/20 min |
| 212 | 129 | 14.4 | 100 | 0-40/20 min |
| 213 | 130 | 15.5 | 100 | 0-40/20 min |
| 214 | 131 | 16.0 | 98.4 | 0-40/20 min |
| 215 | 132 | 18.2 | 100 | 0-40/20 min |
| 216 | 133 | 18.6 | 38.0 | 0-40/20 min |
| 217 | 134 | 17.9 | 95.4 | 0-40/20 min |
| 218 | 135 | 19.6 | 85.1 | 0-40/20 min |
| 219 | 136 | 18.7 | 94.6 | 0-40/20 min |
| 220 | 137 | 13.5 | 70.4 | 0-40/20 min |
| 221 | 138 | 14.1 | 99.7 | 0-40/20 min |
| 222 | 139 | 14.7 | 64.8 | 0-40/20 min |
| 223 | 140 | 15.1 | 100 | 0-40/20 min |
| 224 | 141 | 16.4 | 65.4 | 0-40/20 min |
| 225 | 142 | 15.5 | 97.9 | 0-40/20 min |
| 226 | 143 | 23.0 | 95.0 | 0-40/20 min |
| 227 | 144 | 15.7 | 100 | 0-40/20 min |
| 228 | 145 | 18.2 | 96.9 | 0-40/20 min |
| 229 | 146 | 21.9 | 93.2 | 0-40/20 min |
| 230 | 147 | 18.1 | 99.7 | 0-40/20 min |

### EXAMPLE 231

### In Vitro Plasma Protein Binding

### Part A—Sample Preparation

Mouse, rabbit and human plasma was purchased through a commercial vendor (Biological Specialty Corporation, Colmar, Pa.). Ultrafiltered/deproteinized human plasma, purchased from the same vendor, was used as a protein free control matrix for background subtraction. Radiolabelled compound (Tc-99m or C-14) was added to plasma to achieve a final concentration of 0.6-2.0 uCi/mL or 0.01-0.2 uCi/mL, respectively. Samples were vortexed and incubated at 37° C. for 30 min on a rocker platform. Compound was also prepared in deproteinized plasma and used to determine non-specific binding.

### Part B—Sample Analysis

Plasma or deproteinized plasma (0.025 mL) aliquots (n=3) were transferred to separate vials for pre-filtration counting using a Tri-carb® 2500TR liquid scintillation counter (Perkin Elmer, Gaithersburg, Md.) or Wallac Wizard gamma counter (Perkin Elmer, Boston, Mass.). A 0.3 mL aliquot of plasma or deproteinized plasma was transferred to a Centrifree® micropartition cartridge, MW cutoff of 30,000 daltons (n=3), and centrifuged at 2500×g for 20 min at room temperature. After centrifugation, 0.025 mL aliquots (n=4) of filtrate were transferred to vials and counted for radioactivity.

### Part C—Data Analysis

The percent of compound bound to plasma proteins was calculated using the following equation:

$$\% Bound = \frac{\begin{matrix}\text{Compound Total} - \\ \text{Compound Unbound (filtrate)}\end{matrix}}{\text{Compound Total}} \times 100$$

Where:
Compound Total=Radioactivity (dpm) in 0.025 mL of sample before ultrafiltration.
Compound Unbound=Radioactivity (dpm) in 0.025 mL of filtrate.
   Compound bound to ultrafiltered/deproteinized human plasma was calculated and subtracted as background from all samples incubated in plasma. Data are shown in Table 8.

### EXAMPLE 232

### In Vitro Blood Stability

Radiolabelled test compounds (Tc-99m, C-14) were incubated in fresh heparinized mouse blood (0.2-5.0 uCi/mL) while rocking at 37° C. for 15 minutes. Blood (0.3 mL) was transferred directly into 1 mL of acetonitrile, which inhibited esterase activity and metabolism of the compound. Test compound was also incubated in saline for 15 min to assess non-matrix stability. Samples were vortexed for 30 seconds and centrifuged at 2500×g for 20 min. The supernatant was transferred to a fresh tube where acetonitrile was evaporated to dryness under a stream of nitrogen in a heating block at 37° C. Samples were reconstituted to 0.3 mL with 0.1% formic acid. Aliquots (0.05 mL) were analyzed for compound stability by reversed-phase HPLC with radiochemical detection. Data are shown in Table 8.

### EXAMPLE 233

### In Vivo Blood Stability

Blood samples (0.3 mL) were collected from mice at 15 min following i.v. administration of 0.1-7.0 mCi/kg of radiolabelled test compound (Tc-99m, C-14) and immediately added to 0.9 mL of acetonitrile. Samples were vortexed for 30 seconds and centrifuged at 2500×g for 20 mins. The supernatant was transferred to a fresh tube where acetonitrile was evaporated to dryness under a stream of nitrogen in a heating block at 37° C. Samples were reconstituted to 0.3 mL with 0.1% formic acid. Aliquots (0.05 mL) were analyzed for compound stability by reversed-phase HPLC with radiochemical detection. Data are shown in Table 8.

TABLE 8

MMP-2 and MMP-9 Activity, Protein Binding, and Stability of
Examples 18, 27-30, 32-40, and 148-230.

Stability,
Mouse,
15 Min

| | | | | | |
|---|---|---|---|---|---|
| Example | MMP2 | MMP9 | Protein Binding, | In | In Vivo |

| | | | | | | |
|---|---|---|---|---|---|---|
| # | Sequence | Kcat/Km | Kcat/Km | % (H/R/M/S)¹ | Vitro | Blood |

| | | | | | | |
|---|---|---|---|---|---|---|
| 18 | Ac-Csa-PLG˜Hphe-YL-Ambh-Hynic | 19,465 | 41,623 | | | |
| 27 | Hynic-Ahx-PLG˜Hphe-OLEE-OH | 83,900 | 1,670 | 26/—/28 | | |
| 28 | Hynic-ff-Ahx-PLG˜Hphe-OLEE-OH | 81,562 | 6,675 | | | |
| 29 | Hynic-fff-Ahx-PLG˜Hphe-OLEE-OH | 11,631 | 1,742 | | | |
| 30 | Ac-PLG˜Hphe-OLEE-Ahx-Hynic | 8,025 | 1,986 | | | |
| 32 | Ac-PLG˜Hphe-YL-Ambh-Hynic | 63,172 | 189,715 | 72/80/70 | 100 | 9 |
| 33 | Ac-POG˜Hphe-L-Ambh-Hynic | 77,740 | 22,049 | 44/50/40 | | |
| 34 | Ac-NGlu-PLG˜Hphe-YL-Ambh-Hynic | 326,930 | >100,000 | 76/76/76/30 | 40 | 72 |
| 35 | Ac-PLG˜Hphe-YL-Ahxh-Hynic | >100,000 | >100,000 | 66/83/42 | | |
| 36 | Ac-POG˜Hphe-L-Hynic | 63,685 | 4,453 | 36/62/27 | | |
| 37 | Ac-NGlu-POG˜Hphe-L-Hynic | 265 | 613 | 27/38/18 | | |
| 38 | Ac-PLG˜Hphe-YL-Hynic | 63,199 | >100,000 | | | |
| 39 | Ac-PLG˜Hphe-OL-Ambh-Hynic | 42,684 | 57,730 | 47/75/86 | 92 | 91 |
| 40 | Ac-Ahxh-Hynic | na | na | 8.6/20/4.0 | | |
| 148 | NLys-PLG˜Hphe-YL-Ambh-Hynic | 121,054 | 264,154 | 57/67/45 | 14 | 12 |
| 149 | Ac-P-Cit-G˜Hphe-L-Ahxh-Hynic | 161,025 | 33,268 | 32/55/45 | 36 | 9 |
| 150 | Ac-PHG˜Hphe-L-Ahxh-Hynic | 22,093 | 4,433 | 51/67/59 | | |
| 151 | NLys-NLys-PLG˜Hphe-YL-Ahxh-Hynic | 26,284 | 33,774 | 41/48/49/54 | | |
| 152 | Ac-PRQ˜ITA-Ahxh-Hynic | 2,395 | 3,524 | 13/21/11/4.2 | 13 | |
| 153 | Ac-PRQ˜IT-Ahxh-Hynic | 898 | 1,705 | 15/21/10/−2.4 | | |
| 154 | Ac-PRR˜LTA-Ahxh-Hynic | 10,115 | 33,479 | 16/21/12/−0.75 | 0 | |
| 155 | Ac-P-Cit-G˜Hphe-LA-Ahxh-Hynic | 827,227 | 583,549 | 30/43/25/9.8 | 100 | 0 |
| 156 | Ac-PLG˜Hphe-Cit-L-Ahxh-Hynic | 156,802 | 170,616 | 44/67/62/22 | 30 | 10 |
| 157 | Ac-PLG˜Hphe-OLR-Ahxh-Hynic | 46,574 | 22,065 | 18/56/5.3/46 | 85 | 18 |
| 158 | Ac-POG˜Hphe-LQ-Ahxh-Hynic | >250,000 | 84,763 | 24/36/36/7.6 | 100 | 0 |
| 159 | Ac-PLG˜Hphe-YLA-Ahxh-Hynic | 221,288 | 201,323 | 46/59/43/32 | 0 | 8 |
| 160 | Ac-PLG˜LL-Ahxh-Hynic | >250,000 | 217,677 | 33/50/24/16 | 94 | 11 |
| 161 | Ac-PLG˜Hphe-RLA-Ahxh-Hynic | 64,013 | 57,711 | 26/50/40/15 | 5 | 44 |
| 162 | Ac-PLG˜LYL-Ahxh-Hynic | 65,285 | 94,978 | 32/58/44/18 | 85 | 7 |
| 163 | Ac-P-Cit-G˜Hphe-LT-Ahxh-Hynic | | | 26/40/22/40 | 16 | 0 |
| 164 | Ac-PLG˜Hphe-RL-Ahxh-Hynic | 241,959 | 168,186 | 47/64/46/17 | >20 | 17 |
| 165 | Ac-PLG˜Hphe-OLA-Ahxh-Hynic | 29,512 | 14,104 | 32/50/34/34 | 71 | 0 |
| 166 | Ac-P-Cit-G-Hphe-LA-Hynic | >250,000 | >250,000 | 37/53/28/3.1 | 0 | 0 |
| 167 | Ac-P-Cha-G˜Smc-HA-Ahxh-Hynic | 145,749 | >250,000 | 29/46/39/7.9 | 0 | 0 |
| 168 | Ac-PLG˜LLA-Ahxh-Hynic | 387,194 | 225,526 | 38/40/41/2.4 | 77 | 0 |
| 169 | Ac-POG˜Hphe-L-Nle-Ahxh-Hynic | 401,093 | 51,950 | 47/57/45/26 | 67 | 0 |
| 170 | Ac-PLG˜Hphe-YLR-Ahxh-Hynic | 363,880 | 242,786 | 37/72/18/44 | 84 | 30 |
| 171 | Ac-PLG˜LR-Ahxh-Hynic | 161,793 | 142,848 | 15/38/3.4/15 | 90 | 0 |
| 172 | Ac-PLG˜LHL-Ahxh-Hynic | 73,721 | 156,170 | 29/41/28/24 | 93 | 0 |
| 173 | Ac-POG˜Hphe-Smc-T-Ahxh-Hynic | >500,000 | 85,367 | 32/37/25/15 | 0 | 0 |
| 174 | Ac-PRG˜LLT-Ahxh-Hynic | 98,638 | 73,345 | 26/32/27/8.7 | 0 | 0 |
| 175 | Ac-PRG˜Hphe-LA-Ahxh-Hynic | >500,000 | >500,000 | 34/34/20/22 | 0 | 0 |
| 176 | Ac-PLG˜LRA-Ahxh-Hynic | >500,000 | >500,000 | 20/30/9.0//10 | 0 | 0 |
| 177 | Ac-P-Cit-G˜Hphe-LQ-Hynic | 171,020 | 79,552 | 32/39/31/1.1 | 39 | 0 |
| 178 | Ac-POG˜Hphe-LA-Ahxh-Hynic | >500,000 | >500,000 | 29/31/28/12 | 0 | 0 |
| 179 | Ac-PLG˜LRL-Ahxh-Hynic | 181,818 | 167,012 | 23/38/23/18 | 64 | 10 |
| 180 | Ac-PLG˜LYT-Ahxh-Hynic | 185,624 | 223,422 | 30/68/46/2.9 | 40 | 63 |
| 181 | Ac-PLG˜LWA-Ahxh-Hynic | >500,000 | >500,000 | 46/73/65/−4.4 | 67 | 15 |
| 182 | Ac-PLG˜LOL-Ahxh-Hynic | 91,360 | 39,373 | 23/29/23/15 | 100 | 0 |
| 183 | Ac-POG˜Hphe-LTR-Ahxh-Hynic | >500,000 | 105,427 | 17/33/1.0/16 | 74 | 0 |
| 184 | Ac-POG˜LLA-Ahxh-Hynic | >500,000 | 186,640 | 3.8/14/4.1/0.5 | 0 | 0 |
| 185 | Ac-PLG˜LL-Ambh-Hynic | 33,550 | 36,301 | 46/73/65/−4.4 | 97 | 47 |
| 186 | Ac-P-DArg-R˜LTA-Ahxh-Hynic | 6,882 | 0 | 2.7/na/na/1.4 | 0 | 0 |
| 187 | Ac-P-NLys-R˜LTA-Ahxh-Hynic | 0 | 0 | 23/29/23/15 | 0 | 0 |
| 188 | Ac-PLG˜Hphe-RLA-Ambh-Hynic | 197 | 4,553 | 17/33/1.0/16 | 0 | 0 |
| 189 | Ac-P-Cit-G˜Aib-LA-Ahxh-Hynic | 0 | 772 | 14/24/12/2.2 | 6 | 41 |
| 190 | H-DArg-P-Cit-G˜cLeu-LA-Ahxh-Hynic | 77 | 0 | 18/24/20/−4.2 | 0 | 0 |
| 191 | Ac-P-Cit-G˜Chg-LA-Ahxh-Hynic | 14,415 | 20,717 | 24/38/35/6.7 | 8 | 2 |
| 192 | Ac-NLys-PLG˜LL-Ahxh-Hynic | 76,287 | 83,184 | 24/36/27/0.8 | 78 | 0 |
| 193 | Ac-NLys-PLG˜Hphe-RLA-Ahxh-Hynic | 461,387 | 372,802 | 25/30/9.0/23 | 0 | 0 |
| 194 | Ac-PLG˜LYA-Ahxh-Hynic | 404,019 | 511,527 | 36/61/32/5.9 | 20 | 1 |
| 195 | Ac-PLG˜Hphe-RLT-Ahxh-Hynic | 118,836 | 103,248 | 38/67/26/36 | 34 | 0 |
| 196 | Ac-PLG˜LAL-Ahxh-Hynic | 4,381 | 7,138 | 23/42/22/7.9 | 80 | 6 |
| 197 | Ac-VRW˜LLA-Ahxh-Hynic | 43,043 | 72,248 | 67/62/55/27 | 0 | 0 |
| 198 | Ac-VRW˜LTA-Ahxh-Hynic | 14,449 | 29,875 | 47/44/25/26 | 0 | 0 |
| 199 | Ac-LRY˜Cha-TA-Ahxh-Hynic | 0 | 811 | 49/61/48/24 | 0 | 0 |
| 200 | Ac-P-Cit-Cit-LTA-Ahxh-Hynic | 27,321 | 29,292 | 13/23/12/3.5 | 0 | 0 |
| 201 | Ac-Tic-Cit-G˜Hphe-SA-Ahxh-Hynic | 3,097 | 1,880 | 45/39/32/12 | 0 | 15 |
| 202 | Ac-PRR˜Cha-TA-Ambh-Hynic | 0 | 1,578 | 37/50/22/6.9 | 0 | 0 |
| 203 | Piv-PLG˜LYT-Ahxh-Hynic | 569,232 | 281,385 | 56/94/50/−3.5 | 23 | 2 |
| 204 | Suc-PLG˜LYT-Ahxh-Hynic | 141,057 | 80,965 | 39/63/60/5.7 | 78 | 2 |
| 205 | Ac-P-Cit-G˜Tle-LA-Ahxh-Hynic | 164 | 0 | 18/25/14/−8.1 | 8 | 19 |
| 206 | Ac-PR-Cit˜LSA-Ahxh-Hynic | 14,735 | 40,146 | 17/25/10/2.9 | 0 | 0 |
| 207 | H-γ-DGlu-PLG-LYT-Ahxh-Hynic | 152,578 | 59,385 | 23/34/12/4.8 | 56 | 0 |
| 208 | Ac-Inp-Cit-G˜Hphe-LA-Ahxh-Hynic | 4,582 | 1,651 | 35/47/28/7.3 | 26 | 0 |
| 209 | Ac-P-Cit-Aib˜Hphe-LA-Ahxh-Hynic | 275 | 0 | 33/36/22/−7.7 | 22 | 18 |
| 210 | H-NLys-PLG˜LYT-Ahxh-Hynic | 85,142 | 148,727 | 27/33/18/6.5 | 0 | 0 |
| 211 | Ac-P-Cit-G˜Nle-LA-Ahxh-Hynic | 173,759 | 115,756 | 17/24/13/6.0 | 26 | 0 |
| 212 | Ac-P-Cit-Hse˜Hphe-SA-Ahxh-Hynic | 91,443 | 38,282 | 16/24/11/2.1 | 8 | 12 |
| 213 | Ac-P-Hcit-G˜Hphe-SA-Ahxh-Hynic | 293,419 | 395,665 | 19/26/17/7.8 | 8 | 1 |
| 214 | Ac-Hpro-Cit-G˜Hphe-TA-Ahxh-Hynic | 99,649 | 159,688 | 23/32/21/5.5 | 0 | 7 |
| 215 | Ac-P-O(Me)2-G˜Hphe-L-Nle-Ahxh-Hynic | 566,772 | 26,991 | 35/50/41/8.4 | 61 | 0 |
| 216 | Ac-P-DLeu-G˜LL-Ahxh-Hynic | 5,013 | 438 | 23/47/41/9.2 | 100 | 17 |
| 217 | Ac-P-Cit-G˜Igl-LA-Ahxh-Hynic | 189 | 0 | 34/38/25/7.1 | 10 | 3 |
| 218 | Ac-PLG˜Hphe-KL-Ahxh-Hynic | 78,925 | 93,353 | 41/79/39/11 | 84 | 75 |
| 219 | Ac-PLG˜Hphe-K(Me)2-L-Ahxh-Hynic | 72,015 | 55,017 | 38/82/46/20 | 92 | 83 |
| 220 | Ac-P-NMeArg-R˜LTA-Ambh-Hynic | 879 | 792 | 19/24/14/4.2 | 0 | 0 |
| 221 | Ac-P-Cit-G˜Abu-LA-Ahxh-Hynic | 17,556 | 20,578 | 12/21/13/3.6 | 0 | 0 |
| 222 | Ac-PRG˜Hphe-Dab-A-Ahxh-Hynic | 590,880 | 474,310 | 30/35/26/−0.9 | 0 | 6 |
| 223 | Ac-DAla-PRG˜Ile-LA-Ahxh-Hynic | 130,378 | 61,740 | 15/26/15/−9.3 | 0 | 0 |
| 224 | Ac-DArg-P-Aib-G˜Hphe-LA-Ahxh-Hynic | 18,584 | 0 | 35/38/26/1.5 | 0 | 4 |
| 225 | Ac-P-Cit-Abu˜LTA-Ahxh-Hynic | 55,354 | 77,834 | 19/27/19/−4.7 | 9 | 2 |
| 226 | Ac-P-Cit-G˜Hphe-Cit-L-Ahxh-Hynic | 42,648 | 11,437 | 30/40/40/2.7 | 89 | 33 |
| 227 | Ac-PLG˜S(OBn)-LL-Ahxh-Hynic | 179,973 | 187,202 | 75/84/87/−4.3 | 100 | 51 |
| 228 | Ac-PL-DAla˜LL-Ahxh-Hynic | 0 | 0 | 23/55/36/10 | 100 | 94 |
| 229 | Ac-PLG˜L-Cha-Ahxh-Hynic | 119,777 | 147,465 | 55/76/66/23 | 100 | 45 |
| 230 | Ac-P-Cit-G˜S(OBn)-LA-Ahxh-Hynic | >250,000 | >250,000 | 29/38/44/5.8 | 53 | 38 |

¹H/R/M/S = Human/Rat/Mouse/Saline Control

### EXAMPLES 234-269

### Synthesis of MMP Substrate-Hydrazide Amines

The procedures of Examples 61 and 62 were used to prepare the MMP substrate-hydrazide free amine conjugates of Examples 234-269. Yield and purity data are shown in Table 9, and mass spectrometry data are shown in Table 10.

TABLE 9

Yield and Purity data for Examples 234-269

| | | |
|---|---|---|
| Ex. | Yield, | Purity, |

| | | | |
|---|---|---|---|
| # | Sequence | % | % (HPLC) |

| | | | |
|---|---|---|---|
| 234 | Ac-P-Cit-G˜Hphe-LA-Ahxh-H | 44 | 90 |
| 235 | Ac-PLG˜LL-Ahxh-H | 20 | 100 |
| 236 | Ac-PLG˜LY(t-Bu)T-Ahxh-H | 42 | 95 |
| 237 | Ac-PLG˜LW(Boc)A-Ahxh-H | 35 | 90 |
| 238 | Ac-PO(Boc)G˜Hphe-LTR-Ahxh-H | 55 | 95 |
| 239 | Ac-PLG˜Hphe-K(Boc)L-Ahxh-H | 71 | 100 |
| 240 | Ac-PLG˜S(OBn)-LL-Ahxh-H | 31 | 100 |
| 241 | Ac-PLG˜L-Cha-Ahxh-H | 85 | 100 |
| 242 | Ac-P-Cit-G˜S(OBn)-LA-Ahxh-H | 59 | 100 |
| 243 | Ac-NGlu(t-Bu)-PLG˜Hphe-YL-Ahxh-H | 53 | 100 |
| 244 | Ac-PLG˜Cit-LA-Ahxh-H | 28 | 95 |
| 245 | Ac-P-NLeu-G˜LL-Ahxh-H | 19 | 98 |
| 246 | Ac-PL-NLys(Boc)˜LL-Ahxh-H | 39 | 100 |
| 247 | Ac-P-Cit-G˜Hphe-O(Boc)L-Ahxh-H | 14 | 90 |
| 248 | Ac-PLG˜LY(t-Bu)Q(Trt)-Ahxh-H | 57 | 100 |
| 249 | Ac-Oic-LG˜LL-Ahxh-H | 43 | 90 |
| 250 | Ac-PLG˜Ahp-Y(t-Bu)L-Ahxh-H | 63 | 100 |
| 251 | Ac-PL-Sar˜LL-Ahxh-H | 87 | 100 |
| 252 | Ac-PLG˜Pabu-Cit-L-Ahxh-H | 20 | 100 |
| 253 | Ac-P-Cha-G˜LL-Ahxh-H | 48 | 100 |
| 254 | Ac-P-Cha-G˜Hphe-Cit-L-Ahxh-H | 70 | 100 |
| 255 | Ac-P-Cit-G˜Hphe-Cha-A-Ahxh-H | 15 | 100 |
| 256 | Ac-PL-NLys(boc)˜LL-NHNH-H | 49 | 100 |
| 257 | Ac-PLG˜Hphe-R(Pmc)-Ahxh-H | 53 | 100 |
| 258 | Ac-PLG˜Ahp-O(Boc)L-Ahxh-H | 48 | 100 |
| 259 | Ac-PLG˜LY(t-Bu)-Ahxh-H | 96 | 100 |
| 260 | Ac-PLG˜Hphe-O(Boc)L-Ahxh-H | 14 | 100 |
| 261 | Ac-PLG˜L-Pya-L-Ahxh-H | 58 | 100 |
| 262 | Ac-PLG˜LYS(t-Bu)-Ahxh-H | 45 | 100 |
| 263 | Ac-PLG˜LY(t-Bu)V-Ahxh-H | 65 | 100 |
| 264 | Ac-PL-NLys(Boc)˜Hphe-L-Ahxh-H | 23 | 100 |
| 265 | Ac-PL-NLys(Boc)˜Hphe-R(Pmc)L-Ahxh-H | 36 | 100 |
| 266 | Ac-PL-NLys(Boc)-LL-dLeu-Ahxh-H | 66 | 97 |
| 267 | Ac-PL-NLys(Boc)˜S(OBn)-LL-Ahxh-H | 20 | 95 |
| 268 | Ac-PL-NLys(Boc)˜LL-Ambh-H | 5 | 100 |
| 269 | Ac-PL-NLys(Boc)˜Ahp-Y(t-Bu)L-Ahxh-H | 30 | 95 |

TABLE 10
Mass Spectrometry Data for Examples 234-269

| | Low Resolution MS | | | |
|---|---|---|---|---|
| | Ion 1/Identity/ | Ion 2/Identity/ | High Resolution MS | |
| Ex. # | Intensity | Intensity | Calcd for CxHxNxOxSx [M + H]: | Found |
| 234 | 844.5/M + H/60% | 422.9/M + 2H/100% | | |
| 235 | 681.5/M + H/100% | | | |
| 236 | 944.5/M + H/100% | 416.9/40% | | |
| 237 | 925.5/M + H/100% | 435.4/30% | | |
| 238 | 1409.7/M + H/30% | 705.5/M + 2H/100% | | |
| 239 | 957.6/M + H/100% | 429.5/M + 2H/90% | | |
| 240 | 858.6/M + H/100% | 429.9/M + 2H/10% | | |
| 241 | 721.5/M + H/100% | | | |
| 242 | 860.4/M + H/100% | 430.8/M + 2H/35% | C40H65N11O10 [M + H]: 860.4989 | 860.4988 |
| 243 | 844.4/M + H/100% | | | |
| 244 | 796.5/M + H/100% | | | |
| 245 | 681.5/M + H/100% | | | |
| 246 | 852.6/M + H/100% | | | |
| 247 | 987.6/M + H/30% | 444.5/M + 2H/100% | | |
| 248 | 1157.7/M + H/100% | 915.6/M-trt + H/25% | C64H88N10O10 [M + Na]: 1179.658 | 1179.658 |
| 249 | 735.6/M + H/100% | 368.4/M + 2H/15% | | |
| 250 | 912.6/M + H/100% | 428.9/M + 2H/25% | C47H77N9O9 [M + H]: 912.5917 | 912.5913 |
| 251 | 695.5/M + H/100% | 348.4/M + 2H/5% | C34H62N8O7 [M + H]: 695.4814 | 695.4821 |
| 252 | 887.5/M+/15% | 444.3/M + 2H/100% | | |
| 253 | 721.5/M + H/100% | | C36H64N8O7 [M + H]: 721.4971 | 721.4963 |
| 254 | 926.5/M + H/100% | 463.9/M2H/40% | C46H75N11O9 [M + H]: 926.5822 | 926.5858 |
| 255 | 884.5/M + H/100% | | | |
| 256 | 739.5/M + H/100% | | | |
| 257 | 1038.6/M + H/100% | 519.9/M + 2H/80% | | |
| 258 | 907.7/M + H/100% | 404.4/60% | | |
| 259 | 787.5/M + H/100% | 366.3/10% | | |
| 260 | 943.5/M + H/100% | 422.4/M + 2H/15% | C47H78N10O10 [M + H]: 943.5975 | 943.5971 |
| 261 | 829.7/M + H/25% | 415.4/M + 2H/100% | C41H68N10O8 [M + 2H]: 415.2684 | 415.2684 |
| 262 | 930.6/M + H/100% | | C47H79N9O10 [M + H]: 930.6023 | 930.6008 |
| 263 | 886.7/M + H/100% | | C45H75N9O9 [M + H]: 886.5761 | 886.575 |
| 264 | 900.5/M + H/100% | 400.9/17% | C46H77N9O9 [M + H]: 900.5917 | 900.5913 |
| 265 | 1322.7/M + H/100% | 662.0/M + 2H/100% | C66H107N13O13S [M + H]: 1322.7905 | 1322.788 |
| 266 | 965.5/M + H/100% | 433.4/M + H/20% | | |
| 267 | 1029.6/M + H/100% | | C52H88N10O11 [M + H]: 1029.6707 | 1029.671 |
| 268 | 872.5/M + H/100% | | | |
| 269 | 1083.6/M + H/100% | 492.5/65% | | |

### EXAMPLES 270-305

### Synthesis of [¹⁴C]Acetyl-MMP Substrate-Hydrazide Conjugates

Part A—Preparation of [¹⁴C]Sodium Acetate Solutions

Two hundred fifty millicuries of [1-¹⁴C]Acetic acid, sodium salt, solid 50-60 mCi/mmole specific activity was obtained from General Electric Health Care (formerly Amersham Biosciences). The [1-¹⁴C]Acetic acid, sodium salt, solid was dissolved in 25.0 mL of anhydrous acetonitrile to prepare a ¹⁴C sodium acetate stock solution. The solution was vortex mixed for ten minutes. Aliquots were removed for radioassay using liquid scintillation counter (LSC) method. The LSC radioassays were conducted by distributing a measured aliquot of the radioactive solution into a 10 mL glass scintillation vial containing 5 mL of Perkin Elmer Ultima Gold™ scintillation fluid and subsequently measuring the radioactive content using either a Packard model 2500TR or 1600TR LSC. Subsequent ten fold dilutions were made from this stock solution to prepare solutions used in the reactions. Prior to each reaction LSC radioassays were conducted on the reagent solution.

Part B—Conjugation of [¹⁴C]Sodium Acetate to MMP Substrate-Hydrazides

Acetylation of the MMP substrates and enamides were performed by the coupling of amine with the ¹⁴C containing sodium acetate in a solution of O-Benzotriazol-1yl-N,N,N′,N′-teramethyluronium hexafluorophosphate (HBTU), N,N disopropylethylamine (diisopropylethylamine) in dimethyl formamide (N,N-dimethylformamide) at ambient temperature (25° C.). The contents were combined in a 5 mL conical interior Wheaton™ thick walled reaction vial, and allowed to react for 1 h.

Part C—Deprotection and Final Purification

Side chain protecting groups were removed using one of the following methods.
Method A: 50:50 trifluoroacetic acid:dichloromethane at RT for 15 min.
Method B: 95:2.5:2.5 trifluoroacetic acid:Anisole:water at RT for 45 min.
Method C: 2 mol % Pd(OAc)₂, 4 mol % TPPTS, Et₂NH in 2:1 acetonitrile:water.

The crude reaction mixtures were analyzed using a HPLC interfaced with a mass spectrometer (LC/MS) on a Zorbax Eclipse XDB C-18 (4.6 mm×250 mm) column. The solutions were concentrated under reduced pressure and the crude product was purified by HPLC on a Phenomenex™ LUNA C18(2) column (10 mm×250 mm) using a 4.2%/min gradient of 0 to 63% acetonitrile containing 0.1% trifluoroacetic acid at a flow rate of 5 mL/min. Product fractions were concentrated under reduced pressure and analyzed by LC/MS on a Zorbex Eclipse XDB C-18 column (4.6 mm×250 mm) using a 4.2%/min gradient of 0 to 63% acetonitrile containing 0.1% formic acid. A radioactivity detector was used to confirm RCP. Purity data are shown in Table 11.

TABLE 11
Analytical and Yield Data for [¹⁴C]Acetyl-MMP
Substrate-Hydrazide Conjugates

| | Amine | | | |
|---|---|---|---|---|
| | Precursor | Deprotection | | Retention |
| Example # | Example # | Method | % RCP | Time (min) |
| 270 | 234 | — | 80 | 10.1 |
| 271 | 235 | — | 98 | 10.2 |
| 272 | 236 | B | 96 | 10.7 |
| 273 | 237 | B | 100 | 11.5 |
| 274 | 238 | B | 100 | 10.3 |
| 275 | 239 | A | 100 | 12.6 |
| 276 | 240 | — | 100 | 16.7 |
| 277 | 241 | — | 100 | 16.2 |
| 278 | 242 | — | 100 | 12.9 |
| 279 | 243 | B | 100 | 10.7 |
| 280 | 244 | — | 83 | 12.6 |
| 281 | 245 | A | 95 | 9.4 |
| 282 | 246 | A | 100 | 11.6 |
| 283 | 247 | A | 100 | 11.3 |
| 284 | 248 | B | 95 | 12.5 |
| 285 | 249 | — | 97 | 15.2 |
| 286 | 250 | B | 90 | 13.7 |
| 287 | 251 | — | 90 | 11.5 |
| 288 | 252 | — | 99 | 10.4 |
| 289 | 253 | — | 98 | 14.5 |
| 290 | 254 | — | 98 | 13.6 |
| 291 | 255 | — | 100 | 12.4 |
| 292 | 256 | A | 100 | 11.1 |
| 293 | 257 | B | 100 | 11.8 |
| 294 | 258 | A | 100 | 12.1 |
| 295 | 259 | B | 100 | 11.8 |
| 296 | 260 | A | 90 | 12.6 |
| 297 | 261 | — | 100 | 11.1 |
| 298 | 262 | B | 100 | 12.3 |
| 299 | 263 | B | 100 | 13.3 |
| 300 | 264 | A | 100 | 12.5 |
| 301 | 265 | B | 94 | 11.0 |
| 302 | 266 | A | 92 | 12.6 |
| 303 | 267 | A | 100 | 13.5 |
| 304 | 268 | A | 100 | 12.0 |
| 305 | 269 | B | 100 | 12.2 |

### EXAMPLES 270-305

### MMP Activity, Protein Binding, In Vitro Stability, and In Vivo Stability

¹⁴C-Labeled hydrazide conjugates 270-305 were evaluated as substrates for MMP-9 using the procedures described in Example 45. Protein binding was measured using the procedures described in Example 231. In vitro and in vivo stability were determined according to the procedures of Examples 232 and 233, respectively. These data are collected together in Table 12.

TABLE 12

MMP-2 and MMP-9 Activity, Protein Binding, and Stability of Examples 270-305

Stability,
Mouse,
15 Min

| | | | | | |
|---|---|---|---|---|---|
| Ex | MMP2 | MMP9 | Protein Binding, | In | In Vivo |

| | | | | | | |
|---|---|---|---|---|---|---|
| # | Sequence | Kcat/Km | Kcat/Km | % (H/R/M/S)¹ | Vitro | Blood |

| | | | | | | |
|---|---|---|---|---|---|---|
| 270 | Ac-P-Cit-G˜Hphe-LA-Ahxh-Ac[C14] | >750,000 | 651,807 | −12/−1.2/2.9/5 | 31 | 0 |
| 271 | Ac-PLG˜LL-Ahxh-Ac[C14] | 147,567 | 134,448 | 1.7/1.4/1.7/3.5 | 94 | 0 |
| 272 | Ac-PLG˜LYT-Ahxh-Ac[C14] | 412,626 | >500,000 | 3.8/14/4.1/0.5 | 92 | 0 |
| 273 | Ac-PLG˜LWA-Ahxh-Ac[C14] | 712,271 | >750,000 | 2.7/na/na/1.4 | 36 | 62 |
| 274 | Ac-POG˜Hphe-LTR-Ahxh-Ac[C14] | >500,000 | 246,044 | 3.4/1.4/−0.4/3.4 | 23 | 7 |
| 275 | Ac-PLG˜Hphe-KL-Ahxh-Ac[C14] | 102,664 | 119813 | −2.5/−1.5/−0.7/−0.1 | 100 | 100 |
| 276 | Ac-PLG˜S(OBn)-LL-Ahxh-Ac[C14] | 473,393 | >500,000 | 23/28/61/13 | 88 | 0 |
| 277 | Ac-PLG˜L-Cha-Ahxh-Ac[C14] | 359,036 | 475,032 | | 78 | 0 |
| 278 | Ac-P-Cit-G˜S(OBn)-LA-Ahxh-Ac[C14] | >500,000 | >500,000 | | 29 | 9 |
| 279 | Ac-NGlu-PLG˜Hphe-YL-Ahxh-Ac[C14] | 351,477 | 213,148 | 18/25/30/9.5 | 84 | 0 |
| 280 | Ac-PLG˜Cit-LA-Ahxh-Ac[C14] | 16,271 | 34,046 | −1.3/−1.9/−1.1/0.1 | 93 | 15 |
| 281 | Ac-P-NLeu-G˜LL-Ahxh-Ac[C14] | 0 | 0 | −6.3/4.6/−0.4/2.6 | 100 | 27 |
| 282 | Ac-PL-NLys˜LL-Ahxh-Ac[C14] | 180,012 | 194,529 | −0.5/na/na/−0.3 | 99 | 35 |
| 283 | Ac-P-Cit-G˜Hphe-OL-Ahxh-Ac[C14] | 42,516 | 28,556 | 5.4/7.7/9.8/2.5 | 96 | 72 |
| 284 | Ac-PLG˜LYQ-Ahxh-Ac[C14] | 439,260 | 658,127 | 1.3/6.4/7.4/0.2 | 64 | 0 |
| 285 | Ac-Oic-LG˜LL-Ahxh-Ac[C14] | 0 | 0 | 8.5/29/−9.1/9.1 | 60 | 0 |
| 286 | Ac-PLG˜Ahp-YL-Ahxh-Ac[C14] | 38,691 | 60,351 | 18/14/12/12 | 80 | 28 |
| 287 | Ac-PL-Sar˜LL-Ahxh-Ac[C14] | 110,543 | 142,613 | 0.7/4.5/3.8/3.6 | 89 | 4 |
| 288 | Ac-PLG˜Pabu-Cit-L-Ahxh-Ac[C14] | 0 | 0 | −1.4/1.9/−2.3/10.3 | 93 | 93 |
| 289 | Ac-P-Cha-G˜LL-Ahxh-Ac[C14] | 24,657 | 180,419 | 19/21/46/4.5 | 4 | 2 |
| 290 | Ac-P-Cha-G˜Hphe-Cit-L-Ahxh-Ac[C14] | 22,646 | >500,000 | 33/16/12/15 | 93 | 20 |
| 291 | Ac-P-Cit-G˜Hphe-Cha-A-Ahxh-Ac[C14] | >500,000 | >500,000 | 19/34/41/3.8 | 96 | 8 |
| 292 | Ac-PL-NLys-LL-NHNH-Ac[C14] | 133,549 | 138,091 | 3.1/4.0/2.6/1.6 | 100 | 49 |
| 293 | Ac-PLG˜Hphe-R-Ahxh-Ac[C14] | >500,000 | >500,000 | 7.6/19/10/2.1 | 91 | 66 |
| 294 | Ac-PLG˜Ahp-OL-Ahxh-Ac[C14] | 4,967 | 9,343 | 5.0/5.2/8.0/4.9 | 81 | 66 |
| 295 | Ac-PLG˜LY-Ahxh-Ac[C14] | 282,901 | 336,278 | 8.0/9.1/7.4/−0.1 | 95 | 0 |
| 296 | Ac-PLG-Hphe-OL-Ahxh-Ac[C14] | 53,814 | 77,493 | 1.1/13/8.4/7.1 | 81 | 73 |
| 297 | Ac-PLG˜L-Pya-L-Ahxh-Ac[C14] | 92,966 | 161,687 | −1.8/1.4/−5.9/2.6 | 52 | 0 |
| 298 | Ac-PLG˜LYS-Ahxh-Ac[C14] | >500,000 | >500,000 | 3.3/11/8.4/1.9 | 87 | 0 |
| 299 | Ac-PLG-LYV-Ahxh-Ac[C14] | 396,120 | >500,000 | 3.0/13/12/9.9 | | |
| 300 | Ac-PL-NLys˜Hphe-L-Ahxh-Ac[C14] | 464,638 | 288,845 | 5.8/3.8/7.3/5.5 | 94 | 71 |
| 301 | Ac-PL-NLys˜Hphe-RL-Ahxh-Ac[C14] | 102,471 | 114,096 | 3.0/−2.1/−2.6/4.4 | 72 | 7 |
| 302 | Ac-PL-NLys-LL-dLeu-Ahxh-Ac[C14] | 0 | 0 | 4.8/3.6/1.9/6.1 | 91 | 82 |
| 303 | Ac-PL-NLys˜S(OBn)-LL-Ahxh-Ac[C14] | 165,283 | 271,181 | 2.4/0.9/−10/6.1 | 76 | 16 |
| 304 | Ac-PL-NLys˜LL-Ambh-Ac[C14] | 68,635 | 88,016 | 7.6/6.9/16/5.7 | | |
| 305 | Ac-PL-NLys˜Ahp-YL-Ahxh-Ac[C14] | 0 | 0 | −4.7/−1.4/−5.2/5.9 | 0 | 0 |

¹H/R/M/S = Human/Rat/Mouse/Saline Control

### EXAMPLES 306-331

### Synthesis and Characterization of ¹²C Surrogates of Examples 234-269

The procedures of Examples 61 and 62 were used to prepare ¹²C surrogates for selected compounds from Examples 234-269. Yield and purity data are shown in Table 13, and mass spectrometry data are shown in Table 14.

TABLE 13

Yield and Purity data for Examples 306-331

| | | |
|---|---|---|
| Ex. | Yield, | Purity, |

| | | | |
|---|---|---|---|
| # | Sequence | % | % (HPLC) |

| | | | |
|---|---|---|---|
| 306 | Ac-P-Cit-G˜Hphe-LA-Ahxh-Ac | 81 | 98 |
| 307 | Ac-PLG˜LL-Ahxh-Ac | 98 | 100 |
| 308 | Ac-PLG˜LYT-Ahxh-Ac | 67 | 100 |
| 309 | Ac-PLG˜LWA-Ahxh-Ac | 40 | 100 |
| 310 | Ac-POG˜Hphe-LTR-Ahxh-Ac | 99 | 100 |
| 311 | Ac-PLG˜Hphe-KL-Ahxh-Ac | 47 | 99 |
| 312 | Ac-PLG˜S(OBn)-LL-Ahxh-Ac | 74 | 100 |
| 313 | Ac-NGlu-PLG˜Hphe-YL-Ahxh-Ac | 100 | 100 |
| 314 | Ac-PLG˜Cit-LA-Ahxh-Ac | 86 | 100 |
| 315 | Ac-P-NLeu-G˜LL-Ahxh-Ac | 91 | 100 |
| 316 | Ac-PL-NLys˜LL-Ahxh-Ac | 73 | 100 |
| 317 | Ac-P-Cit-G˜Hphe-OL-Ahxh-Ac | 60 | 100 |
| 318 | Ac-PLG˜LYQ-Ahxh-Ac | 95 | 100 |
| 319 | Ac-Oic-LG˜LL-Ahxh-Ac | 78 | 100 |
| 320 | Ac-PLG˜Ahp-YL-Ahxh-Ac | 94 | 100 |
| 321 | Ac-PL-Sar˜LL-Ahxh-Ac | 60 | 100 |
| 322 | Ac-PLG˜Pabu-Cit-L-Ahxh-Ac | 30 | 100 |
| 323 | Ac-P-Cha-G˜LL-Ahxh-Ac | 94 | 100 |
| 324 | Ac-P-Cha-G˜Hphe-Cit-L-Ahxh-Ac | 93 | 100 |
| 325 | Ac-P-Cit-G˜Hphe-Cha-A-Ahxh-Ac | 95 | 100 |
| 326 | Ac-PL-NLys˜LL-NHNH-Ac | 103 | 100 |
| 327 | Ac-PLG˜Hphe-R-Ahxh-Ac | 59 | 100 |
| 328 | Ac-PLG˜LY-Ahxh-Ac | 64 | 100 |
| 329 | Ac-PLG˜L-Pya-L-Ahxh-Ac | 68 | 100 |
| 330 | Ac-PLG˜LYS-Ahxh-Ac | 50 | 100 |
| 331 | Ac-PLG˜LYV-Ahxh-Ac | 43 | 100 |

TABLE 14
Mass Spectrometry Data for Examples 306-331

| | Low Resolution MS | | | |
|---|---|---|---|---|
| | Ion 1/Identity/ | Ion 2/Identity/ | High Resolution MS | |
| Ex. # | Intensity | Intensity | Calcd for CxHxNxOxSx [M + H]: | Found |
| 306 | 886.5/M + H/60% | 443.9/M + 2H/100% | C42H67N11O10 [M + H]: 886.5145 | 886.515 |
| 307 | 723.5/M + H/100% | 362.3/M + 2H/30% | C35H62N8O8 [M + H]: 723.4763 | 723.4771 |
| 308 | 874.5/M + H/100% | 437.8/M + 2H/60% | C42H67N9O10 [M + H]: 874.5033 | 874.5048 |
| 309 | 867.5/M + H/100% | 434.2/M + 2H/40% | C43H66N10O9 [M + H]: 867.5087 | 867.5071 |
| 310 | 1029.6/M + H/20% | 515.5/M + 2H/100% | C48H80N14O11 [M + 2H]: 515.3130 | 515.3143 |
| 311 | 899.5/M + H/100% | 450.4/M + 2H/98% | | |
| 312 | 900.5/M + H/100% | 450.9/M + 2H/55% | | |
| 313 | 1063.5/M + H/100% | 532.3/M + 2H/30% | C53H78N10O13 [M + H]: 1063.582 | 1063.583 |
| 314 | 838.5/M + H/100% | 419.9/M + 2H/75% | C38H67N11O10 [M + H]: 838.5151 | 838.5153 |
| 315 | 723.5/M + H/100% | | C35H62N8O8 [M + H]: 723.4763 | 723.4773 |
| 316 | 794.5/M + H/100% | 397.8/M + 2H/80% | C39H71N9O8 [M + H]: 794.5498 | 794.5491 |
| 317 | 929.5/M + H/55% | 465.4/M + 2H/100% | | |
| 318 | 901.5/M + H/100% | 451.4/M + 2H/95% | C43H68N10O11 [M + H]: 901.5142 | 901.5132 |
| 319 | 776.6/M + H/100% | | C39H68N8O8 [M + H]: 777.5233 | 777.5233 |
| 320 | 898.5/M + H/90% | 449.4/M + 2H/100% | | |
| 321 | 737.5/M + H/100% | | C36H64N8O8 [M + H]: 737.4920 | 737.491 |
| 322 | 929.5/M + H/20% | 465.4/M + 2H/100% | C44H73N12O10+ [2M + H]: 465.2820 | 465.2828 |
| 323 | 763.5/M + H/100% | | C38H66N8O8 [M + H]: 763.5076 | 763.5084 |
| 324 | 900.6/M + H/100% | 450.9/M + 2H/75% | C43H69N11O10 [M + H]: 900.5301 | 900.5317 |
| 325 | 924.6/M + H/100% | | C45H71N11O10 [M + H]: 926.5458 | 926.5453 |
| 326 | 681.5/M + H/100% | | C33H60N8O7 [M + H]: 681.4658 | 681.4657 |
| 327 | 814.5/M + H/63% | 407.9/M + 2H/100% | | |
| 328 | 773.4/M + H/100% | 387.4/M + 2H/42% | | |
| 329 | 871.5/M + H/100% | 436.4/M + 2H/87% | | |
| 330 | 860.4/M + H/100% | 430.8/M + 2H/48% | | |
| 331 | 872.5/M + H/100% | 436.9/M + 2H/63% | | |

### EXAMPLES 332-344

### Synthesis and APN Activity of Enamides

The procedures of Examples 63 and 64 were used to prepare these additional enamides. Structures of the enamides, yields for the coupling reaction and mass spectronetry data are shown in Table 15. The ability of aminopeptidase-N (APN) to remove the terminal amino acid was determined by using the procedure described in Example 46. Hydrolysis rates are shown in Table 16.

TABLE 15
Yield and Physical Data of Selected Enamides

| | | Coup- | | | | |
|---|---|---|---|---|---|---|
| | | ling | Low Resolution MS | | High Resolution MS | |
| Ex # | Structure | Yield | ion (intensity, identity) | | Calcd. | Found |
| 332 | 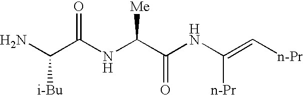 | 40% | 623.5 23, 2M + H 241.4 | 312.3 (100, M + H) | 312.2651 | |
| 333 | 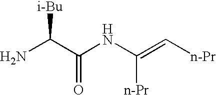 | 32% | (100, M + H) 279.3 | | 241.2281 | |
| 334 | 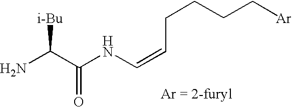 | 20% | (100, M + H) 279.3 | | 279.2073 | |
| 335 | 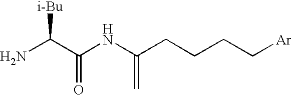 | 8% | (100, M + H) 279.4 | | 279.2073 | |
| 336 | 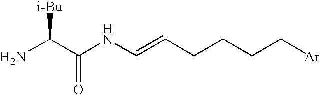 | 85% | (100, M + H) 557.4 | | 279.2073 | 279.2059 |
| 337 | 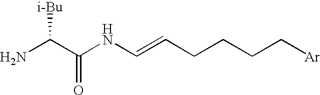 | 56% | (10, 2M + H) 237.3 | (100, M + H) | 279.2073 | 279.2067 |
| 338 | 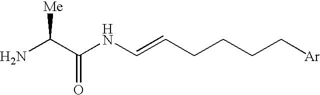 | 18% | (100, M + H) | | 237.1604 | 237.1594 |
| 339 | 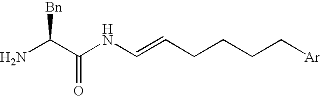 | 69% | (100, M + H) | | 313.1916 | |
| 64 | 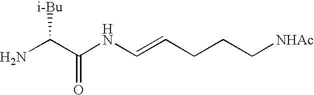 | 88% | (14, 2M + H) | (100, M + H) | 256.2025 | 256.2016 |
| 340 | 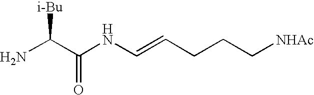 | 44% | | | 214.1556 | |
| 341 | 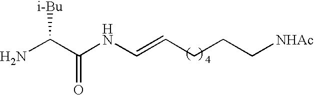 | 79% | 298.4 (100, M + H) | | 298.2495 | |
| 342 | 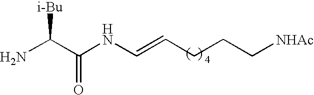 | 87% | 298.4 (100, M + H) | 284.4 (3) | 298.2495 | |
| 343 | 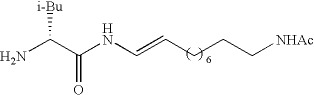 | 93% | 348.5 (9, M + Na) | 326.4 (100, M + H) | 326.2808 | |
| 63 | 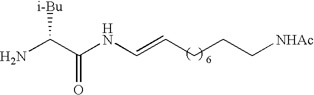 | 84% | 326.4 (100, M + H) | 326.2808 | | |
| 344 | 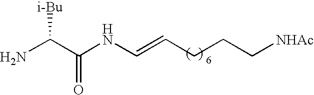 | 55% | 284.2 (100, M + H) | 213.3 (25) | 284.2339 | |

TABLE 16
Hydrolysis of N-Terminal Residue by APN of Selected Enamides

| | Rate (mmol substrate · |
|---|---|
| Example # | min⁻¹ · U enzym⁻¹)^{a} |
| 332 | 1.89 (0.802) |
| 333 | 0.264 (0.209) |
| 334 | 0.095 (0.070) |
| 335 | 0.137 (0.153)^{b} |
| 336 | 0.565 (0.420)^{c} |
| 337 | 0.000 (0.000) |
| 338 | 1.377 (0775)^{d} |
| 339 | 0.345 (0.325) |
| 342 | 0.286 (0.269) |
| 63 | 0.202 (0.167) |
| 344 | 1.183 (0.753) |

^{a)}The APN assay is performed at three enzyme concentrations: 0, 6.5 × 10⁴ and 15.0 × 10³ U. The rate data are given at the 6.5 × 10⁴ U concentration. The value obtained at 15.0 × 10³ U is listed in parenthesis. Enzymatic activity ceased upon dilution with 30% aqueous AcOH. All values have n = 2.
^{b)}Enzyme was denatured with acetonitrile due to the acid-sensitivity of the substrate.
^{c)}Average of three runs.
^{d)}Average of two runs.

### EXAMPLES 345-350

### Synthesis of [¹⁴C]Acetyl-Enamides

The procedures of Examples 270-305 were used to prepare radiolabeled enamides. Purity data are shown in Table 17, and protein binding and stability data are shown in Table 18.

TABLE 17
Analytical and Yield Data for [¹⁴C]Acetyl-Enamides

| | Amine | | | |
|---|---|---|---|---|
| | Precursor | Deprotection | | Retention |
| Example # | Example # | Method | % RCP | Time (min) |
| 345 | 64 | C | 95 | 9.0 |
| 346 | 341 | C | 100 | 11.1 |
| 347 | 342 | C | 100 | 11.2 |
| 348 | 343 | C | 99 | 14.0 |
| 349 | 63 | C | 100 | 13.9 |
| 350 | 344 | C | 100 | 9.2 |

TABLE 18
Protein Binding and Stability Data of Selected [C14]Labeled Enamides

| | Protein Binding (subtracted) | | | | Blood Stability | |
|---|---|---|---|---|---|---|
| Ex. # | human | rabbit | mouse | saline | in vitro | in vivo |
| 345 | 0.8 | 16.8 | 5.3 | 0.2 | 100 | 63 |
| 346 | 26.0 | 68.8 | 52.5 | −1.2 | 97 | 67 |
| 347 | 37.5 | 42.2 | 43.4 | −0.8 | 100 | 19 |
| 348 | 78.6 | 85.2 | 80.9 | −7.8 | 100 | 42 |
| 349 | 81.8 | 74.2 | 77.0 | −8.0 | na | na |
| 350 | 71.8 | 67.6 | 75.1 | −0.9 | 8 | 0 |

### EXAMPLE 351

### Synthesis of (2S)-N-[(N-{(1S)-1-[N-((1S)-1-{N-[7-([¹⁴C]Acetylamino)-2-oxoheptyl]carbamoyl}-3-methylbutyl)carbamoyl]-3-methylbutyl}carbamoyl)methyl]-2-[((2S)-1-acetylpyrrolidin-2-yl)carbonylamino]-N-(4-aminobutyl)-4-methylpentanamide, Trifluoroacetic Acid Salt

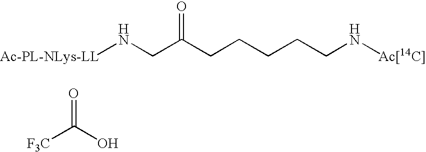

### Part A—Preparation of N-(7-Bromo-6-oxoheptyl)(fluoren-9-ylmethoxy)carboxamide

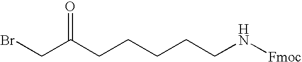

A solution of 6-[(fluoren-9-ylmethoxy)carbonylamino]hexanoic acid and N-methylmorpholine in anhydrous THF is cooled to 0° C. and treated with isobutyl chloroformate. The mixture is stirred for 30 min under nitrogen and filtered through a Celite bed. The filtrate is added to freshly prepared ethereal-diazomethane at 0° C. over 10 min. The resulting solution is stirred for 3 h and a slow stream of nitrogen is bubbled through the solution to remove excess diazomethane. The solution is concentrated on a rotary evaporator at a temperature below 35° C. The residue is dissolved in ether, cooled to −20° C. and treated with 48% aqueous HBr. The solution is stirred for 30 min at −20° C., diluted with ether, and washed with water (3×). The organic layer is dried (Na₂SO₄) and concentrated. The residue is purified by flash chromatography over silica gel to give the title compound.

### Part B—Preparation of (Fluoren-9-ylmethoxy)-N-{6-oxo-7-[N-(oxomethyl)carbonylamino]heptyl}carboxamide

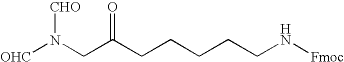

A mixture of the product of Part A and sodium diformylamine in anhydrous acetonitrile is stirred at ambient temperatures under nitrogen until TLC indicates the disappearance of starting material. The mixture is filtered to remove precipitated NaBr and the filtrate is concentrated. The residue is purified by flash chromatography over silica gel to give the title compound.

### Part C—Preparation of N-(7-Amino-6-oxoheptyl)(fluoren-9-ylmethoxy)carboxamide, Trifluoroacetic Acid Salt

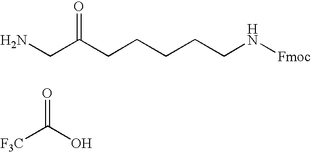

A mixture of the product of Part B and 6 N HCl is heated to reflux for 30 min. The solution is concentrated to dryness and the crude product is purified by HPLC on a C18 column using a water:acetonitrile:0.1% trifluoroacetic acid gradient. The product fraction is lyophilized to give the title compound.

### Part D—Preparation of (2S)-N-{[N-((1S)-1-{N-[(1S)-1-(N-{7-[(fluoren-9-ylmethoxy)carbonylamino]-2-oxoheptyl}carbamoyl)-3-methylbutyl]carbamoyl}-3-methylbutyl)carbamoyl]methyl}-2-[((2S)-1-acetylpyrrolidin-2-yl)carbonylamino]-N-{4-[(tert-butoxy)carbonylamino]butyl}-4-methylpentanamide

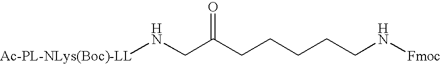

The product of Part C is dissolved in anhydrous N,N-dimethylformamide along with the product of Example 61 Part B, and treated with HBTU, and diisopropylethylamine. The solution is stirred at ambient temperatures under nitrogen for 4 h and concentrated under vacuum. The residue is purified by HPLC on a C18 column using a water:acetonitrile:0.1% trifluoroacetic acid gradient. The product fraction is lyophilized to give the title compound.

### Part E—Preparation of (2S)-N-({N-[(1S)-1-(N-{(1S)-1-[N-(7-Amino-2-oxoheptyl)carbamoyl]-3-methylbutyl}carbamoyl)-3-methylbutyl]carbamoyl}methyl)-2-[((2S)-1-acetylpyrrolidin-2-yl)carbonylamino]-N-{4-[(tert-butoxy)carbonylamino]butyl}-4-methylpentanamide, Trifluoroacetic Acid Salt

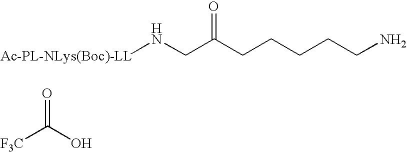

The product of Part D is dissolved in 20% piperidine in N,N-dimethylformamide and stirred at ambient temperatures for 20 min. The solution is concentrated under reduced pressure and dried thoroughly under high vacuum. The crude product is purified by HPLC on a C18 column using a water:acetonitrile:0.1% trifluoroacetic acid gradient. The product fraction is lyophilized to give the title compound.

### Part F—Preparation of (2S)-N-[(N-{(1S)-1-[N-((1S)-1-{N-[7-([¹⁴C]Acetylamino)-2-oxoheptyl]carbamoyl}-3-methylbutyl)carbamoyl]-3-methylbutyl}carbamoyl)methyl]-2-[((2S)-1-acetylpyrrolidin-2-yl)carbonylamino]-N-(4-aminobutyl)-4-methylpentanamide, Trifluoroacetic Acid Salt

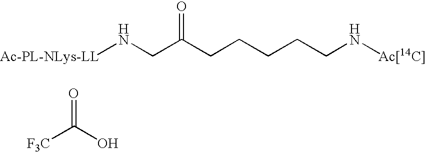

The radiolabeling procedures described in Examples 270-305 are used to prepare the title compound.

General. ¹H NMR spectra were recorded on a Bruker Avance DRX (600 MHz) spectrometer. Chemical shifts are reported in ppm from tetramethylsilane with the residual solvent resonance resulting from incomplete deuteration as the internal standard (CDCl₃: δ 7.25 ppm, C₆D₆: δ7.16 ppm, DMSO-d₆: ε2.50 ppm). Data are reported as follows: chemical shift, integration, multiplicity (s=singlet, d=doublet, t=triplet, q=quartet, quin=quintet, br=broad, m=multiplet), and coupling constants. ¹³C NMR spectra were recorded on a Bruker Avance DRX (150 MHz) with complete proton decoupling. Chemical shifts are reported in ppm from tetramethylsilane with the solvent as the internal reference (CDCl₃: □77.0 ppm, C₆D₆: δ128.4 ppm, DMSO-d₆: δ39.5 ppm). Low-resolution mass spectrometry was performed on an Agilent Technologies 1100 Series LC/MS ESI-MS (positive mode). High-resolution mass spectrometry was performed on a IonSpect FTMS; ESI-MS (positive mode).

Unless otherwise stated, all reactions were conducted in oven- (150° C.) and flame-dried glassware under an inert atmosphere of dry nitrogen. Indicated temperatures refer to those of the reaction bath, while ambient laboratory temperature is noted as 22° C. Anhydrous solvents are obtained for Aldrich.

The following is a description of reagents, which required prior preparation or purification.¹ Oct-7-yn-1-ol was prepared from oct-3-yn-1-ol according to a published procedure.² Both PPh₃ (hexanes) and imidazole (CH₂Cl₂) were purified by recrystallization. N,N′-Dimethylethylenediamine was distilled from solid KOH immediately prior to use. Cuprous iodide was recrystallized from a saturated aqueous solution of sodium iodide. Leucine amides were prepared as the free base in two steps from the corresponding Cbz-protected amino acids: a) EtO₂CCl, Et₃N, NH₄OH; b) H₂, Pd/C. Allyl chloroformate, Et₃N and Et₂NH were distilled from CaH₂ immediately prior to use. (1E)-5-azido-1-iodopent-1-ene was prepared from pent-4-yn-1-ol in an analogous fashion to that described for (1E)-8-azido-1-iodooct-1-ene.³ All other reagents were used as obtained from Aldrich, Fluka or Strem Chemicals.
¹ A general text covering the techniques described herein is available: Armarego, W. L. F.; Perrin, D. D. Purification of Laboratory Chemicals, 4^{th} ed.; Butterworth-Heinemann: Oxford, U. K., 1998.

² Denmark, S. E.; Yang, S.-M. J. Am. Chem. Soc. 2002, 124, 2102.

³ For an alternative preparation, see: Tucker, C. E.; Majid, T. N.; Knochel, P. J. Am. Chem. Soc. 1992, 114, 3983.

### Abbreviations

Abu=2-aminobutyric acid
Ahp=2-amino-6-heptenoic acid
Ahxh=6-aminohexanoylhydrazide
Aib=2-aminoisobutyric acid
Ambh=4-(aminomethyl)benzoylhydrazide
Cha=cyclohexylalanine
Chg=cyclohexylglycine
Dab=2,4-diaminobutyric acid
Hcit=homocitrulline
Hpro=homoproline
Hse=homoserine
Igl=indanylglycine
Inp=Isonipicotic acid
Oic=octahydroindolyl-2-carboxylic acid
Pabu=2-amino-4-(1′-pyridinium)butanoate
Piv=pivaloyl
Pra=propargylglycine
Pya=3-(4′-pyridyl)alanine
Smc=S-methylcysteine
Suc=succinoyl
Tic=1,2,3,4-tetrahydroisoquinoline-3-carboxylic acid
   Standard Amino Acids Represented by Their Single Letter Abbreviation
Ahx=6-aminohexanoic acid
Amb=4-aminomethylbenzoic acid
APMA=amino phenyl mercuric acetate
BAIB=[bis(acetoxy)iodo]benzene
Cit=citrulline
Csa=cysteic acid
DIC=diisopropylcarbodiimide
EEDQ=2-ethoxy-1-ethoxycarbonyl-1,2-dihydroquinoline
GM6001=MMP inhibitor Ilomastat
Hphe=homophenylalanine
Hynic=6-hydrazinonicotinic acid
MPeg3=2-[2-(-Methoxyethoxy)ethoxy]acetic acid
NGlu=the peptoid monomer of glutamic acid
NLys=the peptoid monomer of lysine
PABA=para-aminobenzyl alcohol
TBAF=tetrabutylammonium fluoride
TCN buffer=50 MM Tris-HCl/pH 7.5, 10 mM CaCl₂, 150 mM NaCl
TEA=triethylamine
TEMPO=2,2,6,6-tetramethyl-1-piperidinyloxy, free radical
Tse=trimethylsilylethyl
WSC=1-ethyl-3-(3′-dimethylaminopropyl)carbodiimide
   General

Solid phase peptide synthesis was performed on an Advanced Chemtech Model ACT90 peptide synthesizer.

Chiral amino acid analysis was performed as described in Gerhardt, J.; Nicholson, G. J. Editor(s): Hodges, Robert S.; Smith, John A., Proc. Am. Pept. Symp., 13th (1994), 241-3 with the following slight modification. The N-trifluoroacetyl amino acid methyl esters were separated on a Chirasil-Val (0.25 mm×25 m) capillary column using EI-SIM-mass spectroscopy for detection. The sample was injected at a column temperature of 50° C. and programmed to 200° C. at 4° C./min.

1. A compound, comprising:
a. at least one targeting moiety;
b. an optional chelator;
c. a masked trapping moiety; and
d. an optional linking group;
or a pharmaceutically-acceptable derivative thereof;
wherein said targeting moiety is a matrix metalloproteinase substrate;
wherein said chelator is capable of conjugating to a diagnostic component;
wherein said masked trapping moiety is capable of being unmasked to form an unmasked trapping moiety;
wherein said unmasked trapping moiety is capable of being immobilized at a site of interest in a patient;
wherein, in use, said immobilization of said compound is accomplished through an interaction between said unmasked trapping moiety and a substance associated with a pathological disorder associated with matrix metalloproteinase activity at said site of interest in said patient;
provided that said interaction is non-receptor mediated; and
provided that, in use, when said substance is a protein, said interaction is a covalent bond.

2. A compound, comprising:
a. at least one targeting moiety;
b. an optional chelator;
c. a masked trapping moiety; and
d. an optional linking group;
or a pharmaceutically-acceptable derivative thereof;
wherein said targeting moiety is a matrix metalloproteinase substrate;
wherein said chelator is capable of conjugating to a diagnostic component;
wherein said masked trapping moiety is capable of being unmasked to form an unmasked trapping moiety;
wherein said unmasked trapping moiety is capable of being immobilized at a site of interest in a patient;
wherein, in use, said immobilization of said compound is accomplished through an interaction between said unmasked trapping moiety and a substance associated with a pathological disorder associated with matrix metalloproteinase activity at said site of interest in said patient;
provided that said interaction is non-receptor mediated; and
provided that in use the signal from said diagnostic component is substantially unchanged before and after said unmasked trapping moiety is immobilized.

3. A compound according to claim 1, wherein said pathological disorder is coronary plaque.

4. A compound according to claim 1, wherein said pathological disorder is a cancerous tumor.

5. A compound according to claim 1, wherein said targeting moiety is a substrate of one or more matrix metalloproteinases, wherein said matrix metalloproteinase is selected from the group consisting of MMP-1, MMP-2, MMP-3, MMP-9 and MMP-14.

6. A compound according to claim 1, wherein said matrix metalloproteinase substrate comprises a peptide sequence.

7. A compound according to claim 6, wherein said peptide sequence is derived from collagen, proteoglycan, laminin, fibronectin, gelatin, galectin-3, cartilage link protein, myelin basic protein, kallikrein 14, ladinin 1, endoglin, endothilin receptor, laminin α2 chain, phosphate regulating neutral endopeptidase, ADAM 2, demoglein 3, integrin β5, integrin βv, integrin β6, integrin βx, integrin β9, elastin, perlacan, entactin, vitronectin, tenascin, nidogen, dermatan sulfate, proTNF-α, aggrecan, transin, decorin, tissue factor pathway inhibitor, glycoprotein, NG2 proteoglycan, neurocan, PAI-3, big endothelin-1, brevican/BEHAB, decorin, FGFR-1, IGFBP-3, IL-1β, α₂-macroglobulin, MCP-3, pregnancy zone protein, proMMP-1, proMMP-2, SPARC, Substance P, betaglycan or dentin.

8. A compound according to claim 1, wherein said chelator is a surfactant capable of forming an echogenic substance-filled lipid sphere or microbubble.

9. A compound according to claim 1, wherein said unmasked trapping moiety is capable of forming a covalent bond with a substance associated with said pathological disorder.

10. A compound according to claim 9, wherein said unmasked trapping moiety forms a Michael adduct, a hydrazone, a β-sulphone, a Schiff base, a disulfide, a cyclohexene, a cyclohexene derivative, or an oxime with a moiety in said substance.

11. A compound according to claim 9, wherein said unmasked trapping moiety reacts with an endogenous biological molecule in said substance.

12. A compound according to claim 2, wherein said unmasked trapping moiety is a ligand for a soluble enzymatic protein or a soluble nonenzymatic protein associated with said site of interest in a patient.

13. A compound according to claim 12, wherein said ligand is selected from the group consisting of drugs, lipophilic organic molecules, amphiphilic organic molecules, porphyrins, steroids, lipids, hormones, peptides, proteins, oligonucleotides, and antibodies.

14. A method of preparing a 1,2-dicarbonyl compound, the method comprising:
a. reacting the compound of claim 1 with MMP;
b. reacting the product of step a with APN to form an α-aminoketone; and
c. oxidizing said α-aminoketone with serum amine oxidase.

15. A diagnostic agent, comprising:
a. a compound according to claim 1 or a pharmaceutically acceptable derivative thereof, and
b. a diagnostic component,

16. A diagnostic agent, comprising:
a. a compound according to claim 1 or a pharmaceutically acceptable derivative thereof, and
b. a diagnostic component,
wherein said diagnostic component has a signal that is substantially unchanged upon immobilization of said diagnostic agent.

17. A diagnostic agent according to claim 15, wherein said diagnostic component is an echogenic substance, a non-metallic isotope, an optical reporter, a boron neutron absorber, a paramagnetic metal ion, a ferromagnetic metal, a gamma-emitting radioisotope, a positron-emitting radioisotope, or an x-ray absorber.

18. A diagnostic agent according to claim 17, wherein said diagnostic component is a gamma-emitting radioisotope or positron-emitting radioisotope selected from the group consisting of: ^{99m}Tc, ⁹⁵Tc, ¹¹¹n, ⁶²Cu, ⁶⁴Cu, ⁶⁷Ga, and ⁶⁸Ga.

19. A diagnostic agent according to claim 18, wherein said gamma-emitting radioisotope is ^{99m}Tc.

20. A diagnostic agent according to claim 18, wherein said gamma-emitting radioisotope is ¹¹¹In.

21. A diagnostic agent acording to claim 17, wherein said non-metallic isotope is carbon-11, nitrogen-13, fluorine-18, iodine-123, or iodine-125.

22. A diagnostic agent according to claim 15, further comprising a first ancillary ligand and a second ancillary ligand capable of stabilizing said diagnostic component.

23. A composition, comprising:
a. a compound according to claim 1; and
b. a pharmaceutically-acceptable carrier.

24. A composition, comprising:
a. a diagnostic agent according to claim 15; and
b. a pharmaceutically-acceptable carrier.

25. A kit for detecting, imaging, and/or monitoring the presence of matrix metalloproteinase in a patient comprising:
a. a compound according to claim 1;
b. a diagnostic component;
c. a pharmaceutically-acceptable carrier; and
d. instructions for preparing a composition comprising a diagnostic agent for detecting, imaging, and/or monitoring the presence of matrix metalloproteinase in a patient.

26. A kit according to claim 25 wherein said kit further comprises one or more ancillary ligands and a reducing agent.

27. A kit according to claim 26 wherein said ancillary ligands are tricine and 3-[bis(3-sulfophenyl)phosphine]benzenesulfonic acid.

28. A kit according to claim 26, wherein said reducing agent is tin(II).

29. A kit for forming a diagnostic agent, comprising:
a predetermined quantity of a sterile composition according to claim 24;
a predetermined quantity of sterile, pharmaceutically-acceptable stabilizing coligand selected from a dioxygen chelating agent and a functionalized aminocarboxylate;
a predetermined quantity of a sterile, pharmaceutically-acceptable reducing agent; and
optionally, a predetermined quantity of one or more sterile, pharmaceutically acceptable components selected from buffers, lyophilization aids, stabilization aids, solubilization aids and bacteriostats.

30. A method of detecting, imaging, and/or monitoring the presence of matrix metalloproteinase in a patient, comprising the steps of:
a. administering to said patient a diagnostic agent of claim 15; and
b. acquiring an image of a site of concentration of said diagnostic agent in the patient by a diagnostic imaging technique.

31. A method of detecting, imaging, and/or monitoring a pathological disorder associated with matrix metalloproteinase activity in a patient, comprising the steps of:
a. administering to said patient a diagnostic agent of claim 15; and
b. acquiring an image of a site of concentration of said diagnostic agent in the patient by a diagnostic imaging technique.

32. A method according to claim 30, wherein said pathological disorder is cancer, atherosclerosis, rheumatoid arthritis, osteoarthritis, periodontal disease, inflammation, autoimmune disease, organ transplant rejection, ulcerations, scleroderma, epidermolysis bullosa, endometriosis, kidney disease, or bone disease.

33. A method of identifying a patient at high risk for transient ischemic attacks or stroke, comprising the steps of
a. administering to said patient a diagnostic agent according to claim 15; and
b. determining the degree of active atherosclerosis in said patient, comprising the step of acquiring an image of a site of concentration of said diagnostic agent in the patient by a diagnostic imaging technique.

34. A method of identifying a patient at high risk for acute cardiac ischemia, myocardial infarction or cardiac death, comprising the steps of
a. administering to said patient a diagnostic agent according to claim 15; and
b. determining the degree of active atherosclerosis in said patient, comprising the step of acquiring an image of a site of concentration of said diagnostic agent in the patient by a diagnostic imaging technique.

35. A method of detecting, imaging, and/or monitoring congestive heart failure in a patient, comprising the steps of
a. administering to said patient a diagnostic agent of claim 15; and
b. acquiring an image of a site of concentration of said diagnostic agent in the patient by a diagnostic imaging technique.

36. A method of simultaneous imaging of cardiac perfusion and extracellular matrix degradation in a patient, comprising the steps of
a. administering a diagnostic agent according to claim 15, wherein said diagnostic component is a gamma-emitting radioisotope or positron-emitting radioisotope;
b. administering a cardiac perfusion compound, wherein said compound is radiolabeled with a gamma-emitting radioisotope or positron-emitting radioisotope that exhibits a gamma emission energy or positron emission that is spectrally separable from the gamma emission energy or positron emission energy of said diagnostic component conjugated to the targeting moiety in step a; and
c. acquiring, by a diagnostic imaging technique, simultaneous images of the sites of concentration of the spectrally separable gamma-emission energies or positron-emission energies of the compounds administered in steps a and b.
